(19) United States
(12) Patent Application Publication
(10) Pub. No.: US 2025/0197368 A1
(43) Pub. Date: Jun. 19, 2025

(54) 2-OXO-DIHYDROQUINOLINE-3-CARBOXAMIDE DERIVATIVES AS GABA TYPE A RECEPTOR MODULATORS

(71) Applicant: University College Cardiff Consultants Limited, Cardiff (GB)

(72) Inventors: Samuele Maramai, Siena (IT); Alessandro Mazzacani, Sussex (GB); Michael Paradowski, Cardiff (GB); Simon Ward, Cardiff (GB)

(21) Appl. No.: 18/031,103

(22) PCT Filed: Oct. 13, 2021
(86) PCT No.: PCT/GB2021/052644

§ 371 Date: Apr. 10, 2023

(30) Foreign Application Priority Data
Oct. 13, 2020 (GB) 2016245.9

(51) Int. Cl. C07D 401/04; A61K 31/444; A61K 31/4706; A61K 31/4709; A61K 31/496; A61K 31/501; A61K 31/506; C07D 215/54; C07D 401/12; C07D 401/14; C07D 405/04; C07D 405/10; C07D 405/12; C07D 405/14; C07D 413/04; C07D 413/12; C07D 417/12; C07D 471/04; C07D 471/10; C07D 487/10

(52) U.S. Cl. CPC C07D 401/04; A61K 31/444; A61K 31/4706; A61K 31/4709; A61K 31/496; A61K 31/501; A61K 31/506; C07D 215/54; C07D 401/12; C07D 401/14; C07D 405/04; C07D 405/10; C07D 405/12; C07D 405/14; C07D 413/04; C07D 413/12; C07D 417/12; C07D 471/04; C07D 471/10; C07D 487/10

(57) ABSTRACT

The present invention provides compounds of formula (I), as well as pharmaceutically acceptable salts thereof, wherein X¹, L¹, R¹, R², R³, R⁴, Ring A, n and p are as described herein. The compounds of the present invention have affinity for α2- and/or α3-subunit-containing GABA_{A} receptors. The present invention further provides the manufacture of the compounds of formula (I), pharmaceutical compositions comprising the compounds and their use as medicaments for the treatment of diseases and disorders associated with α2- and/or α3-GABA_{A} receptors, including, for example anxiety disorders.

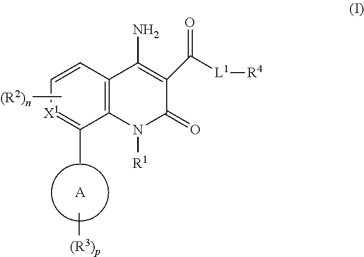

The invention relates to compounds that are γ-aminobutyric acid (GABA) type A receptor (GABA_{A}R) modulators, in particular compounds that selectively activate α2- and/or α3-subunit-containing GABA_{A}Rs over α1-subunit-containing GABA_{A}Rs, their manufacture, pharmaceutical compositions comprising the compounds and their use as medicaments. The compounds of the invention are useful in the treatment of diseases and medical conditions associated with α2- and/or α3-subunit-containing GABA_{A}Rs, including, for example, treatment and/or prevention of anxiety disorders, pain, epilepsy, pruritus and substance abuse conditions.

### BACKGROUND

Gamma-aminobutyric acid (GABA) is the major inhibitory neurotransmitter in the mammalian brain. Three main types of GABA receptors, termed GABA_{A}, GABA_{B} and GABA_{C}, have been identified pharmacologically although the subunit that comprise the GABA_{C} receptors fall within the broader, 19-member GABA_{A} receptor protein family. Compounds targeting GABA_{A} receptors show diverse pharmacology, including anxiolytic, epileptic, hypnotic, anaesthetic, anticonvulsant, antipruritic, analgesic, myorelaxant and cognition and substance abuse modulating effects.

GABA_{A} receptors are heteropentameric ligand-gated chloride channels and their activation modulates the flow of chloride anions across the synaptic junction thereby resulting in a hyperpolarisation of the membrane potential that reduces the probability of the neuron firing an action potential. They are members of the Cys-loop superfamily of ligand-gated ion channels and contain a 13-residue disulphide loop within the large N-terminal domain that is conserved within the Cys-loop superfamily. The GABA_{A} family comprises of 19 members (α1-6, β1-3, γ1-3, δ, ε, θ, π and ρ1-3) with the most abundant forms comprising α, β and γ subunits in a 2:2:1 stoichiometry (Alexander, S. P. et al. The concise guide to pharmacology 2017/18: Ligand-Gated Ion Channels. Br. J. Pharmacol. 2017, 174 (S1), S130-S159). The binding site for the orthosteric ligand (GABA) is found at the interface of the α and β subunits and the binding site for the benzodiazepines (BZ) formed between one of the a subunits and the γ subunit (where the a subunit is α1, α2, α3 or α5 and the γ subunit is γ2).

Compounds that bind at the BZ site may increase the GABA induced chloride currents (positive modulators), decrease the GABA induced chloride current (negative modulators) or be neutral in which case the compounds bind at the allosteric site without modulating the GABA-induced chloride current.

Currently available drugs for modulating GABA_{A} receptor activity include benzodiazepines such as chlordiazepoxide, diazepam and subsequent analogues (lorazepam, triazolam) and other non-benzodiazepines that bind at this same recognition site (e.g., zolpidem, an imidazopyridine derivative). These drugs positively enhance the GABA_{A}R Cl⁻ conductance and are responsible for the efficacy of benzodiazepines in the treatment of a number of disorders including Generalised Anxiety Disorder (GAD), movement disorders, epilepsy, muscle spasms, seizures, psychosis and mood disorders. However, when used as anxiolytics, benzodiazepines commonly also show several undesirable side effects such as sedation, motor incoordination, ataxia, potentiation of alcohol, mental confusion and they induce tolerance and dependence upon chronic administration. All these side effects can interfere with the ability of individuals to perform daily routine, thus benzodiazepines are not optimal for treating chronic disorders. It is worth noting, however, that the side-effects of benzodiazepines in one clinical setting may actually be beneficial in another clinical setting and hence the sedating and myorelaxant effects are useful when benzodiazepines are used as hypnotics or for premedication prior to, for example, endoscopy.

Therefore, there is still an important, unmet need to develop novel non-sedating anxiolytic compounds with reduced side effects associated with the currently available GABA_{A} modulators such as benzodiazepines. Molecular genetic studies with α-subunit knock-out and point-mutation in mice have led to the general concept that α1-GABA_{A}Rs mediate the side effects of benzodiazepines particularly sedation, whereas α2- and/or α3-GABA_{A}Rs are associated with anxiolysis, whilst α5-GABA_{A}Rs play a role in cognitive processes and learning (Rudolph, U.; Möhler, H. GABA-Based Therapeutic Approaches: GABA_{A} Receptor Subtype Functions. Curr. Opin. Pharmacol. 2006, 6 (1), 18-23).

WO2007/073283 and WO2011/021979 disclose certain cinnoline compounds which are stated to be useful as GABA_{A}R modulators.

There is a need for compounds that have a reduced side effect liability (e.g. sedation or hypnosis) compared to benzodiazepines. There is also a need for compounds with a high affinity and/or high functional efficacy for the α2- and/or α3-GABA_{A} receptors whilst having a low efficacy at the other a subunits and in particular the α1-subunit containing GABA_{A} receptors that are known to be associated with sedation.

### BRIEF SUMMARY OF THE DISCLOSURE

In accordance with the present inventions there is provided a compound of the formula (I), or a pharmaceutically acceptable salt thereof:

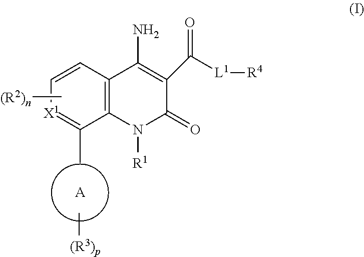

wherein:
X¹ is CH or N;
Ring A is a phenyl or a 5- or 6-membered heteroaryl;
R¹ is selected from: H, C₁₋₄ alkyl, C₁₋₄ haloalkyl, C₃₋₆ cycloalkyl and C₃₋₆ cycloalkyl-C₁₋₄ alkyl-;
each R² is independently selected from: halo, C₁₋₄ alkyl and C₁₋₄ haloalkyl;
each R³ is independently selected from: halo, —CN, —NO₂, ═O, C₁₋₆ alkyl, C₁₋₆ haloalkyl, C₂₋₆ alkenyl, C₂₋₆ alkynyl, —OR⁵, —S(O)ₓR⁵, —NR^{a1}R⁵, —C(O)R⁵, —OC(O)R⁵, —C(O)OR⁵, —NR^{a1}C(O)R⁵, —C(O)NR^{a1}R⁵, —NR^{a1}C(O)OR⁵, —OC(O)NR⁵R^{a1}, —NR^{a1}SO₂R⁵, —SO₂NR^{a1}R⁵ and -L²-Q²,
wherein said C₁₋₆ alkyl, C₂₋₆ alkenyl and C₂₋₆ alkynyl is optionally substituted by one or more substituents selected from: halo, —CN, —OR^{a2}, —S(O)ₓR^{a2} and —NR^{a2}R^{b2}
or two R³ groups attached to the same or adjacent ring atoms in Ring A together form a C₃₋₆ cycloalkyl or 4- to 6-membered heterocyclic spiro or fused ring, wherein said C₃₋₆ cycloalkyl or 4- to 6-membered heterocyclic is optionally substituted with one or more substituents selected from: halo, ═O, C₁₋₄ alkyl and C₁₋₄ haloalkyl;
R⁵ is selected from: H, C₁₋₆ alkyl and C₁₋₆ haloalkyl, wherein said C₁₋₆ alkyl is optionally substituted by one or more substituents selected from: halo, —CN, —OR^{a3}, —S(O)ₓR^{a3} and —NR^{a3}R^{b3},
L² is a bond or is selected from: *—[CR⁶R⁷]ₐ—NR⁸—, *—NR⁸—[CR⁶R⁷]ₐ—, *—[CR⁶R⁷]ₐ—O—, *—O—[CR⁶R⁷]ₐ—, *—C(O)—[CR⁶R⁷]ₐ—, *—[CR⁶R⁷]ₐ—C(O)—, *—S(O)ₓ—[CR⁶R⁷]ₐ—, *—[CR⁶R⁷]ₐ—S(O)ₓ—and —[CR⁶R⁷]ₐ—, wherein * indicates the bond to Ring A,
   each R⁶ and R⁷ is independently selected from: H, halo, C₁₋₃ alkyl and C₁₋₃ haloalkyl,
   or two R⁶ and R⁷ attached to the same carbon atom form a C₃₋₆ cycloalkyl,
   a is 0 to 4,
   R⁸ is selected from: H, C₁₋₄ alkyl and C₁₋₄ haloalkyl;
Q² is selected from: C₃₋₆ cycloalkyl, 4- to 12-membered heterocyclyl, C₆₋₁₀ aryl and 5- to 12-membered heteroaryl,
wherein said C₃₋₆ cycloalkyl and 4- to 12-membered heterocyclyl is optionally substituted by one or more R⁹,
wherein said C₆₋₁₀ aryl and 5- to 12-membered heteroaryl is optionally substituted by one or more R¹⁰;
L¹ is a bond or is selected from: O and NH;
R⁴ is selected from: H, C₁₋₆ alkyl, C₁₋₆ haloalkyl, C₂₋₆ alkenyl, C₂₋₆ alkynyl and -L³-Q³,
   wherein said C₁₋₆ alkyl, C₂₋₆ alkenyl and C₂₋₆ alkynyl is optionally substituted by one or more substituents selected from: halo, —CN, ═O, —OR^{a8}, —S(O)ₓR^{a8} and —NR^{a8}R^{b8}.
L³ is a bond or —[CR¹¹R¹²]_{b}—,
   each R¹¹ and R¹² is independently selected from: H, halo, C₁₋₃ alkyl and C₁₋₃ haloalkyl,
   or two R¹¹ and R¹² attached to the same carbon atom form a C₃₋₆ cycloalkyl,
   b is 1 to 4,
   Q³ is selected from: C₃₋₆ cycloalkyl, 4- to 7-membered heterocyclyl, phenyl and 5- or 6-membered heteroaryl,
wherein said C₃₋₆ cycloalkyl and 4- to 7-membered heterocyclyl is optionally substituted by one or more R¹³,
wherein said phenyl and 5- or 6-membered heteroaryl is optionally substituted by one or more R¹⁴;
each R⁹ and R¹³ is independently selected from: halo, ═O, —CN, —NO₂, C₁₋₄ alkyl, C₁₋₄ haloalkyl, —OR^{a4}, —S(O)₂R^{a4}, —NR^{a4}R^{b4}, —C(O)R^{a4}, —OC(O)R^{a4}, —C(O)OR^{a4}, —NR^{a4}C(O)R^{b4}, —C(O)NR^{a4}R^{b4}, —NR^{a4}C(O)OR^{b4}, —OC(O)NR^{a4}R^{b4}, —NR^{a4}SO₂R^{b4} and —SO₂NR^{a4}R^{b4};
wherein said C₁₋₄ alkyl is optionally substituted by 1 or 2 substituents selected from: halo, —CN, —OR^{a5}, —NR^{a5}R^{b5} and —SO₂R^{a5};
each R¹⁰ and R¹⁴ is independently selected from: halo, ═O, —CN, —NO₂, C₁₋₄ alkyl, C₁₋₄ haloalkyl, —OR^{a6}, —S(O)₂R^{a6}, —NR^{a6}R^{b6}, —C(O)R^{a6}, —OC(O)R^{a6}, —C(O)OR^{a6}, —NR^{a6}C(O)R^{b6}, —C(O)NR^{a6}R^{b6}, —NR^{a6}C(O)OR^{b6}, —OC(O)NR^{a6}R^{b6}, —NR^{a6}SO₂R^{b6} and —SO₂NR^{a6}R^{b6}.
wherein said C₁₋₄ alkyl is optionally substituted by 1 or 2 substituents selected from: halo, —CN, —OR^{a7}, —NR^{a7}R^{b7} and —SO₂R^{a7};
R^{a1}, R^{a2}, R^{b2}, R^{a3}, R^{b3}, R^{a4}, R^{b4}, R^{a5}, R^{b5}, R^{a6}, R^{b6}, R^{a7}, R^{b7}, R^{a8} and R^{b8} are at each occurrence independently selected from: H, C₁₋₄ alkyl and C₁₋₄ haloalkyl,
or any —NR^{a1}R⁵, —NR^{a2}R^{b2}, —NR^{a3}R^{b3}, —NR^{a4}R^{b4}, —NR^{a5}R^{b5}, —NR^{a6}R^{b6}, —NR^{a7}R^{b7} or —NR^{a8}R^{b8} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl;
n is 0, 1, 2 or 3;
p is 0, 1, 2, 3, 4 or 5; and
x at each occurrence is independently 0, 1 or 2.

Also provided is a pharmaceutical composition comprising a compound of the invention, or a pharmaceutically acceptable salt thereof, and a pharmaceutically acceptable excipient.

Also provided is a compound of the invention, or a pharmaceutically acceptable salt thereof, for use as a medicament. In certain embodiments the compound of the invention, or a pharmaceutically acceptable salt thereof, is for use in the treatment of a disease or medical condition mediated by α2- and/or α3-GABA_{A}Rs.

Also provided is a method of treating a disease or medical condition mediated by α2- and/or α3-GABA_{A}Rs in a subject in need thereof, the method comprising administering to the subject an effective amount of a compound of the invention, or a pharmaceutically acceptable salt thereof.

In certain embodiments there is provided a compound of the invention, or a pharmaceutically acceptable salt thereof, for use in the prevention or treatment of a disorder selected from: an anxiety disorder, a mood disorder, pain, a neurodegenerative disorder, a neurodevelopmental disorder, a cognitive disorder and a psychiatric disorder.

In certain embodiments there is provided a compound of the invention, or a pharmaceutically acceptable salt thereof, for use in the prevention or treatment of anxiety or agitation associated with a disorder selected from: a mood disorder, pain, a neurodegenerative disorder, a neurodevelopment disorder, a cognitive disorder and a psychiatric disorder.

### BRIEF DESCRIPTION OF THE DRAWINGS

Embodiments of the invention are further described hereinafter with reference to the accompanying drawings, in which:

FIG. 1 shows the occupancy of rat brain benzodiazepine binding sites by the compound of Example 17. For doses of the compound in the range 0.1 to 10 mg/kg (p.o.), the occupancy of rat brain benzodiazepine binding sites (i.e., the inhibition of in vivo [³H]flumazenil binding) was dose-dependent, with the dose of 10 mg/kg estimated to fully occupy the brain GABA_{A} receptors (101±0.5%, mean±SEM, n=6 rats).

FIG. 2 shows anxiolytic-like effects of the compound of Example 17 in the rat elevated plus maze assay. The percent time spent on open arms by Example 17 treated rats (1, 3, and 10 mg/kg p.o.) is significantly higher than for vehicle (0.5% methylcellulose/0.1% Tween 80) treated rats (n=13-15/group, Mean±SEM). Chlordiazepoxide (CDP; 5 mg/kg i.p.) was used as reference. **p<0.01, ***p<0.001, ****p<0.0001 differences between groups (one-way ANOVA followed by post hoc Dunnett's).

FIG. 3 shows the occupancy of rat brain benzodiazepine binding sites by the compound of Example 40. For doses of the compound in the range 0.1 to 10 mg/kg (p.o.), the occupancy of rat brain benzodiazepine binding sites (i.e., the inhibition of in vivo [³H]flumazenil binding) was dose-dependent, with the dose of 1 mg/kg estimated to occupy the brain GABA_{A} receptors at 97.3±2.0% (mean±SEM, n=7 rats).

FIG. 4 shows anxiolytic-like effects of the compound of Example 40 in the rat elevated plus maze assay. The percent time spent on open arms by Example 40 treated rats (0.3, 1 and 3 mg/kg p.o.) is significantly higher than for vehicle (0.5% methylcellulose/0.1% Tween 80) treated rats (n=13-15/group, Mean±SEM). Chlordiazepoxide (CDP; 5 mg/kg i.p.) was used as reference. **p<0.01, ***p<0.001, ****p<0.0001 differences between groups (one-way ANOVA followed by post hoc Dunnett's).

FIG. 5 shows the occupancy of rat brain benzodiazepine binding sites by the compound of Example 18. For doses of the compound in the range 0.3 to 10 mg/kg (p.o.), the occupancy of rat brain benzodiazepine binding sites (i.e., the inhibition of in vivo [³H]flumazenil binding) was dose-dependent.

FIG. 6 shows anxiolytic-like effects of the compound of Example 18 in the rat elevated plus maze assay. The percent time spent on open arms by Example 18 treated rats (1 and 3 mg/kg p.o.) is significantly higher than for vehicle (0.5% methylcellulose/0.1% Tween 80) treated rats (n=13-15/group, Mean±SEM). Chlordiazepoxide (CDP; 5 mg/kg i.p.) was used as reference. **p<0.01, ***p<0.001, ****p<0.0001 differences between groups (one-way ANOVA followed by post hoc Dunnett's).

### DETAILED DESCRIPTION

### Definitions

Unless otherwise stated, the following terms used in the specification and claims have the following meanings set out below.

Reference herein to a “compound of the invention” is a reference to any of the compounds disclosed herein including compounds of the formulae (I) to (XXVIII), a compound selected from Compound List 1, or a compound described in any of the Examples, or a pharmaceutically acceptable salt, solvate, or salt of a solvate of any thereof.

Reference to a “α2-GABA_{A}R” refers to a GABA_{A}R that comprises at least one α2 subunit, for example one or two α2 subunits.

Reference to a “α3-GABA_{A}R” refers to a GABA_{A}R that comprises at least one α3 subunit, for example one or two α3 subunits.

The term “positive allosteric modulator” or “PAM” refers to an agent that acts at an allosteric site on GABA_{A}Rs and indirectly increases the responsiveness of the receptor to the endogenous ligand (GABA).

The terms “treating”, or “treatment” refer to any beneficial effect in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; modifying the progression of a disease or condition, making the final point of degeneration less debilitating; improving a patient's physical or mental well-being. The treatment or amelioration of symptoms can be based on objective or subjective parameters; including the results of a physical examination, neuropsychiatric examinations, and/or a psychiatric evaluation. The term “treating” and conjugations thereof, includes prevention of an injury, pathology, condition, or disease (i.e. prophylaxis or prevention). For example, the term “treating” and conjugations thereof, include prevention of a pathology, condition, or disease associated with α2- and/or α3-GABA_{A}Rs (e.g. reducing or preventing symptoms or effects of an anxiety disorder).

The term “associated” or “associated with”, “involving” or “mediated by” in the context of a GABA_{A}Rs associated with a disease means that the disease is caused by (in whole or in part), or a symptom of the disease is caused by (in whole or in part) by GABA_{A} receptors, or receptor activity or function. For example, a symptom of a disease or condition associated with α2- and/or α3-GABA_{A}R pathway activity may be a symptom that results (entirely or partially) from a decrease in the level of activity of α2-GABA_{A}R and/or α3-GABA_{A}R protein pathways. As used herein, what is described as being associated with a disease, if a causative agent, could be a target for treatment of the disease. For example, a disease associated with a decrease in the level of α2 and/or α3-GABA_{A}R activity, may be treated with an agent (e.g. compound as described herein) effective for increasing the level of activity of α2- and/or α3-GABA_{A}Rs.

An “effective amount” is an amount sufficient to accomplish a stated purpose. For example an amount sufficient to achieve the effect for which it is administered, treat a disease, reduce enzyme activity, increase enzyme activity, reduce receptor signalling, increase receptor signalling, reduce one or more symptoms of a disease or condition, or to provide a disease modifying effect (i.e. alter the underlying pathophysiology of the disease). An example of an “effective amount” is an amount sufficient to contribute to the treatment, prevention, or reduction of a symptom or symptoms of a disease, or modify the progression of a disease, which could also be referred to as a “therapeutically effective amount.” A “reduction” of a symptom or symptoms means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The therapeutically effective amount of a compound of the invention can be initially estimated from cell culture assays. Target concentrations will be those concentrations of active compound(s) that are capable of achieving the therapeutic effect described herein, as measured using the methods described herein or known in the art.

Therapeutically effective amounts for use in humans can also be determined from animal models using known methods. For example, a dose for humans can be formulated to achieve a concentration that has been found to be effective in animals. The dosage in humans can be adjusted by monitoring compound effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated, or in response to a biomarker or other correlate or surrogate end-point of the disease. This will provide a therapeutic regimen that is commensurate with the severity of the individual's disease state.

A prophylactic or therapeutic treatment regimen is suitably one that does not cause substantial toxicity and yet is effective to treat the clinical symptoms demonstrated by the particular patient. This determination of a dosage regimen is generally based upon an assessment of the active compound by considering factors such as compound potency, relative bioavailability, patient body weight, presence and severity of adverse side effects, preferred mode of administration and the toxicity profile of the selected agent.

The term “halo” or “halogen” refers to one of the halogens, group 17 of the periodic table. In particular the term refers to fluorine, chlorine, bromine and iodine. Preferably, the term refers to fluorine or chlorine.

The term Cₘ₋ₙ refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, iso-butyl, sec-butyl, tert-butyl, n-pentyl and n-hexyl. “C₁₋₄ alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. For example, C₁₋₆ alkylene may be —CH₂—, —CH₂CH₂—, —CH₂CH(CH₃)—, —CH₂CH₂CH₂— or —CH₂CH(CH₃)CH₂—. The alkyl and alkylene groups may be unsubstituted or substituted by one or more substituents. Possible substituents are described herein. For example, substituents for an alkyl or alkylene group may be halogen, e.g. fluorine, chlorine, bromine and iodine, OH, C₁-C₄ alkoxy, —NR′R″ amino, wherein R′ and R″ are independently H or alkyl. Other substituents for the alkyl group may alternatively be used.

The term “C₁₋₆ haloalkyl”, e.g. “C₁₋₄ haloalkyl”, refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be, for example, —CX₃, —CHX₂, —CH₂CX₃, —CH₂CHX₂ or —CX(CH₃)CH₃ wherein X is a halo (e.g. F, Cl, Br or I). A fluoroalkyl group, i.e. a hydrocarbon chain substituted with at least one fluorine atom (e.g. —CF₃, —CHF₂, —CH₂CF₃ or —CH₂CHF₂).

The term “C₂₋₆ alkenyl” includes a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl. Alkenylene groups are divalent alkenyl groups and may likewise be linear or branched and have two points of attachment to the remainder of the molecule. Furthermore, an alkenylene group may, for example, correspond to one of those alkenyl groups listed in this paragraph. For example alkenylene may be —CH═CH—, —CH₂CH═CH—, —CH(CH₃)CH═CH— or —CH₂CH═CH—. Alkenyl and alkenylene groups may unsubstituted or substituted by one or more substituents. Possible substituents are described herein. For example, substituents may be those described above as substituents for alkyl groups.

The term “C₂₋₆ alkynyl” includes a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl. Alkynylene groups are divalent alkynyl groups and may likewise be linear or branched and have two points of attachment to the remainder of the molecule. Furthermore, an alkynylene group may, for example, correspond to one of those alkynyl groups listed in this paragraph. For example alkynylene may be —C≡C—, —CH₂C≡C—, —CH₂C≡CCH₂—, —CH(CH₃)CH≡C— or —CH₂C≡CCH₃. Alkynyl and alkynylene groups may unsubstituted or substituted by one or more substituents. Possible substituents are described herein. For example, substituents may be those described above as substituents for alkyl groups.

The term “C₃₋₆ cycloalkyl” includes a saturated hydrocarbon ring system containing 3, 4, 5 or 6 carbon atoms. For example, the “C₃-C₆ cycloalkyl” may be cyclopropyl, cyclobutyl, cyclopentyl, cyclohexyl, bicyclo[2.1.1]hexane or bicyclo[1.1.1]pentane. Suitably the “C₃-C₆ cycloalkyl” may be cyclopropyl, cyclobutyl, cyclopentyl or cyclohexyl.

The term “heterocyclyl”, “heterocyclic” or “heterocycle” includes a non-aromatic saturated or partially saturated monocyclic or fused, bridged, or spiro bicyclic heterocyclic ring system. Monocyclic heterocyclic rings may contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles may contain from 7 to 12-member atoms in the ring. Bicyclic heterocyclic(s) rings may be fused, spiro, or bridged ring systems. The heterocyclyl group may be a 3-12, for example, a 3- to 9- (e.g. a 3- to 7-) membered non-aromatic monocyclic or bicyclic saturated or partially saturated group comprising 1, 2 or 3 heteroatoms independently selected from O, S and N in the ring system (in other words 1, 2 or 3 of the atoms forming the ring system are selected from O, S and N). By partially saturated it is meant that the ring may comprise one or two double bonds. This applies particularly to monocyclic rings with from 5 to 7 members. The double bond will typically be between two carbon atoms but may be between a carbon atom and a nitrogen atom. Bicyclic systems may be spiro-fused, i.e. where the rings are linked to each other through a single carbon atom; vicinally fused, i.e. where the rings are linked to each other through two adjacent carbon and/or nitrogen atoms; or they may be share a bridgehead, i.e. the rings are linked to each other through two non-adjacent carbon or nitrogen atoms (a bridged ring system). Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 2,5-diaza-bicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person will appreciate, any heterocycle may be linked to another group via any suitable atom, such as via a carbon or nitrogen atom. For example, the term “piperidino” or “morpholino” refers to a piperidin-1-yl or morpholin-4-yl ring that is linked via the ring nitrogen.

The term “bridged ring systems” includes ring systems in which two rings share more than two atoms, see for example Advanced Organic Chemistry, by Jerry March, 4th Edition, Wiley Interscience, pages 131-133, 1992. Suitably the bridge is formed between two non-adjacent carbon or nitrogen atoms in the ring system. The bridge connecting the bridgehead atoms may be a bond or comprise one or more atoms. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, and quinuclidine.

The term “spiro bi-cyclic ring systems” includes ring systems in which two ring systems share one common spiro carbon atom, i.e. the heterocyclic ring is linked to a further carbocyclic or heterocyclic ring through a single common spiro carbon atom. Examples of spiro ring systems include 3,8-diaza-bicyclo[3.2.1]octane, 2,5-diaza-bicyclo[2.2.1]heptane, 6-azaspiro[3.4]octane, 2-oxa-6-azaspiro[3.4]octane, 2-azaspiro[3.3]heptane, 2-oxa-6-azaspiro[3.3]heptane, 6-oxa-2-azaspiro[3.4]octane, 2,7-diaza-spiro[4.4]nonane, 2-azaspiro[3.5]nonane, 2-oxa-7-azaspiro[3.5]nonane and 2-oxa-6-azaspiro[3.5]nonane.

“Heterocyclyl-Cₘ₋ₙ alkyl” includes a heterocyclyl group covalently attached to a Cₘ₋ₙ alkylene group, both of which are defined herein; and wherein the Heterocyclyl-Cₘ₋ₙ alkyl group is linked to the remainder of the molecule via a carbon atom in the alkylene group. The groups “aryl-Cₘ₋ₙ alkyl”, “heteroaryl-Cₘ₋ₙ alkyl” and “cycloalkyl-Cₘ₋ₙ alkyl” are defined in the same way.

“—Cₘ₋ₙ alkyl substituted by —NRR” and “Cₘ₋ₙ alkyl substituted by —OR” similarly refer to an —NRR″ or —OR″ group covalently attached to a Cₘ₋ₙ alkylene group and wherein the group is linked to the remainder of the molecule via a carbon atom in the alkylene group.

The term “aromatic” when applied to a substituent as a whole includes a single ring or polycyclic ring system with 4n+2 electrons in a conjugated π system within the ring or ring system where all atoms contributing to the conjugated π system are in the same plane.

The term “aryl” includes an aromatic hydrocarbon ring system. The ring system has 4n+2 electrons in a conjugated π system within a ring where all atoms contributing to the conjugated π system are in the same plane. An aryl may be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. For example, the “aryl” may be a C₆₋₁₂ aryl, suitably phenyl or naphthyl. The aryl system itself may be substituted with other groups.

The term “heteroaryl” includes an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated π system where all atoms contributing to the conjugated π system are in the same plane.

Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Bicyclic heteroaryl groups can be vicinally fused, i.e. where the rings are linked to each other through two adjacent carbon and/or nitrogen atoms. Each ring may contain up to about four heteroatoms typically selected from nitrogen, sulfur and oxygen. Typically the heteroaryl ring will contain up to 4, for example up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 1H-pyrazolo[4,3-d]-oxazolyl, 4H-imidazo[4,5-d]thiazolyl, pyrazino[2,3-d]pyridazinyl, imidazo[2,1-b]thiazolyl and imidazo[1,2-b][1,2,4]triazinyl. Examples of heteroaryl groups comprising at least one nitrogen in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl.

“Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Partially aromatic heteroaryl bicyclic ring systems can be vicinally fused, i.e. where the rings are linked to each other through two adjacent carbon and/or nitrogen atoms. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five-membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six-membered heteroaryl groups include but are not limited to pyridyl, pyrazinyl, pyridazinyl, pyrimidinyl and triazinyl.

Particular examples of bicyclic heteroaryl groups containing a six-membered ring fused to a five-membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “oxo,” or “═O” as used herein, means an oxygen that is double bonded to a carbon atom.

The term “optionally substituted” includes either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups, which may be the same or different. For example “one or more optional substituents” may refer to 1 or 2 or 3 substituents (e.g. 1 substituent or 2 substituents).

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible and which are not. For example, it will be recognised that when Ring A is pyridyl the ring nitrogen is not substituted and p is 0 to 4, similarly when Ring A is pyrimidyl, p is 0 to 3.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”:

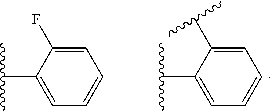 [SMILES: *c1ccccc1F ; *c1ccccc1*]

“Meta” substitution is a substitution pattern where two substituents are on carbons one carbon removed from each other, i.e. with a single carbon atom between the substituted carbons. In other words there is a substituent on the second atom away from the atom with another substituent. For example the groups below are meta substituted:

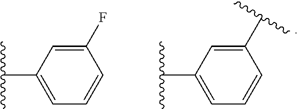 [SMILES: *c1cccc(F)c1 ; *c1cccc(*)c1]

“Para” substitution is a substitution pattern where two substituents are on carbons two carbons removed from each other, i.e. with two carbon atoms between the substituted carbons. In other words there is a substituent on the third atom away from the atom with another substituent. For example the groups below are para substituted:

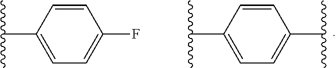 [SMILES: *c1ccc(F)cc1 ; *c1ccc(*)cc1]

When X¹ in formula (I) is CH and n is 1, 2 or 3, R² may substitute the carbon atom represented by X¹. Thus X¹ may be CH or CR² when n is 1, 2 or 3. As will be recognised when X¹ is N the nitrogen atom is not substituted by R² and n is 0, 1 or 2.

Reference to a —NRR′ group forming a 4 to 6 membered heterocyclyl refers to R and R′ together with the nitrogen atom to which they are attached forming a 4 to 6 membered heterocyclyl group. For example, —NR^{a1}R⁵, —NR^{a2}R^{b2}, —NR^{a3}R^{b3}, —NR^{a4}R^{b4}, —NR^{a5}R^{b5}, —NR^{a6}R^{b6}, —NR^{a7}R^{b7} or —NR^{a8}R^{b8} group may form:

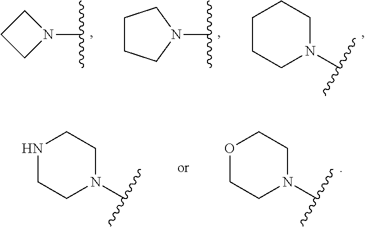

Similarly an —NRR′ group within a substituent may form a carbonyl-linked 4 to 6 membered heterocyclyl, for example a —C(O)NRR′ group may form:

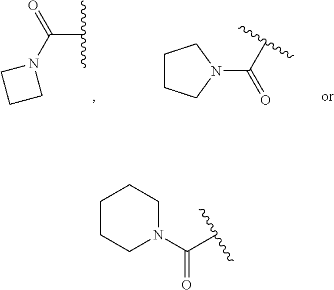 [SMILES: *C(=O)N1CCC1 ; *C(=O)N1CCCC1 ; *C(=O)N1CCCCC1]

—NRR′ groups within substituents such as —OC(O)NRR′, —SO₂NRR′, or —NRC(O)NRR′, may similarly form a 4 to 6 membered heterocyclyl within such substituents.

A bond terminating in a “ [symbol] ” or “*” represents that the bond is connected to another atom that is not shown in the structure. A bond terminating inside a cyclic structure and not terminating at an atom of the ring structure represents that the bond may be connected to any of the atoms in the ring structure where allowed by valency.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550. More preferably, the molecular weight is less than 585 and, for example, is 575 or less.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of the invention may be prepared by for example, one or more of the following methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, for example at least 90%, at least 95% or at least 99%.

The compounds of this invention may possess one or more asymmetric centres; such compounds can therefore be produced as individual (R) or (S) stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E and Z isomers). It is to be understood that the present invention encompasses all optical, diastereoisomers and geometric isomers and mixtures thereof

Z/E (e.g. cis/trans) isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high-pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also, written as “D” for deuterium), ³H (also written as “T” for tritium), ¹¹C, ¹³C, ¹⁴C, ¹⁵O, ¹⁷O, ¹⁸O, ¹³N, ¹⁵N, ¹⁸F, ³⁶Cl, ¹²³I, ²⁵I, ³²P, ³⁵S and the like. The radionuclide that is used will depend on the specific application of that radio-labelled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

The selective replacement of hydrogen with deuterium in a compound may modulate the metabolism of the compound, the PK/PD properties of the compound and/or the toxicity of the compound. For example, deuteration may increase the half-life or reduce the clearance of the compound in vivo. Deuteration may also inhibit the formation of toxic metabolites, thereby improving safety and tolerability. It is to be understood that the invention encompasses deuterated derivatives of compounds of formula (I). As used herein, the term deuterated derivative refers to compounds of the invention where in a particular position at least one hydrogen atom is replaced by deuterium. For example, one or more hydrogen atoms in a C₁₋₄-alkyl group may be replaced by deuterium to form a deuterated C₁₋₄-alkyl group. By way of example, if R⁴ is methyl the invention also encompasses —CD₃, —CHD₂ and —CH₂D.

Certain compounds of the invention may exist in solvated as well as unsolvated forms such as, for example, hydrated forms. It is to be understood that the invention encompasses all such solvated forms.

It is also to be understood that certain compounds of the invention may exhibit polymorphism, and that the invention encompasses all such forms.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

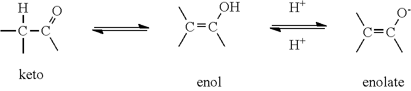

for example

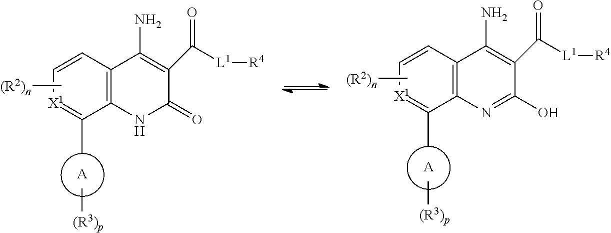

It is to be understood that when X¹ is CH and n is 1, 2, or 3, the R² substituent may be present on the carbon atom represented by X¹ (i.e. X¹ may be CH or CR² when n is 1, 2 or 3).

The in vivo effects of a compound of the invention may be exerted in part by one or more metabolites that are formed within the human or animal body after administration of a compound of the invention.

It is further to be understood that a suitable pharmaceutically-acceptable pro-drug of a compound of the formula (I) also forms an aspect of the present invention. Accordingly, the compounds of the invention encompass pro-drug forms of the compounds and the compounds of the invention may be administered in the form of a pro-drug (i.e. a compound that is broken down in the human or animal body to release a compound of the invention). A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo-cleavable ester derivatives that may be formed at a carboxy group or a hydroxy group in a compound of the invention and in vivo-cleavable amide derivatives that may be formed at a carboxy group or an amino group in a compound of the invention.

Accordingly, the present invention includes those compounds of the invention as defined herein when made available by organic synthesis and when made available within the human or animal body by way of cleavage of a pro-drug thereof. Accordingly, the present invention includes those compounds of the formula (I) that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically-acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the formula (I) that possesses a carboxy group is, for example, an in vivo-cleavable ester thereof. An in vivo-cleavable ester of a compound of the invention containing a carboxy group is, for example, a pharmaceutically-acceptable ester which is cleaved in the human or animal body to produce the parent acid. Suitable pharmaceutically-acceptable esters for carboxy include C₁₋₆ alkyl esters such as methyl, ethyl and tert-butyl, C₁₋₆ alkoxymethyl esters such as methoxymethyl esters, C₁₋₆ alkanoyloxymethyl esters such as pivaloyloxymethyl esters, 3-phthalidyl esters, C₃₋₈ cycloalkylcarbonyloxy-C₁₋₆ alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆ alkoxycarbonyloxy-C₁₋₆ alkyl esters such as methoxycarbonyloxymethyl and 1-methoxycarbonyloxyethyl esters.

A suitable pharmaceutically-acceptable pro-drug of a compound of the invention that possesses a hydroxy group is, for example, an in vivo-cleavable ester or ether thereof. An in vivo-cleavable ester or ether of a compound of the invention containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include C₁₋₁₀ alkanoyl groups such as acetyl, benzoyl, phenylacetyl and substituted benzoyl and phenylacetyl groups, C₁₋₁₀ alkoxycarbonyl groups such as ethoxycarbonyl, N,N—(C₁₋₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl) piperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the invention that possesses a carboxy group is, for example, an in vivo-cleavable amide thereof, for example an amide formed with an amine such as ammonia, a C₁₋₄ alkylamine such as methylamine, a (C₁₋₄ alkyl)₂amine such as dimethylamine, N-ethyl-N-methylamine or diethylamine, a C₁₋₄ alkoxy-C₂₋₄ alkylamine such as 2-methoxyethylamine, a phenyl-C₁₋₄ alkylamine such as benzylamine and amino acids such as glycine or an ester thereof.

A suitable pharmaceutically-acceptable pro-drug of a compound of the invention that possesses an amino group is, for example, an in vivo-cleavable amide or carbamate derivative thereof. Suitable pharmaceutically-acceptable amides from an amino group include, for example an amide formed with C₁₋₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N, N-dialkylaminomethyl, morpholinomethyl, piperazin-1-ylmethyl and 4-(C₁₋₄ alkyl) piperazin-1-ylmethyl. Suitable pharmaceutically-acceptable carbamates from an amino group include, for example acyloxyalkoxycarbonyl and benzyloxycarbonyl groups.

### Compounds

The following paragraphs are applicable to the compounds of the invention.

In certain embodiments the compound of the formula (I) is a compound of the formula (II), or a pharmaceutically acceptable salt thereof:

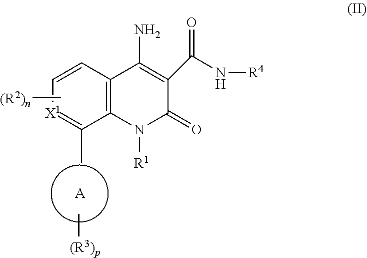

In certain embodiments the compound of the formula (I) is a compound of the formula (III), or a pharmaceutically acceptable salt thereof:

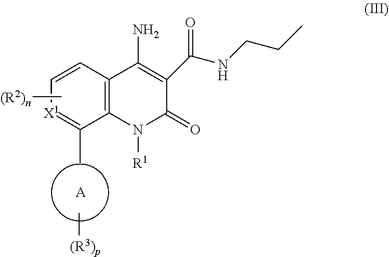

In certain embodiments the compound of the formula (I) is a compound of the formula (IV), or a pharmaceutically acceptable salt thereof:

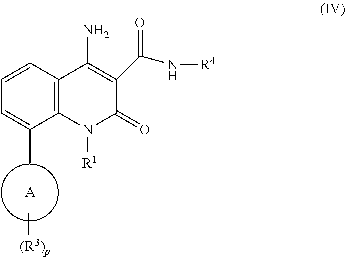

In certain embodiments the compound of the formula (I) is a compound of the formula (V), or a pharmaceutically acceptable salt thereof:

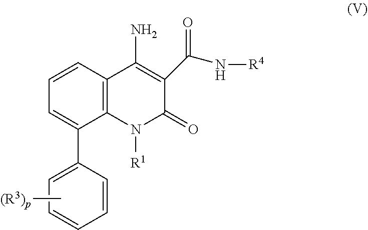

In certain embodiments the compound of the formula (I) is a compound of the formula (VI), or a pharmaceutically acceptable salt thereof:

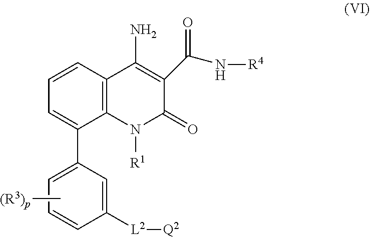

In certain embodiments the compound of the formula (I) is a compound of the formula (VII), or a pharmaceutically acceptable salt thereof:

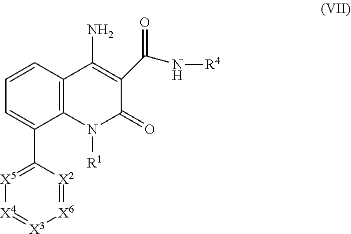

wherein X², X³, X⁴, X⁵ and X⁶ is each independently selected from N, CH and CR³, provided at least 1 but no more than 2 of X², X³, X⁴, X⁵ and X⁶ is N.

In certain embodiments the compound of the formula (I) is a compound of the formula (VIII), or a pharmaceutically acceptable salt thereof:

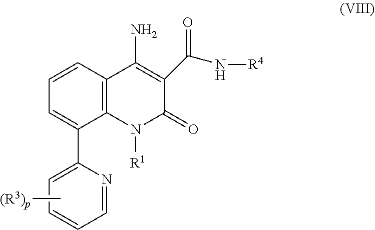

In certain embodiments the compound of the formula (I) is a compound of the formula (IX), or a pharmaceutically acceptable salt thereof:

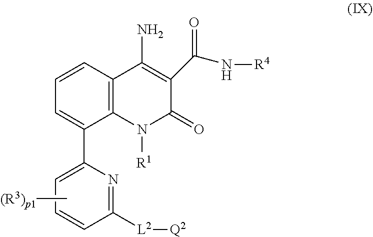

wherein p1 is 0, 1, 2 or 3.

In certain embodiments the compound of the formula (I) is a compound of the formula (X), or a pharmaceutically acceptable salt thereof:

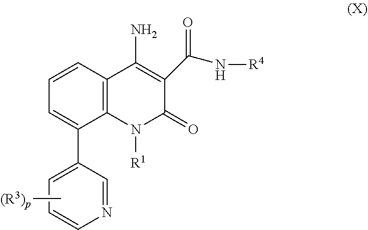

In certain embodiments the compound of the formula (I) is a compound of the formula (XI), or a pharmaceutically acceptable salt thereof:

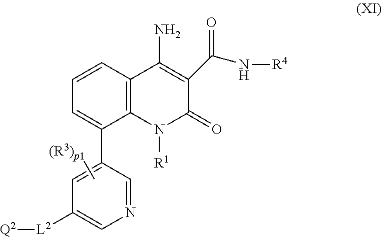

wherein p1 is 0, 1, 2 or 3.

In certain embodiments the compound of the formula (I) is a compound of the formula (XII), or a pharmaceutically acceptable salt thereof:

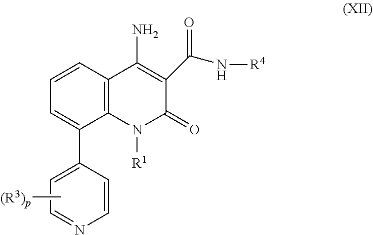

In certain embodiments the compound of the formula (I) is a compound of the formula (XIII), or a pharmaceutically acceptable salt thereof:

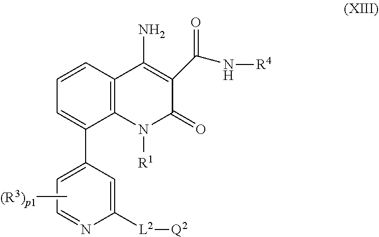

wherein p1 is 0, 1, 2 or 3.

In certain embodiments the compound of the formula (I) is a compound of the formula (XIV), or a pharmaceutically acceptable salt thereof:

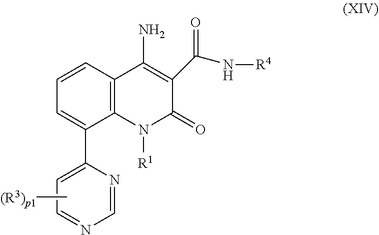

wherein p1 is 0, 1, 2 or 3.

In certain embodiments the compound of the formula (I) is a compound of the formula (XV), or a pharmaceutically acceptable salt thereof:

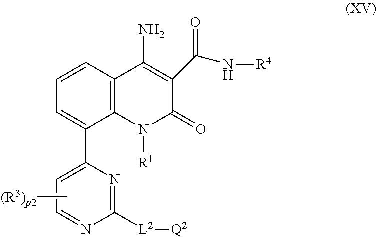

wherein p2 is 0, 1 or 2.

In certain embodiments the compound of the formula (I) is a compound of the formula (XVI), or a pharmaceutically acceptable salt thereof:

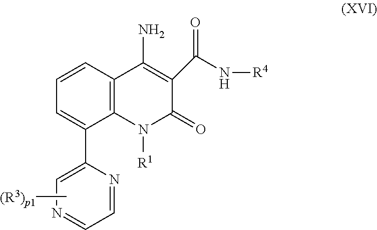

wherein p1 is 0, 1, 2 or 3.

In certain embodiments the compound of the formula (I) is a compound of the formula (XVII), or a pharmaceutically acceptable salt thereof:

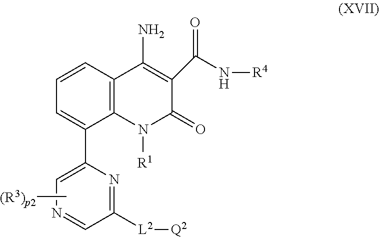

wherein p2 is 0, 1 or 2.

In certain embodiments the compound of the formula (I) is a compound of the formula (XVIII), or a pharmaceutically acceptable salt thereof:

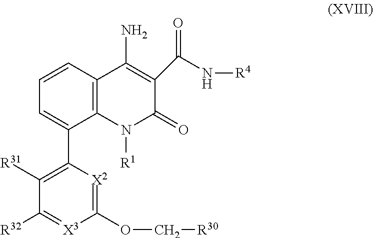

wherein
X² and X³ are each independently N or CH;
R³⁰ is selected from H, C₁₋₄ alkyl, C₁₋₄ haloalkyl, and Q²;
R³¹ and R³² are each independently selected from: H and R³.

In certain embodiments the compound of the formula (I) is a compound of the formula (XIX), or a pharmaceutically acceptable salt thereof:

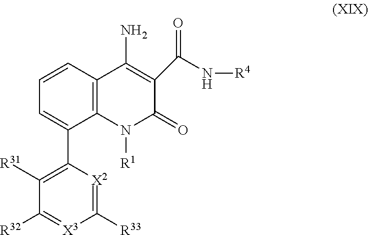

wherein
X² and X³ are each independently N or CH;
R³¹ and R³² are each independently selected from: H and R³; and
R³³ is selected from: C₁₋₄ alkyl (e.g. methyl), —O—C₁₋₄ alkyl (e.g. methoxy) and -L²-Q².

In certain embodiments the compound of the formula (I) is a compound of the formula (XX), or a pharmaceutically acceptable salt thereof:

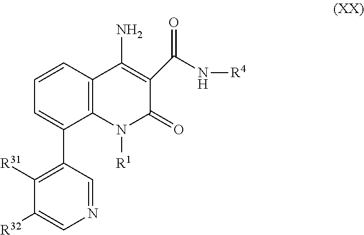

wherein
R³¹ and R³² are each independently H or R³.

In certain embodiments the compound of the formula (I) is a compound of the formula (XXI), or a pharmaceutically acceptable salt thereof:

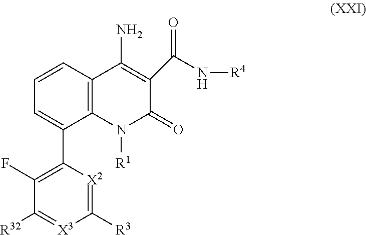

wherein
X² and X³ are each independently N or CH; and
R³² is selected from: H and R³.

In certain embodiments the compound of the formula (I) is a compound of the formula (XXII), or a pharmaceutically acceptable salt thereof:

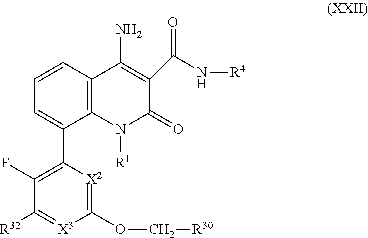

wherein
X² and X³ are each independently N or CH;
R³⁰ is selected from H, C₁₋₄ alkyl, C₁₋₄ haloalkyl and Q² (preferably wherein Q² is phenyl or a 5- or 6-membered heteroaryl optionally substituted by one or more R¹⁰); and
R³² is selected from: H and R³.

In certain embodiments the compound of the formula (I) is a compound of the formula (XXIII), or a pharmaceutically acceptable salt thereof:

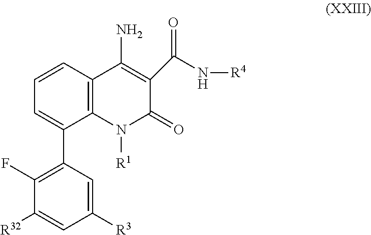

wherein R³² is selected from: H and R³.

In certain embodiments the compound of the formula (I) is a compound of the formula (XXIV), or a pharmaceutically acceptable salt thereof:

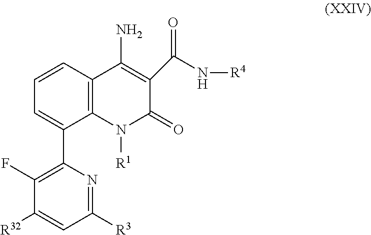

wherein R³² is selected from: H and R³.

In certain embodiments the compound of the formula (I) is a compound of the formula (XXV), or a pharmaceutically acceptable salt thereof:

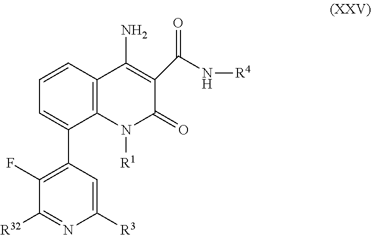

wherein R³² is selected from: H and R³.

In certain embodiments the compound of the formula (I) is a compound of the formula (XXVI), or a pharmaceutically acceptable salt thereof:

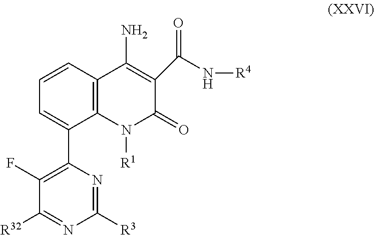

wherein R³² is selected from: H and R³.

In certain embodiments the compound of the formula (I) is a compound of the formula (XXVII), or a pharmaceutically acceptable salt thereof:

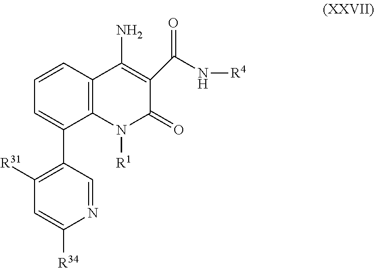

wherein R³¹ and R³⁴ are each independently H or R³.

In certain embodiments the compound of the formula (I) is a compound of the formula (XXVIII), or a pharmaceutically acceptable salt thereof:

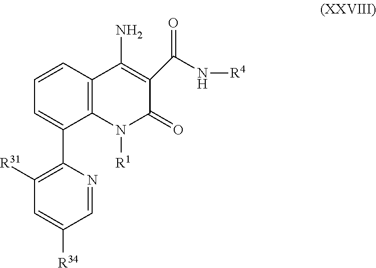

wherein R³¹ and R³⁴ are each independently H or R³.

In certain embodiments compounds of the invention include, for example, compounds of formulae (I) to (XXVIII), or a pharmaceutically acceptable salt thereof, wherein, unless otherwise stated, each of Ring A, R¹, R², R³, L¹, n and p has any of the meanings defined hereinbefore or in any of the following statements in the numbered paragraphs (1) to (118) hereinafter. These statements are independent and interchangeable. In other words, any of the features described in any one of the following statements may (where chemically allowable) be combined with the features described in one or more other statements below. In particular, where a compound is exemplified or illustrated in this specification, any two or more of the statements below which describe a feature of that compound, expressed at any level of generality, may be combined so as to represent subject matter which is contemplated as forming part of the disclosure of this invention in this specification.
1. R¹ is selected from: H and C₁₋₄ alkyl.
2. R¹ is selected from: H and C₁₋₃ alkyl.
3. R¹ is selected from: H, methyl, ethyl and isopropyl.
4. R¹ is selected from: H and methyl.
5. R¹ is methyl.
6. R¹ is H.
7. each R² is independently selected from: halo, C₁₋₃ alkyl and C₁₋₃ haloalkyl.
8. each R² is independently selected from: halo, methyl and C₁-haloalkyl.
9. each R² is independently selected from: F, Cl, methyl and —CF₃.
10. each R² is independently selected from halo.
11. each R² is independently selected from F and Cl.
12. R² is F.
13. n is 0, 1 or 2.
14. n is 0, 1 or 2 and each R² is independently as defined in any one of 7 to 12.
15. n is 0 or 1 and R² is as defined in any one of 7 to 12.
16. n is 1 and R² is as defined in any one of 7 to 12.
17. n is 0.
18. X¹ is N.
19. X¹ is CH.
20. Ring A is selected from: phenyl and a 5- or 6-membered heteroaryl, wherein said heteroaryl contains 1, 2, 3 or 4 ring heteroatoms selected from O, S and N, wherein Ring A is optionally substituted by one or more R³.
21. Ring A is phenyl, wherein Ring A is optionally substituted by one or more R³.
22. Ring A is a 5- or 6-membered heteroaryl containing 1 ring nitrogen heteroatom and optionally 1, 2 or 3 ring heteroatoms selected from O, S and N, wherein Ring A is optionally substituted by one or more R³.
23. Ring A is a 5-membered heteroaryl containing 1, 2, 3 or 4 ring heteroatoms selected from O, S and N, wherein Ring A is optionally substituted by one or more R³.
24. Ring A is a 5-membered heteroaryl containing 1 ring nitrogen heteroatom and optionally 1, 2 or 3 ring heteroatoms selected from O, S and N, wherein Ring A is optionally substituted by one or more R³.
25. Ring A is a 6-membered heteroaryl containing 1 or 2 ring nitrogen heteroatoms, wherein Ring A is optionally substituted by one or more R³.
26. Ring A is selected from: phenyl, pyrrolyl, imidazolyl, pyrazolyl, oxazolyl, isoxazolyl, furanyl, thiophenyl, thiazolyl, isothiazolyl, triazolyl, oxadiazolyl, thiadiazolyl, pyridyl, pyrimidyl, pyridazinyl and pyrazinyl, wherein Ring A is optionally substituted by one or more R³.
27. Ring A is selected from: phenyl, pyrrolyl, imidazolyl, pyrazolyl, oxazolyl, isoxazolyl, triazolyl, pyridyl, pyrimidyl, pyridazinyl and pyrazinyl, wherein Ring A is optionally substituted by one or more R³.
28. Ring A is selected from: pyrrolyl, imidazolyl, pyrazolyl, oxazolyl, isoxazolyl and triazolyl, wherein Ring A is optionally substituted by one or more R³.
29. Ring A is selected from: pyridyl, pyrimidyl and pyrazinyl, wherein Ring A is optionally substituted by one or more R³.
30. Ring A is selected from:

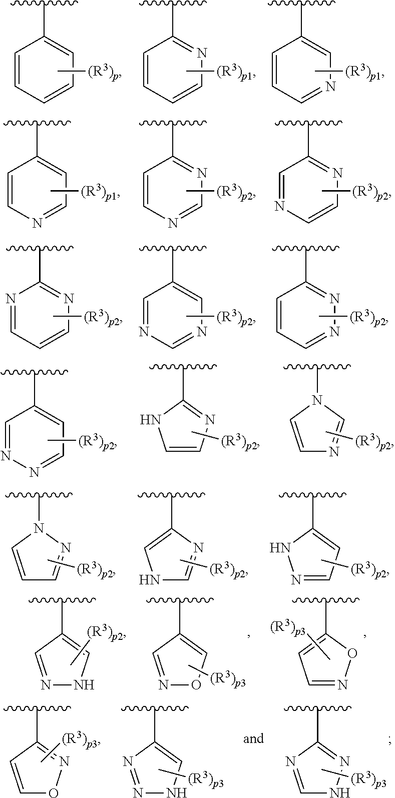

wherein p1 is selected from 0, 1, 2, 3 and 4;
p2 is selected from 0, 1, 2 and 3; and
p3 is selected from 0, 1 and 2.
31. Ring A is selected from:

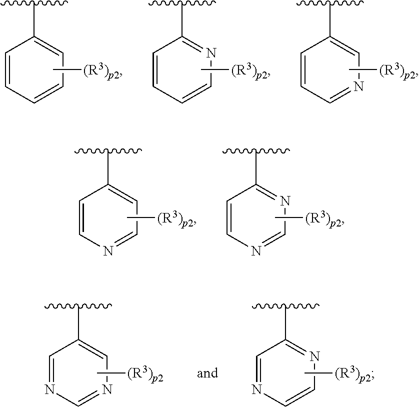

wherein p2 is selected from 0, 1, 2 and 3.
32. Ring A is selected from:

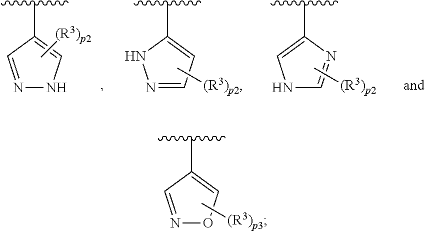

wherein p2 is selected from 0, 1, 2 and 3 and p3 is 0, 1 or 2.
33. Each R³ is independently selected from: halo, —CN, C₁₋₄ alkyl, C₁₋₄ haloalkyl, —OR⁵, —NR^{a1}R⁵, —SO₂R⁵, —C(O)NR^{a1}R⁵, —OC(O)NR⁵R^{a1}, —SO₂NR^{a1}R⁵ and -L²-Q²,
   wherein said C₁₋₄ alkyl is optionally substituted by one or more substituents selected from: halo, —CN, —OR^{a2}, —SO₂R^{a2} and —NR^{a2}R^{b2}
   or two R³ groups attached to adjacent ring atoms in Ring A together form a 4- to 6-membered heterocyclic fused ring, wherein said 4- to 6-membered heterocyclic fused ring is optionally substituted with one or more substituents selected from: halo, ═O, C₁₋₄ alkyl and C₁₋₄ haloalkyl;
   R⁵ is selected from: H, C₁₋₄ alkyl and C₁₋₄ haloalkyl, wherein said C₁₋₄ alkyl is optionally substituted by one or more substituents selected from: halo, —CN, —OR^{a3}, —SO₂R^{a3} and —NR^{a3}R^{b3}.
   L² is a bond or is selected from: *—[CR⁶R⁷]ₐ—NR⁸—, *—NR⁸—[CR⁶R⁷]ₐ—, *—[CR⁶R⁷]ₐ—O—, *—O—[CR⁶R⁷]ₐ—, *—C(O)—[CR⁶R⁷]ₐ—, *—[CR⁶R⁷]ₐ—C(O)—, *—SO₂—[CR⁶R⁷]ₐ—, *—[CR⁶R⁷]ₐ—SO₂— and —[CR⁶R⁷]ₐ—, wherein * indicates the bond to Ring A,
      each R⁶ and R⁷ is independently selected from: H, halo, C₁₋₃ alkyl,
      or two R⁶ and R⁷ attached to the same carbon atom form a C₃₋₆ cycloalkyl,
      a is 0 to 3,
      R⁸ is selected from: H and C₁₋₃ alkyl;
   Q² is selected from: C₃₋₆ cycloalkyl, 4- to 12-membered heterocyclyl, phenyl and 5-to 12-membered heteroaryl,
   wherein said C₃₋₆ cycloalkyl and 4- to 12-membered heterocyclyl is optionally substituted by one or more R⁹,
   wherein said phenyl and 5- to 12-membered heteroaryl is optionally substituted by one or more R¹⁰.

Thus it may be that Ring A is selected from any one of 20 to 32, wherein each R³ is independently as defined in this paragraph.
34. Each R³ is independently selected from: halo, —CN, C₁₋₄ alkyl, C₁₋₄ haloalkyl, —OR⁵, —NR^{a1}R⁵, and -L²-Q²,
   wherein said C₁₋₄ alkyl is optionally substituted by one or more substituents selected from: —OR^{a2} and —NR^{a2}R^{b2}
   or two R³ groups attached to adjacent ring atoms in Ring A together form a 4- to 6-membered heterocyclic fused ring containing 1, 2 or 3 ring heteroatoms selected from O, S and N, wherein said 4- to 6-membered heterocyclic fused ring is optionally substituted with one or more substituents selected from: halo, ═O, C₁₋₄ alkyl and C₁₋₄ haloalkyl;
   R⁵ is selected from: H, C₁₋₄ alkyl and C₁₋₄ haloalkyl, wherein said C₁₋₄ alkyl is optionally substituted by one or more substituents selected from: —OR^{a3} and —NR^{a3}R^{b3};
   L² is a bond or is selected from: *—[CR⁶R⁷]ₐ—NR⁸—, *—NR⁸—[CR⁶R⁷]ₐ—, *—[CR⁶R⁷]ₐ—O—, *—O—[CR⁶R⁷]ₐ—, *—C(O)—[CR⁶R⁷]ₐ—, *—[CR⁶R⁷]ₐ—C(O)—, *—SO₂—[CR⁶R⁷]ₐ—, *—[CR⁶R⁷]ₐ—SO₂— and —[CR⁶R⁷]ₐ—, wherein * indicates the bond to Ring A,
      each R⁶ and R⁷ is independently selected from: H, halo, C₁₋₃ alkyl,
      or two R⁶ and R⁷ attached to the same carbon atom form a C₃₋₆ cycloalkyl,
      a is 0, 1 or 2,
      R⁸ is selected from: H and C₁₋₃ alkyl;
   Q² is selected from: C₃₋₆ cycloalkyl, 4- to 10-membered heterocyclyl containing 1 ring nitrogen heteroatom and 1 or 2 ring heteroatoms selected from O, S and N, phenyl and 5- to 10-membered heteroaryl,
   wherein said C₃₋₆ cycloalkyl and 4- to 10-membered heterocyclyl is optionally substituted by one or more R⁹,
   wherein said phenyl and 5- to 10-membered heteroaryl is optionally substituted by one or more R¹⁰.

Thus it may be that Ring A is selected from any one of 20 to 32, wherein each R³ is independently as defined in this paragraph.
35. Each R³ is independently selected from: halo, —CN, C₁₋₄ alkyl, C₁₋₄ haloalkyl, —OR⁵, —NR^{a1}R⁵, —SO₂R⁵, —C(O)NR^{a1}R⁵, —OC(O)NR⁵R^{a1}, —SO₂NR^{a1}R⁵ and -L²-Q²,
   wherein said C₁₋₄ alkyl is optionally substituted by one or more substituents selected from: halo, —CN, —OR^{a2}, —SO₂R^{a2} and —NR^{a2}R^{b2}
   or two R³ groups attached to adjacent ring atoms in Ring A together form a 4- to 6-membered heterocyclic fused ring, wherein said 4- to 6-membered heterocyclic fused ring is optionally substituted with one or more substituents selected from: halo, ═O, C₁₋₄ alkyl and C₁₋₄ haloalkyl;
   R⁵ is selected from: H, C₁₋₄ alkyl and C₁₋₄ haloalkyl, wherein said C₁₋₄ alkyl is optionally substituted by one or more substituents selected from: halo, —CN, —OR^{a3}, —SO₂R^{a3} and —NR^{a3}R^{b3}. Thus it may be that Ring A is selected from any one of 20 to 32, wherein each R³ is independently as defined in this paragraph.
36. Each R³ is independently selected from: halo, —CN, C₁₋₄ alkyl, C₁₋₄ haloalkyl, —OR⁵, and -L²-Q²,
   wherein said C₁₋₄ alkyl is optionally substituted by one or more substituents selected from —OR^{a2}
   or two R³ groups attached to adjacent ring atoms in Ring A together form a 4- to 6-membered heterocyclic fused ring containing 1, 2 or 3 ring heteroatoms selected from O, S and N, wherein said 4- to 6-membered heterocyclic fused ring is optionally substituted with one or more substituents selected from: halo, ═O, C₁₋₄ alkyl and C₁₋₄ haloalkyl;
   R⁵ is selected from: H, C₁₋₄ alkyl and C₁₋₄ haloalkyl, wherein said C₁₋₄ alkyl is optionally substituted by one or more substituents selected from: —OR^{a3},
   L² is a bond or is selected from: *—[CR⁶R⁷]ₐ—O—, *—O—[CR⁶R⁷]ₐ—, *—C(O)—[CR⁶R⁷]ₐ—, *—[CR⁶R⁷]ₐ—C(O)—, *—SO₂—[CR⁶R⁷]ₐ—, *—[CR⁶R⁷]ₐ—SO₂— and —[CR⁶R⁷]ₐ—, wherein * indicates the bond to Ring A,
      each R⁶ and R⁷ is independently selected from: H, halo, C₁₋₃ alkyl,
      or two R⁶ and R⁷ attached to the same carbon atom form a C₃₋₆ cycloalkyl,
      a is 0, 1 or 2,
   Q² is selected from: phenyl and 5- to 10-membered heteroaryl,
   wherein said phenyl and 5- to 10-membered heteroaryl is optionally substituted by one or more R¹⁰.

Suitably no more that no more than one R³ is -L²-Q². It may be that Ring A is selected from any one of 20 to 32, wherein each R³ is independently as defined in this paragraph.
37. Each R³ is independently selected from: halo, —CN, C₁₋₄ alkyl, C₁₋₄ haloalkyl, —OR⁵, and -L²-Q²,
   wherein said C₁₋₄ alkyl is optionally substituted by one or more substituents selected from —OR^{a2}
      R⁵ is selected from: H, C₁₋₄ alkyl and C₁₋₄ haloalkyl, wherein said C₁₋₄ alkyl is optionally substituted by one or more substituents selected from: —OR^{a3};
   L² is a bond or is selected from: *—[CR⁶R⁷]ₐ—O—, *—O—[CR⁶R⁷]ₐ—, *—C(O)—[CR⁶R⁷]ₐ—, *—[CR⁶R⁷]ₐ—C(O)—, *—SO₂—[CR⁶R⁷]ₐ—, *—[CR⁶R⁷]ₐ—SO₂— and —[CR⁶R⁷]ₐ—, wherein * indicates the bond to Ring A,
      each R⁶ and R⁷ is independently selected from: H, halo, C₁₋₃ alkyl,
      or two R⁶ and R⁷ attached to the same carbon atom form a C₃₋₆ cycloalkyl,
      a is 0, 1 or 2,
   Q² is selected from: phenyl and 5- to 10-membered heteroaryl,
   wherein said phenyl and 5- to 10-membered heteroaryl is optionally substituted by one or more R¹⁰.

Suitably no more that no more than one R³ is -L²-Q². It may be that Ring A is selected from any one of 20 to 32, wherein each R³ is independently as defined in this paragraph.
38. Each R³ is independently selected from: halo, —CN, C₁₋₄ alkyl, C₁₋₄ haloalkyl, —OR⁵, —NR^{a1}R⁵, and -L²-Q²,
   wherein said C₁₋₄ alkyl is optionally substituted by one or more substituents selected from: —OR^{a2} and —NR^{a2}R^{b2}
   or two R³ groups attached to adjacent ring atoms in Ring A together form a 4- to 6-membered heterocyclic fused ring containing 1, 2 or 3 ring heteroatoms selected from O, S and N, wherein said 4- to 6-membered heterocyclic fused ring is optionally substituted with one or more substituents selected from: halo, ═O, C₁₋₄ alkyl and C₁₋₄ haloalkyl;
   R⁵ is selected from: H, C₁₋₄ alkyl and C₁₋₄ haloalkyl, wherein said C₁₋₄ alkyl is optionally substituted by one or more substituents selected from: —OR^{a3} and —NR^{a3}R^{b3};
   L² is a bond or is selected from: *—[CR⁶R⁷]ₐ—NR⁸—, *—NR⁸—[CR⁶R⁷]ₐ—, *—[CR⁶R⁷]ₐ—O—, *—O—[CR⁶R⁷]ₐ—, *—C(O)—[CR⁶R⁷]ₐ—, *—[CR⁶R⁷]ₐ—C(O)—, *—SO₂—[CR⁶R⁷]ₐ—, *—[CR⁶R⁷]ₐ—SO₂— and —[CR⁶R⁷]ₐ—, wherein * indicates the bond to Ring A,
      each R⁶ and R⁷ is independently selected from: H, halo, C₁₋₃ alkyl,
      or two R⁶ and R⁷ attached to the same carbon atom form a C₃₋₆ cycloalkyl,
      a is 0, 1 or 2,
      R⁸ is selected from: H and C₁₋₃ alkyl;
   Q² is selected from: is selected from: 4- to 10-membered heterocyclyl containing 1 ring nitrogen heteroatom and 1 or 2 ring heteroatoms selected from O, S and N,
   wherein said 4- to 10-membered heterocyclyl is optionally substituted by one or more R⁹.

Suitably no more that no more than one R³ is -L²-Q². Thus it may be that Ring A is selected from any one of 20 to 32, wherein each R³ is independently as defined in this paragraph.
39. Each R³ is independently selected from: halo, —CN, C₁₋₄ alkyl, C₁₋₄ haloalkyl and —OR⁵, wherein said C₁₋₄ alkyl is optionally substituted by one or more substituents selected from —OR^{a2},
   or two R³ groups attached to adjacent ring atoms in Ring A together form a fused ring of the formula: *—O—(CH₂)ₜ—O—**, wherein * and ** are the attachment points to adjacent ring atoms in Ring A, and t is 1 to 3, wherein said fused ring is optionally substituted by 1 or 2 substituents selected from halo, C₁₋₄ alkyl and C₁₋₄ haloalkyl;
   R⁵ is selected from: H, C₁₋₄ alkyl and C₁₋₄ haloalkyl, wherein said C₁₋₄ alkyl is optionally substituted by one or more substituents selected from: —OR^{a3}.

Thus it may be that each R³ is independently as defined in this paragraph and Ring A is selected from any one of 20 to 32.
40. Each R³ is independently selected from: halo, —CN, C₁₋₄ alkyl, —C₁₋₄ alkyl-OR^{a2}, C₁₋₄ haloalkyl and —OR⁵,
   R⁵ is selected from: H, C₁₋₄ alkyl and C₁₋₄ haloalkyl, wherein said C₁₋₄ alkyl is optionally substituted by one or more substituents selected from: —OR^{a3}.

Thus it may be that Ring A is selected from any one of 20 to 32, wherein each R³ is independently as defined in this paragraph
41. Each R³ is independently selected from: halo, C₁₋₄ alkyl, —C₁₋₄ alkyl-OR^{a2}, C₁₋₄ haloalkyl and —OR⁵,
   R⁵ is selected from: H, C₁₋₄ alkyl, —C₁₋₄ alkyl-OR^{a3} and C₁₋₄ haloalkyl.

Thus it may be that each R³ is independently as defined in this paragraph and Ring A is selected from any one of 20 to 32.
42. p is 0.
43. p is 1. Thus it may be that p is 1 and R³ is as defined in any one of 33 to 40.
44. p is 2. Thus it may be that p is 2 and each R³ is independently as defined in any one of 33 to 40.
45. p is 3. Thus it may be that p is 2 and each R³ is independently as defined in any one of 33 to 40.
46. Two R³ groups attached to adjacent ring atoms in Ring A together form a fused ring of the formula: *—O—(CH₂)ₜ—O—**, wherein * and ** are the attachment points to adjacent ring atoms in Ring A, and t is 1 to 3, wherein said fused ring is optionally substituted by 1 or 2 substituents selected from halo, C₁₋₄ alkyl and C₁₋₄ haloalkyl. Thus it may be that Ring A is selected from:

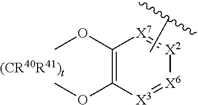

wherein
each R⁴⁰ and each R⁴¹ are independently selected from: H, halo, C₁₋₄ alkyl and C₁₋₄ haloalkyl;
t is 1, 2 or 3; and
X², X³, X⁶ and X⁷ are each independently selected from: N, CH and CR³, wherein each R³ independently has any of the values defined herein (for example R³ is independently selected from: halo, —CN, C₁₋₄ alkyl, C₁₋₄ haloalkyl, —OC₁₋₄ alkyl and —OC₁₋₄ haloalkyl). Suitably no more than 2 of X², X³, X⁶ and X⁷ are N.
47. Ring A is substituted by one R³ group selected from: —NR^{a1}R⁵, C₁₋₄ alkyl-NR^{a2}R^{b2}, and -L²-Q², wherein Q² is selected from a 4- to 10-membered heterocyclyl containing 1 ring nitrogen heteroatom and 1 or 2 ring heteroatoms selected from O, S and N,
   wherein said 4- to 10-membered heterocyclyl is optionally substituted by one or more R⁹;
   and wherein Ring A is optionally substituted by 1 or 2 additional R³ independently selected from: halo, —CN, C₁₋₄ alkyl, C₁₋₄ haloalkyl and —OR⁵. Thus it may be that Ring A is selected from any one of 20 to 32, wherein each R³ is independently as defined in this paragraph.
48. Ring A is substituted with one R³ selected from-L²-Q² and optionally one or more R³ substituents independently selected from: halo, —CN, C₁₋₄ alkyl, C₁₋₄ haloalkyl, —OR⁵ and —NR^{a1}R⁵,
   wherein said C₁₋₄ alkyl is optionally substituted by one or more substituents selected from: —OR^{a2} and —NR^{a2}R^{b2},
   R⁵ is selected from: H, C₁₋₄ alkyl and C₁₋₄ haloalkyl, wherein said C₁₋₄ alkyl is optionally substituted by one or more substituents selected from: —OR^{a3} and —NR^{a3}R^{b3}. Thus it may be that Ring A is selected from any one of 20 to 32, wherein Ring A is substituted with the R³ group(s) defined in this paragraph.
49. Ring A is substituted with one R³ selected from -L²-Q² and optionally one or two R³ substituents independently selected from: halo, —CN, C₁₋₄ alkyl, —C₁₋₄ alkyl-OR^{a2}, C₁₋₄ haloalkyl and —OR⁵,
   R⁵ is selected from: H, C₁₋₄ alkyl, —C₁₋₄ alkyl-OR^{a3} and C₁₋₄ haloalkyl. Thus it may be that Ring A is selected from any one of 20 to 32, wherein Ring A is substituted with the R³ group(s) defined in this paragraph.
50. Ring A is substituted with one or two R³ substituents independently selected from: halo, —CN, C₁₋₄ alkyl, C₁₋₄ haloalkyl, —C₁₋₄ alkyl-OR^{a2}, —O—C₁₋₄ alkyl and —O—C₁₋₄ haloalkyl. Thus it may be that Ring A is selected from any one of 20 to 32, wherein Ring A is substituted with the R³ group(s) defined in this paragraph.
51. Ring A is substituted with one R³ selected from: —C₁₋₄ alkyl-OR^{a2}—O—C₁₋₄ alkyl and —O—C₁₋₄ haloalkyl, and optionally one or two R³ independently selected from: halo, —CN and C₁₋₄ alkyl. Thus it may be that Ring A is selected from any one of 20 to 32, wherein Ring A is substituted with the R³ group(s) defined in this paragraph.
52. L² is a bond.
53. L² is selected from: *—[CR⁶R⁷]ₐ—NR⁸-, *—NR⁸—[CR⁶R⁷]ₐ—, *—[CR⁶R⁷]ₐ—O—, *—O—[CR⁶R⁷]ₐ—, *—C(O)—[CR⁶R⁷]ₐ—, *—[CR⁶R⁷]ₐ—C(O)—, *—S(O)ₓ—[CR⁶R⁷]ₐ—, *—[CR⁶R⁷]ₐ—S(O)ₓ— and —[CR⁶R⁷]ₐ—, wherein * indicates the bond to Ring A.
54. L² is selected from: *—[CR⁶H]ₐ—NR⁸—, *—NR⁸—[CR⁶H]ₐ—, *—[CR⁶H]ₐ—O—, *—O—[CR⁶H]ₐ—, *—C(O)—[CR⁶H]ₐ—, *—[CR⁶H]ₐ—C(O)—, *—S(O)ₓ—[CR⁶H]ₐ—, *—[CR⁶H]ₐ—S(O)ₓ— and —[CR⁶H]ₐ—, wherein * indicates the bond to Ring A.
55. L² is selected from: *—[CH₂]ₐ—NR⁸—, *—NR⁸—[CH₂]ₐ—, *—[CH₂]ₐ—O—, *—O—[CH₂]ₐ—, *—C(O)—[CH₂]ₐ—, *—[CH₂]ₐ—C(O)—, *—S(O)ₓ—[CH₂]ₐ—, *—[CH₂]ₐ—S(O)ₓ— and —[CH₂]ₐ—, wherein * indicates the bond to Ring A.
56. L² is selected from: *—[CR⁶R⁷]ₐ—NR⁸—, *—NR⁸—[CR⁶R⁷]ₐ—. Thus it may be that L² is selected from: *—(CH₂)₂—NR⁸—, *—CH₂—NR⁸—, *—NR⁸—(CH₂)₂— and *—NR⁸—CH₂—, wherein R⁸ is selected from H and methyl, wherein * indicates the bond to Ring A. It may be that L² is —NR⁸—, for example-NH—.
57. L² is selected from: *—CH₂—NH— and *—NH—CH₂—, wherein * indicates the bond to Ring A. Thus is may be that L² is *—NH—CH₂—.
58. L² is selected from: *—[CR⁶R⁷]ₐ—O—, *—O—[CR⁶R⁷]ₐ—. Thus it may be that L² is selected from: *—(CH₂)₂—O—, *—CH₂—O—, *—O—(CH₂)₂— and *—O—CH₂—, wherein * indicates the bond to Ring A. It may be that L² is selected from *—O—(CH₂)₂— and *—O—CH₂—. It may be that L² is O.
59. L² is selected from: *—CH₂—O— and *—O—CH₂—, wherein * indicates the bond to Ring A. Thus is may be that L² is *—O—CH₂—. It may be that L² is —CH₂—O—.
60. L² is selected from: *—SO₂—[CH₂]ₐ—, *—[CH₂]ₐ—SO₂—, wherein * indicates the bond to Ring A. Thus it may be that L² is selected from: *—SO₂—(CH₂)₂—, *—SO₂—CH₂—, *—(CH₂)₂—SO₂— and *—CH₂—SO₂—. It may be that L² is selected from: *—SO₂—CH₂— and *—CH₂—SO₂—. It may be that L² is —SO₂—.
61. L² is selected from: *—C(O)—(CH₂)₂—, *—C(O)—CH₂—, *—(CH₂)₂—C(O)— and *—CH₂—C(O)—, wherein* indicates the bond to Ring A. Thus it may be that L² is selected from: *—C(O)—CH₂— and *—CH₂—C(O)—. It may be that L² is —C(O)—.
62. L² is —[CR⁶H]ₐ—. Thus it may be that L² is selected from: —CH₂—, —(CH₂)₂—, —C(Me)HCH₂—, —C(Me)₂CH₂—, —CH₂C(Me)H—, —CH₂C(Me)₂- and —(CH₂)₂—. It may be that L² is selected from: —CH₂—, and —(CH₂)₂—. It may be that L² is —CH₂—.
63. L² is selected from: *—(CH₂)₂—O—, *—CH₂—O—, *—O—(CH₂)₂—, *—O—CH₂—, *—(CH₂)₂—NH—, *—CH₂—NH—, *—NH—(CH₂)₂—, *—NH—CH₂—, —CH₂— and —(CH₂)₂—, wherein * indicates the bond to Ring A.
64. L² is selected from: *—CH₂—O— and *—CH₂—O—, —CH₂— and —(CH₂)₂— wherein * indicates the bond to Ring A. Thus it may be that L² is selected from: *—O—CH₂— and —CH₂—.
65. Q² is selected from: C₃₋₆ cycloalkyl, 4- to 12-membered heterocyclyl, phenyl and 5- to 10-membered heteroaryl, wherein said C₃₋₆ cycloalkyl and 4- to 12-membered heterocyclyl is optionally substituted by one or more R⁹, and said phenyl and 5- to 10-membered heteroaryl is optionally substituted by one or more R¹⁰. Thus it may be that Q² is as defined in this paragraph and L² is as defined in any one of 52 to 64.
66. Q² is selected from: phenyl and 5- to 10-membered heteroaryl, wherein said phenyl and 5- to 10-membered heteroaryl is optionally substituted by one or more R¹⁰. Thus it may be that Q² is as defined in this paragraph and L² is as defined in any one of 52 to 64.
67. Q² is phenyl optionally substituted by one or more R¹⁰. Thus it may be that Q² is as defined in this paragraph and L² is as defined in any one of 52 to 64.
68. Q² is selected from: a 5- or 6-membered heteroaryl containing 1 to 4 ring heteroatoms selected from: O, S and N, wherein said 5- or 6-membered heteroaryl is optionally substituted by one or more R¹⁰. Thus it may be that Q² is as defined in this paragraph and L² is as defined in any one of 52 to 64.
69. Q² is selected from: a 5-membered heteroaryl optionally substituted by one or more R¹⁰. Thus it may be that Q² is selected from: a 5-membered heteroaryl containing 1 ring nitrogen heteroatom and optionally 1 to 3 additional ring heteroatoms selected from: O, S and N, wherein said 5-membered heteroaryl is optionally substituted by one or more R¹⁰. Thus it may be that Q² is as defined in this paragraph and L² is as defined in any one of 52 to 64.
70. Q² is selected from: pyrrolyl, pyrazolyl, furanyl, thienyl, imidazolyl, furazanyl, oxazolyl, isoxazolyl, oxadiazolyl, oxatriazolyl, thiazolyl, isothiazolyl, thiadiazolyl, triazolyl and tetrazolyl, wherein said Q² is optionally substituted by one or more R¹⁰. Thus it may be that Q² is as defined in this paragraph and L² is as defined in any one of 52 to 64.
71. Q² is selected from: pyrrolyl, pyrazolyl, furanyl, imidazolyl, oxazolyl, isoxazolyl, oxadiazolyl, thiazolyl, isothiazolyl and triazolyl and tetrazolyl, wherein said Q² is optionally substituted by one or more R¹⁰. Thus it may be that Q² is as defined in this paragraph and L² is as defined in any one of 52 to 64.
72. Q² is selected from: a 6-membered heteroaryl optionally substituted by one or more R¹⁰. Thus it may be that Q² is selected from: a 6-membered heteroaryl containing 1 ring nitrogen heteroatom and optionally 1 or 2 additional ring heteroatoms selected from: O, S and N, wherein said 6-membered heteroaryl is optionally substituted by one or more R¹⁰. Thus it may be that Q² is as defined in this paragraph and L² is as defined in any one of 52 to 64.
73. Q² is selected from: pyridyl, pyrazinyl, pyridazinyl, pyrimidinyl and triazinyl, wherein Q² is optionally substituted by one or more R¹⁰. Thus it may be that Q² is as defined in this paragraph and L² is as defined in any one of 52 to 64.
74. Q² is selected from: a 9- or 10-membered bicyclic heteroaryl optionally substituted by one or more R¹⁰. Thus it may be that Q² is selected from: a 9- or 10-membered bicyclic heteroaryl containing 1 ring nitrogen heteroatom and optionally 1 or 2 additional ring heteroatoms selected from: O, S and N, wherein said 9- or 10-membered heteroaryl is optionally substituted by one or more R¹⁰. Thus it may be that Q² is as defined in this paragraph and L² is as defined in any one of 52 to 64.
75. Q² is selected from: a 4- to 12-membered saturated or partially saturated heterocyclyl containing 1 to 4 heteroatoms selected from O, S and N, wherein Q² is optionally substituted by one or more R⁹. Thus it may be that is selected from: a 4- to 12-membered saturated or partially saturated heterocyclyl containing 1 to 4 heteroatoms selected from O, S and N, wherein Q² is optionally substituted by one or more R⁹. Thus it may be that Q² is as defined in this paragraph and L² is as defined in any one of 52 to 64.
76. Q² is selected from: a 4- to 7-membered saturated or partially saturated heterocyclyl containing 1 to 4 heteroatoms selected from O, S and N, wherein Q² is optionally substituted by one or more R⁹. Thus it may be that Q² is selected from: a 4- to 7-membered saturated heterocyclyl containing 1 to 4 heteroatoms selected from O, S and N, wherein Q² is optionally substituted by one or more R⁹. Thus it may be that Q² is as defined in this paragraph and L² is as defined in any one of 52 to 64.
77. Q² is selected from: a 4- to 7-membered saturated or partially saturated heterocyclyl containing 1 ring nitrogen heteroatom and 1 to 3 additional heteroatoms selected from O, S and N, wherein Q² is optionally substituted by one or more R⁹. Thus it may be that Q² is selected from: a 4- to 7-membered saturated heterocyclyl containing 1 ring nitrogen heteroatom and 1 to 3 additional heteroatoms selected from O, S and N, wherein Q² is optionally substituted by one or more R⁹. Thus it may be that Q² is as defined in this paragraph and L² is as defined in any one of 52 to 64.
78. Q² is selected from: azetidinyl, pyrrolidinyl, imidazolidinyl, piperidinyl, piperazinyl, morpholinyl, thiomorpholinyl, thiomorpholinyl 1,1-dioxide, homopiperidinyl and homopiperazinyl, wherein Q² is optionally substituted by one or more R⁹. Thus it may be that Q² is as defined in this paragraph and L² is as defined in any one of 52 to 64.
79. Q² is selected from: oxetanyl, tetrahydrofuranyl, dioxanyl and tetrahydropyranyl, wherein Q² is optionally substituted by one or more R⁹. Thus it may be that Q² is as defined in this paragraph and L² is as defined in any one of 52 to 64.
80. Q² is selected from: a 7- to 12-membered saturated or partially saturated bicyclic or spirocyclic heterocyclyl containing 1 to 4 heteroatoms selected from O, S and N, wherein Q² is optionally substituted by one or more R⁹. Thus it may be that Q² is selected from: a 7- to 12-membered saturated bicyclic or spirocyclic heterocyclyl containing 1 to 4 heteroatoms selected from O, S and N, wherein Q² is optionally substituted by one or more R⁹. Thus it may be that Q² is as defined in this paragraph and L² is as defined in any one of 52 to 64.
81. Q² is selected from:

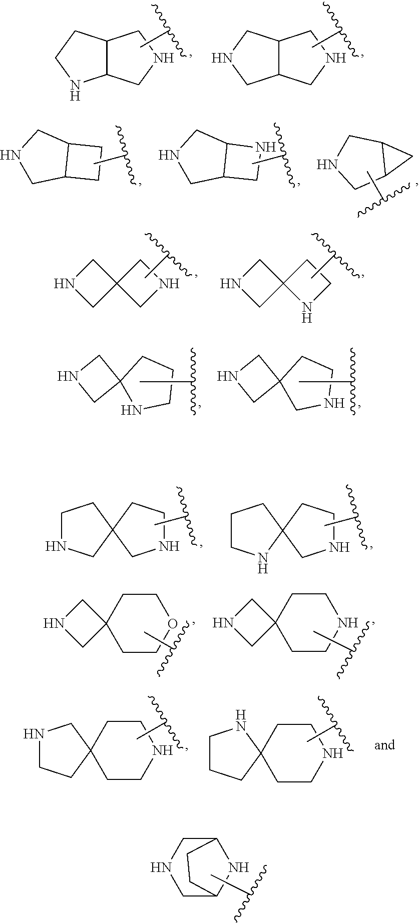

wherein each Q² is optionally substituted by one or more R⁹ and wherein Q² is bonded to L² by any available ring carbon or ring nitrogen atom. Thus it may be that Q² is as defined in this paragraph and L² is as defined in any one of 52 to 64.
82. Ring A is substituted by one R³ group selected from:—NR^{a2}R^{b2}, —C₁₋₄ alkyl-NR^{a2}R^{b2} and -L²-Q², wherein Q² is selected from azetidinyl, pyrrolidinyl, imidazolidinyl, piperidinyl, piperazinyl, morpholinyl, homopiperidinyl, homopiperazinyl and a group defined in 81, wherein Q² is optionally substituted by one or more R⁹; and wherein Ring A is optionally further substituted by one or two R³ independently selected from: halo, —CN, C₁₋₄ alkyl, C₁₋₄ haloalkyl, —OR⁵ and —NR^{a1}R⁵.

Thus it may be that Q² is as defined in this paragraph and L² is as defined in any one of 52 to 64. It may be that Ring A is selected from any one of 20 to 32, wherein Ring A is substituted with the R³ group(s) defined in this paragraph; and wherein L² is selected from: *—[CH₂]ₐ—NR⁸—, *—NR⁸—[CH₂]ₐ—, *—[CH₂]ₐ—O—, *—O—[CH₂]ₐ—, *—C(O)—[CH₂]ₐ—, *—[CH₂]ₐ—C(O)—, *—SO₂—[CH₂]ₐ—, *—[CH₂]ₐ—SO₂— and —[CH₂]ₐ—, wherein * indicates the bond to Ring A (for example L² is selected from: *—O—[CH₂]ₐ—, *—NH—[CH₂]ₐ— and —[CH₂]ₐ—).
83. Each R⁹ and R¹³ is independently selected from: halo, ═O, —CN, C₁₋₄ alkyl, C₁₋₄ haloalkyl, —OR^{a4}, —S(O)₂R^{a4}, —NR^{a4}R^{b4}, —C(O)R^{a4}, —C(O)OR^{a4}, —C(O)NR^{a4}R^{b4}, and —SO₂NR^{a4}R^{b4;}
   wherein said C₁₋₄ alkyl is optionally substituted by 1 or 2 substituents selected from: halo, —CN, —OR^{a5}, —NR^{a5}R^{b5} and —SO₂R^{a5}.
84. Each R⁹ and R¹³ is independently selected from: halo, ═O, C₁₋₄ alkyl, C₁₋₄ haloalkyl, —OR^{a4}, —NR^{a4}R^{b4} and —C(O)R^{a4}, wherein said C₁₋₄ alkyl is optionally substituted by —OR^{a5}.
85. Each R⁹ and R¹³ is independently selected from: halo, ═O, C₁₋₄ alkyl and C₁₋₄ haloalkyl, wherein said C₁₋₄ alkyl is optionally substituted by —OR^{a5}.
86. Each R¹⁰ and R¹⁴ is independently selected from: halo, —CN, C₁₋₄ alkyl, —C₁₋₄ alkyl-OR^{a7}, C₁₋₄ haloalkyl, —OR^{a6}, —S(O)₂R^{a6} and —NR^{a6}R^{b6}, wherein said C₁₋₄ alkyl is optionally substituted by —OR^{a7}.
87. Each R¹⁰ and R¹⁴ is independently selected from: halo, —CN, C₁₋₄ alkyl, C₁₋₄ haloalkyl and —OR^{a6}, wherein said C₁₋₄ alkyl is optionally substituted by —OR^{a7}.
88. -L²-Q² is selected from:

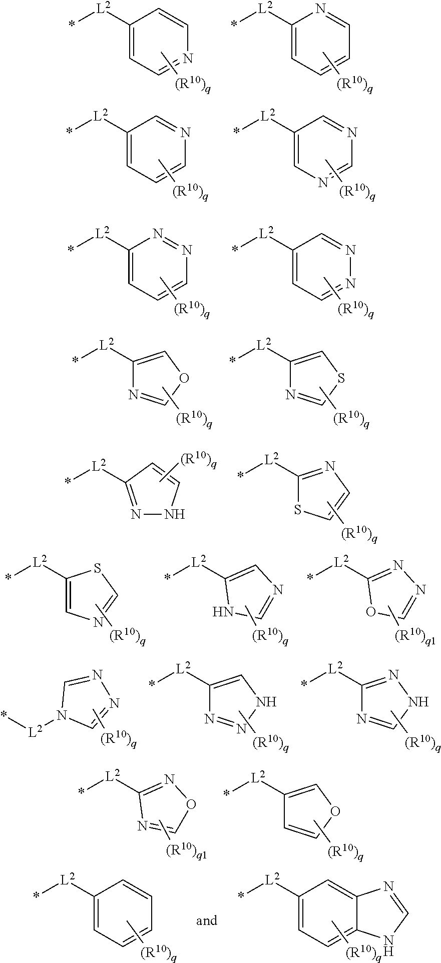

wherein q is 0, 1 or 2; q1 is 0 or 1;
L² is as defined in any one of 52 to 64; and
*indicates the point of attachment to Ring A.

Suitably L² is selected from: *—(CH₂)₂—O—, *—CH₂—O—, *—O—(CH₂)₂—, *—O—CH₂—, —O—, *—(CH₂)₂—NH—, *—CH₂—NH—, *—NH—(CH₂)₂—, *—NH—CH₂—, —NH—, —CH₂— and —(CH₂)₂—. Preferably L² is selected from: *—(CH₂)₂—O—, *—CH₂—O—, *—O—(CH₂)₂—, and *—O—CH₂—. More preferably L² is *—O—CH₂—.
89. -L²-Q² is selected from:

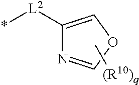

wherein
*indicates the point of attachment to Ring A; and q is 0, 1 or 2.

Suitably L² is selected from: *—(CH₂)₂—O—, *—CH₂—O—, *—O—(CH₂)₂—, *—O—CH₂—, —O—, *—(CH₂)₂—NH—, *—CH₂—NH—, *—NH—(CH₂)₂—, *—NH—CH₂—, —NH—, —CH₂— and —(CH₂)₂—. Preferably L² is selected from: *—(CH₂)₂—O—, *—CH₂—O—, *—O—(CH₂)₂—, and *—O—CH₂—. More preferably L² is *—O—CH₂—. Thus it may be that -L²-Q² is selected from:

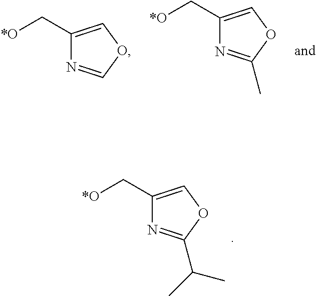

90. -L²-Q² is selected from:

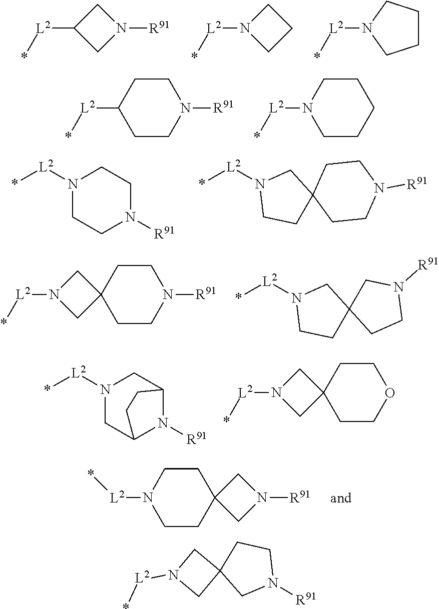

wherein L² is as defined in any one of 52 to 64, provided that when Q² is bonded to L² via a ring nitrogen, Q² is not bonded to a nitrogen or oxygen atom in L²;
R⁹¹ is selected from: H, C₁₋₄ alkyl and C₁₋₄ haloalkyl; and wherein Q² is optionally substituted by one or two substituents selected from: halo, ═O, C₁₋₄ alkyl and C₁₋₄ haloalkyl;
*indicates the point of attachment to Ring A.

Suitably L² is selected from: *—O—(CH₂)₂—, *—NH—(CH₂)₂—, —CH₂—, —(CH₂)₂—, —C(O)—, *—C(O)—(CH₂)₂—, *—C(O)—CH₂—, *—(CH₂)₂—C(O)—, *—CH₂—C(O)—, —SO₂—, *—SO₂—(CH₂)₂—, *—SO₂—CH₂—, *—(CH₂)₂—SO₂— and *—CH₂—SO₂—. Preferably L² is selected from: —CH₂—, —(CH₂)₂— and —C(O)—. More preferably L² is —CH₂— or —(CH₂)₂—. Still more preferably L² is —CH₂—.
91. -L²-Q² is selected from:

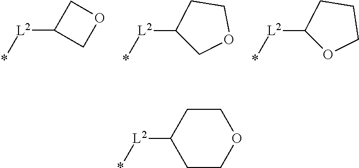

wherein L² is as defined in any one of 52 to 64;
*indicates the point of attachment to Ring A; and
wherein Q² is optionally substituted by one or two substituents selected from: halo, ═O, C₁₋₄ alkyl and C₁₋₄ haloalkyl.

Suitably L² is selected from: *—(CH₂)₂—O—, *—CH₂—O—, *—O—(CH₂)₂—, *—O—CH₂—, *—(CH₂)₂—NH—, *—CH₂—NH—, *—NH—(CH₂)₂—, *—NH—CH₂—, —CH₂— and —(CH₂)₂—. Preferably L² is selected from: *—(CH₂)₂—O—, *—CH₂—O—, *—O—(CH₂)₂—, and *—O—CH₂—. More preferably L² is *—O—CH₂—.
92.-L²-Q² is selected from:

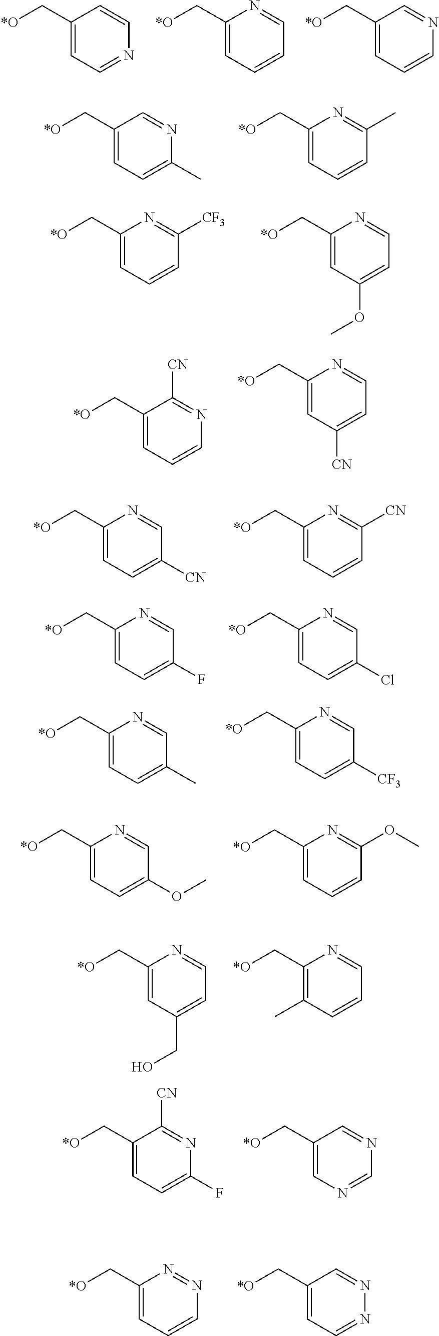

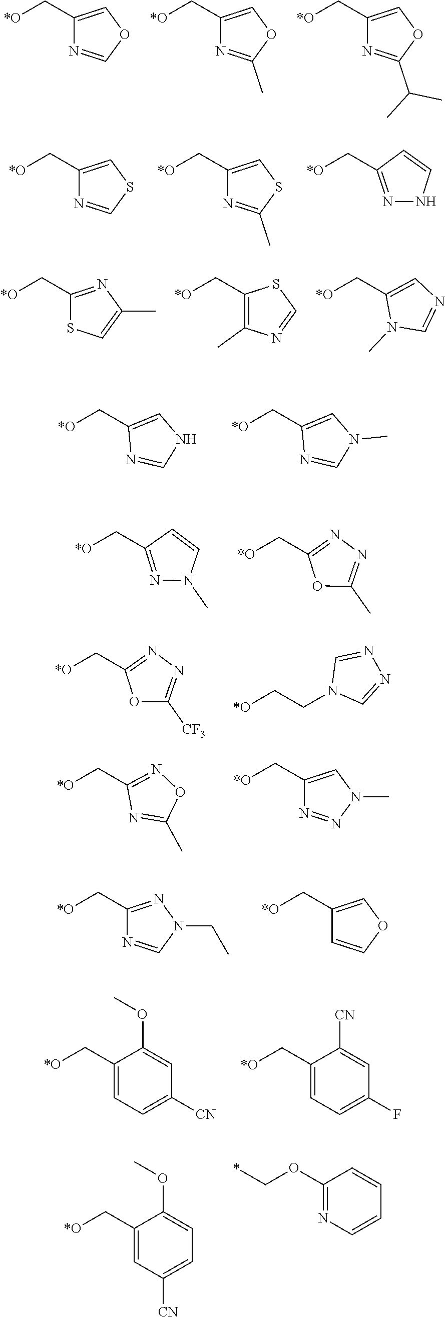

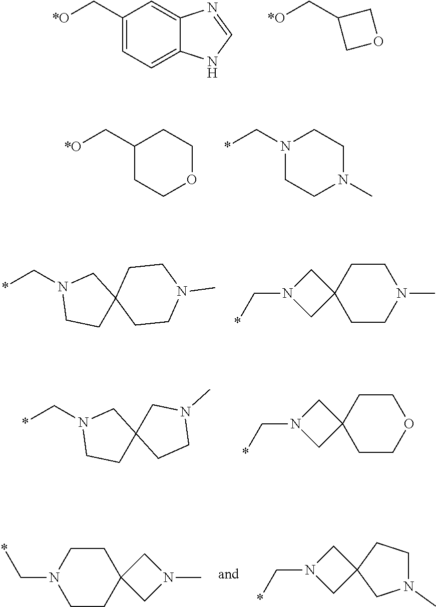

wherein *indicates the point of attachment to Ring A.
93. -L²-Q² is selected from:

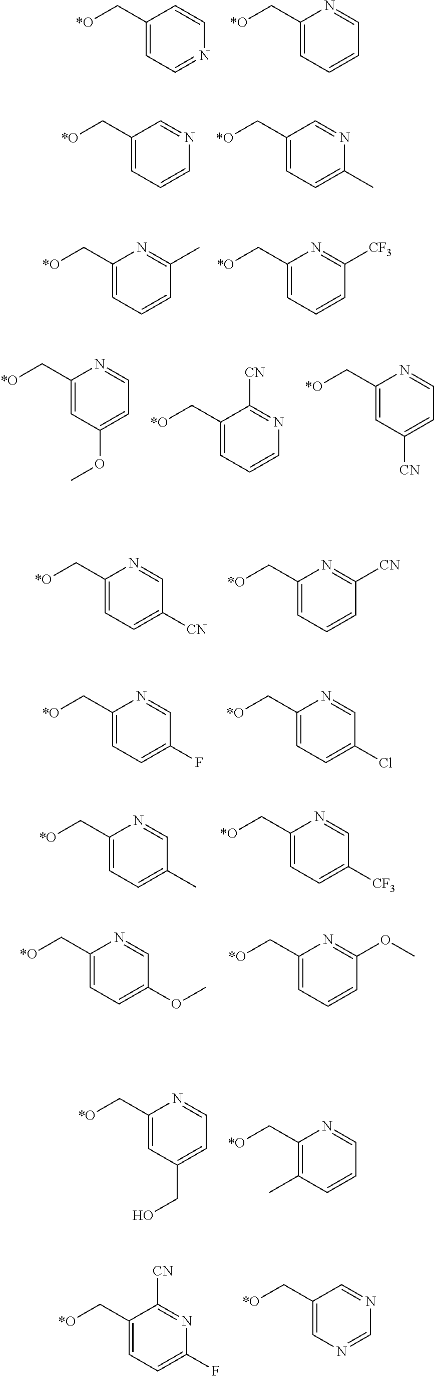

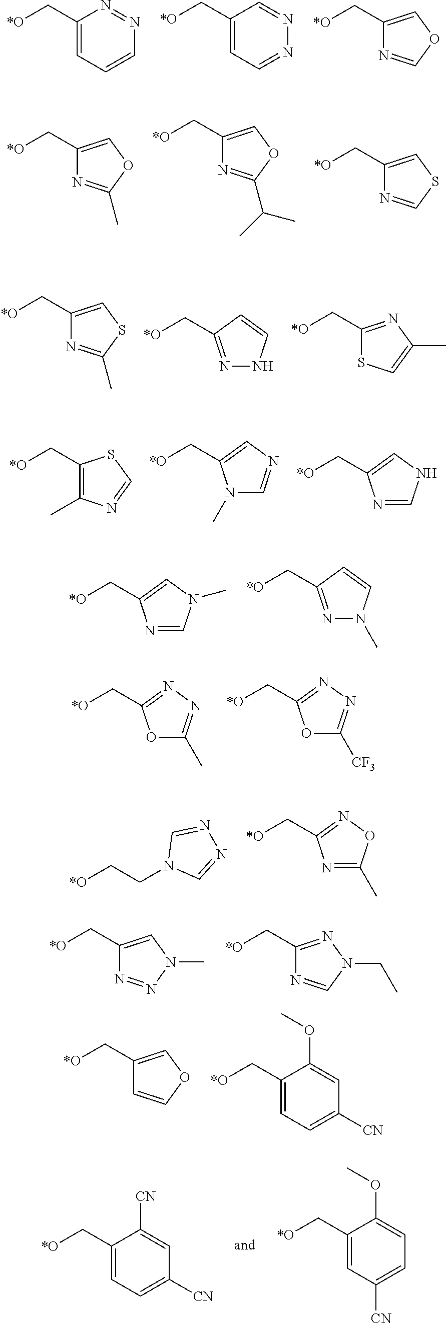

wherein *indicates the point of attachment to Ring A.
94. Ring A is substituted with one R³ selected from -L²-Q² as defined in any one of 88 to 93 and Ring A is optionally further substituted by one or two (preferably one) R³ selected from: halo, —CN, C₁₋₄ alkyl, —C₁₋₄ alkyl-OR^{a2}, C₁₋₄ haloalkyl, —O—C₁₋₄ alkyl, —O—C₁₋₄ haloalkyl and —NR^{a2}R^{b2}. It may be that Ring A is as defined in 20, wherein Ring A is substituted with the R³ group(s) defined in this paragraph. It may be that Ring A is as defined in 21, wherein Ring A is substituted with the R³ group(s) defined in this paragraph. It may be that Ring A is as defined in 22, wherein Ring A is substituted with the R³ group(s) defined in this paragraph. It may be that Ring A is as defined in 23, wherein Ring A is substituted with the R³ group(s) defined in this paragraph. It may be that Ring A is as defined in 24, wherein Ring A is substituted with the R³ group(s) defined in this paragraph. It may be that Ring A is as defined in 25, wherein Ring A is substituted with the R³ group(s) defined in this paragraph. It may be that Ring A is as defined in 26, wherein Ring A is substituted with the R³ group(s) defined in this paragraph. It may be that Ring A is as defined in 27, wherein Ring A is substituted with the R³ group(s) defined in this paragraph. It may be that Ring A is as defined in 28, wherein Ring A is substituted with the R³ group(s) defined in this paragraph. It may be that Ring A is as defined in 29, wherein Ring A is substituted with the R³ group(s) defined in this paragraph. It may be that Ring A is as defined in 30, wherein Ring A is substituted with the R³ group(s) defined in this paragraph. It may be that Ring A is as defined in 31, wherein Ring A is substituted with the R³ group(s) defined in this paragraph. It may be that Ring A is as defined in 32, wherein Ring A is substituted with the R³ group(s) defined in this paragraph.
95. Ring A is selected from:

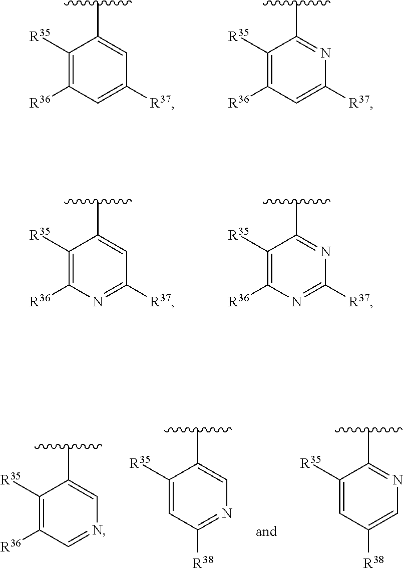

R³⁵, R³⁶ and R³⁸ are independently selected from: H, halo, C₁₋₄ alkyl, —C₁₋₄ alkyl-OR^{a2}, C₁₋₄ haloalkyl, —O—C₁₋₄ alkyl and —O—C₁₋₄ haloalkyl;
R³⁷ is C₁₋₄ alkyl, —C₁₋₄ alkyl-OR^{a2}, C₁₋₄ haloalkyl, —O—C₁₋₄ alkyl, —O—C₁₋₄ alkyl-OR^{a3}, —O—C₁₋₄ haloalkyl and -L²-Q²;
   L² is selected from: —(CH₂)₂—, —CH₂—, —(CH₂)₂—O—*, —CH₂—O—*, —O—(CH₂)₂—*, —O—CH₂—*, —(CH₂)₂—NR⁸—*, —CH₂—NR⁸—*, —NR⁸—(CH₂)₂—* and —NR⁸—CH₂—*, wherein R⁸ is selected from H and methyl, and * indicates the bond to Q²; and
   Q² is selected from any one of 65 to 81.

It may be that R³⁵ and R³⁶ are not both H.

It may be that R³⁵ and R³⁸ are not both H.

It may be that R³⁵ is selected from: halo, C₁₋₄ alkyl, —C₁₋₄ alkyl-OR^{a2}, C₁₋₄ haloalkyl, —O—C₁₋₄ alkyl and —O—C₁₋₄ haloalkyl, and R³⁶ is H.

It may be that R³⁶ is selected from; halo, C₁₋₄ alkyl, —C₁₋₄ alkyl-OR^{a2}, C₁₋₄ haloalkyl, —O—C₁₋₄ alkyl and —O—C₁₋₄ haloalkyl, and R³⁵ is H.

It may be that R³⁵ is selected from: —C₁₋₄ alkyl-OR^{a2}, C₁₋₄ haloalkyl, —O—C₁₋₄ alkyl and —O—C₁₋₄ haloalkyl, and R³⁶ is selected from: H, halo and C₁₋₄ alkyl.

It may be that R³⁶ is selected from: —C₁₋₄ alkyl-OR^{a2}, C₁₋₄ haloalkyl, —O—C₁₋₄ alkyl and —O—C₁₋₄ haloalkyl, and R³⁵ is selected from: H, halo and C₁₋₄ alkyl.

It may be that R³⁵ and R³⁶ are independently selected from H, halo, C₁₋₄ alkyl, —C₁₋₄ alkyl-OH, —C₁₋₄ alkyl-O—C₁₋₃ alkyl, C₁₋₄ haloalkyl, —O—C₁₋₄ alkyl and —O—C₁₋₄ haloalkyl, provided that R³⁵ and R³⁶ are not both H.

It may be that R³⁵ and R³⁶ are independently selected from H, halo, C₁₋₄ alkyl, —C₁₋₄ alkyl-OH, —C₁₋₄ alkyl-O—C₁₋₃ alkyl, —O—C₁₋₄ alkyl, provided that R³⁵ and R³⁶ are not both H.

It may be that R³⁵ and R³⁶ are independently selected from H, halo and C₁₋₄ alkyl, provided that R³⁵ and R³⁶ are not both H.

It may be that R³⁵ is halo (e.g. F) and R³⁶ is selected from: H, halo, C₁₋₄ alkyl, —C₁₋₄ alkyl-OH, —C₁₋₄ alkyl-O—C₁₋₃ alkyl, C₁₋₄ haloalkyl, —O—C₁₋₄ alkyl and —O—C₁₋₄ haloalkyl.

It may be that R³⁶ is halo (e.g. F) and R³⁵ is selected from: H, halo, C₁₋₄ alkyl, —C₁₋₄ alkyl-OH, —C₁₋₄ alkyl-O—C₁₋₃ alkyl, C₁₋₄ haloalkyl, —O—C₁₋₄ alkyl and —O—C₁₋₄ haloalkyl.

It may be that R³⁵ is halo (e.g. F) and R³⁶ is H.

It may be that R³⁶ is halo (e.g. F) and R³⁵ is H.

It may be that R³⁵ is selected from: halo, C₁₋₄ alkyl, —C₁₋₄ alkyl-OR^{a2}, C₁₋₄ haloalkyl, —O—C₁₋₄ alkyl and —O—C₁₋₄ haloalkyl, and R³⁸ is H.

It may be that R³⁸ is selected from; halo, C₁₋₄ alkyl, —C₁₋₄ alkyl-OR^{a2}, C₁₋₄ haloalkyl, —O—C₁₋₄ alkyl and —O—C₁₋₄ haloalkyl, and R³⁵ is H.

It may be that R³⁵ is selected from: —C₁₋₄ alkyl-OR^{a2}, C₁₋₄ haloalkyl, —O—C₁₋₄ alkyl and —O—C₁₋₄ haloalkyl, and R³⁸ is selected from: H, halo, C₁₋₄ alkyl and —C₁₋₄ alkyl-OR^{a2}.

It may be that R³⁸ is selected from: C₁₋₄ alkyl, —C₁₋₄ alkyl-OR^{a2}, C₁₋₄ haloalkyl, —O—C₁₋₄ alkyl and —O—C₁₋₄ haloalkyl, and R³⁵ is selected from: H, halo, C₁₋₄ alkyl and —O—C₁₋₄ alkyl.

It may be that R³⁸ is selected from: C₁₋₄ alkyl and —C₁₋₄ alkyl-OR^{a2}, and R³⁵ is selected from:

H and —O—C₁₋₄ alkyl.

It may be that R³⁸ is selected from: C₁₋₄ alkyl and —C₁₋₄ alkyl-OR^{a2}, and R³⁵ is-O—C₁₋₄ alkyl.

It may be that R³⁸ is C₁₋₄ alkyl, and R³⁵ is selected from: H and —O—C₁₋₄ alkyl.

It may be that R³⁸ is C₁₋₄ alkyl, and R³⁵ is-O—C₁₋₄ alkyl.

It may be that R³⁷ is selected from: C₁₋₄ alkyl, —O—C₁₋₄ alkyl, —C₁₋₄ alkyl-OH, —C₁₋₄ alkyl-O—C₁₋₃ alkyl and -L²-Q²;
L² is selected from: —(CH₂)₂—O—*, —CH₂—O—*, —O—(CH₂)₂—* and —O—CH₂—*, wherein * indicates the bond to Q²;
Q² is selected from any one of 65 to 81; and R³⁵ and R³⁶ independently have any of the values defined in this paragraph.

It may be that R³⁷ is selected from: C₁₋₄ alkyl, —O—C₁₋₄ alkyl, and -L²-Q², wherein -L²-Q² is as defined in any one of 88 to 93; and R³⁵ and R³⁶ independently have any of the values defined in this paragraph.

It may be that R³⁷ is -L²-Q², wherein -L²-Q² is as defined in any one of 88 to 93; and R³⁵ and R³⁶ independently have any of the values defined in this paragraph.
96. Ring A is

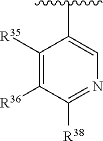

wherein R³⁵ is selected from: —O—C₁₋₄ alkyl, —C₁₋₄ alkyl-OH and —C₁₋₄ alkyl-O—C₁₋₃ alkyl; and
R³⁶ and R³⁸ are each independently selected from: H, halo, C₁₋₄ alkyl, —O—C₁₋₄ alkyl, —O—C₁₋₄ haloalkyl —C₁₋₄ alkyl-OH, —C₁₋₄ alkyl-O—C₁₋₃ alkyl and C₁₋₄ haloalkyl.

Thus it may be that Ring A is:

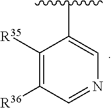

Thus it may be that Ring A is:

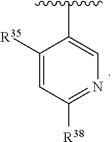

It may be that R³⁵ is —O—C₁₋₄ alkyl. It may be that R³⁵ is selected from: —OMe, —CH₂—OH and —CH₂—OMe. It may be that R³⁵ is —OMe.

It may be that R³⁵ is —O—C₁₋₄ alkyl (e.g. —OMe) and R³⁶ is selected from: H, halo, C₁₋₄ alkyl and —C₁₋₄ haloalkyl.

It may be that R³⁵ is —O—C₁₋₄ alkyl (e.g. —OMe) and R³⁸ is selected from: H, halo, C₁₋₄ alkyl, —C₁₋₄ alkyl-OH, —C₁₋₄ alkyl-O—C₁₋₃ alkyl and —C₁₋₄ haloalkyl.

It may be that R³⁵ is selected from: —O—C₁₋₄ alkyl (e.g. —OMe), and R³⁸ is selected from: halo, C₁₋₄ alkyl, —C₁₋₄ alkyl-OH, —C₁₋₄ alkyl-O—C₁₋₃ alkyl and —C₁₋₄ haloalkyl.

It may be that R³⁵ is —O—C₁₋₄ alkyl (e.g. —OMe) and R³⁸ is selected from: halo, C₁₋₄ alkyl, —C₁₋₄ alkyl-OH and —C₁₋₄ haloalkyl.

It may be that R³⁵ is —O—C₁₋₄ alkyl (e.g. —OMe) and R³⁸ is C₁₋₄ alkyl (e.g. Me).

It may be that R³⁵ is F or methoxy, R³⁶ is H, and R³⁸ is methyl.
97. L¹ is a bond.
98. L¹ is O.
99. L¹ is NH.
100. R⁴ is selected from: H, C₁₋₆ alkyl, C₁₋₆ haloalkyl and -L³-Q³,
   wherein said C₁₋₆ alkyl, is optionally substituted by one or more substituents selected from: halo, —CN, —OR^{a8}, —SO₂R^{a8} and —NR³⁸R^{b8},
   L³ is a bond or —[CR¹¹R¹²]_{b}—,
   each R¹¹ and R¹² is independently selected from: H, halo, C₁₋₃ alkyl and C₁₋₃ haloalkyl,
   or two R¹¹ and R¹² attached to the same carbon atom form a C₃₋₆ cycloalkyl,
   b is 1 to 4,
   Q³ is selected from: C₃₋₆ cycloalkyl, 4- to 7-membered heterocyclyl, phenyl and 5- or 6-membered heteroaryl,
   wherein said C₃₋₆ cycloalkyl and 4- to 7-membered heterocyclyl is optionally substituted by one or more R¹³, and
   wherein said phenyl and 5- or 6-membered heteroaryl is optionally substituted by one or more R¹⁴.
101. R⁴ is selected from: C₁₋₆ alkyl, C₁₋₆ haloalkyl and -L³-Q³,
   wherein said C₁₋₆ alkyl, is optionally substituted by one or more substituents selected from: —CN, —OR^{a8}, —SO₂R^{a8} and —NR³⁸R^{b8}.
   L³ is a bond or —[CR¹¹R¹²]_{b}—,
   each R¹¹ and R¹² is independently selected from: H and C₁₋₃ alkyl,
   or two R¹¹ and R¹² attached to the same carbon atom form a C₃₋₆ cycloalkyl,
   b is 1 to 4,
   Q³ is selected from: C₃₋₆ cycloalkyl, 4- to 7-membered heterocyclyl containing 1 to 3 ring heteroatoms selected from O, S and N, phenyl and 5- or 6-membered heteroaryl containing 1 or 2 ring nitrogen atoms,
   wherein said C₃₋₆ cycloalkyl and 4- to 7-membered heterocyclyl is optionally substituted by one or more R¹³, and
   wherein said phenyl and 5- or 6-membered heteroaryl is optionally substituted by one or more R¹⁴.
102. R⁴ is selected from: C₁₋₆ alkyl and C₁₋₆ haloalkyl, wherein said C₁₋₆ alkyl, is optionally substituted by one or more substituents selected from: —CN, —OR^{a8} and —NR^{a8}R^{b8}.
103. R⁴ is selected from: C₁₋₄ alkyl and C₁₋₄ haloalkyl, wherein said C₁₋₆ alkyl, is optionally substituted by one or more substituents selected from: —CN and —OR^{a8}.
104. R⁴ is -L³-Q³, wherein L³ is a bond or —[CR¹¹R¹²]_{b}—,
   each R¹¹ and R¹² is independently selected from: H and C₁₋₃ alkyl,
   or two R¹¹ and R¹² attached to the same carbon atom form a C₃₋₆ cycloalkyl,
   b is 1 to 4,
   Q³ is selected from: C₃₋₆ cycloalkyl, 4- to 7-membered heterocyclyl containing 1 to 3 ring heteroatoms selected from O, S and N, phenyl and 5- or 6-membered heteroaryl containing 1 to 3 ring heteroatoms selected from O, S and N,
   wherein said C₃₋₆ cycloalkyl and 4- to 7-membered heterocyclyl is optionally substituted by one or more R¹³,
   wherein said phenyl and 5- or 6-membered heteroaryl is optionally substituted by one or more R¹⁴
105. R⁴ is -L³-Q³, wherein L³ is a bond or —[CR¹¹R¹²]_{b}—,
   each R¹¹ and R¹² is independently selected from: H and C₁₋₃ alkyl,
   or two R¹¹ and R¹² attached to the same carbon atom form a C₃₋₆ cycloalkyl,
   b is 1 to 4,
   Q³ is selected from: C₃₋₆ cycloalkyl, 4- to 6-membered heterocyclyl containing 1 to 3 ring heteroatoms selected from O, S and N, phenyl and 5- or 6-membered heteroaryl containing 1 to 3 ring heteroatoms selected from O, S and N,
   wherein said C₃₋₆ cycloalkyl and 4- to 6-membered heterocyclyl is optionally substituted by one or more R¹³,
   wherein said phenyl and 5- or 6-membered heteroaryl is optionally substituted by one or more R¹⁴.
106. R⁴ is -L³-Q³, wherein L³ is a bond or —[CR¹¹R¹²]_{b}—,
   each R¹¹ and R¹² is independently selected from: H and C₁₋₃ alkyl,
   b is 1 to 4, and
   Q³ is C₃₋₆ cycloalkyl optionally substituted by one or more R¹³.
107. R⁴ is -L³-Q³, wherein L³ is a bond or —[CR¹¹R¹²]_{b}—,
   each R¹¹ and R¹² is independently selected from: H and C₁₋₃ alkyl,
   b is 1 to 4,
   Q³ is a 5-membered heteroaryl containing 1 to 3 ring heteroatoms selected from O, S and N, wherein said 5-membered heteroaryl is optionally substituted with one or more R¹⁴. Thus it may be that Q⁵ is selected from: pyrrolyl, imidazolyl, pyrazolyl, oxazolyl, isoxazolyl and triazolyl, each or which is optionally substituted by one or more R¹⁴. For example, it may be that Q³ is isoxazolyl optionally substituted by one or more R¹⁴.
108. R⁴ is -L³-Q³, wherein L³ is a bond or —[CR¹¹R¹²]_{b}—,
   each R¹¹ and R¹² is independently selected from: H and C₁₋₃ alkyl,
   b is 1 to 4, and
   Q³ is selected from oxetanyl, tetrahydrofuranyl and tetrahydropyranyl, wherein Q³ is optionally substituted by one or more R¹³.
109. R⁴ is selected from: C₁₋₆ alkyl, C₁₋₆ haloalkyl, —C₁₋₆ alkyl-CN, —C₁₋₆ alkyl-OR^{a8}, —C₁₋₄ alkyl-C₁₋₆ cycloalkyl, C₁₋₆ cycloalkyl, —C₁₋₄ alkyl-oxetanyl, oxetanyl, —C₁₋₄ alkyl-tetrahydrofuranyl, tetrahydrofuranyl, —C₁₋₄ alkyl-tetrahydropyranyl and —C₁₋₄ alkyl-tetrahydropyranyl, wherein any C₁₋₆ cycloalkyl, oxetanyl, tetrahydrofuranyl and tetrahydropyranyl in R⁴ is optionally substituted by one or two substituents selected from halo, ═O, and C₁₋₄ alkyl.
110. R⁴ is selected from: C₁₋₄ alkyl, C₁₋₄ haloalkyl, —C₁₋₄ alkyl-OR^{a8}, —C₁₋₄ alkyl-C₁₋₆ cycloalkyl, C₁₋₆ cycloalkyl, —C₁₋₄ alkyl-oxetanyl and oxetanyl.
111. R⁴ is selected from:

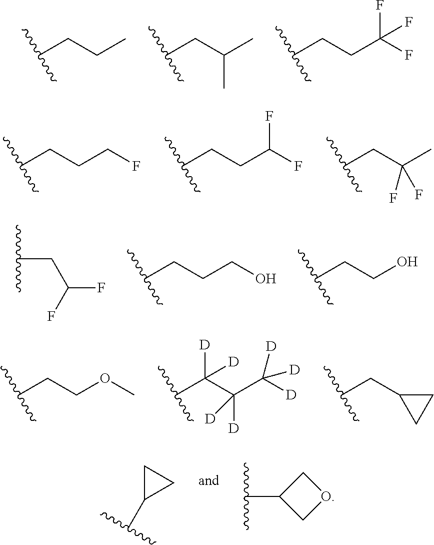

112. R⁴ is C₁₋₄ alkyl.
113. R⁴ is —CH₂CH₂CH₃.
114. Ring A is selected from:

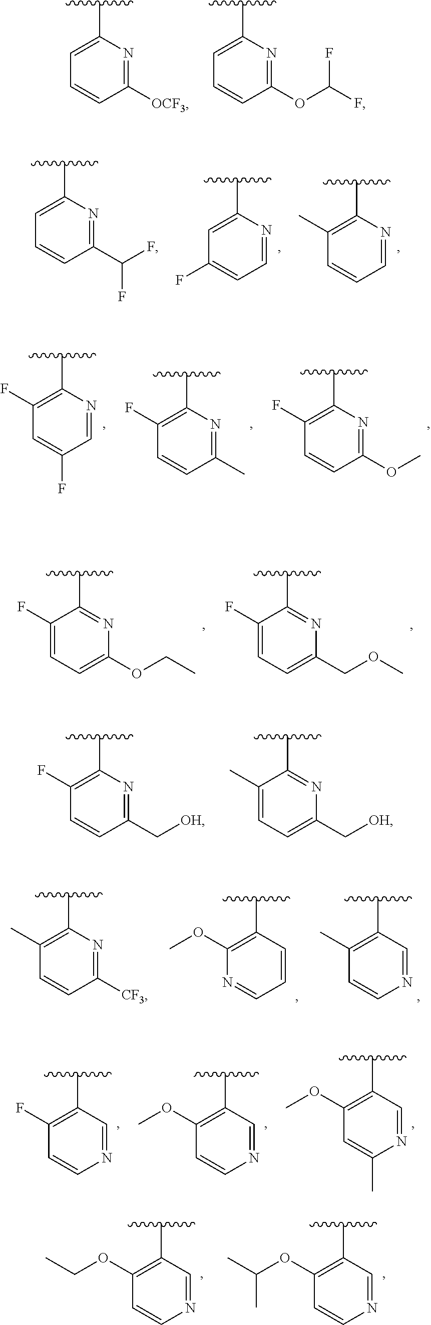

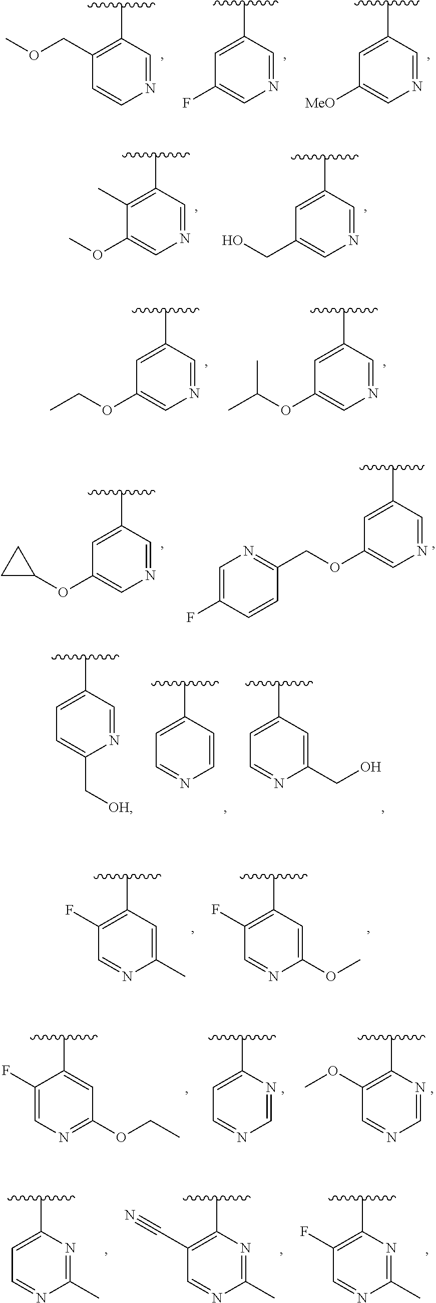

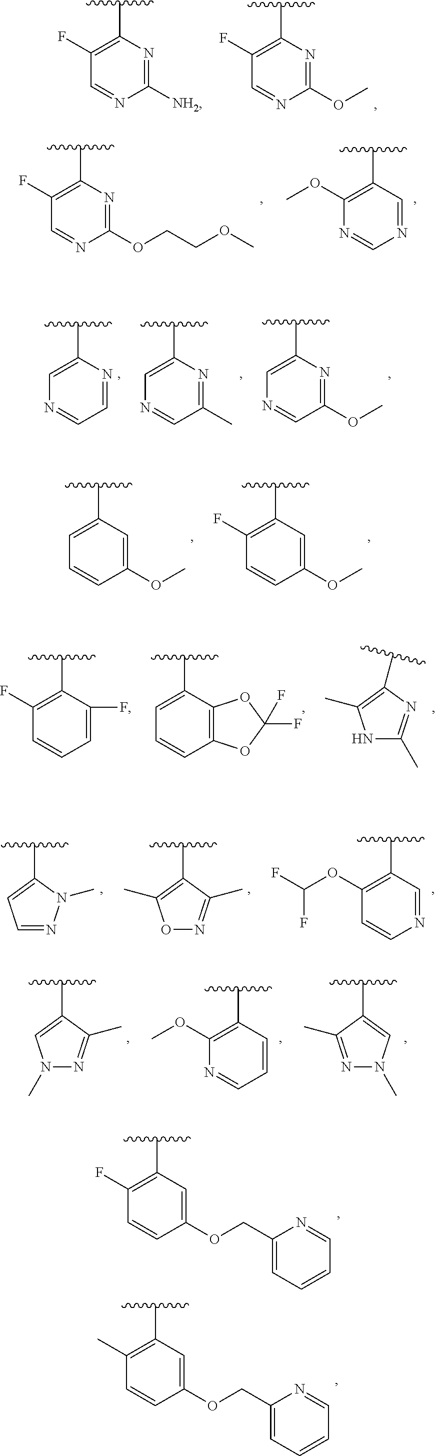

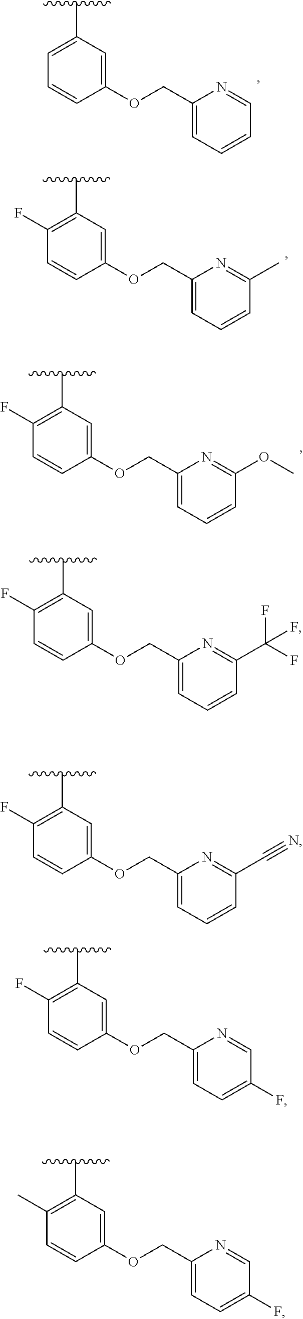

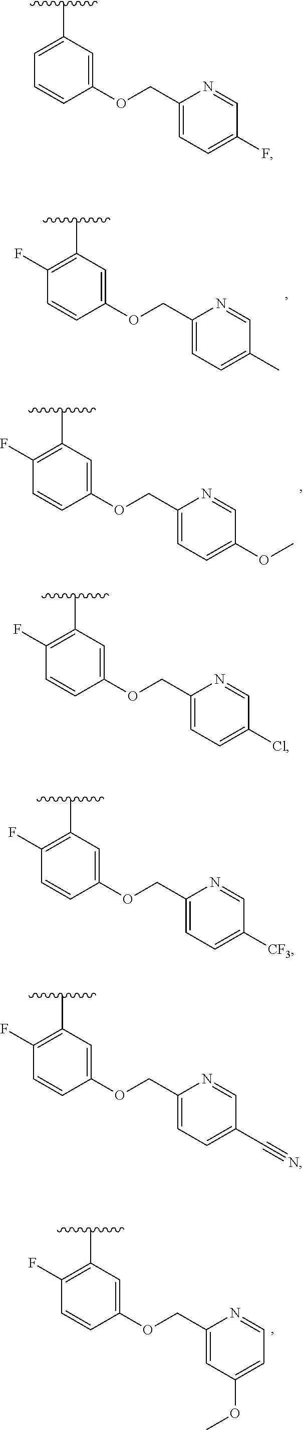

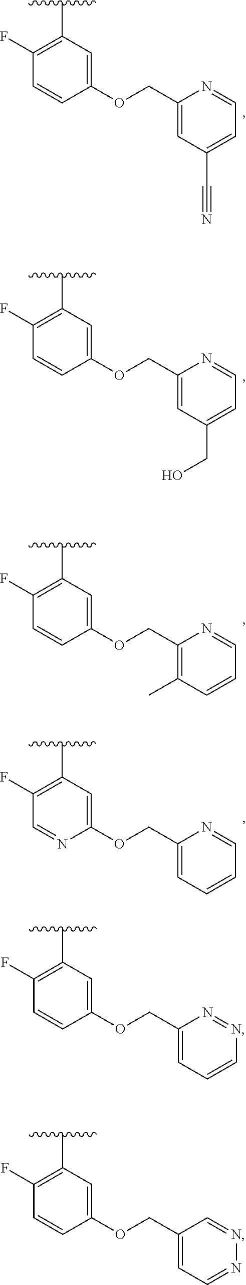 [SMILES: *c1cc(OCc2cc(C#N)ccn2)ccc1F ; *c1cc(OCc2cc(CO)ccn2)ccc1F ; *c1cc(OCc2ncccc2C)ccc1F ; *c1cc(OCc2ccccn2)ncc1F ; *c1cc(OCc2cccnn2)ccc1F ; *c1cc(OCc2ccnnc2)ccc1F]

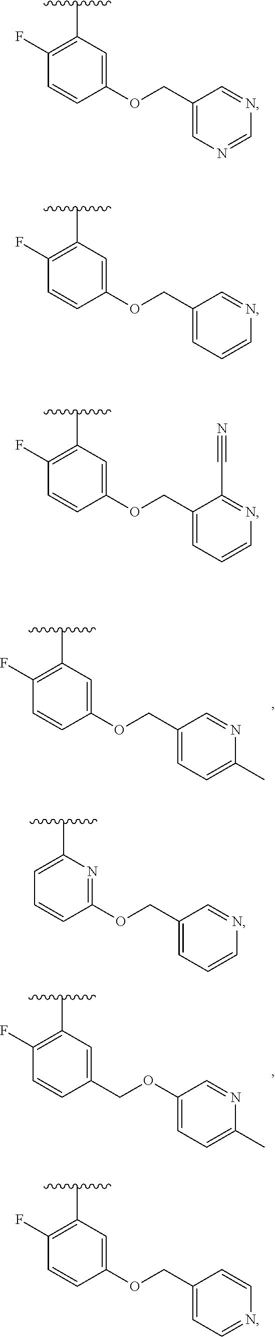

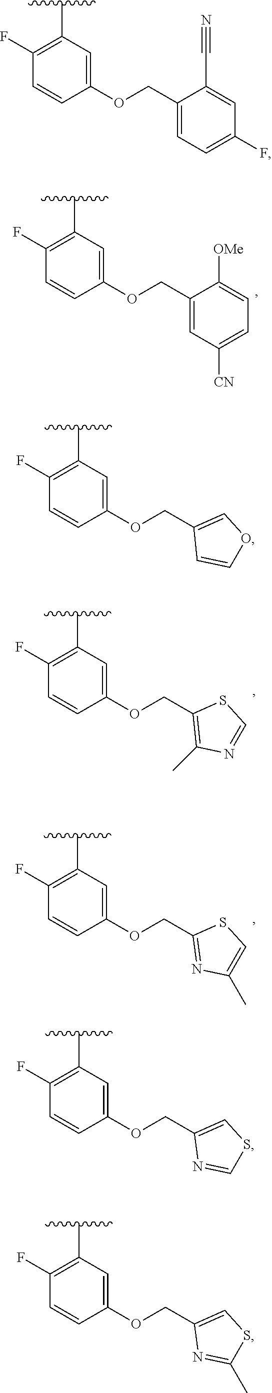

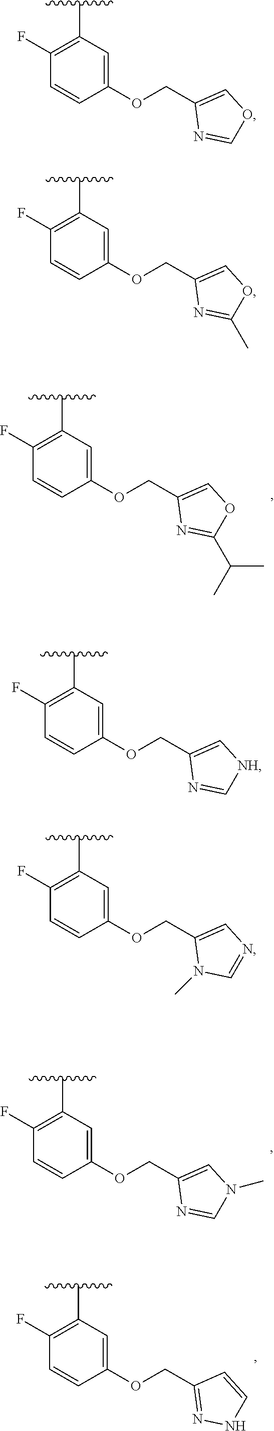

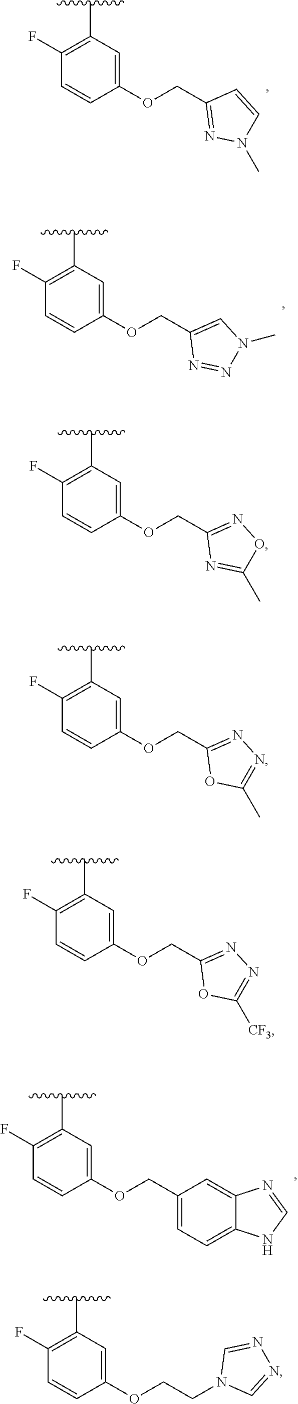

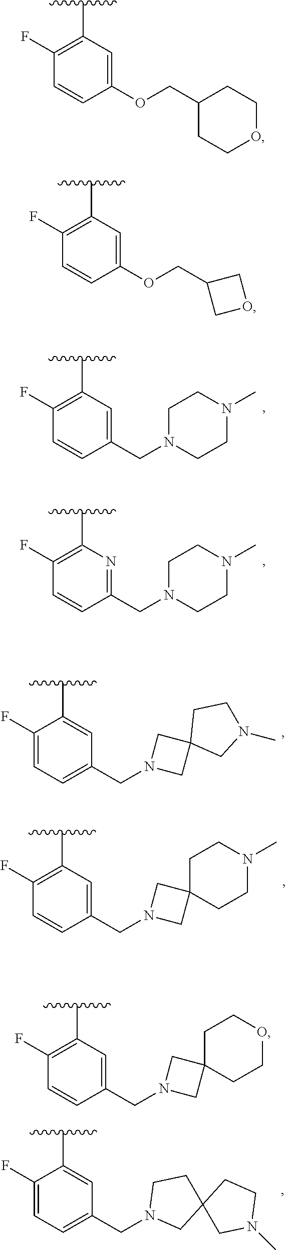

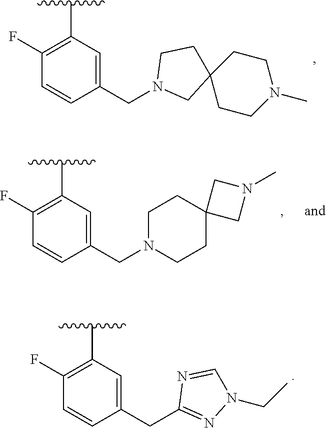 [SMILES: *c1cc(CN2CCC3(CCN(C)CC3)C2)ccc1F ; *c1cc(CN2CCC3(CC2)CN(C)C3)ccc1F ; *c1cc(Cc2ncn(CC)n2)ccc1F]

115. L¹ is NH and R⁴ is as defined in any one of 100 to 113.
116. L¹ is a bond and R⁴ is azetidin-1-yl, pyrrolidin-1-yl, piperidin-1-yl, piperazin-1-yl and morpholin-4-yl, wherein R⁴ is optionally substituted by one or two substituents selected from: halo, ═O, and C₁₋₄ alkyl.
117. X¹ is CH, n is 0, L¹ is NH, R⁴ is as defined in 111 and Ring A is as defined in any one of 20 to 32 or 114.
118. X¹ is CH, n is 0, L¹ is NH, R⁴ is as defined in 111 and Ring A is as defined in 95, 96 or 114.

In certain embodiments the compound of the invention is a compound of the formula (I), or a pharmaceutically acceptable salt thereof, wherein:
X¹ is CH;
Ring A is phenyl or a 5- or 6-membered heteroaryl;
R¹ is H;
n is 0;
each R³ is independently selected from: halo, —CN, C₁₋₆ alkyl, C₁₋₆ haloalkyl, —OR⁵, —NR^{a1}R⁵, and -L²-Q²,
wherein said C₁₋₆ alkyl is optionally substituted by one or more substituents selected from: —CN, —OR^{a2}, and —NR^{a2}R^{b2}
or two R³ groups attached to the same or adjacent ring atoms in Ring A together form a 4- to 6-membered heterocyclic fused ring, wherein said 4- to 6-membered heterocyclic is optionally substituted with one or more substituents selected from: halo, ═O, C₁₋₄ alkyl and C₁₋₄ haloalkyl;
R⁵ is selected from: H, C₁₋₆ alkyl and C₁₋₆ haloalkyl, wherein said C₁₋₆ alkyl is optionally substituted by one or more substituents selected from: —CN, —OR^{a3}, and —NR^{a3}R^{b3};
L² is a bond or is selected from: *—[CR⁶R⁷]ₐ—NR⁸—, *—NR⁸—[CR⁶R⁷]ₐ—, *—[CR⁶R⁷]ₐ—O—, *—O—[CR⁶R⁷]ₐ—, and —[CR⁶R⁷]ₐ—, wherein * indicates the bond to Ring A,
   each R⁶ and R⁷ is independently selected from: H, and C₁₋₃ alkyl,
   or two R⁶ and R⁷ attached to the same carbon atom form a C₃₋₆ cycloalkyl,
   a is 0 to 4,
   R⁸ is selected from: H and C₁₋₄ alkyl;

Q² is selected from: C₃₋₆ cycloalkyl, 4- to 12-membered heterocyclyl, phenyl and 5-to 12-membered heteroaryl,
wherein said C₃₋₆ cycloalkyl and 4- to 12-membered heterocyclyl is optionally substituted by one or more R⁹,
wherein said phenyl and 5- to 12-membered heteroaryl is optionally substituted by one or more R¹⁰;
L¹ is a bond or is selected from: O and NH;
R⁴ is H, C₁-6 alkyl, C₁₋₆ haloalkyl, and -L³-Q³,
   wherein said C₁₋₆ alkyl is optionally substituted by one or more substituents selected from: —CN, —OR^{a8} and —NR^{a8}R^{b8}.
L³ is a bond or —[CR¹¹R¹²]_{b}—,
each R¹¹ and R¹² is independently selected from: H and C₁₋₃ alkyl,
   or two R¹¹ and R¹² attached to the same carbon atom form a C₃₋₆ cycloalkyl,
   b is 1 to 4,
   Q³ is selected from: C₃₋₆ cycloalkyl and 4- to 6-membered heterocyclyl,
wherein said C₃₋₆ cycloalkyl and 4- to 6-membered heterocyclyl is optionally substituted by one or more R¹³
   each R⁹ and R¹³ is independently selected from: halo, ═O, C₁₋₄ alkyl, C₁₋₄ haloalkyl, —OR^{a4}, —S(O)₂R^{a4}, —NR^{a4}R^{b4}, —C(O)R^{a4}, —C(O)NR^{a4}R^{b4} and —SO₂NR^{a4}R^{b4};
each R¹⁰ is independently selected from: halo, —CN, C₁₋₄ alkyl, C₁₋₄ haloalkyl, —OR^{a6}, and —NR^{a6}R^{b6}.
wherein said C₁₋₄ alkyl is optionally substituted by 1 or 2 substituents selected from: halo, —CN, —OR^{a7}, —NR^{a7}R^{b7} and —SO₂R^{a7};
R^{a1}, R^{a2}, R^{b2}, R^{a3}, R^{b3}, R^{a4}, R^{b4}, R^{a6}, R^{b6}, R^{a7}, R^{b7}, R^{a8} and R^{b8} are at each occurrence independently selected from: H, C₁₋₄ alkyl and C₁₋₄ haloalkyl,
or any —NR^{a1}R⁵, —NR^{a2}R^{b2}, —NR^{a3}R^{b3}, —NR^{a4}R^{b4}, —NR^{a6}R^{b6}, —NR^{a7}R^{b7} or —NR^{a8}R^{b8} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl;
p is 0, 1, 2, 3 or 4; and
x at each occurrence is independently 0, 1 or 2.

In certain embodiments the compound of the invention is a compound of the formula (I) to (XXVIII), or a pharmaceutically acceptable salt thereof, wherein R¹ is H.

In certain embodiments the compound of the invention is a compound of the formula (I), (II), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII) or (XXVIII), or a pharmaceutically acceptable salt thereof, wherein R¹ is H and R⁴ is as defined in any one of 100 to 113. For example, it may be that R¹ is H and R⁴ is —CH₂CH₂CH₃.

In certain embodiments the compound of the invention is a compound of the formula (I), (II), (III) and (IV), or a pharmaceutically acceptable salt thereof, wherein R¹ is H; and Ring A is as defined in any of 20 to 32.

It may be that Ring A is as defined in any of 20 to 32, wherein Ring A is substituted by one or two R³.

It may be that Ring A is as defined in any of 20 to 32, wherein Ring A is substituted by one or two R³ as defined in 36.

It may be that Ring A is as defined in any of 20 to 32, wherein Ring A is substituted by R³ as defined in 38.

It may be that Ring A is as defined in any of 20 to 32, wherein Ring A is substituted by one or two R³ as defined in 49.

It may be that Ring A is as defined in any of 20 to 32, wherein Ring A is substituted by one or two R³ as defined in 50

It may be that Ring A is as defined in any of 20 to 32, wherein Ring A is substituted by one or two R³ as defined in 51.

It may be that Ring A is as defined in any of 20 to 32, wherein Ring A is substituted by one or two R³ as defined in 82.

It may be that Ring A is as defined in any of 20 to 32, wherein Ring A is substituted by one R³ which is -L²-Q² (e.g. as defined in any one of 88 to 93) and wherein Ring A is optionally further substituted with one or two R³ selected from: halo, C₁₋₄ alkyl, C₁₋₄ haloalkyl and —OR^{a2}.

It may be that Ring A is as defined in any of 20 to 32, wherein Ring A is substituted by 1 or 2 R³ selected from: —CN, halo (e.g. F or Cl), —CF₃, methyl, ethyl, propyl, isopropyl, hydroxymethyl, methoxymethyl, methoxy, ethoxy, propoxy, isopropoxy, cyclopropyloxy, fluoromethoxy, difluoromethoxy, trifluoromethoxy, 2-methoxyethoxy, —NH₂ and -L²-Q² (e.g. as defined in any one of 88 to 93).

It may be that in these embodiments R⁴ is as defined in 111, for example wherein R⁴ is —CH₂CH₂CH₃.

In certain embodiments the compound of the invention is a compound of the formula (I), (II), (III) or (IV), or a pharmaceutically acceptable salt thereof, wherein Ring A is substituted by one R³ selected from: —C₁₋₄ alkyl-OR^{a2}, —O—C₁₋₄ alkyl, —O—C₁₋₄ haloalkyl and -L²-Q², wherein -L²-Q² is as defined in any one of 88 to 93 and wherein Ring A is optionally further substituted with one or two R³ selected from: halo, —CN, C₁₋₄ alkyl, C₁₋₄ haloalkyl and —OR^{a2}.

In certain embodiments the compound of the invention is a compound of the formula (I), (II), (III) and (IV), or a pharmaceutically acceptable salt thereof, wherein Ring A is as defined in 95 and R⁴ is as defined in any one of 100 to 113. For example, it may be that R⁴ is as defined in 111, preferably —CH₂CH₂CH₃. Suitably in these embodiments R¹ is H.

In certain embodiments the compound of the invention is a compound of the formula (I), (II), (III) and (IV), or a pharmaceutically acceptable salt thereof, wherein Ring A is as defined in 96 and R⁴ is as defined in any one of 100 to 113. For example, it may be that R⁴ is as defined in 111, preferably —CH₂CH₂CH₃. Suitably in these embodiments R¹ is H.

In certain embodiments the compound of the invention is a compound of the formula (I), (II) or (IV), or a pharmaceutically acceptable salt thereof, wherein Ring A is as defined in 114 and R⁴ is as defined in any one of R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (I), (II), (III), (IV), (V), (VIII), (X), (XII), (XIV), (XVI), or a pharmaceutically acceptable salt thereof, wherein the compound is substituted with at least one R³ (i.e. p or p1 is at least 1).

In certain embodiments the compound of the invention is a compound of the formula (XIX), (XX), (XXVII) or (XXVIII), or a pharmaceutically acceptable salt thereof, wherein at least one of R³¹, R³², R³³ or R³⁴ is not H.

In certain embodiments the compound of the invention is a compound of the formula (VII), or a pharmaceutically acceptable salt thereof, wherein at least one of X², X³, X⁴, X⁵ and X⁶ is CR³.

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XXIII), (XXIV), (XXV), and (XXVI), or a pharmaceutically acceptable salt thereof, wherein each R³ is as defined in any one of 33 to 41. Suitably in these embodiments R⁴ is as defined in any one of 100 to 113. For example, it may be that R⁴ is as defined in 111, preferably —CH₂CH₂CH₃. Suitably in these embodiments R¹ is H.

In certain embodiments the compound of the invention is a compound of the formula (VI), (IX), (XI), (XIII), (XV) and (XVII), or a pharmaceutically acceptable salt thereof, wherein L² is selected from: *—(CH₂)₂—O—, *—CH₂—O—, *—O—(CH₂)₂— and *—O—CH₂—, wherein * indicates the bond to Ring A and Q² is as defined in any one of 65 to 81. Thus it may be that -L²-Q² is as defined in any one of 88, 89, 91 or 93. Suitably in these embodiments R⁴ is as defined in any one of 100 to 113. For example, it may be that R⁴ is as defined in 111, preferably —CH₂CH₂CH₃. Suitably in these embodiments R¹ is H.

In certain embodiments the compound of the invention is a compound of the formula (VI), (IX), (XI), (XIII), (XV) and (XVII), or a pharmaceutically acceptable salt thereof, wherein L² is selected from: *—(CH₂)₂—NH—, *—CH₂—NH—, *—NH—(CH₂)₂—, *—NH—CH₂—, —CH₂— and —(CH₂)₂— wherein * indicates the bond to Ring A and Q² is as defined in any one of 65 to 81. Thus it may be that -L²-Q² is as defined in 90. Suitably in these embodiments R⁴ is as defined in any one of 100 to 113. For example, it may be that R⁴ is as defined in 111, preferably —CH₂CH₂CH₃. Suitably in these embodiments R¹ is H.

In certain embodiments the compound of the invention is a compound of the formula (XVIII), or a pharmaceutically acceptable salt thereof, wherein:
R³⁰ is selected from: H, C₁₋₄ alkyl, C₁₋₄ haloalkyl and Q², wherein Q² is as defined in any one of 65 to 81; and
R³¹ and R³² are independently selected from: H, halo, C₁₋₄ alkyl, C₁₋₄ haloalkyl, —O—C₁₋₄ alkyl and —O—C₁₋₄ haloalkyl.

It may be that R³¹ and R³² are independently selected from: H, halo (e.g. F) and C₁₋₄ alkyl (e.g. Me).

Preferably R³¹ and R³² are not both H.

It may be in this embodiment that R³¹ is halo or C₁₋₄ alkyl and R³² is selected from: H, halo, C₁₋₄ alkyl, C₁₋₄ haloalkyl, —O—C₁₋₄ alkyl and —O—C₁₋₄ haloalkyl.

It may be in this embodiment that R³² is halo or C₁₋₄ alkyl and R³¹ is selected from: H, halo, C₁₋₄ alkyl, C₁₋₄ haloalkyl, —O—C₁₋₄ alkyl and —O—C₁₋₄ haloalkyl.

It may be in this embodiment that R⁴ is as defined in any one of 100 to 113. For example, it may be that R⁴ is as defined in 111, preferably —CH₂CH₂CH₃. Suitably in this embodiment R¹ is H.

In certain embodiments the compound of the invention is a compound of the formula (XIX) or (XX), or a pharmaceutically acceptable salt thereof, wherein: R³¹ and R³² are independently selected from: H, halo, C₁₋₄ alkyl, —C₁₋₄ alkyl-OR^{a2}, C₁₋₄ haloalkyl, —O—C₁₋₄ alkyl and —O—C₁₋₄ haloalkyl.

It may be that R³¹ and R³² are independently selected from: H, halo (e.g. F), C₁₋₄ alkyl (e.g. Me), and —O—C₁₋₄ alkyl (e.g. methoxy). Preferably R³¹ and R³² are not both H.

It may be that R³³ is -L²-Q². It may be that R³³ is -L²-Q² wherein L² is selected from: —(CH₂)₂—O—*, —CH₂—O—*, —O—(CH₂)₂—*, —O—CH₂—*, —(CH₂)₂—NH—*, —CH₂—NH—*, —NH—(CH₂)₂—*, —NH—CH₂—*, —CH₂— and —(CH₂)₂— wherein * indicates the bond to Q², and Q² is as defined in any one of 65 to 81.

It may be that R³³ is selected from: C₁₋₃ alkyl (e.g. methyl), —C₁₋₄ alkyl-OH, —C₁₋₄ alkyl-O—C₁₋₃ alkyl and —O—C₁₋₄ alkyl (e.g. methoxy).

It may be in this embodiment that R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (XIX), or a pharmaceutically acceptable salt thereof, wherein:
X² and X³ are N;
R³¹ is selected from: halo, C₁₋₄ alkyl, —C₁₋₄ alkyl-OR^{a2}, C₁₋₄ haloalkyl, —O—C₁₋₄ alkyl and —O—C₁₋₄ haloalkyl;
R³² is H;
R³³ is selected from: C₁₋₄ alkyl and —O—C₁₋₄ alkyl; and
R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (XIX), or a pharmaceutically acceptable salt thereof, wherein:
X² and X³ are N;
R³¹ is selected from: halo, C₁₋₄ alkyl, and —O—C₁₋₄ alkyl and —O—C₁₋₄ haloalkyl; R³³ is C₁₋₃ alkyl; and
R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (XIX), or a pharmaceutically acceptable salt thereof, wherein:
X² and X³ are N;
R³¹ is halo (e.g. F);
R³³ is C₁₋₃ alkyl; and
R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (XXI), (XXIII), (XXIV), (XXV) or (XXVI), or a pharmaceutically acceptable salt thereof, wherein
R³² is selected from: H, halo, C₁₋₄ alkyl, C₁₋₄ haloalkyl, —O—C₁₋₄ alkyl and —O—C₁₋₄ haloalkyl; and
R³ is as defined in any one of 33 to 41.

It may be that R³² is H. It may be that R³² is selected from: halo, C₁₋₄ alkyl, C₁₋₄ haloalkyl, —O—C₁₋₄ alkyl and —O—C₁₋₄ haloalkyl.

It may be that R³ is -L²-Q², wherein L² is selected from: —(CH₂)₂—O—*, —CH₂—O—*, —O—(CH₂)₂—*, —O—CH₂—*, —(CH₂)₂—NH—*, —CH₂—NH—*, —NH—(CH₂)₂—*, —NH—CH₂—*, —CH₂— and —(CH₂)₂— wherein * indicates the bond to Q², and Q² is as defined in any one of 65 to 81.

It may be that R³ is Q²-L²., wherein Q²-L². is as defined in any one of 88 to 93.

It may be in this embodiment that R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (XXII), or a pharmaceutically acceptable salt thereof, wherein:
R³⁰ is selected from: H, C₁₋₄ alkyl, C₁₋₄ haloalkyl and Q², wherein Q² is as defined in any one of 65 to 81; and
R³² is selected from: H, halo, C₁₋₄ alkyl, C₁₋₄ haloalkyl, —O—C₁₋₄ alkyl and —O—C₁₋₄ haloalkyl. For example it may be that R³² is H. It may be that R³² is selected from: halo, C₁₋₄ alkyl, C₁₋₄ haloalkyl, —O—C₁₋₄ alkyl and —O—C₁₋₄ haloalkyl. It may be that R³² is selected from: H, halo and C₁₋₄ alkyl.

It may be in this embodiment that R⁴ is as defined in any one of 100 to 113. For example, it may be the R⁴ is as defined in 111, preferably —CH₂CH₂CH₃.

Preferably in this embodiment R¹ is H.

In certain embodiments the compound of the invention is a compound of the formula (XXVI), or a pharmaceutically acceptable salt thereof, wherein:
R³² is H;
R³ is as defined in any one of 33 to 41; and
R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (XXVI), or a pharmaceutically acceptable salt thereof, wherein:
R³² is H;
R³ is selected from: C₁₋₄ alkyl and —OC₁₋₄ alkyl; and
R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (XXVI), or a pharmaceutically acceptable salt thereof, wherein:
R³² is H;
R³ is C₁₋₄ alkyl; and
R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (XXVI), or a pharmaceutically acceptable salt thereof, wherein:
R³² is H;
R³ is methyl; and
R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (XXVII) or (XXVIII), or a pharmaceutically acceptable salt thereof, wherein:
R³¹ is H or R³;
R³⁴ is R³;
each R³ is independently as defined in any one of 33 to 41; and
R⁴ is as defined in any one of 100 to 113. For example, it may be that R⁴ is as defined in 111, preferably —CH₂CH₂CH₃.

Preferably in this embodiment R¹ is H.

In certain embodiments the compound of the invention is a compound of the formula (XXVII), or a pharmaceutically acceptable salt thereof, wherein:
R³¹ is selected from H, halo, C₁₋₄ alkyl and —OC₁₋₄ alkyl;
R³⁴ is C₁₋₄ alkyl; and
R⁴ is as defined in any one of 100 to 113. For example, it may be that R⁴ is as defined in 111, preferably —CH₂CH₂CH₃.

Preferably in this embodiment R¹ is H.

Thus it may be in this embodiment that R³¹ is selected from: halo, C₁₋₄ alkyl and —OC₁₋₄ alkyl; and R³⁴ is C₁₋₄ alkyl. It may be in this embodiment that R³¹ is —OC₁₋₄ alkyl (e.g.-OMe); and R³⁴ is C₁₋₄ alkyl (e.g. Me). It may be in this embodiment that R³¹ is selected from F and methoxy, and R³⁴ is methyl. It may be in this embodiment that R³¹ is methoxy and R³⁴ is methyl.

In certain embodiments the compound of the invention is a compound of the formula (XXVIII), or a pharmaceutically acceptable salt thereof, wherein:
R³¹ is selected from H, halo, C₁₋₄ alkyl and —OC₁₋₄ alkyl;
R³⁴ is C₁₋₄ alkyl; and
R⁴ is as defined in any one of 100 to 113. For example, it may be that R⁴ is as defined in 111, preferably —CH₂CH₂CH₃.

Preferably in this embodiment R¹ is H.

Thus it may be in this embodiment that R³¹ is selected from halo, C₁₋₄ alkyl and —OC₁₋₄ alkyl; and R³⁴ is C₁₋₄ alkyl. It may be in this embodiment that R³¹ is —OC₁₋₄ alkyl (e.g. —OMe); and R³⁴ is C₁₋₄ alkyl (e.g. Me).

In another embodiment the compound of formula (I) is of the formula (XXIX), or a pharmaceutically acceptable salt thereof:

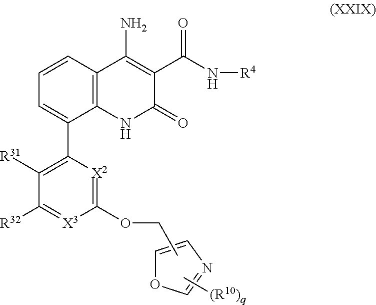

wherein
X² and X³ are each independently N or CH;
R³¹ and R³² are each independently selected from: H and R³;
q is 0, 1 or 2; and
R³, R⁴ and R¹⁰ are as defined for formula (I).

In this embodiment it may be that R³¹ and R³² are independently H or R³, wherein R³ is as defined in any one of 33 to 41.

In this embodiment it may be R³¹ and R³² are independently selected from: H, halo, C₁₋₄ alkyl, —C₁₋₄ alkyl-OH, —C₁₋₄ alkyl-O—C₁₋₃ alkyl, C₁₋₄ haloalkyl, —O—C₁₋₄ alkyl and —O—C₁₋₄ haloalkyl.

It may be that R³¹ and R³² are independently selected from: H, halo (e.g. F), C₁₋₄ alkyl (e.g. Me), and —O—C₁₋₄ alkyl (e.g. methoxy)

Preferably R³¹ and R³² are not both H.

It may be in this embodiment that R³¹ is halo or C₁₋₄ alkyl and R³² is selected from: H, halo, C₁₋₄ alkyl, C₁₋₄ haloalkyl, —O—C₁₋₄ alkyl and —O—C₁₋₄ haloalkyl.

It may be in this embodiment that R³² is halo or C₁₋₄ alkyl and R³¹ is selected from: H, halo, C₁₋₄ alkyl, C₁₋₄ haloalkyl, —O—C₁₋₄ alkyl and —O—C₁₋₄ haloalkyl.

In this embodiment it may be that the group:

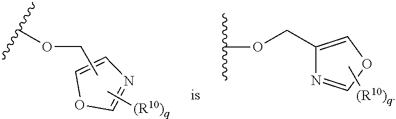

For example, the group

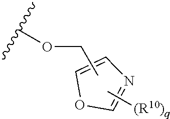

is

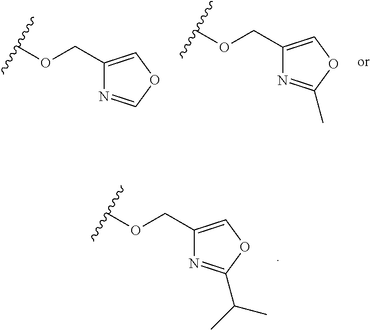

It may be in this embodiment that R⁴ is as defined in any one of 100 to 113. For example, it may be the R⁴ is as defined in 111, preferably —CH₂CH₂CH₃—

In another embodiment there is provided a compound selected from Compound List 1, or a pharmaceutically acceptable salt thereof:

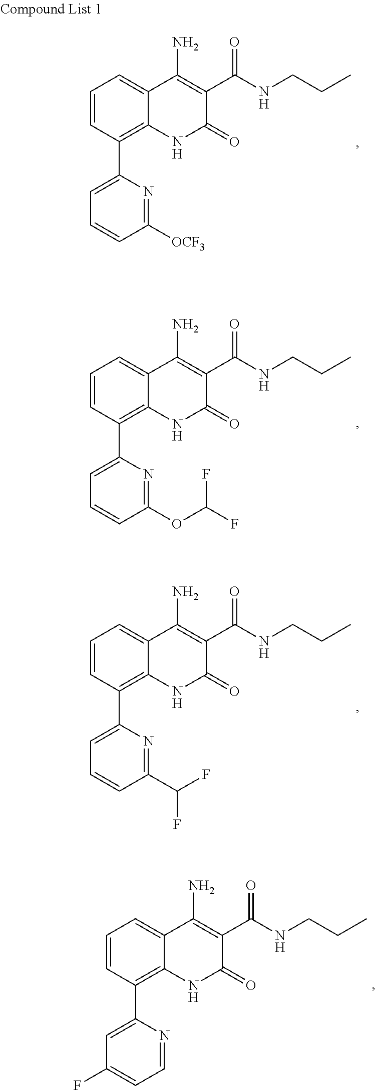 [SMILES: CCCNC(=O)c1c(N)c2cccc(-c3cccc(OC(F)(F)F)n3)c2[nH]c1=O ; CCCNC(=O)c1c(N)c2cccc(-c3cccc(OC(F)F)n3)c2[nH]c1=O ; CCCNC(=O)c1c(N)c2cccc(-c3cccc(C(F)F)n3)c2[nH]c1=O ; CCCNC(=O)c1c(N)c2cccc(-c3cc(F)ccn3)c2[nH]c1=O]

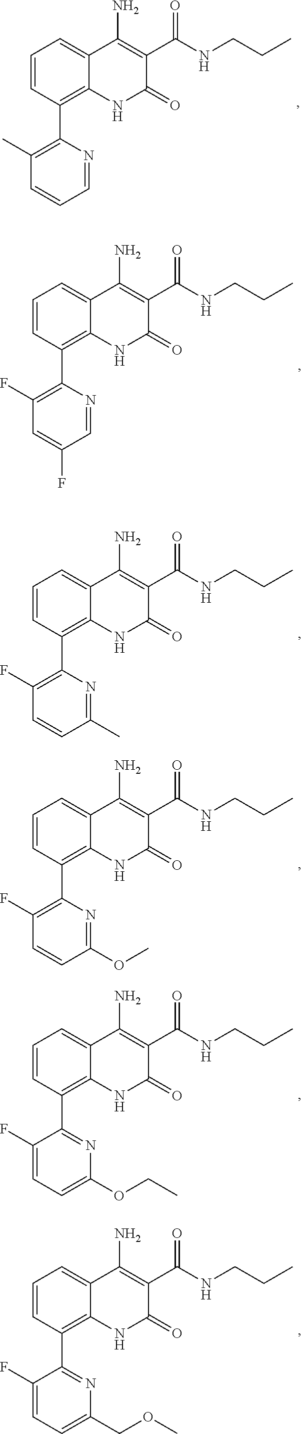

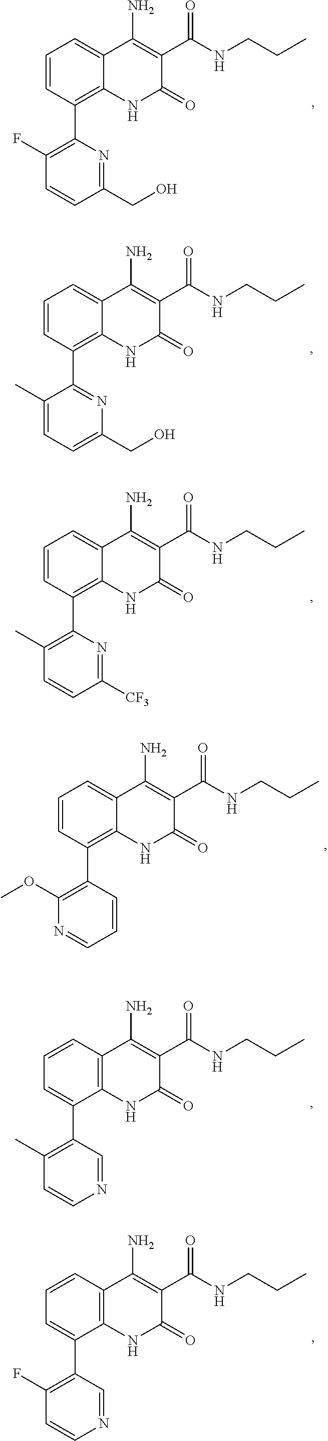

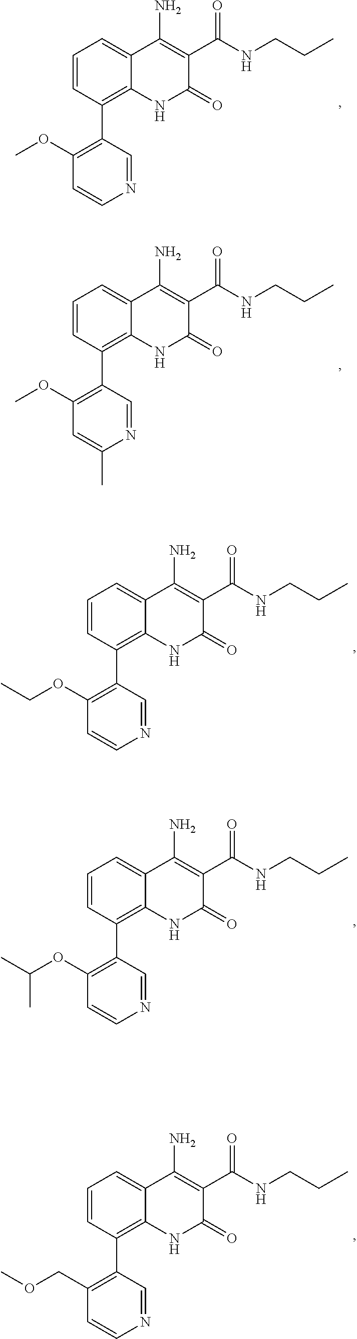

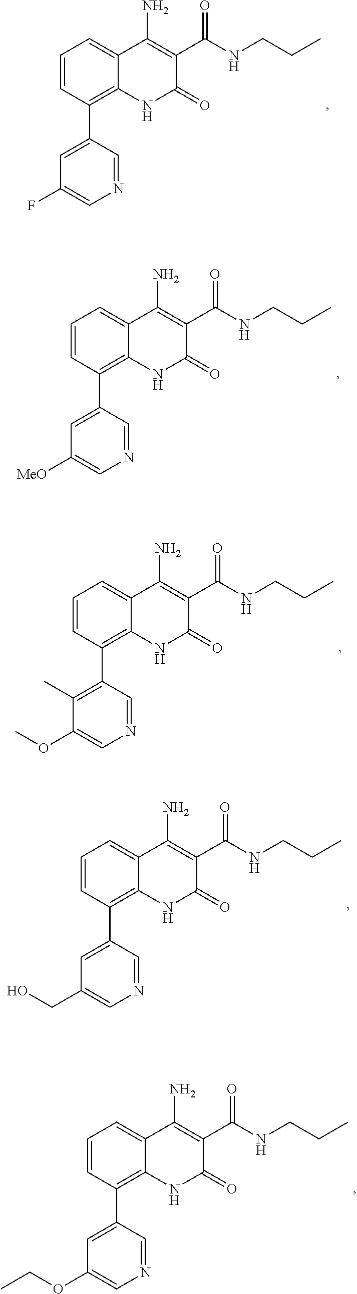

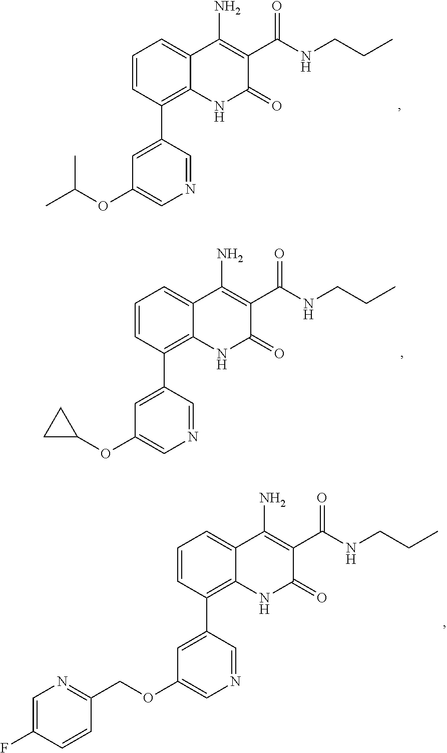 [SMILES: CCCNC(=O)c1c(N)c2cccc(-c3cncc(OC(C)C)c3)c2[nH]c1=O ; CCCNC(=O)c1c(N)c2cccc(-c3cncc(OC4CC4)c3)c2[nH]c1=O ; CCCNC(=O)c1c(N)c2cccc(-c3cncc(OCc4ccc(F)cn4)c3)c2[nH]c1=O]

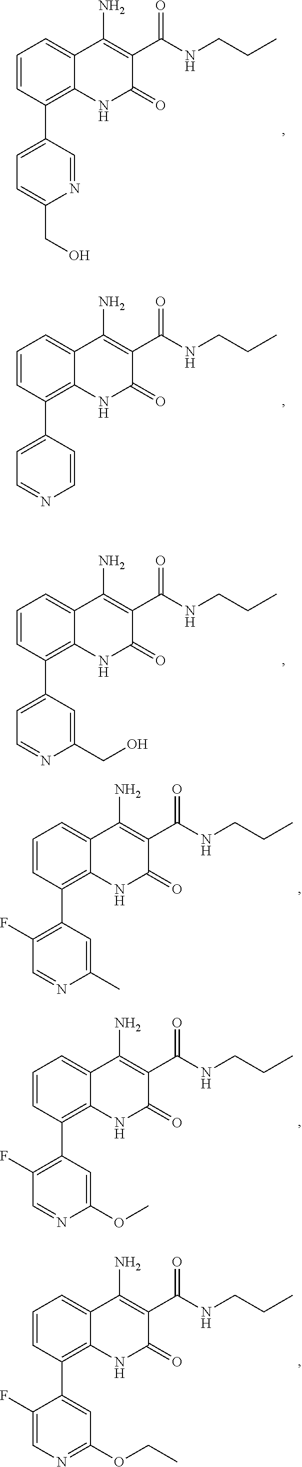

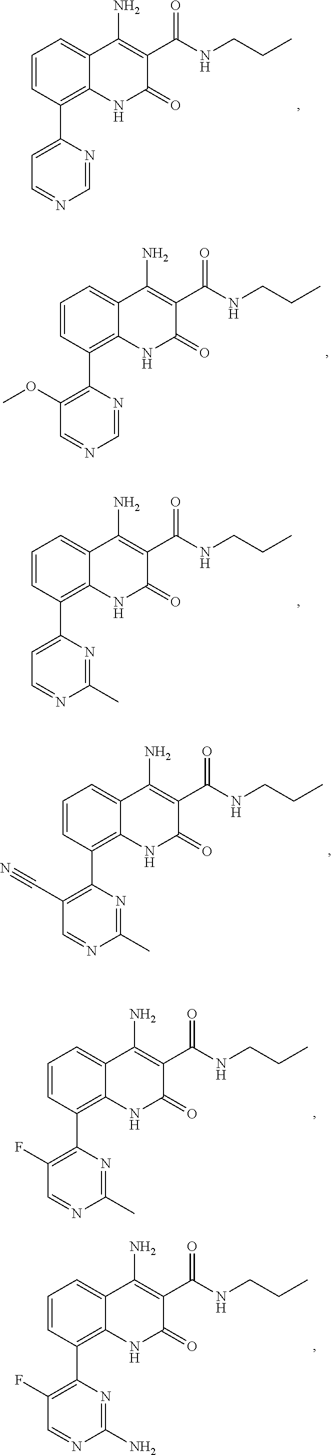

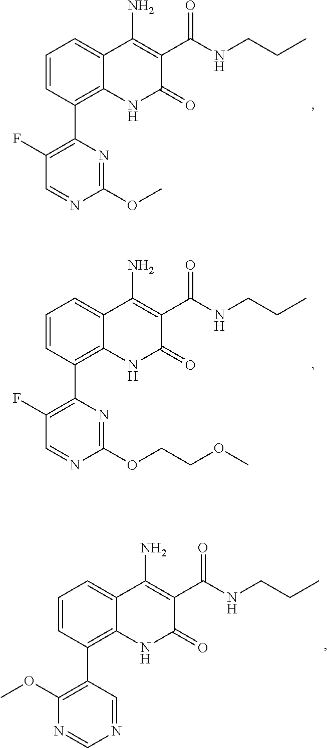

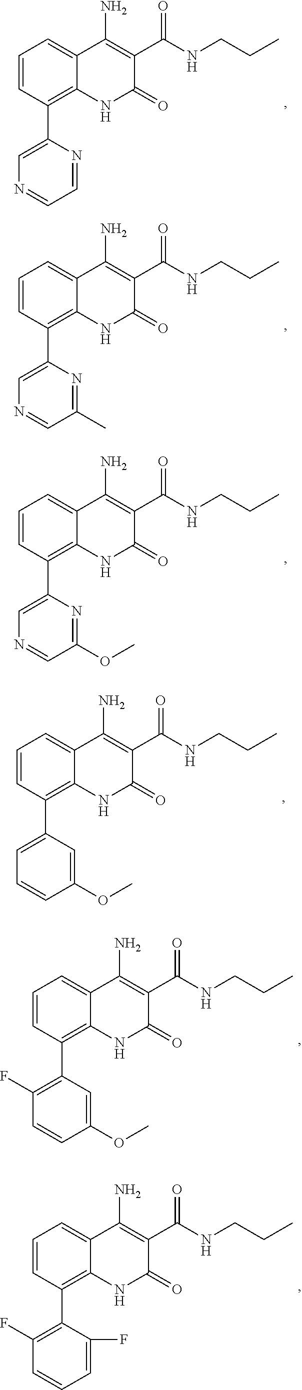

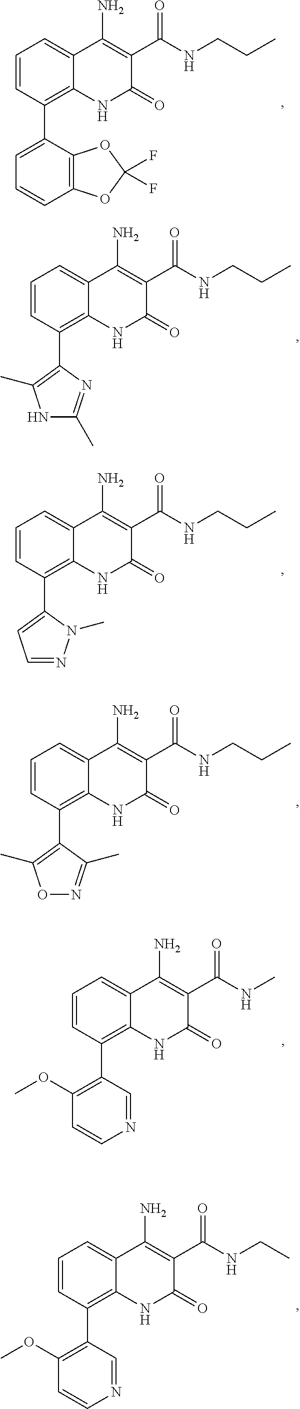

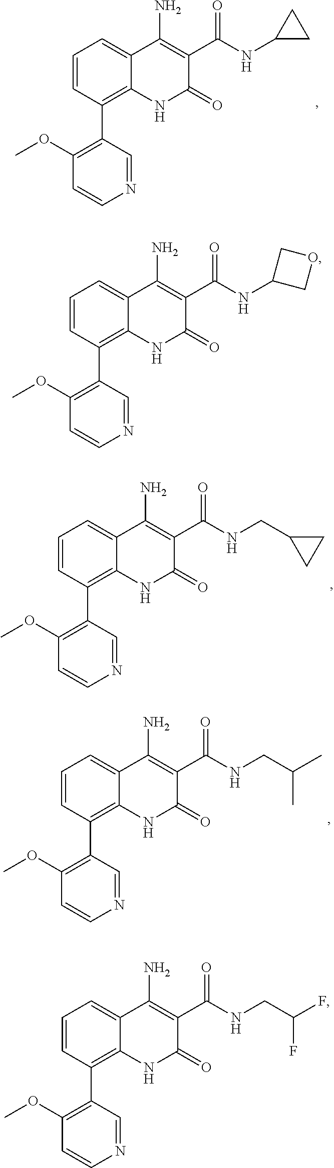

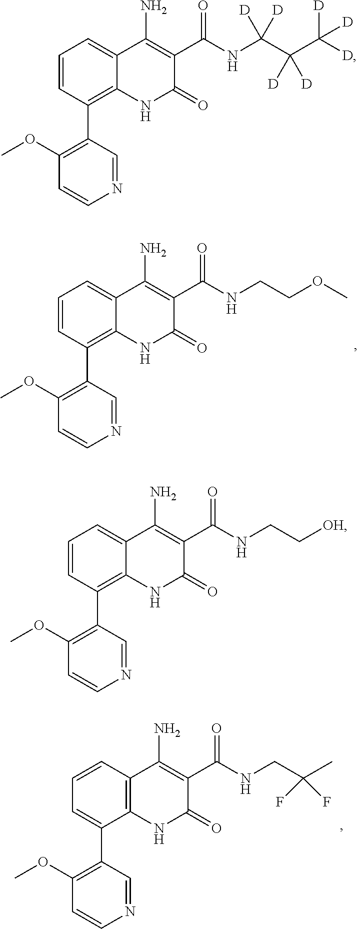

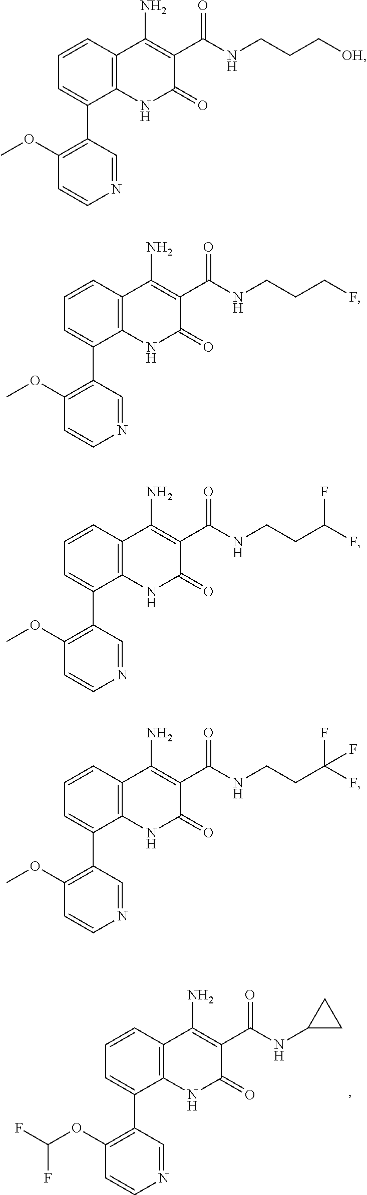 [SMILES: COc1ccncc1-c1cccc2c(N)c(C(=O)NCCCO)c(=O)[nH]c12 ; COc1ccncc1-c1cccc2c(N)c(C(=O)NCCCF)c(=O)[nH]c12 ; COc1ccncc1-c1cccc2c(N)c(C(=O)NCCC(F)F)c(=O)[nH]c12 ; COc1ccncc1-c1cccc2c(N)c(C(=O)NCCC(F)(F)F)c(=O)[nH]c12 ; Nc1c(C(=O)NC2CC2)c(=O)[nH]c2c(-c3cnccc3OC(F)F)cccc12]

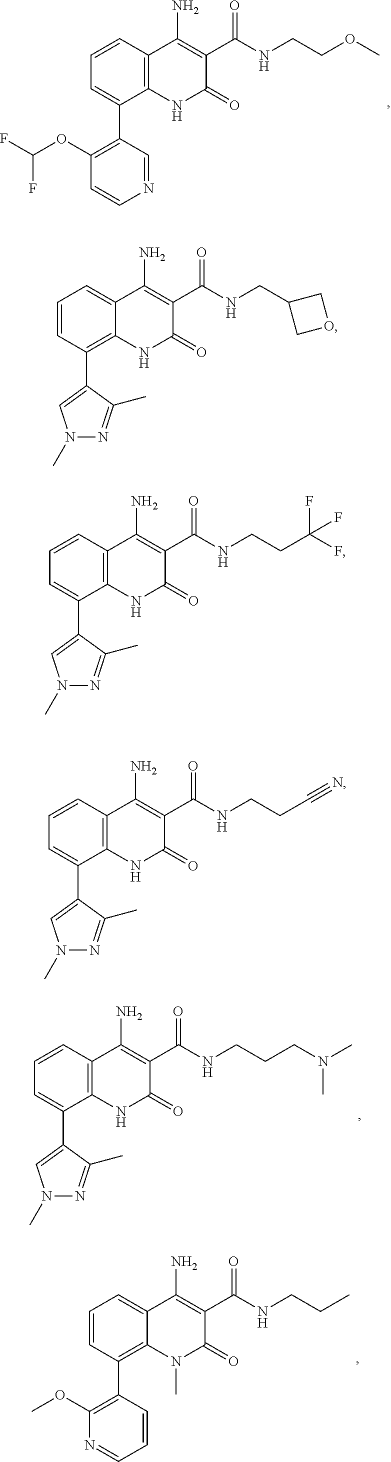

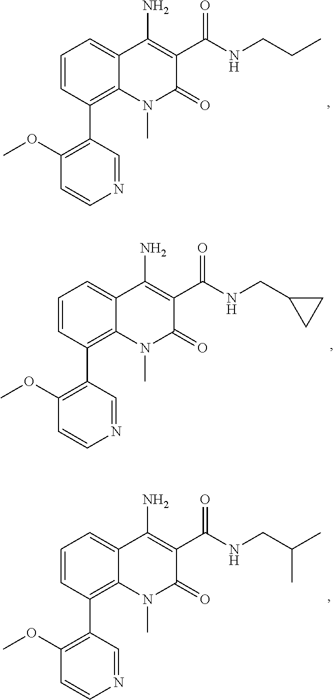

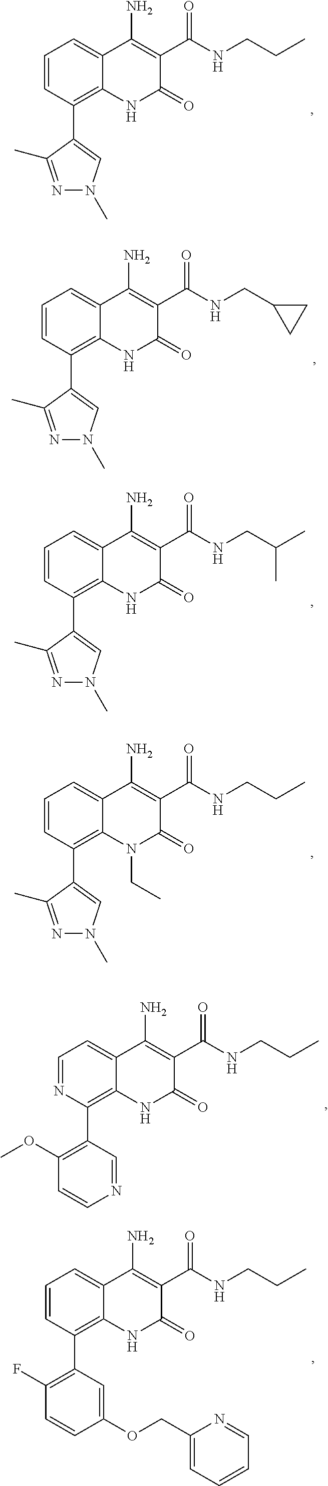

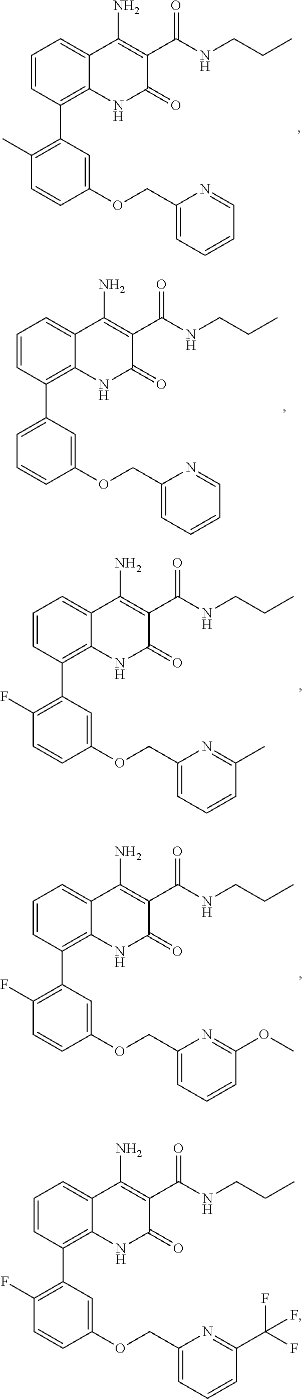

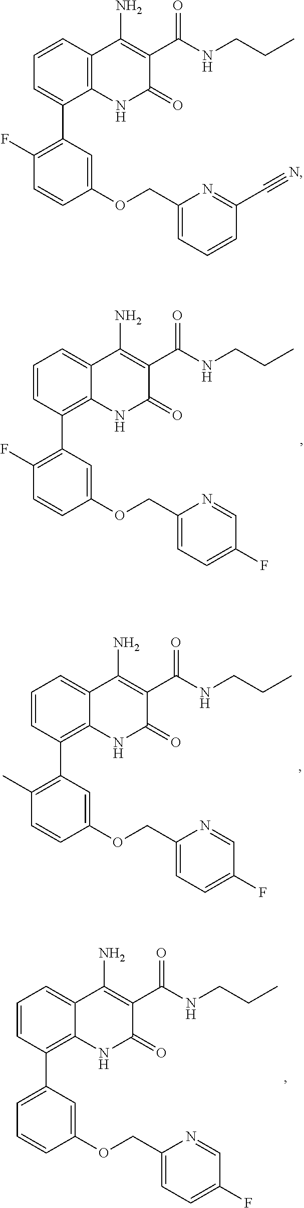

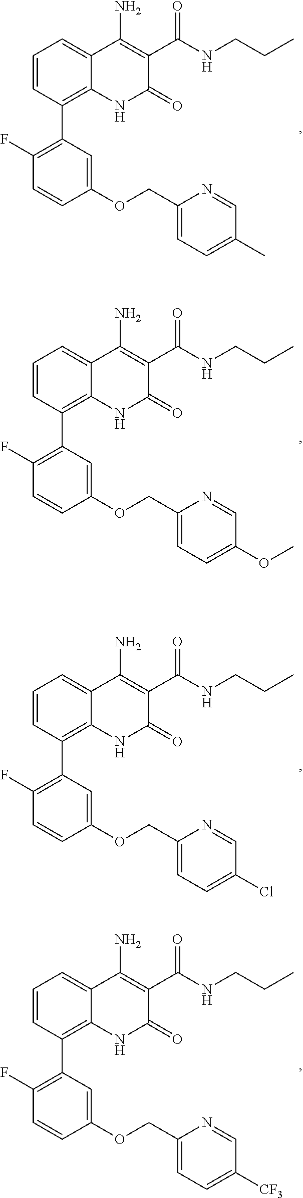 [SMILES: CCCNC(=O)c1c(N)c2cccc(-c3cc(OCc4ccc(C)cn4)ccc3F)c2[nH]c1=O ; CCCNC(=O)c1c(N)c2cccc(-c3cc(OCc4ccc(OC)cn4)ccc3F)c2[nH]c1=O ; CCCNC(=O)c1c(N)c2cccc(-c3cc(OCc4ccc(Cl)cn4)ccc3F)c2[nH]c1=O ; CCCNC(=O)c1c(N)c2cccc(-c3cc(OCc4ccc(C(F)(F)F)cn4)ccc3F)c2[nH]c1=O]

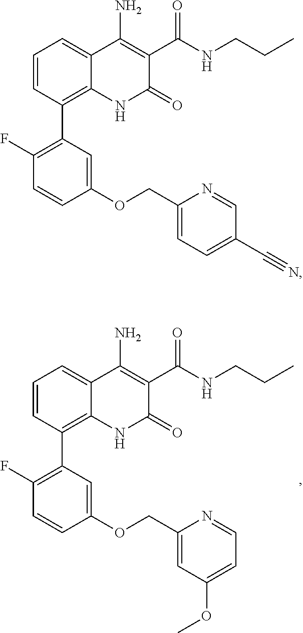 [SMILES: CCCNC(=O)c1c(N)c2cccc(-c3cc(OCc4ccc(C#N)cn4)ccc3F)c2[nH]c1=O ; CCCNC(=O)c1c(N)c2cccc(-c3cc(OCc4cc(OC)ccn4)ccc3F)c2[nH]c1=O]

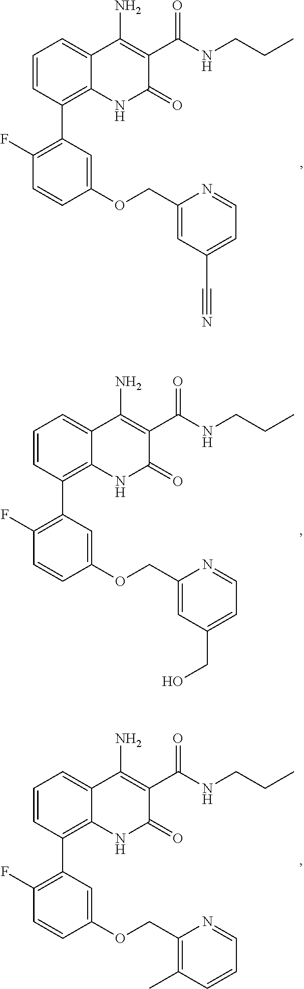 [SMILES: CCCNC(=O)c1c(N)c2cccc(-c3cc(OCc4cc(C#N)ccn4)ccc3F)c2[nH]c1=O ; CCCNC(=O)c1c(N)c2cccc(-c3cc(OCc4cc(CO)ccn4)ccc3F)c2[nH]c1=O ; CCCNC(=O)c1c(N)c2cccc(-c3cc(OCc4ncccc4C)ccc3F)c2[nH]c1=O]

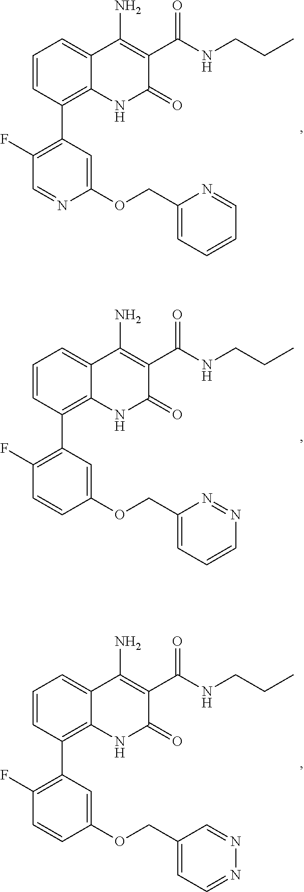 [SMILES: CCCNC(=O)c1c(N)c2cccc(-c3cc(OCc4ccccn4)ncc3F)c2[nH]c1=O ; CCCNC(=O)c1c(N)c2cccc(-c3cc(OCc4cccnn4)ccc3F)c2[nH]c1=O ; CCCNC(=O)c1c(N)c2cccc(-c3cc(OCc4ccnnc4)ccc3F)c2[nH]c1=O]

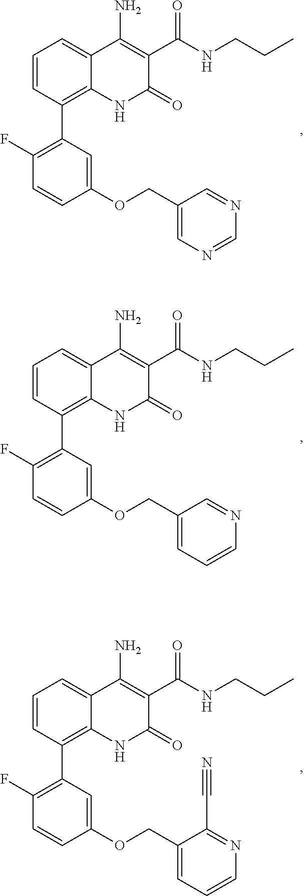 [SMILES: CCCNC(=O)c1c(N)c2cccc(-c3cc(OCc4cncnc4)ccc3F)c2[nH]c1=O ; CCCNC(=O)c1c(N)c2cccc(-c3cc(OCc4cccnc4)ccc3F)c2[nH]c1=O ; CCCNC(=O)c1c(N)c2cccc(-c3cc(OCc4cccnc4C#N)ccc3F)c2[nH]c1=O]

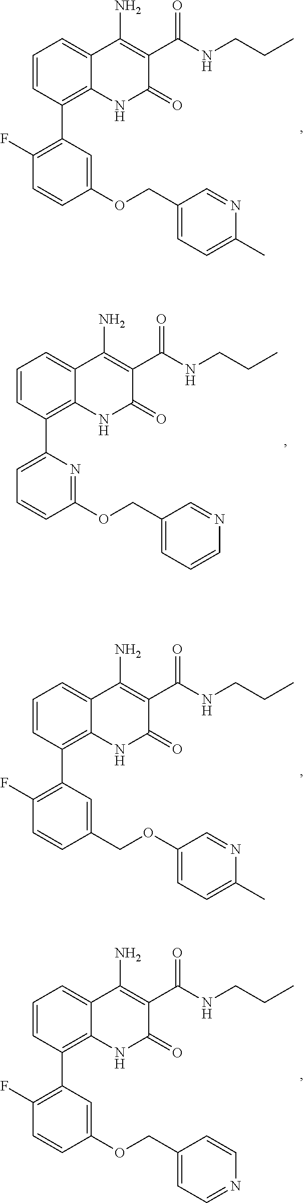 [SMILES: CCCNC(=O)c1c(N)c2cccc(-c3cc(OCc4ccc(C)nc4)ccc3F)c2[nH]c1=O ; CCCNC(=O)c1c(N)c2cccc(-c3cccc(OCc4cccnc4)n3)c2[nH]c1=O ; CCCNC(=O)c1c(N)c2cccc(-c3cc(COc4ccc(C)nc4)ccc3F)c2[nH]c1=O ; CCCNC(=O)c1c(N)c2cccc(-c3cc(OCc4ccncc4)ccc3F)c2[nH]c1=O]

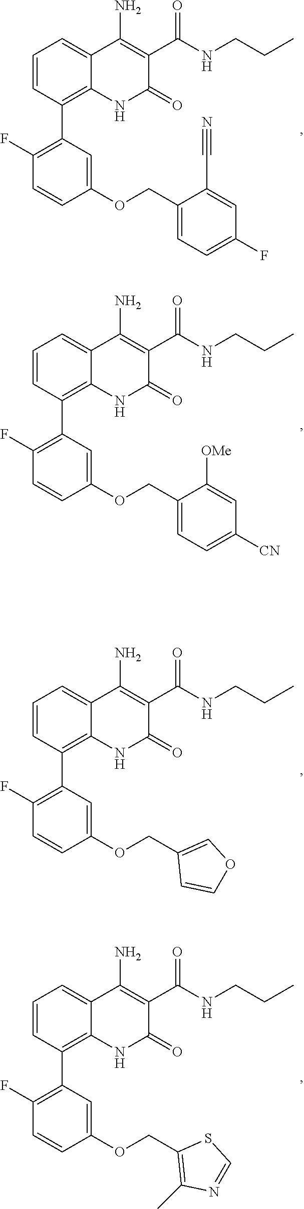 [SMILES: CCCNC(=O)c1c(N)c2cccc(-c3cc(OCc4ccc(F)cc4C#N)ccc3F)c2[nH]c1=O ; CCCNC(=O)c1c(N)c2cccc(-c3cc(OCc4ccc(C#N)cc4OC)ccc3F)c2[nH]c1=O ; CCCNC(=O)c1c(N)c2cccc(-c3cc(OCc4ccoc4)ccc3F)c2[nH]c1=O ; CCCNC(=O)c1c(N)c2cccc(-c3cc(OCc4scnc4C)ccc3F)c2[nH]c1=O]

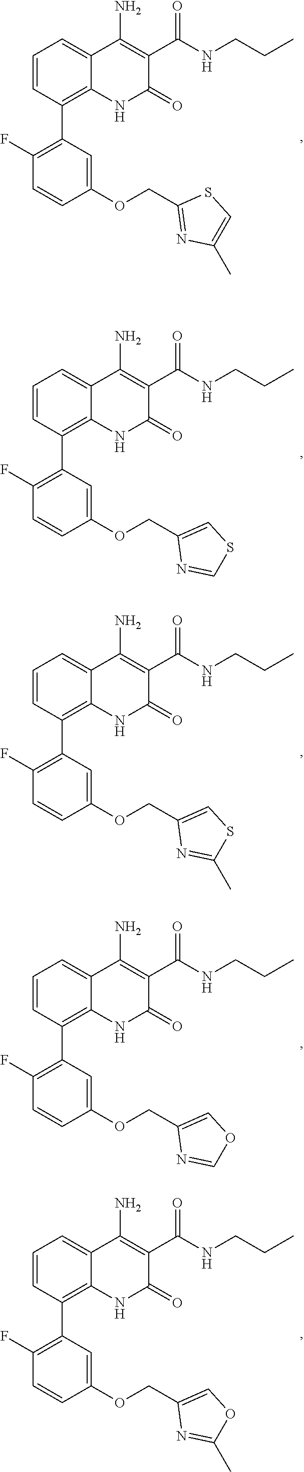

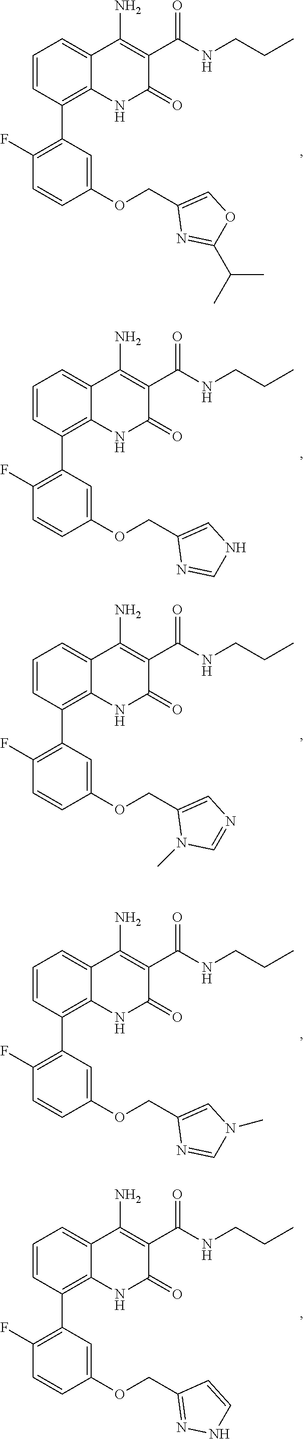

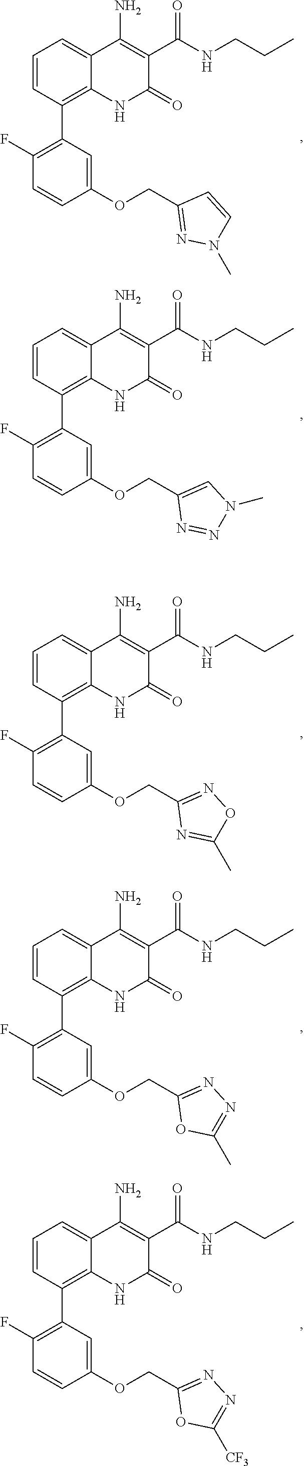 [SMILES: CCCNC(=O)c1c(N)c2cccc(-c3cc(OCc4ccn(C)n4)ccc3F)c2[nH]c1=O ; CCCNC(=O)c1c(N)c2cccc(-c3cc(OCc4cn(C)nn4)ccc3F)c2[nH]c1=O ; CCCNC(=O)c1c(N)c2cccc(-c3cc(OCc4noc(C)n4)ccc3F)c2[nH]c1=O ; CCCNC(=O)c1c(N)c2cccc(-c3cc(OCc4nnc(C)o4)ccc3F)c2[nH]c1=O ; CCCNC(=O)c1c(N)c2cccc(-c3cc(OCc4nnc(C(F)(F)F)o4)ccc3F)c2[nH]c1=O]

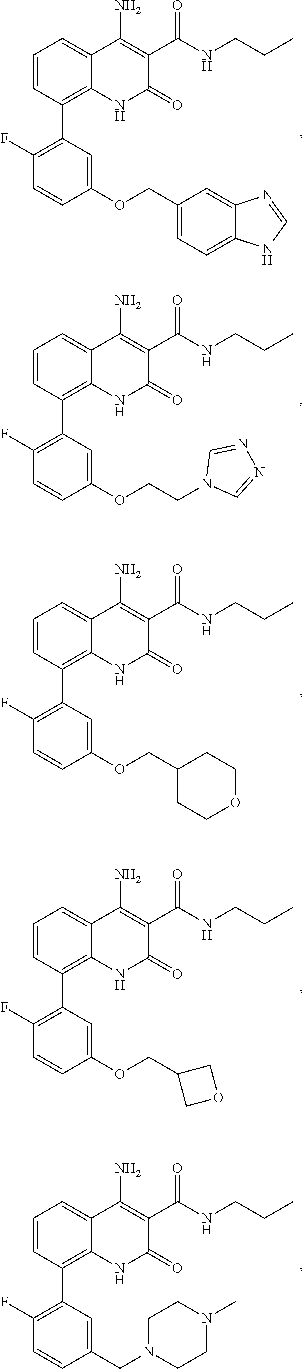 [SMILES: CCCNC(=O)c1c(N)c2cccc(-c3cc(OCc4ccc5[nH]cnc5c4)ccc3F)c2[nH]c1=O ; CCCNC(=O)c1c(N)c2cccc(-c3cc(OCCn4cnnc4)ccc3F)c2[nH]c1=O ; CCCNC(=O)c1c(N)c2cccc(-c3cc(OCC4CCOCC4)ccc3F)c2[nH]c1=O ; CCCNC(=O)c1c(N)c2cccc(-c3cc(OCC4COC4)ccc3F)c2[nH]c1=O ; CCCNC(=O)c1c(N)c2cccc(-c3cc(CN4CCN(C)CC4)ccc3F)c2[nH]c1=O]

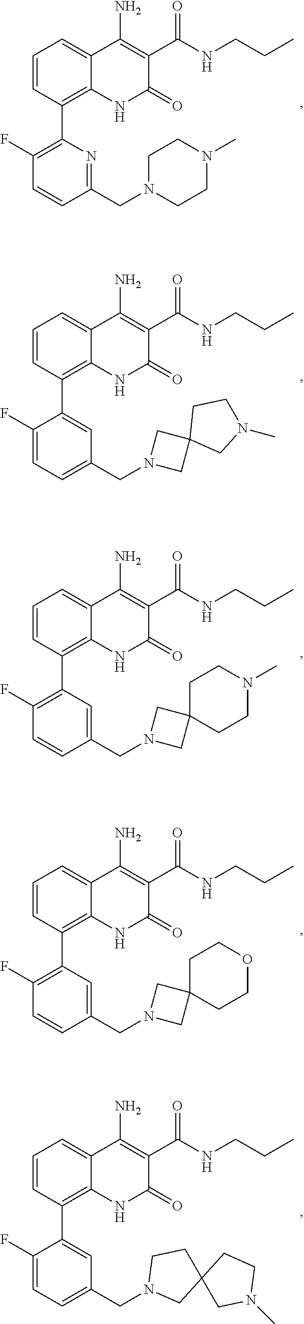 [SMILES: CCCNC(=O)c1c(N)c2cccc(-c3nc(CN4CCN(C)CC4)ccc3F)c2[nH]c1=O ; CCCNC(=O)c1c(N)c2cccc(-c3cc(CN4CC5(CCN(C)C5)C4)ccc3F)c2[nH]c1=O ; CCCNC(=O)c1c(N)c2cccc(-c3cc(CN4CC5(CCN(C)CC5)C4)ccc3F)c2[nH]c1=O ; CCCNC(=O)c1c(N)c2cccc(-c3cc(CN4CC5(CCOCC5)C4)ccc3F)c2[nH]c1=O ; CCCNC(=O)c1c(N)c2cccc(-c3cc(CN4CCC5(CCN(C)C5)C4)ccc3F)c2[nH]c1=O]

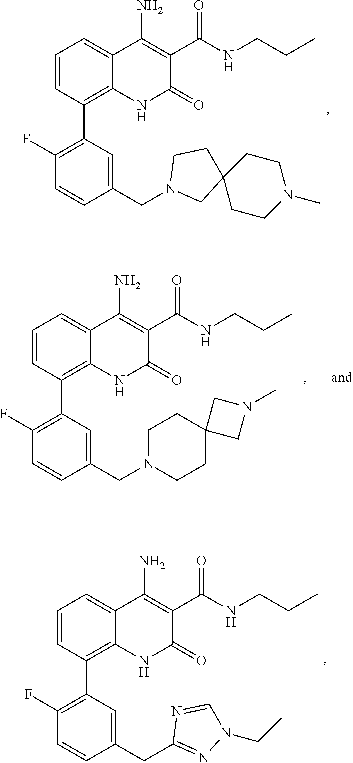 [SMILES: CCCNC(=O)c1c(N)c2cccc(-c3cc(CN4CCC5(CCN(C)CC5)C4)ccc3F)c2[nH]c1=O ; CCCNC(=O)c1c(N)c2cccc(-c3cc(CN4CCC5(CC4)CN(C)C5)ccc3F)c2[nH]c1=O ; CCCNC(=O)c1c(N)c2cccc(-c3cc(Cc4ncn(CC)n4)ccc3F)c2[nH]c1=O]

or a pharmaceutically acceptable salt thereof.

In another embodiment there is provided a compound selected from any one of the Examples herein, or a pharmaceutically acceptable salt thereof.

Particular compounds of the invention are those that have an affinity (Ki) for α2-GABA_{A}Rs and/or α3-GABA_{A}Rs of less than 30 nM (e.g. 10 nM, or less) when measured in the in vitro radioligand binding assay described herein. Preferred compounds of the invention have binding affinities and/or efficacy that are selective for α2-GABA_{A}Rs and/or α3-GABA_{A}Rs over GABA_{A} receptors containing α1 subunits. Particular compounds of the invention exhibit improved α2-GABA_{A}Rs and/or α3-GABA_{A}Rs functional activity compared to diazepam in the in vitro α2-GABA_{A}R Relative Efficacy assay described herein.

### PHARMACEUTICAL COMPOSITIONS

In accordance with another aspect, the present invention provides a pharmaceutical composition comprising a compound of the invention, or a pharmaceutically acceptable salt thereof, and a pharmaceutically acceptable excipient.

Conventional procedures for the selection and preparation of suitable pharmaceutical compositions are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs), for sublingual use, for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intraperitoneal dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.1 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, a daily dose selected from 0.1 mg/kg to 100 mg/kg, 1 mg/kg to 75 mg/kg, 1 mg/kg to 50 mg/kg, 1 mg/kg to 20 mg/kg or 5 mg/kg to 10 mg/kg body weight is received, given if required in divided doses. In general, lower doses will be administered when a parenteral route is employed. Thus, for example, for intravenous, subcutaneous, intramuscular or intraperitoneal administration, a dose in the range, for example, 0.1 mg/kg to 30 mg/kg body weight may be suitable. Similarly, for administration by inhalation, a dose in the range, for example, 0.05 mg/kg to 25 mg/kg body weight may be suitable. When administered orally a total daily dose of a compound of the invention may be, for example, selected from: 1 mg to 1000 mg, 5 mg to 1000 mg, 10 mg to 750 mg or 25 mg to 500 mg. Typically, unit dosage forms will contain about 0.5 mg to 0.5 g of a compound of the invention. In a particular embodiment the compound of the invention is administered parenterally, for example by intravenous administration. In another particular embodiment the compound of the invention is administered orally.

### Therapeutic Uses and Applications

In accordance with another aspect, the present invention provides a compound of the invention, or a pharmaceutically acceptable salt thereof, for use as a medicament.

A further aspect of the invention provides a compound of the invention, or a pharmaceutically acceptable salt thereof, for use in the prevention or treatment of a disease or medical disorder mediated by α2-GABA_{A}Rs and/or α3-GABA_{A}Rs. It may be that the disease or medical disorder mediated by α2-GABA_{A}Rs. It may be that the disease or medical disorder mediated by α3-GABA_{A}Rs.

Also provided is a method of preventing or treating a disease or medical disorder mediated by α2-GABA_{A}Rs and/or α3-GABA_{A}Rs in a subject, the method comprising administering to the subject an effective amount of a compound of the invention, or a pharmaceutically acceptable salt thereof. It may be that the disease or medical disorder mediated by α2-GABA_{A}Rs. It may be that the disease or medical disorder mediated by α3-GABA_{A}Rs.

Also provided is the use of a compound of the invention, or a pharmaceutically acceptable salt thereof, for the manufacture of a medicament for the prevention or treatment of a disease or medical disorder mediated by α2-GABA_{A}Rs and/or α3-GABA_{A}Rs. It may be that the disease or medical disorder mediated by α2-GABA_{A}Rs. It may be that the disease or medical disorder mediated by α3-GABA_{A}Rs.

In the following sections of the application reference is made to a compound of the invention, or a pharmaceutically acceptable salt thereof for use in the prevention or treatment of certain diseases or medical disorders. It is to be understood that any reference herein to a compound for a particular use is also intended to be a reference to (i) the use of the compound of the invention, or pharmaceutically acceptable salt thereof, in the manufacture of a medicament for the prevention or treatment of that disease or disorder; and (ii) a method for the prevention or treatment of the disease or disorder in a subject, the method comprising administering to the subject a therapeutically effective amount of the compound of the invention, or pharmaceutically acceptable salt thereof.

In certain embodiments there is provided a compound of the invention, or a pharmaceutically acceptable salt thereof, for use in the prevention or treatment of a medical disorder selected from: an anxiety disorder, a mood disorder, pain, a neurodegenerative disorder, a neurodevelopmental disorder, a cognitive disorder and a psychiatric disorder.

In certain embodiments there is provided a compound of the invention, or a pharmaceutically acceptable salt thereof, for use in the prevention or treatment of an anxiety disorder selected from: panic disorder, panic disorder without agoraphobia, panic disorder with agoraphobia, agoraphobia without history of panic disorder, specific phobia, social phobia, social anxiety disorder, obsessive-compulsive disorder, posttraumatic stress disorder, acute stress disorder, generalized anxiety disorder, and generalized anxiety disorder due to a general medical condition.

In certain embodiments there is provided a compound of the invention, or a pharmaceutically acceptable salt thereof, for use in the prevention or treatment of a mood disorder selected from: major depressive disorder, dysthymic disorder, bipolar depression and/or bipolar mania, bipolar I with or without manic, depressive or mixed episodes, bipolar II, cyclothymic disorder, mood disorder due to a general medical condition, manic episodes associated with bipolar disorder, and mixed episodes associated with bipolar disorder.

In certain embodiments there is provided a compound of the invention, or a pharmaceutically acceptable salt thereof, for use in the prevention or treatment of pain, for example in the treatment or prevention of neuropathic pain.

In certain embodiments there is provided a compound of the invention, or a pharmaceutically acceptable salt thereof, for use in the prevention or treatment of a neurodegenerative disorder (e.g. Alzheimer's disease, Parkinson's disease, Huntington's disease or amyotrophic lateral sclerosis). Thus in certain embodiments there is provided a compound of the invention, or a pharmaceutically acceptable salt thereof, for use in the prevention or treatment of Alzheimer's disease.

In certain embodiments there is provided a compound of the invention, or a pharmaceutically acceptable salt thereof, for use in the prevention or treatment of a cognitive disorder (e.g. dementia, dementia due to Alzheimer's disease, and dementia due to Parkinson's disease).

In certain embodiments there is provided a compound of the invention, or a pharmaceutically acceptable salt thereof, for use in the prevention or treatment of a neurodevelopmental disorder (e.g. Down's syndrome, autism, fragile X syndrome, or attention deficit/hyperactivity disorder (ADHD)).

In certain embodiments there is provided a compound of the invention, or a pharmaceutically acceptable salt thereof, for use in the prevention or treatment of a psychiatric disorder (e.g. schizophrenia).

In certain embodiments there is provided a compound of the invention, or a pharmaceutically acceptable salt thereof, for use in the prevention or treatment of General Anxiety Disorder, panic disorder, seizures, movement disorders, epilepsy, seizures, psychosis, mood disorders, muscle spasms, addiction (e.g. alcohol or drug dependency), substance abuse, withdrawal symptoms, autism, fragile X syndrome, pain and pruritus.

The compounds of the invention are expected to provide an anxiolytic effect. Accordingly in certain embodiments there is provided a compound of the invention, or a pharmaceutically acceptable salt thereof, for use in the prevention or treatment of anxiety associated with a medical disorder. For example, a compound of the invention, or a pharmaceutically acceptable salt thereof, for use in the prevention or treatment of anxiety associated with a medical disorder selected from: a mood disorder, pain, a neurodegenerative disorder, a neurodevelopment disorder, a cognitive disorder and a psychiatric disorder. In those embodiments where the anxiety is associated with a mood disorder, the mood disorder may be any of the mood disorders described herein. In those embodiments where the anxiety is associated with a neurodegenerative disorder, the neurodegenerative disorder may be any of the neurodegenerative disorders described herein. In those embodiments where the anxiety is associated with a neurodevelopmental disorder, the neurodevelopmental disorder may be any of the neurodevelopmental disorders described herein. For example, a compound of the invention or a pharmaceutically acceptable salt thereof if for use in the treatment or prevention of anxiety or agitation associated with fragile X syndrome. In those embodiments when the anxiety is associated with a cognitive disorder, the cognitive disorder may be any of the cognitive disorders described herein. In those embodiments when the anxiety is associated with a psychiatric disorder, the cognitive disorder may be any of the psychiatric disorder described herein. Thus it may be that a compound of the invention or a pharmaceutically acceptable salt thereof if for use in the treatment or prevention of anxiety or agitation associated with schizophrenia.

Without wishing to be bound by theory it is expected that the selective modulation of α2-GABA_{A}Rs and/or α3-GABA_{A}Rs relative to 1-GABA_{A}Rs will provide compounds with a desirable therapeutic effect whilst avoiding or minimising the side effects associated with non-selective GABA_{A} modulators such as benzodiazepines (e.g. diazepam). For example a compound of the invention may avoid or reduce undesirable side effects. A compound of the invention may therefore reduce or avoid one or more of addiction, drowsiness, poor concentration, ataxia, dysarthria, motor incoordination, diplopia, muscle weakness, vertigo, and mental confusion compared to the use of benzodiazepines such as diazepam.

### Selectivity

Non-selective inhibition of GABA_{A}Rs can result in undesirable side-effects, for example as discussed above. Selective α2-GABA_{A}R/α3-GABA_{A}R modulation (e.g. activation) is expected to provide beneficial therapeutic effects, for example anxiolytic effects, whilst avoiding or minimising the risk of undesirable side effects associated with non-selective GABA_{A}R modulation, particularly α1-GABA_{A}R modulation.

Accordingly, preferred compounds of the invention, have selective affinity for, and/or activate the function of, α2 and/or α3 subunit containing GABA_{A}Rs over GABA_{A}Rs that contain an α1 subunit. In certain embodiments a compound of the invention has an affinity (Ki) for α2-GABA_{A}Rs that is at least 5-fold, at least 10-fold, at least 15-fold, at least 20-fold, at least 50-fold, at least 100-fold, at least 500-fold, or at least 1000-fold lower (e.g. about 20 to about 1000 fold lower) than the Ki for GABA_{A} receptors containing α1-, -subunits when measured using the in vitro radioligand binding assay described herein. In certain embodiments a compound of the invention has an affinity (Ki) for α3-GABA_{A}Rs that is at least 5-fold, at least 10-fold, at least 15-fold, at least 20-fold, at least 50-fold, at least 100-fold, at least 500-fold, or at least 1000-fold lower (e.g. about 20 to about 1000 fold lower) than the Ki for GABA_{A} receptors containing α1-subunits when measured using the in vitro radioligand binding assay described herein.

In some embodiments compounds of the invention exhibit α2-GABA_{A}R PAM activity when measured in the in vitro electrophysiological recording assay described herein. Preferred compounds of the invention selectively potentiate the function of α2- and/or α3-GABA_{A}Rs over GABA_{A}Rs containing α1, subunits when measured in the in vitro electrophysiological recording assay described herein.

In certain embodiments such selective compounds may be used in the treatment or prevention of any of the diseases or medical conditions described herein.

### Combination Therapies

The compounds of the invention may be used alone to provide a therapeutic effect. The compounds of the invention may also be used in combination with one or more additional therapeutic agents.

In some embodiments the additional therapeutic agent is selected from one or more of:
antidepressants, for example, a serotonergic agents (e.g., a Selective Serotonin Reuptake Inhibitor (SSRI, e.g. bupropion, citalopram, escitalopram, fluoxetine, fluvoxamine, paroxetine, sertraline, trazodone), a serotonin-norepinephrine reuptake Inhibitor (SNRI, e.g., duloxetine or venlafaxine), a tricyclic antidepressant (e.g. amitriptyline, amoxapine, clomipramine, desipramine, doxepin, imipramine, maprotiline, nortriptyline, trimipramine, or protriptyline), a monoamine oxidase inhibitor (e.g., phenelzine or tranylcypromaine), elzasonan, gepirone, ipsapirone, mirtazapine, reboxetine, robalzotan, thionisoxetine, nefazodone, or buspirone;
antipsychotics, for example, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, lithium, loxapine, mesoridazine, olanzapine, paliperidone, perlapine, perphenazine, phenothiazine, phenylbutlypiperidine, pimozide, prochlorperazine, quetiapine, risperidone, quetiapine, sertindole, sulpiride, suproclone, suriclone, thioridazine, trifluoperazine, trimetozine, valproate, valproic acid, zopiclone, zotepine, or ziprasidone;
an adrenergic inhibitor (e.g., propranolol, prazosin, clonidine or guanfacine);
an antihistamines (e.g., hydroxyzine or diphenhydramine);
anticonvulsants (e.g., gabapentin, pregabalin, lamotrigine, topiramate or valproate);
an antipsychotic (e.g., quetiapine, olanzapine, risperidone),
an acetylcholinesterase inhibitor;
a Parkinson's therapy;
a migraine therapy;
a neuropathic pain therapy, for example, gabapentin, lidoderm or pregabalin;
an insomnia therapy;
an Alzheimer's therapy, for example donepezil, memantine or tacrine.

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

### Biological Assays

The biological effects of the compounds may be assessed using one of more of the assays described herein.

### In Vitro Radioligand Binding Assay (α2-GABA_{A}R Ki)

The affinity (Ki) of compounds for the benzodiazepine site of human recombinant GABA_{A}Rs was measured by their ability to inhibit the binding of the selective benzodiazepine antagonist [³H]Ro15-1788 ([³H] flumazenil).

Mouse L(tk⁻) cells stably expressing human α1β3γ2, α2β3γ2, α3β3γ2, α5β3γ2 GABA_{A}Rs were generated by transfection of the individual subunits in the dexamethasone-inducible expression vector pMSGneo in mouse L(tk⁻) cells (Hadingham et al., 1993, Mol. Pharmacol. 43:970-975 and 1993, Mol. Pharmacol. 44:1211-1218).

L(tk⁻) cells stably expressing human α1β3γ2, α2β3γ2, α3β3γ2, α5β3γ2 GABA_{A}Rs were maintained in DMEM F12 medium supplemented with 10% Foetal Bovine Serum, 1% Penicillin/Streptomycin and 1 mg/mL Geneticin G418 in an incubator at 37° C. with a humidified atmosphere with 5% CO₂. Dexamethasone was added to the culture medium to induce GABA_{A}R expression.

L(tk−) cells were harvested and membranes were prepared for each receptor combination in either TE buffer (10 mM Tris·CI/0.1 mM EDTA, pH 7.5) or phosphate buffer (K₂PO₄ 10 mM, pH 7.0) (Hadingham et al. (1992) Proc. Natl. Acad. Sci. USA; 89 (14): 6378-82). Protein concentration, receptor expression and the Kd of [³H]Ro15-1788 were determined before Ki value of the compounds were evaluated. For Kd evaluation, saturation binding curves were obtained by incubating membrane with various concentrations of [³H]Ro15-1788 (82.5 Ci/mM), with nonspecific binding measured in the presence of 1-3 μM TP003.

[³H]Ro15-1788 ([³H] flumazenil) is tritiated on the N-methyl group as shown:

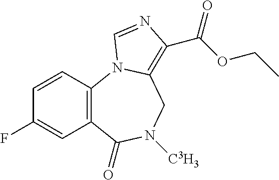

TP003 is a non-selective GABA_{A}R benzodiazepine site agonist of the formula:

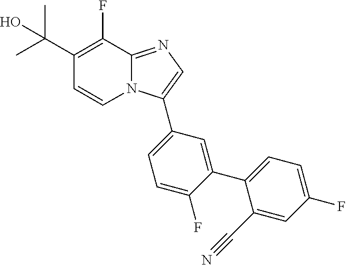 [SMILES: CC(C)(O)c1ccn2c(-c3ccc(F)c(-c4ccc(F)cc4C#N)c3)cnc2c1F]

For Ki evaluation, cell membranes were incubated with 4 nM [³H]Ro15-1788 along with a range of concentrations of test compound. Nonspecific binding was determined using 1-3 μM TP003. All incubations were performed for 1 hour at 4° C. in assay buffer. The total assay volume was 0.5 mL, containing 40 μg/well α1β3γ2, 30 μg/well α2β3γ2, 40 μg/well α3β3γ2 and 30 μg/well for α5β3γ2 of membrane protein. Incubations were terminated by filtration and washing with ice cold Tris-HCl buffer (50 mM, pH=7.4) over Whatman GF/B filters and the radioactivity of the filters was measured using liquid scintillation counting.

The % inhibition of [³H]Ro15-1788 binding was plotted as a function of compound concentration and the IC₅₀ calculated. From the IC₅₀, the affinity (Ki) was calculated using the method of Cheng and Prusoff using the Kd values obtained for [³H]Ro15-1788.

The compounds of the present invention tested in the above described assay were found to have high affinity for GABA_{A}Rs. Preferred compounds have a Ki<30 nM for the α2- and α3-GABA_{A}RS.

### In Vitro Electrophysiological Recording Assay (α2-GABA_{A}R Relative Efficacy)

The efficacy of modulators was evaluated using the automated patch-clamp platforms QPatch16 (Sophion, Copenhagen, Denmark) or SyncroPatch 384PE (Nanion, Germany).

For QPatch electrophysiology testing L(tk⁻) cells stably expressing human α1β3γ2, α2β3γ2, α3β3γ2, α5β3γ2 GABA_{A}Rs were used. HEK293 cells stably expressing human α1β3γ2L, α2β3γ2L, α3β3γ2L and α5β3γ2L GABA_{A}Rs were used for the SyncroPatch 384PE experiments.

All experiments were carried out at room temperature (20-22° C.) using a standard whole cell procedure and physiological solutions. Gigaseals were formed upon execution of a combined suction/voltage protocol with subsequent increased suction leading to the whole-cell configuration.

The currents recorded were acquired at either 1 KHz or 2 KHz and filtered using a Bessel filter. Whole-cell currents were measured at a holding potential of-65 mV (QPatch) or −80 mV (SyncroPatch).

For QPatch experiments, the extracellular solution contained of: 145 mM NaCl, 4 mM KCl, 1 mM MgCl₂, 2 mM CaCl₂), 10 mM HEPES, 10 mM D-glucose (pH 7.4), and the intracellular solution consisted of: 96 mM KCl, 28 mM CsCl, 25 mM KOH, 4.3 mM CaCl₂), 1.4 mM MgCl₂, 10 mM EGTA, 10 mM HEPES, 3 mM MgATP (pH 7.2). The osmolarities of the extracellular and intracellular solutions were 305 and 295 mOsm respectively.

For SyncroPatch experiments, the extracellular recording solution contained: 140 mM NaCl, 4 mM KCl, 2 mM CaCl₂), 1 mM MgCl₂, 10 mM HEPES, 5 mM glucose (pH 7.4 with NaOH and osmolarity of c. 300-310 mOsm). The intracellular recording solution consisted of: 90 mM KCl, 50 mM KF, 1.5 mM MgCl₂, 11.1 mM EGTA and 10 mM HEPES (pH 7.2 with KOH and osmolarity of c. 300 mOsm). 2 mM of NaATP was added to the intracellular solution on the day of testing.

The effects of modulators were evaluated in the presence of a submaximal GABA concentration, giving typically less than 30% activation of the response elicited by a saturating GABA concentration. To check and ensure baseline current stability before compound addition, two to five consecutive applications of GABA EC₁₀₋₂₀ separated by wash steps were performed before compound addition. The test compound was applied 1 min at least prior to co-application with GABA EC₁₀₋₂₀. Following a further wash step, a saturating concentration of GABA was applied at the end of each recording, allowing the accurate evaluation for each cell of the percentage baseline activation elicited by the submaximal GABA applied.

The compounds were first dissolved in DMSO as a 10 mM stock and then further diluted to the testing concentrations so that the final DMSO concentration in the extracellular recording solution was 0.1% (QPatch) or 0.2% (SyncroPatch).

The % efficacy of modulators was determined from the GABA elicited currents recorded in the presence and absence of the test compound, using the formula:

[((compound peak current−leak)−(GABA peak current-leak))/(GABA peak current−leak)]*100,

where ‘leak’ is the leak baseline current at the holding potential, ‘compound peak current’ is the current elicited by co-application of compound and GABA, and ‘GABA peak current’ is the current elicited by GABA alone at the last GABA application before compound addition. The results were presented as ‘relative efficacy’ for each compound generally at a concentration equal or higher than 100 times their determined Ki. The relative efficacy of a compound was calculated by normalising its % efficacy to the corresponding % efficacy of diazepam that was separately determined.

The compounds of the invention tested in the above described assay were found to possess α2- and α3-GABA_{A}R PAM activity and selectivity over the α1-GABA_{A}R.

### Rat Brain Receptor Occupancy Assay

The occupancy of rat brain benzodiazepine binding sites on GABA_{A}R by compound was measured by its ability to inhibit the ex vivo binding of [³H]Ro15-1788 ([³H] flumazenil) in brain samples from rats dosed with the test compound.

Male Sprague Dawley rats (c. 200-300 g) were used for the studies. All animals were acclimatized 5-7 days before dosing and were handled and weighed prior to treatments.

Rats (3 to 6 per group) were dosed p.o. with either the vehicle or the test compound suspended in vehicle (doses of 0.1 to 10 mg/kg) at a volume of 5 mL per kg of body weight. To block all benzodiazepine binding sites and therefore define the level of non-specific binding of [³H]Ro15-1788, a separate group of animals received a dose of 5 mg/kg p.o. of bretazenil (5 mL/kg) made up in 70% PEG, 30 minutes before the animals were killed.

At predetermined time intervals, animals were culled via stunning and cervical dislocation. Brain tissues were harvested promptly, and each left and right hemisphere were snap frozen and stored at −80° C.

On the day of the brain samples analysis, each left hemisphere was weighed and homogenized in 8 volumes of ice-cold 10 mM potassium phosphate buffer (pH 7.4). 300 μL of the mixture was further diluted by adding it to a tube containing 1050 μL of assay buffer and [³H] flumazenil further added to make a total volume of 1500 μL and achieve a final concentration of 4 nM, mixing immediately and incubating on ice.

Following 20 seconds incubation time, the sample was rapidly filtered through Whatman GF/B filter in triplicates (500 μL/filter) using a vacuum filtration manifold. The filters were subsequently washed with 10 mL of ice cold 50 mM Tris buffer (pH 7.4) and then placed into scintillation vials containing scintillation fluid. Filter-bound radioactivity was counted on a TriCarb2900 scintillation counter.

In each experiment, the value of nonspecific binding defined using bretazenil was subtracted from all groups to give values of specific binding.

The percent GABA_{A}R occupancy of the test compound at a specific dose and treatment time was calculated as the percentage by which the specific binding in the vehicle treated group was inhibited by the drug. Therefore, the % occupancy of the modulator in drug-treated animals was expressed as: 100−((Average test compound−Average bretazenil)/(Average vehicle−Average Bretazenil)*100) where ‘average test compound’, ‘average bretazenil’ and ‘average vehicle’ are the average counts in test compound-, bretazenil- and vehicle-treated animals, respectively.

Certain compounds of the invention were tested in the above described assay and showed brain GABA_{A}R engagement when dosed orally.

### Elevated Plus Maze Assay

The method, which detects anxiolytic activity, follows that described by Handley and Mithani (Naunyn. Schmied. Arch. Pharmacol., 327, 1-5, 1984). Rodents avoid open spaces (the open arms of an elevated plus-maze). Anxiolytics increase exploratory activity in the open arms, as indicated by increased time spent on the open arms and/or by increased % open-arm entries.

The maze was made from black matte acrylic and consisted of 4 arms of equal length and width (50×10 cm) arranged in the form of a plus sign (+). Two opposite arms were enclosed by 40 cm high walls (closed arms). The two other arms have no walls (open arms). The maze was raised approximately 65 cm above the floor.

Male Sprague Dawley rats (c. 200-300 g) were used for the study. 13 to 15 rats were studied per group.

Compounds were evaluated at three or four doses, administered p.o. 30 minutes to 2 hours before the test, at 5 mL per kg of body weight and its effects compared with vehicle-treated group.-Chlordiazepoxide hydrochloride (5 mg/kg, prepared in 0.9% saline), administered i.p. 30 minutes before the test, was used as reference substance to compare with the vehicle treated group.

The day before the test, rats were weighed. The compounds were prepared at least 24 hours before the test day. 30 minutes after dosing, each rat was placed in the centre of the apparatus and its behaviour was recorded for 5 minutes. The animals' behaviour on the maze was tracked using a camera mounted above the maze and the image analysed using EthoVision XT 13 video tracking software.

The percentage of time spent in the open arms was calculated by dividing the time spent on open arms to the total time in the maze.

Data were analysed by comparing treated groups with vehicle control group using one-way ANOVA followed by post-hoc Dunnett's tests.

Certain compounds of the invention were tested in the above described assay and showed significant anxiolytic-like effects when dosed orally.

### Synthesis

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl or trifluoroacetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃·OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, or sodium hydroxide, or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

### General Synthetic Routes

Compounds of the invention may be prepared by a number of synthetic routes, including but not limited to the following.

Compounds of formula (II) may be prepared by the process illustrated in Scheme 1.

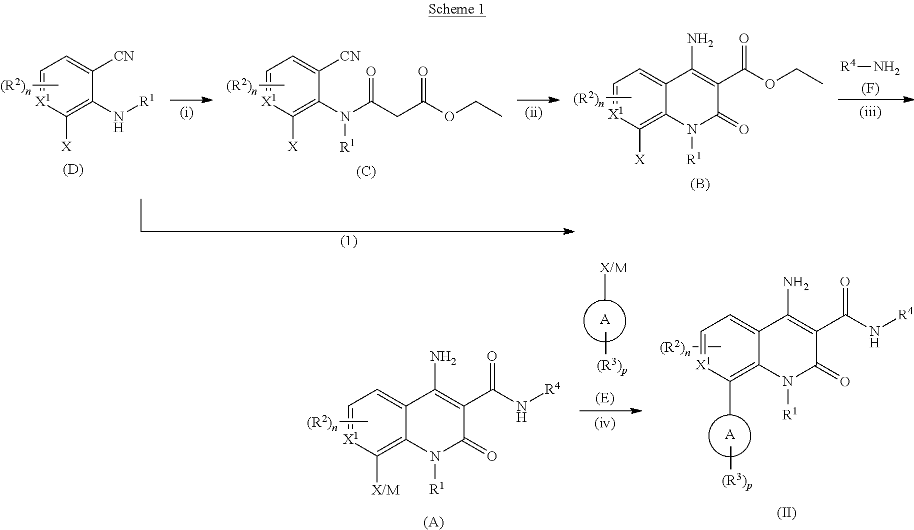

wherein X is Cl or Br; X¹ is C or N; M is boronic acid or an ester thereof; ring A comprises six- and five-membered heteroaryl and phenyl;
and R¹, R², R³, R⁴, and n have any of the meanings defined herein.

Compounds of formulae (D), (E) and (F) are commercially available or may be synthesised by those skilled in the art according to the literature or preparations described herein.

Compounds of formula (II) may be prepared from compounds of formula (A) according to process step (iv), typically via a Suzuki cross-coupling reaction with compounds of formula (E). Typical conditions for the metal-catalysed cross-coupling reaction comprise a palladium catalyst such as [1,1′-bis(di-tert-butylphosphino)ferrocene]dichloropalladium(II) or [1,1′-bis(diphenylphosphino)ferrocene]dichloro palladium(II) or tris(dibenzylideneacetone)dipalladium(0) with a suitable base such as sodium, potassium or cesium carbonate or potassium fluoride or potassium phosphate tribasic in solvents such as dioxane/water or acetonitrile/water at temperatures ranging from room temperature to 100° C. If heating is required, this is performed either thermally or under microwave irradiation. Wherein tris (dibenzylideneacetone) dipalladium (0) is used, a phosphine ligand is required such as tri-tert-butylphosphine. During this step if compounds of formula (A) need to be converted to the boronic acid or boronate ester, additional step may be used to convert X to M. Typical conditions comprise [1,1′-bis(diphenylphosphino)ferrocene]dichloro palladium(II) with potassium acetate in solvent such as dioxane at 90° C.

Compounds of the formula (E) are either commercially available or may be synthesised by those skilled in the art according to the literature or preparations described herein.

Compounds of formula (A) may be prepared from compounds of formula (B) according to process step (iii), by formation of an amide bond directly from the ester of formula (B). Typical conditions comprise heating compounds of formula (B) with amines of formula (F) in the presence of triethyl aluminium at temperatures up to 100° C. in a solvent such as toluene or dichloroethane. Alternative conditions comprise heating compounds of formula (B) and amines of formula (F) with 1,5,7-triazabicyclo[4.4.0]dec-5-ene in a solvent such as acetonitrile at temperatures ranging from 50° C. to 80° C.

Compounds of formula (F) are commercially available or may be synthesised by those skilled in the art according to the literature.

Compounds of formula (B) may be prepared from compounds of formula (C) according to process step (ii), a cyclisation reaction. Typical conditions comprise heating compounds of formula (C) with a base such as sodium methoxide or potassium fluoride in a solvent such as ethanol or acetonitrile/water at elevated temperatures ranging from 60° C.-100° C.

Compounds of formula (C) may be prepared from compounds of formula (D) according to process step (i), an amide bond formation with ethyl malonyl chloride. Typical conditions comprise stirring compounds of formula (D) with ethyl malonyl chloride either in toluene and heated to 110° C. or in the presence of triethylamine in solvent such as dichloromethane at room temperature.

Compounds of formula (B) may also be prepared in a single step reaction from compounds of formula (D) according to process step (1). Typical conditions comprise stirring compounds of formula (D) with diethyl malonate in the presence of tin (IV) chloride in a solvent such as toluene and heated at temperature up to 110° C.

Compounds of formula (D) are either commercially available or may be synthesised by those skilled in the art according to the literature or preparations described herein.

Compounds of formula (II) may also be prepared by the process illustrated in Scheme 2.

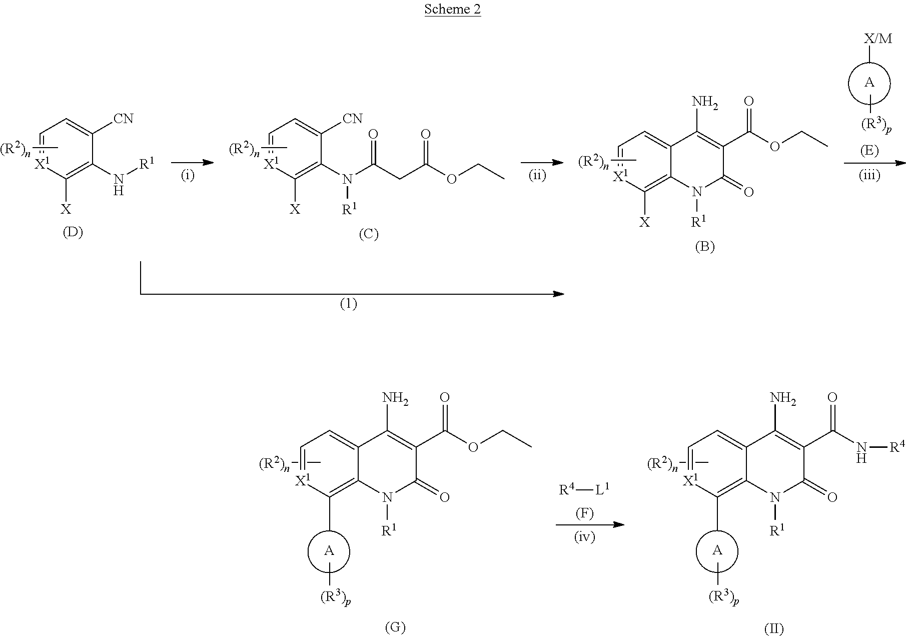

wherein X, X¹, M, ring A, R¹, R², R³, R⁴, and n have any of the meanings as defined herein.

Compounds of formula (II) may be prepared from compounds of formula (G) according to process step (iv), by formation of an amide bond directly from the ester of formula (G). Reaction conditions for the amide bond formation are analogous to those described for reaction scheme 1.

Compounds of formula (G) may be prepared from compounds of formula (B) according to process step (iii), typically via a Suzuki cross-coupling reaction with compounds of formula (E). Typical conditions for the metal-catalysed cross-coupling reaction are analogous to those described for reaction scheme 1.

Reaction conditions for the synthesis of compounds of formula (B), and (C) are analogous to those described for reaction scheme 1 above. Compounds of formulae (D), (E) and (F) are commercially available or may be synthesised by those skilled in the art according to the literature or preparations described herein.

Compounds of formula (III) and (IV) may be prepared using conditions analogous to those described for scheme 1 and scheme 2 above.

Compounds of formula (V) may be prepared by the process illustrated in Scheme 3.

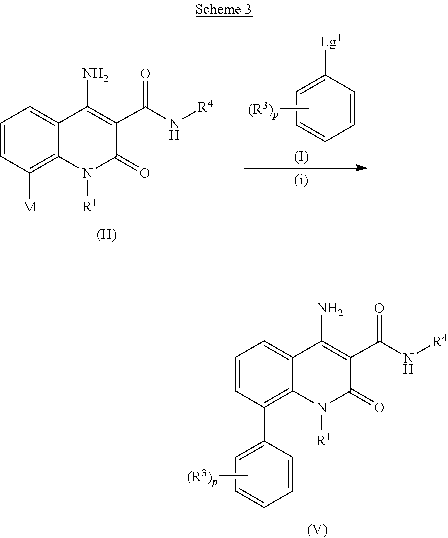

wherein R¹, R³, R⁴, and p have any of the meanings defined herein; M is a boronic acid or an ester thereof, or a trifluoroborate salt; and Lg¹ is a halo, particularly Cl or Br.

Compounds of formula (V) may be prepared from compounds of formula (H) according to process step (i), a Suzuki cross-coupling reaction with compounds of formula (I). Typical conditions for the metal-catalysed cross-coupling reaction are analogous to those described for reaction scheme 1.

Compounds of formula (I) are either commercially available or may be synthesised by those skilled in the art according to the literature or preparations described herein.

Compounds of formula (VI) may be prepared by the process illustrated in Scheme 4:

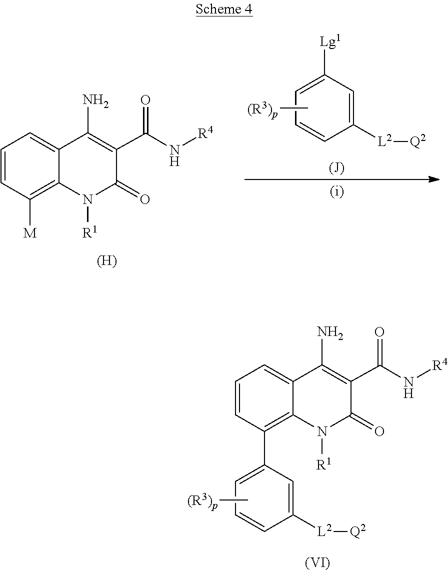

wherein R¹, R³, R⁴, L², Q² and p have any of the meanings defined herein; M is a boronic acid or an ester thereof, or a trifluoroborate salt; and Lg¹ is a halo, particularly Cl or Br.

Compounds of formula (VI) may be prepared from compounds of formula (H) according to process step (i), a Suzuki cross-coupling reaction with compounds of formula (J). Typical conditions for the metal-catalysed cross-coupling reaction are analogous to those described for reaction scheme 1.

Compounds of formula (J) are either commercially available or may be prepared according to Scheme 5:

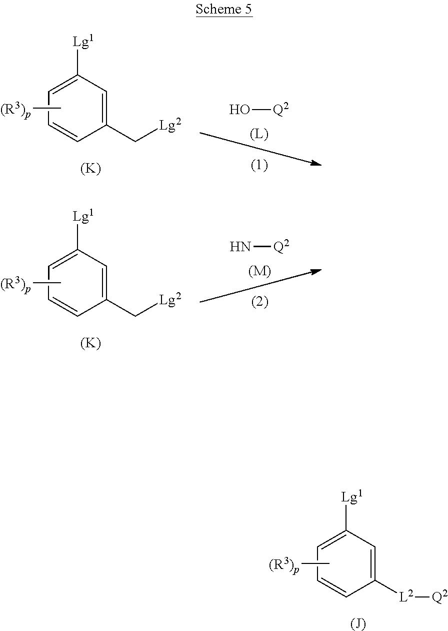

wherein R³, L², Q² and p have any of the meanings defined herein; Lg¹ is halo, particularly Cl or Br; and Lg² is halo or alcohol.

Compounds of formula (J) may be prepared from compounds of formula (K) according to process (1), a Mitsunobu reaction with alcohols of formula (L). Typical conditions comprise reacting compounds of formula (K) with alcohols of formula (L), triphenylphosphine, an azodicarboxylate such as diethyl azodicarboxylate or diisopropyl azodicarboxylate in solvent such as THF or dichloromethane at temperatures ranging from room temperature to 70° C.

Compounds of formula (L) are either commercially available or may be synthesised by those skilled in the art according to the literature or preparations described herein.

Compounds of formula (J) may also be prepared from compounds of formula (K) according to process (2), an SN₂ substitution with an amine of formula (M). Typical conditions comprise reacting compounds of formula (K) and amines of formula (M) in a solvent such as dichloromethane, acetonitrile or acetone. When necessary, bases such as DIPEA may also be added.

Compounds of formula (M) are either commercially available or may be synthesised by those skilled in the art according to the literature or preparations described herein.

Compounds of formula (K) are either commercially available or may be synthesised by those skilled in the art according to the literature or preparations described herein.

Compounds of formula (VII) and (VIII) may be prepared using conditions analogous to those described for scheme 3 above.

Compounds of formula (IX) may be prepared by the process illustrated in Scheme 6:

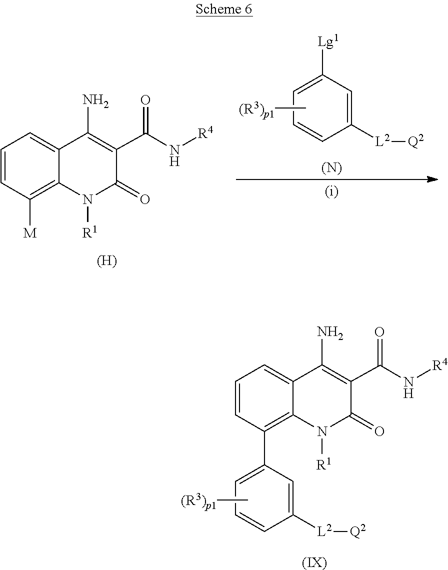

wherein R¹, R³, R⁴, L², Q², and p1 have any of the meanings defined herein; M is a boronic acid or an ester thereof, or a trifluoroborate salt; and Lg¹ is a halo, particularly Cl or Br.

Compounds of formula (IX) may be prepared from compounds of formula (H) according to process step (i), a Suzuki cross-coupling reaction with compounds of formula (N). Typical conditions for the metal-catalysed cross-coupling reaction are analogous to those described for reaction scheme 1.

Compounds of formula (N) are either commercially available or may be prepared according to Scheme 7:

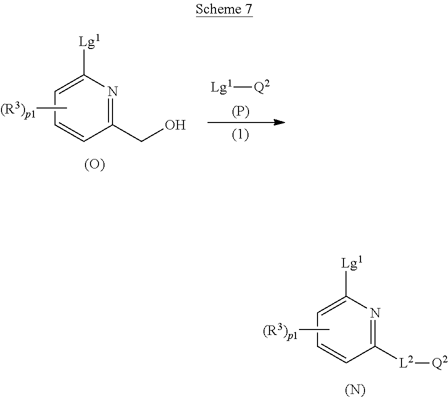

wherein R³, L², Q² and p1 have any of the meanings defined herein; Lg¹ is halo.

Compounds of formula (N) may be prepared from compounds of formula (O) according to process (1), an SN₂ substitution with an alkyl halide of formula (P). Typical conditions comprise reacting compounds of formula (O) and alkylating agent of formula (P) with a base such as sodium hydride, DIPEA, triethylamine or potassium carbonate in a solvent such as THF, dichloromethane, acetonitrile or acetone. Compounds of formula (O) are either commercially available or may be synthesised by those skilled in the art according to the literature or preparations described herein. In particular, the corresponding carboxylic acid may be used to obtain the desired alcohol. Typical conditions for the reduction reaction comprise reacting the corresponding carboxylic acid in presence of reducing agents such as borane, in solvent such as THF at temperatures ranging from room temperature to 80° C. Alternatively, compounds of formula (O) may be prepared from the corresponding carboxylic acids, via an initial conversion to the corresponding ester and subsequent reduction to alcohol, using reducing agent such as sodium borohydride in solvent such as ethanol.

Compounds of formula (P) are either commercially available or may be synthesised by those skilled in the art according to the literature or preparations described herein.

Compounds of formulae (X), (XII), (XIV) may be prepared using conditions analogous to those described for scheme 3 above.

Compounds of formulae (XI) and (XIII) may be prepared using conditions analogous to those described for scheme 7 above.

Compounds of formula (XV) may be prepared by the process illustrated in Scheme 8.

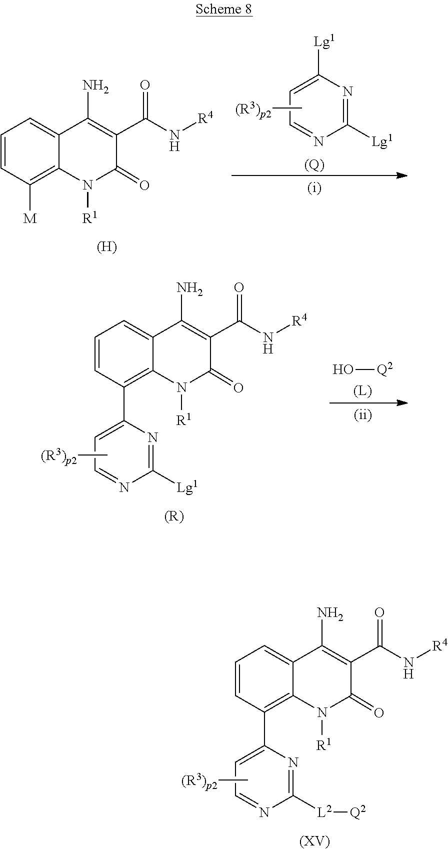

wherein R¹, R³, R⁴, L², Q², and p2 have any of the meanings defined herein; M is a boronic acid or an ester thereof, or a trifluoroborate salt; Lg¹ is halo, particularly Cl or Br.

Compounds of formula (XV) may be prepared from compounds of formula (R) according to process step (ii), a nucleophilic aromatic substitution with an alcohol of formula (L). Typical conditions comprise reacting compounds of formula (R) and alcohol of formula (L) with a base such as sodium hydride in solvent such as THF.

Compounds of formula (L) are either commercially available or may be synthesised by those skilled in the art according to the literature or preparations described herein.

Compounds of formula (R) may be prepared from compounds of formula (H) according to process step (i), a Suzuki cross-coupling reaction with compounds of formula (Q). Typical conditions for the metal-catalysed cross-coupling reaction are analogous to those described for reaction scheme 1.

Compounds of formula (Q) are either commercially available or may be synthesised by those skilled in the art according to the literature or preparations described herein.

Compounds of formula (XVI) and (XVII) may be prepared using conditions analogous to those described for scheme 3 above.

Compounds of formula (XVIII) may be prepared by the process illustrated in Scheme 9.

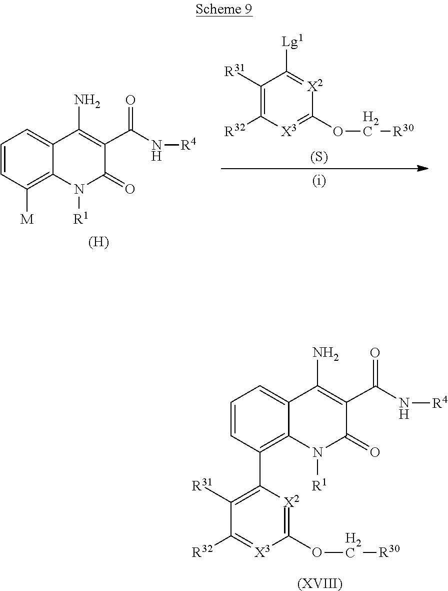

wherein R¹, R⁴, R³⁰, R³¹, and R³² have any of the meanings defined herein; X² and X³ are each independently N or CH; M is a boronic acid or an ester thereof, or a trifluoroborate salt; and Lg¹ is a halo, particularly Cl or Br.

Compounds of formula (XVIII) may be prepared from compounds of formula (H) according to process step (i), a Suzuki cross-coupling reaction with compounds of formula (S). Typical conditions for the metal-catalysed cross-coupling reaction are analogous to those described for reaction scheme 1.

Compounds of formula(S) are either commercially available or prepared according to Scheme 10:

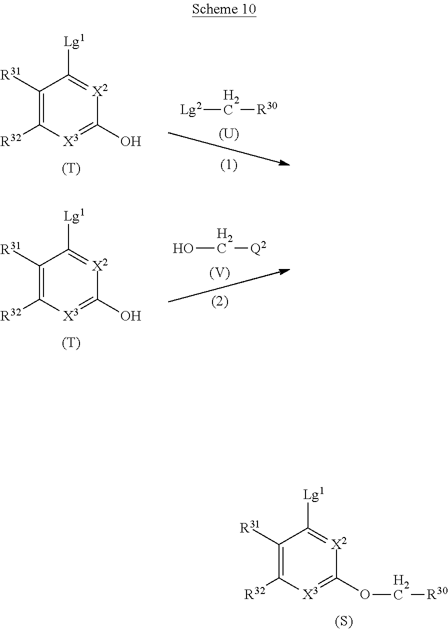

wherein R³⁰, R³¹, and R³² have any of the meanings defined herein; X¹ and X² are each independently N or CH; Lg¹ is halo, particularly Cl or Br; and Lg² is halo or mesylate.

Compounds of formula(S) may be prepared from compounds of formula (T) according to process (1), a SN₂ substitution with an alkyl halide or mesylate of formula (U). Typical conditions comprise reacting compounds of formula (T) and alkylating agent of formula (U) with a base such as DIPEA, triethylamine or potassium carbonate in a solvent such as dichloromethane, acetonitrile or acetone.

Compounds of formula (U) are either commercially available or may be synthesised by those skilled in the art according to the literature or preparations described herein. In particular when Lg² is a mesylate, the corresponding alcohol may be used to convert into the good leaving group. Typical conditions comprise reacting the alcohol with methanesulfonyl chloride in the presence of a base such as DIPEA or triethylamine in dichloromethane.

Compounds of formula(S) may also be prepared from compounds of formula (T) according to process (2), a Mitsunobu reaction with an alcohol of formula (V). Typical conditions for both processes are analogous to those described for reaction scheme 5.

Compounds of formula (T) are either commercially available or may be synthesised by those skilled in the art according to the literature or preparations described herein.

Compounds of formula (XIX), (XX) and (XXI) may be prepared using conditions analogous to those described for scheme 3 above.

Compounds of formula (XXII) may be prepared by the process illustrated in Scheme 11.

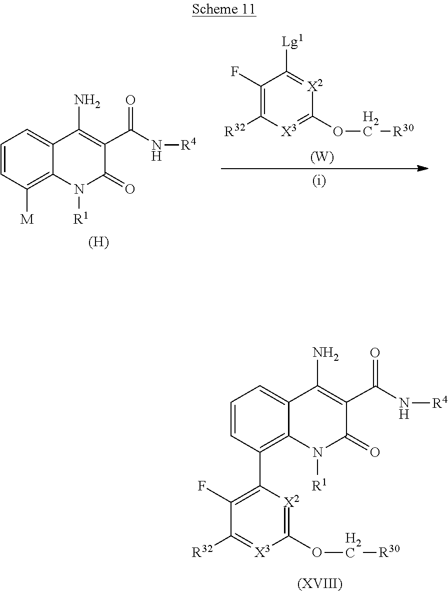

wherein R¹, R⁴, R³⁰ and R³² have any of the meanings defined herein; X² and X³ are each independently N or CH; M is a boronic acid or an ester thereof, or a trifluoroborate salt; and Lg¹ is a halo.

Compounds of formula (XVIII) may be prepared from compounds of formula (H) according to process step (i), a Suzuki cross-coupling reaction with compounds of formula (W). Typical conditions for the metal-catalysed cross-coupling reaction are analogous to those described for reaction scheme 1.

Compounds of formula (W) may be prepared using conditions analogous to those described for scheme 10 above. In particular, when X² is N and X³ is CH, or when X² is CH and X³ is N, compounds of formula W may also be prepared according to Scheme 12:

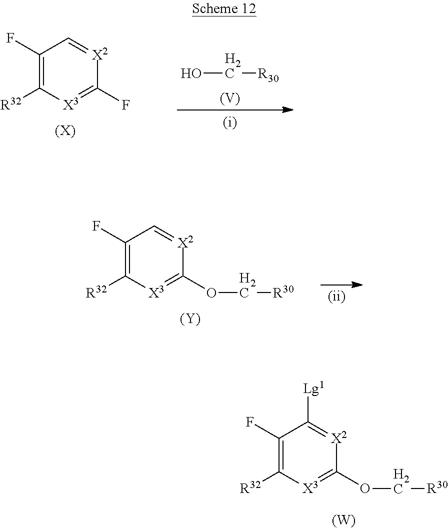

wherein R³⁰ and R³² have any of the meanings defined herein; X² and X³ are each independently N or CH; Lg¹ is halo, particularly Cl or Br.

Compounds of formula (W) may be prepared from compounds of formula (Y) according to process step (ii), a halogenation reaction. Typical conditions comprise an initial oxidation of compounds of formula (Y), obtained in presence of an oxidising agent such as mCPBA in solvent such as chloroform, followed by halogenation using reagents such as POCl₃ at temperatures ranging between 50° C. and 100° C.

Compounds of formula (Y) may be prepared from compounds of formula (X) according to process step (i), an aromatic nucleophilic substitution with an alcohol of formula (V). Typical conditions comprise reacting compounds of formula (X) and alcohol of formula (V) with a base such as sodium hydride in a solvent such as THF.

Compounds of formula (X) are either commercially available or may be synthesised by those skilled in the art according to the literature or preparations described herein.

Compounds of formula (XXIII), (XXIV), (XXV) and (XXVI) may be prepared using conditions analogous to those described for scheme 3 above.

### EXAMPLES

### Abbreviations

app Apparent
Aq. Aqueous
B₂pin₂ bis (Pinacolato)diboron
° C. degree Celsius
CDI 1,1′-Carbonyldiimidazole
DCE 1,2-Dichloroethane
DIAD Diisopropyl azodicarboxylate
DIPEA N,N-Diisopropylethylamine
Dioxane 1,4-Dioxane
dppf 1,1′-bis(Diphenylphosphino) ferrocene
DMSO Dimethylsulfoxide
Et₃N Triethylamine
EtOAc Ethyl acetate
EtOH Ethanol
Et₂O Diethyl ether
h Hour(s)
HCl Hydrochloric Acid
K₂CO₃ Potassium carbonate
KF Potassium fluoride
KOAc Potassium acetate
mCPBA 3-Chloroperbenzoic acid
MeCN Acetonitrile
MeOH Methanol
Min minute(s)
N₂ Nitrogen gas
Na₂CO₃ Sodium carbonate
NaHCO₃Sodium hydrogen carbonate
Na₂SO₄ Sodium sulfate
NH₄Cl Ammonium chloride
Pd-118 [1,1′-bis(Di-tert-butylphosphino)ferrocene]dichloropalladium(II)
Pd₂(dba)₃ tris(Dibenzylideneacetone)dipalladium(0)
Pd(dppf)Cl₂ [1,1′-bis(Diphenylphosphino)ferrocene]dichloropalladium(II)
Pet. ether Petroleum ether 40-60%
POCl₃ Phosphorus (V) oxychloride
rt Room temperature
Sat. Saturated
TBAF Tetrabutylammonium fluoride
TBD 1,5,7-Triazabicyclo[4.4.0]dec-5-ene
TBDMSCl tert-Butyldimethylsilyl chloride
THF Tetrahydrofuran

### Naming

The exemplified compounds were named using Dotmatics ELN. Other compounds, particularly commercial reagents, either use names generated by Dotmatics ELN or names commonly found in online databases and catalogues.

### NMR

All NMR spectra were obtained using Varian VNMRS 600; Varian VNMRS 500; Bruker Avance III 500 or Bruker Avance 400 spectrometers. Chemical shifts are denoted in parts per million (ppm, δ) relative to residual isotopic solvent as described in, for example, Gottlieb et al. J. Org Chem. (1997) 62 7512. The observed multiplicity of certain signals are abbreviated by: s (singlet); br (broad); d (doublet); t (triplet); q (quartet); m (multiplet); or combinations thereof. The number of protons (n) for a given resonance signal is indicated by nH. Coupling constants (J) are designated in Hz and reported to 1 decimal place.

### Mass Spectrometry

Mass spectrometry data were recorded as part of LCMS analysis obtained using a Waters 2695 HPLC coupled to a Thermo LCQ ESI-MS or APCI-MS mass spectrometer; a Shimadzu Prominence Series coupled to a LCMS-2020 ESI and APCI mass spectrometer or Waters Acquity H-class plus UPLC coupled to a Waters Acquity UPLC PDA detector and a Waters Acquity QDa API-ES mass detector. Only molecular ions, fractions from molecular ions and other major peaks are reported as mass/charge (m/z) ratios.

### GENERAL PROCEDURES

### General Procedure 1

A microwave vial was charged with intermediate 3 or intermediate 4 (1.0 eq.), aryl halide (1.0-1.1 eq.), Pd₂(dba)₃ (2.5-5.0 mol %), PtBu₃·HBF₄ (0.1-0.2 eq.), KF (3.0-5.0 eq.) and MeCN/water (0.12 M, 10:1) and degassed by sparging with N₂. The reaction mixture was heated at 95° C. for 30 min under microwave irradiation. Upon cooling, the reaction mixture was extracted with EtOAc (3 x) and the combined organic extracts were concentrated under reduced pressure. The crude material was subjected to specified purification protocols to afford the cross-coupled product.

### General Procedure 2

A suspension of aryl halide (1.0-1.25 eq.), Pd₂(dba)₃ (5.0-10 mol %), PtBu₃·HBF₄ (0.1-0.2 eq.) and KF (9.0-12.0 eq.) in MeCN/water (0.05 M, 1:1) was degassed by sparging with N₂. The reaction mixture was heated to 95° C., before a degassed suspension of intermediate 4 (or similar) (1.0 eq.) in MeCN (0.05 M) or MeCN with a few drops of DMF was added dropwise. The reaction mixture was stirred at 95° C. for 16 h. Upon cooling, the reaction mixture was extracted with EtOAc (3×) and the combined organic extracts were concentrated under reduced pressure. The crude material was subjected to specified purification protocols to afford the cross-coupled product.

### General Procedure 3

A mixture of intermediate 1, 2 or 7 (or similar) (1.0 eq.), an aryl boronic acid or boronate ester (1.0-1.25 eq.), Pd₂(dba)₃ (5.0-10 mol %), PtBu₃·HBF₄ (0.1-0.2 eq.) and KF (3.0-5.0 eq.) in MeCN/water (0.05, 3:1) was degassed by sparging with N₂ and stirred at 90-95° C. for 1 h to 4 h. The crude material was subjected to specified work up and purification protocols to afford the cross-coupled product.

### General Procedure 4

To a solution of phenol or pyridin-2-ol (1.0 eq.), an alcohol (1.2-1.5 eq.) and triphenylphosphine (1.2-1.5 eq.) in THF (0.25 M) at 0° C., was added DIAD (1.2-1.5 eq.) and the reaction mixture was stirred at rt. After 16 h, the reaction mixture was concentrated under reduced pressure and the crude material was subjected to specified purification protocols to afford the alkylated product.

### General Procedure 5

To a solution of amine (5.0 eq.) in DCE (0.2 M) at 0° C. was added a solution of triethylaluminium in hexanes (1.0-1.3 M, 5.0 eq.) and the reaction mixture was stirred for 10 min. The solution was added to a suspension of ethyl ester (1.0 eq.) in DCE (0.1 M) at 0° C. and the reaction mixture was stirred at 85° C. for 2 h and a further 16 h at rt if the reaction was not complete. Upon completion of the reaction, the mixture was quenched with 1 M HCl and diluted with CH₂Cl₂. The layers were separated and the aqueous layer was extracted with CH₂Cl₂ (2×). The combined organic extracts were washed with brine and the layers separated via a phase separation cartridge. The organic filtrate was concentrated under reduced pressure and the crude material was subjected to specified purification protocol to afford the amide product.

### General Procedure 6

To a solution of ethyl ester (1.0 eq.) and amine (2.0-5.0 eq.) in MeCN (0.1-0.2 M) was added TBD (2.0-4 eq.) and the reaction mixture was stirred at 80-85° C. for 16 h. After this time, the reaction mixture was concentrated under reduced pressure and the crude material was subjected to specified purification protocols to afford the amide product.

### General Procedure 7

The mesylate intermediate was prepared by treating a solution of the corresponding alcohol (1.0 eq.) and DIPEA (1.5 eq.) in CH₂Cl₂ (0.4-0.5 M) at 0° C. with methanesulfonyl chloride (1.2 eq.). The reaction mixture was stirred at rt for 2-3 h. After this time, water was added and the biphasic mixture was separated via a phase separation cartridge. The organic filtrate was concentrated under reduced pressure to afford the crude mesylate intermediate, which was used without further purification.

Phenol (1.0 eq.), arylmethyl/alkylmethyl halide or mesylate (1.1-1.2 eq.), K₂CO₃ (2.0 eq.) and potassium iodide (0.1 eq.) were suspended in solvent (0.10-0.25 M) and the reaction mixture heated under reflux for 16 h. After this time, the reaction mixture was concentrated under reduced pressure, the residue partitioned between CH₂Cl₂ and water and the layers separated through a phase separation cartridge, washing the aqueous layer with further CH₂Cl₂. The organic filtrate was concentrated under reduced pressure and the crude material subjected to specified purification protocols to afford the alkylated product.

### General Procedure 8

A suspension of intermediate 4 (1.0 eq.), aryl halide (1.0-1.05 eq.), potassium phosphate basic (or other base specified) (3.0 eq.) and dioxane/water (0.12 M, 10:1) was degassed by sparging with N₂ for 10 min. Pd-118 (0.1 eq.) was added and the reaction mixture stirred at rt for 16 h. After this time, the reaction mixture was extracted with EtOAc (3×) and the combined organic extracts were concentrated under reduced pressure. The crude material was subjected to specified purification protocols to afford the cross-coupled product.

### General Procedure 9

To 2-bromo-4-(bromomethyl)-1-fluorobenzene (1.0 eq.) and amine (1.0-1.1 eq.) in CH₂Cl₂ (0.05 M) was added DIPEA (1.5-3.0 eq.) and the reaction mixture stirred at rt for 16 h. After this time, the reaction mixture was concentrated under reduced pressure and subjected to specified work up and purification protocols to afford the product.

### Example 1:4-Amino-2-oxo-N-propyl-8-[6-(trifluoromethoxy)-2-pyridyl]-1H-quinoline-3-carboxamide

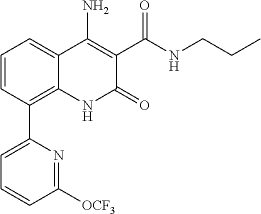 [SMILES: CCCNC(=O)c1c(N)c2cccc(-c3cccc(OC(F)(F)F)n3)c2[nH]c1=O]

### Step 1

To 2-amino-3-bromobenzonitrile (150 g, 0.76 mol) in toluene (3.15 L) under N₂ was added ethyl malonyl chloride (146 mL, 1.14 mol) over 20 min and the reaction mixture was stirred under reflux for 1 h. This reaction was repeated on identical scale and the two batches were combined and concentrated under reduced pressure. The residue was slurried in Et₂O (750 mL), filtered and washed with Et₂O (2×250 mL). The solid were slurried again in Et₂O (500 mL), filtered and washed with Et₂O (300 mL). The solids were dried under vacuum to give ethyl 3-(2-bromo-6-cyano-anilino)-3-oxo-propanoate (362 g, 76%) as an orange solid. ¹H NMR (500 MHZ, Chloroform-d) δ 9.66-9.54 (br s, 1H), 7.90-7.85 (dd, J=8.0, 1.1 Hz, 1H), 7.71-7.66 (dd, J=8.0, 1.1 Hz, 1H), 7.30-7.27 (t, J=8.0 Hz, 1H), 4.37-4.30 (q, J=7.2 Hz, 2H), 3.63 (s, 2H), 1.41-1.35 (t, J=7.2 Hz, 3H). m/z 312.9 [M+H]⁺.

### Step 2: Synthesis of Intermediate 1: Ethyl 4-amino-8-bromo-2-oxo-1H-quinoline-3-carboxylate

To ethyl 3-(2-bromo-6-cyano-anilino)-3-oxo-propanoate (181 g, 0.58 mol) in EtOH (2.8 L) under N₂ was added portionwise sodium ethoxide (40.9 g, 0.76 mmol) and the reaction mixture was stirred at 70° C. for 16 h. After this time, the reaction mixture was cooled to rt and split into two portions. Each portion was poured onto an ice-cold solution of 0.4 M HCl (3.0 L) over 30 min, the resulting solution was stirred for a further 15 min and the solids were collected by vacuum filtration. The reaction was repeated on identical scale and the solids were combined, washed with water (5×1.0 L and dried under vacuum to give intermediate 1 (334 g, 92%) as a yellow solid. ¹H NMR (500 MHZ, DMSO-d₆) δ 9.08 (br s, 1H), 8.40 (br s, 2H), 8.21-8.14 (d, J=8.0 Hz, 1H), 7.93-7.85 (d, J=8.0 Hz, 1H), 7.19-7.10 (t, J=8.0 Hz, 1H), 4.32-4.19 (q, J=7.1 Hz, 2H), 1.34-1.21 (t, J=7.1 Hz, 3H). m/z 312.9 [M+H]⁺.

### Step 3: Synthesis of Intermediate 2:4-Amino-8-bromo-2-oxo-N-propyl-1H-quinoline-3-carboxamide

To propylamine (88 mL, 1.07 mol) in DCE (700 mL) at 0° C., was added triethylaluminium (25 wt % in toluene, 576 mL, 1.07 mol) over 1 h under N₂ and the reaction mixture was stirred at rt for 15 min. The reaction mixture was slowly added to a solution of intermediate 1 (111 g, 0.36 mol) in DCE (1.25 L) at 0° C. under N₂ and stirred at rt for 15 min and then at 85° C. for 16 h. After this time, the reaction mixture was cooled to 0° C., quenched with 2 M HCl (20 mL) and stirred for 15 min at 0° C. Further 2 M HCl (110 mL) was added over 5 min and effervescence allowed to settle. The reaction mixture was then poured into 2 M HCl (2.25 L) at 5° C. and stirred for 30 min. The resulting solids were collected by vacuum filtration, washed with 1 M HCl (750 mL), water (750 mL) and MeOH (750 mL) and dried under vacuum to give intermediate 2 (86 g, 74%) as a yellow solid. ¹H NMR (500 MHZ, DMSO-d₆) δ 11.11-10.88 (br s, 1H), 10.41-10.35 (t, J=5.6 Hz, 1H), 9.51 (s, 1H), 8.37-8.21 (br s, 1H), 8.21-8.16 (d, J=8.0 Hz, 1H), 7.96-7.91 (d, J=8.0 Hz, 1H), 7.23-7.16 (t, J=8.0 Hz, 1H), 3.28-3.21 (app. q, J=6.6 Hz, 2H), 1.58-1.48 (h, J=7.3 Hz, 2H), 0.96-0.89 (t, J=7.3 Hz, 3H). m/z 372.0 [M+H]⁺.

### Step 4: Synthesis of Intermediate 3: (4-Amino-2-oxo-3-(propylcarbamoyl)-1,2-dihydroquinolin-8-yl) boronic acid and Intermediate 4:4-Amino-2-oxo-N-propyl-8-(4,4,5,5-tetramethyl-1,3,2-dioxaborolan-2-yl)-1H-quinoline-3-carboxamide

To intermediate 2 (130 g, 0.40 mol) in dioxane (1 L) was added KOAc (197 g, 2.00 mol) and the reaction mixture was degassed by sparging with N₂ for 30 min. Pd(dppf)Cl₂·CH₂Cl₂ (29.3 g, 0.04 mol), B₂pin₂ (122 g, 0.48 mol) and dioxane (600 mL) were added and the reaction mixture was stirred at 95° C. for 12 h. After this time, the reaction mixture was cooled and filtered under vacuum. The solids were washed with CH₂Cl₂ (600 mL) and the organic filtrate was washed with water (600 mL) and brine (600 mL). The organic phase was dried over Na₂SO₄, passed through celite, washed with hexane and concentrated under reduced pressure to give intermediate 3 as a grey solid (80 g, 70% purity, 48% yield). ¹H NMR (500 MHZ, Chloroform-d) 11.29-10.92 (br s, 1H), 10.39 (s, 1H), 9.94 (s, 1H), 7.99-7.93 (d, J=7.6 Hz, 1H), 7.68-7.61 (d, J=7.6 Hz, 1H), 7.17-7.11 (t, J=7.6 Hz, 1H), 5.85-5.59 (br s, 1H), 3.46-3.40 (app. q, J=6.6 Hz, 2H), 1.62-1.54 (h, J=7.3 Hz, 2H), 0.98-0.90 (t, J=7.3 Hz, 3H).

The filtered solids were washed with EtOAc (2×500 mL) and concentrated under reduced pressure. The residue was dissolved in chloroform (1 L), washed with water (1 L) and brine (1 L), dried over Na₂SO₄ and concentrated under reduced pressure. The black residue was passed through a silica plug (1 kg, elution with 0-30% EtOAc/CH₂Cl₂ gradient) to give intermediate 4 as a light brown solid (75 g, 80% purity, 40% yield). ¹H NMR (500 MHZ, Chloroform-d) o 11.29-10.92 (br s, 1H), 10.39 (s, 1H), 9.94 (s, 1H), 7.99-7.93 (d, J=7.6 Hz, 1H), 7.68-7.61 (d, J=7.6 Hz, 1H), 7.17-7.11 (t, J=7.6 Hz, 1H), 5.85-5.59 (br s, 1H), 3.35-3.27 (app. q, J=6.5 Hz, 2H), 1.62-1.54 (h, J=7.3 Hz, 2H), 1.33 (s, 12H), 0.98-0.90 (t, J=7.3 Hz, 3H). m/z 325.9 [M+H]⁺.

### Step 5

General procedure 1 was followed using intermediate 4 (100 mg, 0.27 mmol) and 2-chloro-6-(trifluoromethoxy) pyridine (37 μL, 0.28 mmol). The material was purified by flash column chromatography on an ISCO system (10 g silica, elution with 0-100 EtOAc/pet. ether gradient), followed by reverse phase chromatography on an ISCO system (12 g RediSep® Rf Reversed-phase C18, elution with a 10-100% MeOH/water gradient) to give the title compound (19 mg, 16%) as a grey solid. 1H NMR (500 MHZ, DMSO-d₆) δ 11.39 (s, 1H), 10.99 (s, 1H), 10.41 (t, J=5.5 Hz, 1H), 8.31 (d, J=8.2 Hz, 1H), 8.28-8.18 (m, 3H), 8.10 (d, J=7.4 Hz, 1H), 7.42-7.36 (m, 2H), 3.24 (td, J=7.0, 5.6 Hz, 2H), 1.53 (app. sextet, J=7.3 Hz, 2H), 0.91 (t, J=7.4 Hz, 3H). m/z 407.2 [M+H]⁺.

### Example 2: 4-Amino-8-(6-(difluoromethoxy) pyridin-2-yl)-2-oxo-N-propyl-1,2-dihydroquinoline-3-carboxamide

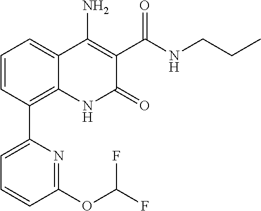 [SMILES: CCCNC(=O)c1c(N)c2cccc(-c3cccc(OC(F)F)n3)c2[nH]c1=O]

General procedure 1 was followed using intermediate 4 (250 mg, 0.67 mmol) and 2-chloro-6-(difluoromethoxy) pyridine (133 mg, 0.74 mmol). The material was purified by flash column chromatography on an ISCO system (10 g silica, elution with a 0-100% EtOAc/pet. ether gradient) and further purification by flash column chromatography (10 g silica, elution with a 0-20% MeOH/CH₂Cl₂ gradient), followed by reverse phase chromatography on an ISCO ACCQPrep system (20 mm×150 mm C18 column, elution with a 10-100% MeOH/water gradient) to give the title compound (36 mg, 13%) as a colourless solid. 1H NMR (500 MHZ, DMSO-d₆) δ 11.41 (s, 1H), 11.00 (s, 1H), 10.47 (t, J=5.4 Hz, 1H), 8.33-8.28 (m, 1H), 8.24 (s, 1H), 8.19-8.12 (m, 2H), 7.93-7.85 (m, 1H), 7.73 (m, 1H), 7.39 (dd, J=8.2, 7.6 Hz, 1H), 7.22 (dd, J=8.2, 0.6 Hz, 1H), 3.30-3.21 (m, 2H), 1.61-1.50 (m, 2H), 0.94 (t, J=7.4 Hz, 3H). m/z 389.2 [M+H]⁺.

### Example 3: 4-Amino-8-[6-(difluoromethyl)-2-pyridyl]-2-oxo-N-propyl-1H-quinoline-3-carboxamide

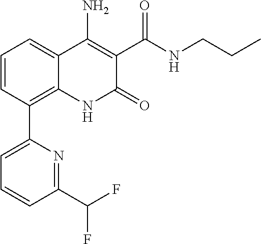 [SMILES: CCCNC(=O)c1c(N)c2cccc(-c3cccc(C(F)F)n3)c2[nH]c1=O]

General procedure 1 was followed using intermediate 4 (100 mg, 0.27 mmol) and 2-bromo-6-(difluoromethyl)pyridine (59 mg, 0.28 mmol). The material was purified by flash column chromatography on an ISCO system (10 g silica, elution with a 0-100% EtOAc/pet. ether gradient), then recrystallisation from EtOAc to give the title compound (50 mg, 47%) as a yellow solid. 1H NMR (500 MHZ, DMSO-d₆) δ 12.46 (s, 1H), 10.96 (s, 1H), 10.46 (t, J=5.5 Hz, 1H), 8.35-8.29 (m, 3H), 8.26-8.17 (m, 2H), 7.77 (d, J=7.5 Hz, 1H), 7.40 (dd, J=8.2, 7.7 Hz, 1H), 7.15 (t, J=54.9 Hz, 1H), 3.24 (td, J=7.0, 5.5 Hz, 2H), 1.53 (app. sextet, J=7.3 Hz, 2H), 0.92 (t, J=7.4 Hz, 3H). m/z 373.2 [M+H]⁺.

### Example 4: 4-Amino-8-(4-fluoro-2-pyridyl)-2-oxo-N-propyl-1H-quinoline-3-carboxamide

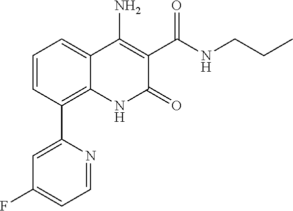 [SMILES: CCCNC(=O)c1c(N)c2cccc(-c3cc(F)ccn3)c2[nH]c1=O]

General procedure 2 was followed using intermediate 4 (106 mg, 0.20 mmol) and 2-chloro-4-fluoropyridine (25 μL, 0.25 mmol). The material was purified by flash column chromatography on an ISCO system (10 g silica, elution with a 0-100% EtOAc/n-hexane gradient) to yield a solid, which was then triturated with EtOAc to give the title compound (41 mg, 57%) as a colourless solid. 1H NMR (500 MHZ, Chloroform-d) δ 13.01 (s, 1H), 11.23 (s, 1H), 10.51 (s, 1H), 8.73 (dd, J=5.7, 8.7 Hz, 1H), 7.98 (dd, J=0.9, 7.7 Hz, 1H), 7.72 (d, J=8.2 Hz, 1H), 7.59 (dd, J=2.3, 10.5 Hz, 1H), 7.31 (t, J=8.0 Hz, 1H), 7.09 (ddd, J=2.3, 5.7, 8.0 Hz, 1H), 5.80 (s, 1H), 3.38 (dt, J=5.6, 10.4 Hz, 2H), 1.69-1.63 (m, 2H), 1.02 (t, J=7.4 Hz, 3H). m/z 341.1 [M+H]⁺.

### Example 5: 4-Amino-8-(3-methylpyridin-2-yl)-2-oxo-N-propyl-1,2-dihydroquinoline-3-carboxamide

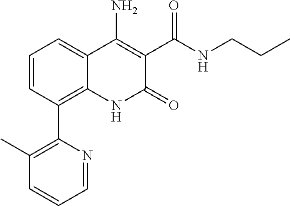 [SMILES: CCCNC(=O)c1c(N)c2cccc(-c3ncccc3C)c2[nH]c1=O]

General procedure 1 was followed using intermediate 3 (300 mg, 0.81 mmol) and 2-chloro-3-methylpyridine (133 mg, 0.89 mmol). The material was purified by flash column chromatography on an ISCO system (10 g silica, elution with a 0-100% EtOAc/pet. ether gradient) and additional purification using reverse phase chromatography on an ISCO ACCQPrep system (20 mm×150 mm C18 column, elution with a 10-100% MeOH/water gradient) to give the title compound (52 mg, 18%) as a pale brown solid. 1H NMR (500 MHZ, DMSO-d₆) δ 10.96 (s, 1H), 10.43 (t, J=5.5 Hz, 1H), 9.74 (s, 1H), 8.57 (ddd, J=4.8, 1.8, 0.7 Hz, 1H), 8.27-8.21 (m, 2H), 7.85 (ddd, J=7.8, 1.8, 0.8 Hz, 1H), 7.63 (dd, J=7.4, 1.2 Hz, 1H), 7.42 (dd, J=7.8, 4.7 Hz, 1H), 7.33 (dd, J=8.2, 7.4 Hz, 1H), 3.27-3.20 (m, 2H), 2.22 (s, 3H), 1.56-1.43 (m, 2H), 0.90 (t, J=7.4 Hz, 3H). m/z 337.2 [M+H]⁺.

### Example 6: 4-Amino-8-(3,5-difluoro-2-pyridyl)-2-oxo-N-propyl-1H-quinoline-3-carboxamide

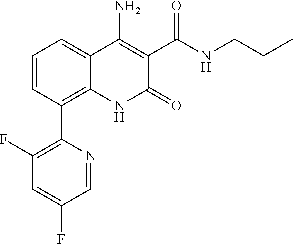 [SMILES: CCCNC(=O)c1c(N)c2cccc(-c3ncc(F)cc3F)c2[nH]c1=O]

General procedure 2 was followed using intermediate 4 (106 mg, 0.20 mmol) and 2-bromo-3,5-difluoropyridine (78 mg, 0.40 mmol). The material was purified by flash column chromatography on an ISCO system (10 g silica, elution with a 0-100% EtOAc/n-hexane gradient) to yield a solid, which was then triturated with EtOAc to give the title compound (37 mg, 49%) as a colourless solid. 1H NMR (500 MHZ, Chloroform-d) δ 11.27 (s, 1H), 10.99 (s, 1H), 10.39 (s, 1H), 8.53 (d, J=2.4 Hz, 1H), 7.96 (ddd, J=1.0, 3.4, 7.7 Hz, 1H), 7.72 (d, J=8.2 Hz, 1H), 7.46 (ddd, J=2.5, 7.9, 10.5 Hz, 1H), 7.31 (t, J=8.0 Hz, 1H), 5.85 (s, 1H), 3.38 (td, J=7.0, 5.7 Hz, 2H), 1.65 (app. sextet, J=9.1 Hz, 2H), 1.00 (t, J=7.4 Hz, 3H). m/z 359.1 [M+H]⁺.

### Example 7: 4-Amino-8-(3-fluoro-6-methyl-2-pyridyl)-2-oxo-N-propyl-1H-quinoline-3-carboxamide

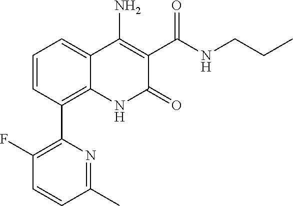 [SMILES: CCCNC(=O)c1c(N)c2cccc(-c3nc(C)ccc3F)c2[nH]c1=O]

General procedure 2 was followed using intermediate 4 (100 mg, 0.22 mmol) and 2-bromo-3-fluoro-6-methylpyridine (41 mg, 0.22 mmol). The material was purified by flash column chromatography on an ISCO system (10 g silica, elution with a 50-100% EtOAc/pet. ether gradient) to give the title compound (28 mg, 36%) as a colourless solid. ¹H NMR (600 MHZ, Chloroform-d) δ 11.64 (s, 1H), 11.23 (s, 1H), 10.44 (s, 1H), 8.09-7.95 (m, 1H), 7.76-7.63 (m, 1H), 7.58-7.44 (m, 1H), 7.36-7.28 (m, 1H), 7.25-7.15 (m, 1H), 5.84 (s, 1H), 3.38 (app. q, J=6.8 Hz, 2H), 2.68 (s, 3H), 1.65 (app. sextet, J=7.5 Hz, 2H), 1.00 (t, J=7.6 Hz, 3H). m/z 355.0 [M+H]⁺.

### Example 8: 4-Amino-8-(3-fluoro-6-methoxypyridin-2-yl)-2-oxo-N-propyl-1,2-dihydroquinoline-3-carboxamide

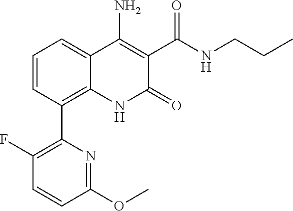 [SMILES: CCCNC(=O)c1c(N)c2cccc(-c3nc(OC)ccc3F)c2[nH]c1=O]

General procedure 1 was followed using intermediate 4 (400 mg, 1.07 mmol) and 2-bromo-3-fluoro-6-methoxypyridine (244 mg, 1.18 mmol). The material was purified by flash column chromatography on an ISCO system (10 g silica, elution with a 0-100% EtOAc/pet. ether gradient) to give the title compound (220 mg, 52%) as a colourless solid. ¹H NMR (500 MHZ, DMSO-d₆) δ 11.02 (s, 1H), 10.97 (s, 1H), 10.43 (t, J=5.5 Hz, 1H), 8.30-8.25 (m, 1H), 8.24 (s, 1H), 7.93-7.86 (m, 2H), 7.36 (dd, J=8.2, 7.6 Hz, 1H), 7.00 (dd, J=9.0, 2.8 Hz, 1H), 3.93 (s, 3H), 3.28-3.20 (m, 2H), 1.58-1.47 (m, 2H), 0.91 (t, J=7.4 Hz, 3H). m/z 371.2 [M+H]⁺.

### Example 9:4-Amino-8-(6-ethoxy-3-fluoropyridin-2-yl)-2-oxo-N-propyl-1,2-dihydroquinoline-3-carboxamide

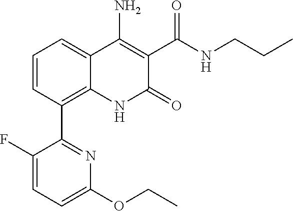 [SMILES: CCCNC(=O)c1c(N)c2cccc(-c3nc(OCC)ccc3F)c2[nH]c1=O]

### Step 1

To an ice cold solution of NaH (626 mg, 15.64 mmol) in THF (7 mL) was added EtOH (0.9 mL, 15.64 mmol) and the reaction mixture was allowed to stir at rt for 10 min. After this time, 2,5-difluoropyridine (1.50 g, 13.03 mmol) was added dropwise and the reaction mixture was stirred at rt for 16 h. After this time, the reaction mixture was diluted with water (10 mL) and extracted with CH₂Cl₂ (3×10 mL). The combined organic extracts were dried over Na₂SO₄, filtered and concentrated under reduced pressure to give 2-ethoxy-5-fluoro-pyridine (1.63 g, 82%) as a yellow oil. ¹H NMR (500 MHZ, Chloroform-d) δ 7.99 (s, 1H), 7.38-7.26 (m, 1H), 6.70 (d, J=6.9 Hz, 1H), 4.33 (q, J=7.0, 2H), 1.40 (t, J=7.0 Hz, 3H).

### Step 2

To 2-ethoxy-5-fluoro-pyridine (300 mg, 1.98 mmol) in chloroform (4 mL) was added mCPBA (341 mg, 1.98 mmol) and the reaction mixture stirred at 50° C. for 16 h. After this time, a further equivalent of mCPBA was added and the reaction stirred at 50° C. for a further 16 h. After this time, the reaction mixture was diluted with chloroform (10 mL), treated with K₂CO₃ (2 g) and the resulting suspension was stirred at rt for 1 h. The obtained suspension was filtered and the filtrate was dried over Na₂SO₄, filtered and concentrated under reduced pressure. The material was purified by flash column chromatography on an ISCO system (10 g silica, elution with a 0-10% MeOH/CH₂Cl₂ gradient) to give 2-ethoxy-5-fluoro-1-oxido-pyridin-1-ium (166 mg, 51%) as a yellow oil. ¹H NMR (500 MHZ, Chloroform-d) δ 8.07 (s, 1H), 7.03-6.95 (m, 1H), 6.81-6.74 (m, 1H), 4.18 (q, J=7.0 Hz, 2H), 1.38 (d, J=7.0 Hz, 3H).

### Step 3

To 2-ethoxy-5-fluoro-1-oxido-pyridin-1-ium (160 mg, 0.82 mmol) was added POCl₃ (1.85 mL, 19.80 mmol) and the reaction mixture stirred at 100° C. for 6 h. After this time, the reaction mixture was slowly poured into 10 mL of ice-cold water and basified to pH 10 using 10 M NaOH. The aqueous layer was extracted with CH₂Cl₂ (3×20 mL) and the combined organic extracts were dried over Na₂SO₄, filtered and concentrated under reduced pressure. The material was purified by flash column chromatography on an ISCO system (10 g silica, elution with a 0-10% MeOH/CH₂Cl₂ gradient) and further purification by flash column chromatography (10 g silica, elution with a 0-100% EtOAc/pet. ether gradient) to give a mixture of 2-chloro-6-ethoxy-3-fluoropyridine and 4-chloro-2-ethoxy-5-fluoropyridine (70:30 ratio) (100 mg, 50%) as a colourless oil and taken as is into the next step. ¹H NMR of major species (400 MHZ, Chloroform-d) δ 7.41-7.32 (m, 1H), 6.60 (d, J=8.8 Hz, 1H), 4.35-4.23 (m, 2H), 1.39-1.30 (m, 3H). m/z 176.0 [M+H]⁺.

### Step 4

General procedure 1 was followed using intermediate 4 (150 mg, 0.30 mmol) and the mixture of 2-chloro-6-ethoxy-3-fluoropyridine: 4-chloro-2-ethoxy-5-fluoropyridine 70:30 (74 mg, 0.30 mmol). The material was purified by reverse phase chromatography on an ISCO system (12 g RediSep® Rf Reversed-phase C18, elution with a 10-100% MeOH/water gradient) and further purification by flash column chromatography on an ISCO system (10 g silica, elution with a 0-100% EtOAc/pet. ether gradient), followed by reverse phase chromatography on an ISCO ACCQPrep system (20 mm×150 mm C18 column, elution with a 10-100% MeOH/water gradient) to give the title compound (5 mg, 4%) as a colourless solid. ¹H NMR (500 MHZ, Chloroform-d) δ 11.28 (s, 1H), 10.40 (s, 1H), 8.02 (d, J=6.2 Hz, 1H), 7.71 (d, J=8.2 Hz, 1H), 7.61-7.53 (m, 1H), 7.36-7.28 (m, 2H), 6.82 (dd, J=9.0, 5.0 Hz, 1H), 5.88 (s, 1H), 4.52-4.44 (m, 2H), 3.39 (q, J=6.7 Hz, 2H), 1.73-1.63 (m, 2H), 1.49 (t, J=7.0 Hz, 3H), 1.02 (d, J=6.7 Hz, 3H). m/z 385.2 [M+H]⁺.

### Example 10: 4-Amino-8-(3-fluoro-6-(methoxymethyl)pyridin-2-yl)-2-oxo-N-propyl-1,2-dihydroquinoline-3-carboxamide

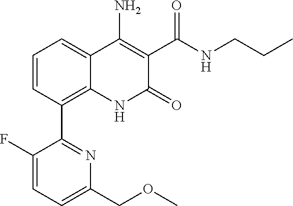 [SMILES: CCCNC(=O)c1c(N)c2cccc(-c3nc(COC)ccc3F)c2[nH]c1=O]

### Step 1

To 6-bromo-5-fluoro-2-pyridinecarboxylic acid (500 mg, 2.27 mmol) in THF (3 mL) at 0° C., was added 1 M borane solution in THF (5.68 mL, 5.68 mmol) and the reaction mixture was stirred at 80° C. for 16 h. After this time, the reaction mixture was quenched with MeOH (3 mL) and concentrated under reduced pressure. The material was purified by flash column chromatography on an ISCO system (10 g silica, elution with a 0-10% MeOH/CH₂Cl₂ gradient) to give (6-bromo-5-fluoro-2-pyridyl)methanol (400 mg, 43%) as a yellow oil. ¹H NMR (500 MHZ, Chloroform-d) δ 7.39-7.30 (m, 2H), 4.59 (s, 2H). m/z 208.0 [M+H]⁺.

### Step 2

To (6-bromo-5-fluoro-2-pyridyl)methanol (200 mg, 0.48 mmol) in THF (3 mL) at 0° C., was added NaH (23.2 mg, 0.58 mmol) and the reaction mixture was stirred at rt for 30 min. After this time, the reaction mixture was cooled to 0° C., iodomethane (0.03 mL, 0.53 mmol) was added and the reaction mixture stirred at rt. After 16 h, the reaction mixture was then quenched with water (3 mL) and the aqueous layer was extracted with EtOAc (3×10 mL) and the combined organic extracts were washed successively with water (5 mL) and brine (5 mL), dried over Na₂SO₄, filtered and concentrated under reduced pressure to give 2-bromo-3-fluoro-6-(methoxymethyl)pyridine (100 mg, 87%) as a yellow oil. ¹H NMR (400 MHZ, Chloroform-d) δ 7.47-7.41 (m, 2H), 4.53 (s, 2H), 3.46 (s, 3H). m/z 222.0 [M+H]⁺.

### Step 3

General procedure 1 was followed using intermediate 4 (180 mg, 0.36 mmol) and 2-bromo-3-fluoro-6-(methoxymethyl)pyridine (103 mg, 0.46 mmol). The material was purified by reverse phase chromatography on an ISCO system (12 g RediSep® Rf Reversed-phase C18, elution with a 10-100% MeOH/water gradient) and further purification by flash column chromatography (10 g silica, elution with a 0-100% EtOAc/pet. ether gradient) were followed to give the title compound (79 mg, 57%) as a colourless solid. ¹H NMR (400 MHZ, Chloroform-d) δ 11.49 (s, 1H), 11.17 (s, 1H), 10.43 (s, 1H), 8.00 (dd, J=7.9, 2.9 Hz, 1H), 7.78 (d, J=8.2 Hz, 1H), 7.67-7.57 (m, 1H), 7.53-7.46 (m, 1H), 7.29-7.21 (m, 1H), 6.26 (s, 1H), 4.68 (s, 2H), 3.52 (s, 3H), 3.36 (q, J=6.6 Hz, 2H), 1.71-1.54 (m, 2H), 0.99 (t, J=7.4 Hz, 3H). m/z 385.3 [M+H]⁺.

### Example 11: 4-Amino-8-[3-fluoro-6-(hydroxymethyl)-2-pyridyl]-2-oxo-N-propyl-1H-quinoline-3-carboxamide

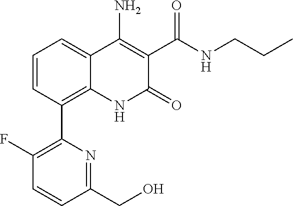 [SMILES: CCCNC(=O)c1c(N)c2cccc(-c3nc(CO)ccc3F)c2[nH]c1=O]

General procedure 1 was followed using intermediate 4 (400 mg, 1.08 mmol) and (6-bromo-5-fluoro-2-pyridyl)methanol (244 mg, 1.19 mmol). The material was purified by flash column chromatography on an ISCO system (10 g silica, elution with a 0-10% MeOH/CH₂Cl₂ gradient) to yield a solid, which was then triturated with EtOAc to give the title compound (130 mg, 31%) as a colourless solid. ¹H NMR (500 MHZ, DMSO-d₆) δ 10.96 (s, 1H), 10.49-10.37 (m, 1H), 8.32-8.17 (m, 2H), 7.99-7.85 (m, 2H), 7.62 (dd, J=8.6, 3.2 Hz, 1H), 7.40-7.34 (m, 1H), 5.76-5.48 (m, 1H), 4.65 (d, J=5.4 Hz, 2H), 3.24 (q, J=6.6 Hz, 2H), 1.57-1.47 (m, 2H), 0.91 (t, J=7.4 Hz, 3H). m/z 371.3 [M+H]⁺.

### Example 12: 4-Amino-8-(6-(hydroxymethyl)-3-methylpyridin-2-yl)-2-oxo-N-propyl-1,2-dihydroquinoline-3-carboxamide

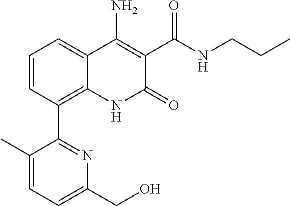 [SMILES: CCCNC(=O)c1c(N)c2cccc(-c3nc(CO)ccc3C)c2[nH]c1=O]

### Step 1

To 6-chloro-5-methylpyridine-2-carboxylic acid (100 mg, 0.58 mmol) in THF (3 mL) at 0° C., was added 1 M borane solution in THF (1.46 mL, 1.46 mmol) and the reaction mixture was stirred at 80° C. for 16 h. After this time, the reaction mixture was quenched with MeOH (10 mL) and concentrated under reduced pressure. The material was purified by flash column chromatography on an ISCO system (10 g silica, elution with a 0-100% EtOAc/pet. ether gradient) to give (6-chloro-5-methyl-2-pyridyl)methanol (30 mg, 16%) as a colourless oil. ¹H NMR (500 MHZ, Chloroform-d) δ 7.56 (d, J=7.6 Hz, 1H), 7.18 (d, J=7.6 Hz, 1H), 4.71 (s, 2H), 3.29 (s, 1H), 2.39 (s, 3H). m/z 158.0 [M+H]+.

### Step 2

General procedure 1 was followed using intermediate 4 (40 mg, 0.08 mmol) and (6-chloro-5-methyl-2-pyridyl)methanol (28 mg, 0.08 mmol). The material was purified by reverse phase chromatography on an ISCO system (12 g RediSep® Rf Reversed-phase C18, elution with a 10-100% MeOH/water gradient), followed by flash column chromatography on an ISCO system (10 g silica, elution with a 0-10% MeOH/CH₂Cl₂ gradient) to give the title compound (7 mg, 22%) as a colourless solid. ¹H NMR (500 MHZ, Methanol-d₄) δ 8.15 (d, J=8.2 Hz, 1H), 7.91 (d, J=7.9 Hz, 1H), 7.63 (d, J=7.3 Hz, 1H), 7.59 (d, J=7.3 Hz, 1H), 7.43-7.36 (m, 1H), 4.76 (s, 2H), 3.36-3.29 (m, 2H), 2.25 (s, 3H), 1.68-1.57 (m, 2H), 1.00 (t, J=7.5 Hz, 3H). m/z 367.2 [M+H]⁺.

### Example 13:4-Amino-8-[3-methyl-6-(trifluoromethyl)-2-pyridyl]-2-oxo-N-propyl-1H-quinoline-3-carboxamide

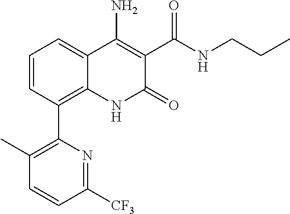 [SMILES: CCCNC(=O)c1c(N)c2cccc(-c3nc(C(F)(F)F)ccc3C)c2[nH]c1=O]

General procedure 1 was followed using intermediate 4 (100 mg, 0.27 mmol) and 2-chloro-3-methyl-6-(trifluoromethyl)pyridine (55 mg, 0.28 mmol). The material was purified by flash column chromatography on an ISCO system (10 g silica, elution with a 30-100% EtOAc/pet. ether gradient), followed by reverse phase chromatography on an ISCO system (12 g RediSep® Rf Reversed-phase C18, elution with a 10-100% MeOH/water gradient) to give the title compound (50 mg, 44%) as an off-white solid. 1H NMR (500 MHz, DMSO-d₆) δ10.95 (s, 1H), 10.48 (t, J=5.5 Hz, 1H), 10.08 (s, 1H), 8.38-8.11 (m, 2H), 8.05 (d, J=8.0 Hz, 1H), 7.86 (d, J=8.0 Hz, 1H), 7.54 (dd, J=7.4, 1.3 Hz, 1H), 7.33 (dd, J=8.3, 7.4 Hz, 1H), 3.23 (td, J=6.8, 5.6 Hz, 2H), 2.19 (s, 3H), 1.50 (app. sextet, J=7.3 Hz, 2H), 0.90 (t, J=7.4 Hz, 3H). m/z 405.2 [M+H]⁺.

### Example 14: 4-Amino-8-(2-methoxypyridin-3-yl)-2-oxo-N-propyl-1,2-dihydroquinoline-3-carboxamide

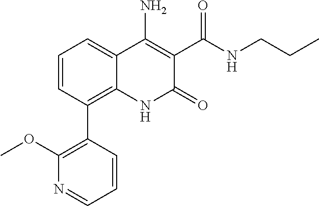 [SMILES: CCCNC(=O)c1c(N)c2cccc(-c3cccnc3OC)c2[nH]c1=O]

General procedure 1 was followed using intermediate 3 (300 mg, 0.81 mmol) and 3-bromo-2-methoxypyridine (167 mg, 0.89 mmol). The material was purified by flash column chromatography on an ISCO system (10 g silica, elution with a 0-100% EtOAc/pet. ether gradient) to give the title compound (165 mg, 55%) as a cream solid. ¹H NMR (500 MHZ, DMSO-d₆) δ 10.92 (s, 1H), 10.52 (t, J=5.6 Hz, 1H), 9.65 (s, 1H), 8.28 (dd, J=5.0, 1.9 Hz, 1H), 8.20-8.14 (m, 2H), 7.64 (dd, J=7.2, 1.9 Hz, 1H), 7.41 (dd, J=7.3, 1.3 Hz, 1H), 7.27 (dd, J=8.3, 7.3 Hz, 1H), 7.11 (dd, J=7.2, 5.0 Hz, 1H), 3.81 (s, 3H), 3.27-3.20 (m, 2H), 1.57-1.46 (m, 2H), 0.91 (t, J=7.4 Hz, 3H). m/z 353.1 [M+H]⁺.

### Example 15: 4-Amino-8-(4-methylpyridin-3-yl)-2-oxo-N-propyl-1,2-dihydroquinoline-3-carboxamide

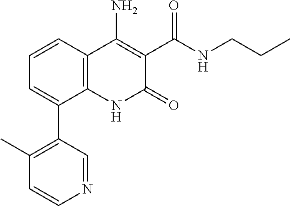 [SMILES: CCCNC(=O)c1c(N)c2cccc(-c3cnccc3C)c2[nH]c1=O]

General procedure 1 was followed using intermediate 4 (77 mg, 0.22 mmol) and 3-bromo-4-methylpyridine (59 mg, 0.34 mmol). The material was purified by reverse phase chromatography on an ISCO system (12 g RediSep® Rf Reversed-phase C18, elution with a 10-100% MeOH/water gradient) to give the title compound (23 mg, 30%) as an off-white solid. ¹H NMR (400 MHZ, Methanol-d₄) δ 10.46 (s, 1H), 8.53 (d, J=5.2 Hz, 1H), 8.39 (s, 1H), 8.16 (dd, J=8.3, 1.3 Hz, 1H), 7.52-7.45 (m, 2H), 7.40 (app t, J=7.8 Hz, 1H), 3.35-3.27 (m, 2H), 2.17 (s, 3H), 1.62 (h, J=7.3 Hz, 2H), 0.99 (t, J=7.4 Hz, 3H). m/z 337.1 [M+H]⁺.

### Example 16: 4-Amino-8-(4-fluoropyridin-3-yl)-2-oxo-N-propyl-1,2-dihydroquinoline-3-carboxamide

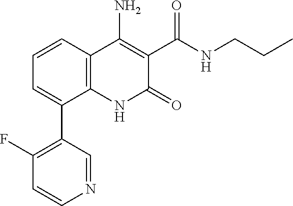 [SMILES: CCCNC(=O)c1c(N)c2cccc(-c3cnccc3F)c2[nH]c1=O]

General procedure 1 was followed using intermediate 4 (77 mg, 0.22 mmol) and 3-bromo-4-fluoropyridine (35 μg, 0.34 mmol). The material was purified by reverse phase chromatography on an ISCO system (12 g RediSep® Rf Reversed-phase C18, elution with a 10-100% MeOH/water gradient) to give the title compound (23 mg, 30%) as a colourless solid. ¹H NMR (400 MHZ, Methanol-d₄) δ 10.49 (s, 1H), 8.71 (dd, J=7.6, 5.7 Hz, 1H), 8.64 (d, J=9.7 Hz, 1H), 8.19 (dd, J=8.4, 1.3 Hz, 1H), 7.62-7.55 (m, 1H), 7.49-7.36 (m, 2H), 3.36-3.27 (m, 3H), 1.63 (m, 2H), 1.00 (t, J=7.4 Hz, 3H). m/z 341.1 [M+H]⁺.

### Example 17:4-Amino-8-(4-methoxy-3-pyridyl)-2-oxo-N-propyl-1H-quinoline-3-carboxamide

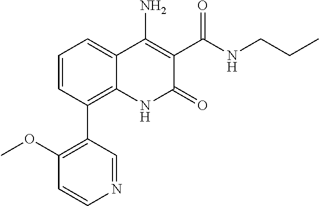 [SMILES: CCCNC(=O)c1c(N)c2cccc(-c3cnccc3OC)c2[nH]c1=O]

General procedure 3 was followed using intermediate 2 (208 mg, 0.64 mmol) and (4-methoxypyridin-3-yl) boronic acid hydrate (220 mg, 1.28 mmol. After this time, the reaction mixture was diluted with EtOAc (75 mL) and washed with brine (5×15 mL). The organic layer was passed through a phase separator cartridge and concentrated under reduced pressure. The material was purified by flash column chromatography on an ISCO system (10 g silica, elution with a 0-10% EtOAc/MeOH gradient) to yield a solid, which was then triturated with Et₂O to give the title compound (210 mg, 88%) as a colourless solid. ¹H NMR (500 MHZ, Chloroform-d) δ 10.91 (s, 1H), 10.48 (t, J=5.6 Hz, 1H), 9.48 (s, 1H), 8.51 (d, J=5.8 Hz, 1H), 8.22 (s, 1H), 8.20-8.00 (m, 2H), 7.39 (dd, J=7.3, 1.3 Hz, 1H), 7.25 (dd, J=8.3, 7.3 Hz, 1H), 7.16 (d, J=5.9 Hz, 1H), 3.76 (s, 3H), 3.20 (p, J=5.9 Hz, 2H), 1.48 (h, J=7.2 Hz, 2H), 0.88 (t, J=7.4 Hz, 3H). m/z 353.1 [M+H]⁺.

### Example 18:4-Amino-8-(4-methoxy-6-methyl-3-pyridyl)-2-oxo-N-propyl-1H-quinoline-3-carboxamide

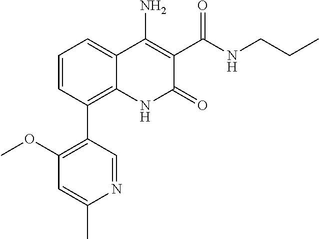 [SMILES: CCCNC(=O)c1c(N)c2cccc(-c3cnc(C)cc3OC)c2[nH]c1=O]

General procedure 2 was followed using intermediate 4 (108 mg, 0.29 mmol) and 5-bromo-4-methoxy-2-methylpyridine (50 mg, 0.29 mmol). The material was purified by flash column chromatography on an ISCO system (10 g silica, elution with a 0-100 EtOAc/MeOH gradient) to give the title compound (42 mg, 37%) as a grey solid. ¹H NMR (500 MHZ, Chloroform-d) δ 10.90 (s, 1H), 10.47 (t, J=5.6 Hz, 1H), 9.33 (s, 1H), 8.14 (m, 2H), 8.08 (s, 1H), 7.37 (d, J=7.3 Hz, 1H), 7.24 (t, J=8.3 Hz, 1H), 7.05 (s, 1H), 3.74 (s, 3H), 3.20 (q, J=6.1 Hz, 2H), 2.50 (s, 3H), 1.48 (h, J=7.3 Hz, 2H), 0.87 (t, J=7.4 Hz, 3H). m/z 367.1 [M+H]⁺.

### Example 19: 4-Amino-8-(4-ethoxypyridin-3-yl)-2-oxo-N-propyl-1,2-dihydroquinoline-3-carboxamide

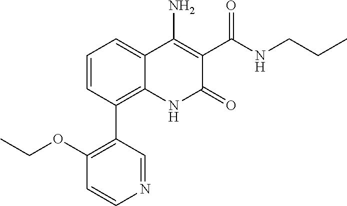 [SMILES: CCCNC(=O)c1c(N)c2cccc(-c3cnccc3OCC)c2[nH]c1=O]

### Step 1

To EtOH (0.09 mL, 1.56 mmol) in THF (3 mL) at 0° C., was added NaH (54 mg, 1.35 mmol) and the reaction mixture was stirred at rt for 30 min. After this time, the reaction mixture was cooled to 0° C., 3-bromo-4-chloropyridine (200 mg, 1.04 mmol) was added and the reaction mixture stirred under reflux for a further 16 h. After this time, the reaction was quenched with water (3 mL) and concentrated under reduced pressure. The aqueous layer was extracted with CH₂Cl₂ (3×10 mL) and the combined organic extracts were dried over Na₂SO₄, filtered and concentrated under reduced pressure to give 3-bromo-4-ethoxy pyridine (145 mg, 66%) as a yellow oil. ¹H NMR (400 MHZ, Chloroform-d) δ 8.59 (dd, J=10.4, 5.3 Hz, 1H), 8.38 (d, J=5.6 Hz, 1H), 6.81 (d, J=5.6 Hz, 1H), 4.20 (q, J=7.0 Hz, 2H), 1.53 (t, J=7.0 Hz, 3H). m/z 204.0 [M+H]⁺.

### Step 2

General procedure 1 was followed using intermediate 4 (200 mg, 0.54 mmol) and 3-bromo-4-ethoxy-pyridine (131 mg, 0.64 mmol). The material was purified by reverse phase chromatography on an ISCO system (12 g RediSep® Rf Reversed-phase C18, elution with a 10-100% MeOH/water gradient) to give the title compound (115 mg, 57%) as an off-white solid. ¹H NMR (500 MHZ, DMSO-d₆) δ 10.93 (s, 1H), 10.51 (t, J=5.5 Hz, 1H), 9.39 (s, 2H), 8.51 (m, 1H), 8.28 (s, 1H), 8.19 (d, J=8.3 Hz, 1H), 7.45 (d, J=7.5 Hz, 1H), 7.32-7.26 (m, 1H), 7.18 (d, J=5.8 Hz, 1H), 4.20-4.11 (m, 2H), 3.27-3.15 (m, 2H), 1.51 (h, J=7.2 Hz, 2H), 1.17 (t, J=7.0 Hz, 3H), 0.91 (t, J=7.4 Hz, 3H). m/z 366.4 [M+H]⁺

### Example 20: 4-Amino-8-(4-isopropoxypyridin-3-yl)-2-oxo-N-propyl-1,2-dihydroquinoline-3-carboxamide

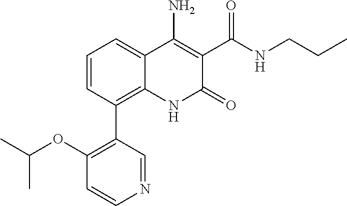 [SMILES: CCCNC(=O)c1c(N)c2cccc(-c3cnccc3OC(C)C)c2[nH]c1=O]

### Step 1

To propan-2-ol (0.12 mL, 1.56 mmol) in THF (3 mL) at 0° C. was added NaH (54 mg, 1.35 mmol) and the reaction mixture stirred at rt for 30 min. After this time, the reaction mixture was cooled to 0° C., 3-bromo-4-chloropyridine (200 mg, 1.04 mmol) was added and the reaction mixture was heated under reflux for a further 16 h. After this time, the reaction mixture was quenched with water (3 mL) and concentrated under reduced pressure. The aqueous layer was extracted with CH₂Cl₂ (3×10 mL) and the combined organic extracts were dried over Na₂SO₄, filtered and concentrated under reduced pressure to give 3-bromo-4-isopropoxy-pyridine (220 mg, 98%) as an orange oil. ¹H NMR (400 MHZ, Chloroform-d) δ 8.37 (s, 1H), 8.14 (d, J=6.0 Hz, 1H), 6.58 (d, J=6.0 Hz, 1H), 4.48 (h, J=6.0 Hz, 1H), 1.22 (d, J=6.0 Hz, 6H). m/z 218.0 [M+H]⁺.

### Step 2

General procedure 1 was followed using intermediate 4 (300 mg, 0.60 mmol) and 3-bromo-4-isopropoxy-pyridine (157 mg, 0.72 mmol). The material was purified by reverse phase chromatography on an ISCO system (12 g RediSep® Rf Reversed-phase C18, elution with a 10-100% MeOH/water gradient) to give the title compound (106 mg, 45%) as an off-white solid. ¹H NMR (500 MHZ, DMSO-d₆) δ 10.93 (s, 1H), 10.53-10.47 (m, 1H), 9.23 (s, 1H), 8.49 (d, J=5.8 Hz, 1H), 8.28 (s, 1H), 8.21-8.16 (m, 2H), 7.47-7.42 (m, 1H), 7.29 (t, J=7.8 Hz, 1H), 7.19 (d, J=5.8 Hz, 1H), 4.80 (h, J=6.1 Hz, 1H), 3.27-3.15 (m, 2H), 1.57-1.46 (m, 2H), 1.22 (d, J=6.0 Hz, 3H), 1.14 (d, J=6.0 Hz, 3H), 0.90 (t, J=7.4 Hz, 3H). m/z 381.1 [M+H]⁺.

### Example 21: 4-Amino-8-(4-(methoxymethyl)pyridin-3-yl)-2-oxo-N-propyl-1,2-dihydroquinoline-3-carboxamide

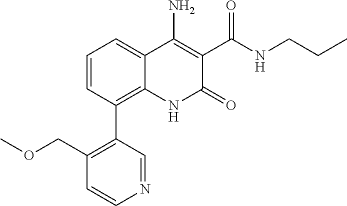 [SMILES: CCCNC(=O)c1c(N)c2cccc(-c3cnccc3COC)c2[nH]c1=O]

### Step 1

To 3-bromopyridine-4-methanol (200 mg, 1.06 mmol) in THF (4 mL) at 0° C. was added NaH (51 mg, 1.27 mmol) and the reaction mixture stirred at rt for 30 min. After this time, the reaction mixture was cooled to 0° C., iodomethane (0.07 mL, 1.17 mmol) was added and the reaction mixture was stirred at rt for a further 16 h. After this time, the reaction was quenched with water (3 mL) and concentrated under reduced pressure. The aqueous layer was extracted with EtOAc (3×10 mL) and the combined organic extracts were washed with water (10 mL) and brine (10 mL), dried over Na₂SO₄, filtered and concentrated under reduced pressure. The material was purified by flash column chromatography on an ISCO system (10 g silica, elution with a 0-100% EtOAc/pet. ether gradient) to give 3-bromo-4-(methoxymethyl)pyridine (50 mg, 22%) as a colourless oil. ¹H NMR (500 MHZ, Chloroform-d) δ 8.65 (s, 1H), 8.52 (d, J=4.9 Hz, 1H), 7.44 (d, J=4.9 Hz, 1H), 4.50 (s, 2H), 3.51 (s, 3H). m/z 203.9 [M+H]⁺.

### Step 2

General procedure 1 was followed using intermediate 4 (90 mg, 0.18 mmol) and 3-bromo-4-(methoxymethyl)pyridine (47 mg, 0.21 mmol). The material was purified by reverse phase chromatography on an ISCO system (12 g RediSep® Rf Reversed-phase C18, elution with a 10-100% MeOH/water gradient), followed by reverse phase chromatography on an ISCO ACCQPrep system (20 mm×150 mm C18 column, elution with a 10-100% MeOH/water gradient) to give the title compound (18 mg, 27%) as a colourless solid. ¹H NMR (500 MHZ, DMSO-d₆) δ 10.96 (s, 1H), 10.51-10.46 (m, 1H), 9.64 (s, 1H), 8.64 (d, J=5.1 Hz, 1H), 8.36 (s, 1H), 8.26-8.20 (m, 1H), 7.52 (d, J=5.1 Hz, 1H), 7.43 (dd, J=7.3, 1.4 Hz, 1H), 7.36-7.28 (m, 1H), 4.18 (d, J=13.8 Hz, 1H), 4.11 (d, J=13.8 Hz, 1H), 3.20 (s, 3H), 1.56-1.45 (m, 2H), 1.32-1.26 (m, 2H), 0.90 (t, J=7.4 Hz, 3H). m/z 367.1 [M+H]⁺.

### Example 22: 4-Amino-8-(5-fluoro-3-pyridyl)-2-oxo-N-propyl-1H-quinoline-3-carboxamide

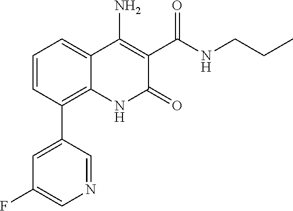 [SMILES: CCCNC(=O)c1c(N)c2cccc(-c3cncc(F)c3)c2[nH]c1=O]

General procedure 3 was followed using intermediate 2 (70 mg, 0.21 mmol) and (5-fluoropyridin-3-yl) boronic acid (40 mg, 0.29 mmol). The layers were separated and the organic layer was concentrated under reduced pressure. The residue was dissolved in CH₂Cl₂ (10 mL) and water (5 mL) and the layers separated via a phase separation cartridge. The organic layer was concentrated under reduced pressure and the material was purified by flash column chromatography on an ISCO system (10 g silica, elution with a 50-100% EtOAc/pet. ether gradient) to give the title compound (43 mg, 59%) as a colourless solid. ¹H NMR (500 MHZ, Chloroform-d) δ 11.30 (s, 1H), 10.10 (t, J=5.6 Hz, 1H), 8.56 (d, J=2.7 Hz, 1H), 8.49 (s, 1H), 8.35 (s, 1H), 7.75 (d, J=8.2 Hz, 1H), 7.49 (dt, J=8.8, 2.3 Hz, 1H), 7.45 (d, J=7.3 Hz, 1H), 7.31 (t, J=7.8 Hz, 1H), 6.14 (s, 1H), 3.33 (q, J=6.6 Hz, 2H), 1.60 (q, J=7.3 Hz, 2H), 0.95 (t, J=7.4 Hz, 3H). m/z 341.2 [M+H]⁺.

### Example 23: 4-Amino-8-(5-methoxy-3-pyridyl)-2-oxo-N-propyl-1H-quinoline-3-carboxamide

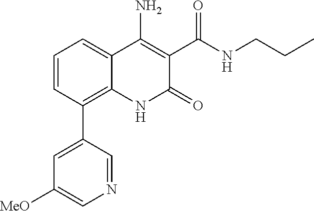 [SMILES: CCCNC(=O)c1c(N)c2cccc(-c3cncc(OC)c3)c2[nH]c1=O]

General procedure 1 was followed using intermediate 3 (400 mg, 1.38 mmol) and 3-bromo-5-methoxypyridine (273 mg, 1.45 mmol). The material was purified by flash column chromatography on an ISCO system (10 g silica, elution with a 50-100% EtOAc/pet. ether gradient) to give the title compound (212 mg, 41%) as a colourless solid. ¹H NMR (500 MHZ, DMSO-d₆) δ 10.95 (s, 1H), 10.48 (t, J=5.6 Hz, 1H), 9.67 (s, 1H), 8.36 (d, J=2.7 Hz, 1H), 8.28-8.09 (m, 3H), 7.52 (dd, J=7.3, 1.2 Hz, 1H), 7.44 (dd, J=2.9, 1.8 Hz, 1H), 7.32 (dd, J=8.3, 7.3 Hz, 1H), 3.87 (s, 3H), 3.23 (td, J=7.1, 5.6 Hz, 2H), 1.50 (app. sextet, J=7.3 Hz, 2H), 0.90 (t, J=7.4 Hz, 3H). m/z 353.2 [M+H]⁺.

### Example 24: 4-Amino-8-(5-methoxy-4-methylpyridin-3-yl)-2-oxo-N-propyl-1,2-dihydroquinoline-3-carboxamide

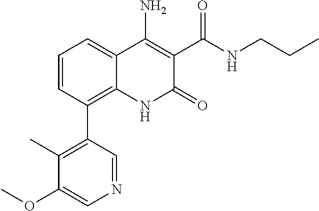 [SMILES: CCCNC(=O)c1c(N)c2cccc(-c3cncc(OC)c3C)c2[nH]c1=O]

### Step 1

To 3,5-dibromo-4-methylpyridine (200 mg, 0.80 mmol) in DMF (2 mL), was slowly added NaOMe in MeOH (30% w/v) (0.17 mL, 0.90 mmol) and the reaction mixture was stirred at 70° C. for 16 h. After this time, the reaction mixture was cooled to rt, diluted with water (10 mL) and extracted with Et₂O (3×10 mL). The combined organic extracts were dried over Na₂SO₄, filtered and concentrated under reduced pressure. The material was purified by flash column chromatography on an ISCO system (10 g silica, elution with a 0-100% EtOAc/pet. ether gradient) to give 3-bromo-5-methoxy-4-methyl-pyridine (108 mg, 64%) as a colourless solid. ¹H NMR (400 MHZ, DMSO-d₆) δ 8.33 (s, 1H), 8.28 (s, 1H), 3.93 (s, 3H), 2.26 (s, 3H). m/z 204.0 [M+H]⁺.

### Step 2

General procedure 1 was followed using intermediate 3 (140 mg, 0.48 mmol) and 3-bromo-5-methoxy-4-methyl-pyridine (103 mg, 0.48 mmol). The material was purified by flash column chromatography on an ISCO system (10 g silica, elution with a 0-100% EtOAc/pet. ether gradient) to give the title compound (52 mg, 28%) as an off-white solid. ¹H NMR (500 MHZ, DMSO-d₆) δ 10.96 (s, 1H), 10.48 (t, J=5.6 Hz, 1H), 9.56 (s, 1H), 8.35 (s, 1H), 8.25-8.17 (m, 2H), 8.00 (s, 1H), 7.44-7.38 (m, 1H), 7.35-7.28 (m, 1H), 3.97 (s, 3H), 3.27-3.18 (m, 2H), 1.87 (s, 3H), 1.50 (h, J=7.2 Hz, 2H), 0.90 (t, J=7.4 Hz, 3H). m/z 367.2 [M+H]⁺.

### Example 25: 4-Amino-8-[5-(hydroxymethyl)-3-pyridyl]-2-oxo-N-propyl-1H-quinoline-3-carboxamide

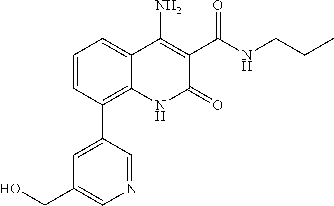 [SMILES: CCCNC(=O)c1c(N)c2cccc(-c3cncc(CO)c3)c2[nH]c1=O]

General procedure 2 was followed using intermediate 4 (100 mg, 0.22 mmol) and 3-bromo-5-pyridinemethanol (41 mg, 0.22 mmol). The material was purified by flash column chromatography on an ISCO system (10 g silica, elution with a 25-100% EtOAc/pet. ether gradient) to give the title compound (43 mg, 55%) as a yellow solid. ¹H NMR (500 MHZ, Chloroform-d) δ 11.34 (s, 1H), 10.19 (s, 1H), 8.70 (s, 1H), 8.59 (s, 1H), 8.09 (s, 1H), 7.82 (s, 1H), 7.69 (d, J=8.3 Hz, 1H), 7.48 (d, J=7.3 Hz, 1H), 7.33 (t, J=7.8 Hz, 1H), 5.94 (s, 1H), 4.84 (d, J=4.3 Hz, 2H), 3.36 (app. q, J=6.6 Hz, 2H), 2.41 (s, 1H), 1.69-1.61 (m, 2H), 0.98 (t, J=7.4 Hz, 3H). m/z 353.1 [M+H]⁺.

### Example 26: 4-Amino-8-(5-ethoxypyridin-3-yl)-2-oxo-N-propyl-1,2-dihydroquinoline-3-carboxamide

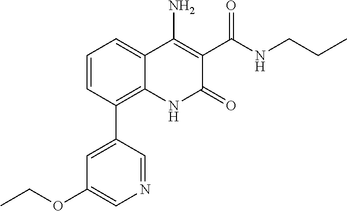 [SMILES: CCCNC(=O)c1c(N)c2cccc(-c3cncc(OCC)c3)c2[nH]c1=O]

General procedure 1 was followed using intermediate 4 (150 mg, 0.4 mmol) and 3-bromo-5-ethoxypyridine (90 mg, 0.44 mmol). The material was purified by flash column chromatography on an ISCO system (10 g silica, elution with a 0-100% EtOAc/pet. ether gradient) to give the title compound (46 mg, 30%) as an off-white solid. ¹H NMR (500 MHZ, DMSO-d₆) δ 10.96 (s, 1H), 10.48 (t, J=5.6 Hz, 1H), 9.68 (s, 1H), 8.34 (d, J=2.8 Hz, 1H), 8.23-8.16 (m, 3H), 7.53 (dd, J=7.3, 1.3 Hz, 1H), 7.42 (dd, J=2.8, 1.8 Hz, 1H), 7.32 (dd, J=8.3, 7.3 Hz, 1H), 4.16 (q, J=7.0 Hz, 2H), 3.27-3.20 (m, 2H), 1.57-1.46 (m, 2H), 1.36 (t, J=7.0 Hz, 3H), 0.91 (t, J=7.4 Hz, 3H). m/z 367.2 [M+H]⁺.

### Example 27: 4-Amino-8-(5-isopropoxypyridin-3-yl)-2-oxo-N-propyl-1,2-dihydroquinoline-3-carboxamide

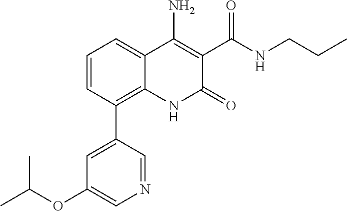 [SMILES: CCCNC(=O)c1c(N)c2cccc(-c3cncc(OC(C)C)c3)c2[nH]c1=O]

### Step 1

General procedure 4 was followed using propan-2-ol (65 mg, 1.07 mmol) and 5-bromopyridin-3-ol (150 mg, 0.86 mmol). The material was purified by flash column chromatography on an ISCO system (10 g silica, elution with a 0-100% EtOAc/pet. ether gradient) to give 3-bromo-5-isopropoxy-pyridine (179 mg, 91%) as a colourless oil. ¹H NMR (400 MHZ, Chloroform-d) δ 8.29-8.19 (m, 2H), 7.36 (s, 1H), 4.65-4.51 (m, 1H), 1.38 (d, J=6.0 Hz, 6H). m/z 218.0 [M+H]⁺.

### Step 2

General procedure 1 was followed using intermediate 3 (200 mg, 0.69 mmol) and 3-bromo-5-isopropoxy-pyridine (165 mg, 0.73 mmol). The material was purified by flash column chromatography on an ISCO system (10 g silica, elution with a 0-100% EtOAc/pet. ether gradient) to give the title compound (120 mg, 43%) as a colourless solid. ¹H NMR (500 MHZ, DMSO-d₆) δ 10.96 (s, 1H), 10.49 (t, J=5.6 Hz, 1H), 9.70 (s, 1H), 8.31 (s, 1H), 8.23-8.18 (m, 2H), 8.16 (s, 1H), 7.53 (dd, J=7.3, 1.3 Hz, 1H), 7.45-7.40 (m, 1H), 7.32 (dd, J=8.3, 7.3 Hz, 1H), 4.77 (h, J=6.0 Hz, 1H), 3.27-3.16 (m, 2H), 1.57-1.47 (m, 2H), 1.32 (d, J=6.0 Hz, 6H), 0.91 (t, J=7.4 Hz, 3H). m/z 381.2 [M+H]⁺.

### Example 28: 4-Amino-8-(5-cyclopropoxypyridin-3-yl)-2-oxo-N-propyl-1,2-dihydroquinoline-3-carboxamide

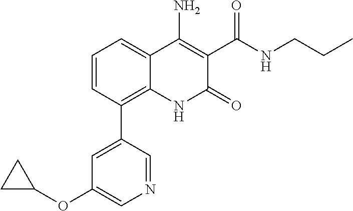 [SMILES: CCCNC(=O)c1c(N)c2cccc(-c3cncc(OC4CC4)c3)c2[nH]c1=O]

### Step 1

To 5-bromopyridin-3-ol (200 mg, 1.15 mmol) in DMF (5 mL) was added bromocyclopropane (0.18 mL, 2.3 mmol), NaI (17 mg, 2.3 mmol) and Cs₂CO₃ (1.13 g, 3.45 mmol) and the reaction mixture was stirred at 150° C. for 16 h. After this time, the reaction mixture was cooled to rt, diluted with water (10 mL) and extracted with EtOAc (3×10 mL). The combined organic extracts were dried over Na₂SO₄, filtered and concentrated under reduced pressure. The material was purified by flash column chromatography on an ISCO system (10 g silica, elution with a 0-100% EtOAc/pet. ether gradient) to give 3-bromo-5-(cyclopropoxy)pyridine (117 mg, 43%) as a colourless oil. ¹H NMR (500 MHZ, Chloroform-d) δ 8.32-8.21 (m, 2H), 7.54-7.49 (m, 1H), 3.79-3.72 (m, 1H), 0.92-0.73 (m, 4H). m/z 216.0 [M+H]⁺.

### Step 2

General procedure 1 was followed using intermediate 3 (130 mg, 0.45 mmol) and 3-bromo-5-(cyclopropoxy) pyridine (107 mg, 0.45 mmol). The material was purified by flash column chromatography on an ISCO system (10 g silica, elution with a 0-100% EtOAc/pet. ether gradient) to give the title compound (67 mg, 37%) as a colourless solid. ¹H NMR (500 MHZ, DMSO-d₆) δ 10.96 (s, 1H), 10.49 (t, J=5.5 Hz, 1H), 9.71 (s, 1H), 8.43 (d, J=2.7 Hz, 1H), 8.24-8.18 (m, 3H), 7.56-7.51 (m, 2H), 7.33 (dd, J=8.3, 7.3 Hz, 1H), 4.05-3.97 (m, 1H), 3.24 (td, J=7.0, 5.6 Hz, 2H), 1.57-1.43 (m, 2H), 0.91 (t, J=7.4 Hz, 3H), 0.88-0.78 (m, 2H), 0.77-0.71 (m, 2H). m/z 379.2 [M+H]⁺.

### Example 29: 4-Amino-8-(5-((5-fluoropyridin-2-yl)methoxy) pyridin-3-yl)-2-oxo-N-propyl-1,2-dihydroquinoline-3-carboxamide

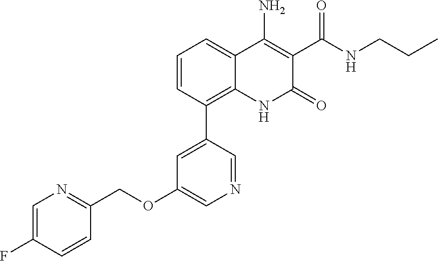 [SMILES: CCCNC(=O)c1c(N)c2cccc(-c3cncc(OCc4ccc(F)cn4)c3)c2[nH]c1=O]

### Step 1

General procedure 4 was followed using 5-fluoro-2-hydroxymethylpyridine (150 mg, 1.18 mmol) and 5-bromopyridin-3-ol (188 mg, 1.08 mmol). The material was purified by flash column chromatography on an ISCO system (10 g silica, elution with a 0-20% MeOH/CH₂Cl₂ gradient) to give 2-[(5-bromo-3-pyridyl)oxymethyl]-5-fluoro-pyridine (112 mg, 29%) as a brown solid. ¹H NMR (500 MHZ, Chloroform-d) δ 8.40 (dt, J=2.8, 0.6 Hz, 1H), 8.28-8.20 (m, 2H), 7.43 (ddq, J=8.7, 4.5, 0.6 Hz, 1H), 7.41-7.38 (m, 2H), 5.17-5.09 (s, 2H). m/z 285.0 [M+H]⁺.

### Step 2

General procedure 1 was followed using intermediate 4 (100 mg, 0.27 mmol) and 2-[(5-bromo-3-pyridyl)oxymethyl]-5-fluoro-pyridine (100 mg, 0.28 mmol). The material was purified by flash column chromatography on an ISCO system (10 g silica, elution with a 0-100% EtOAc/pet. ether gradient) to yield a solid, which was then triturated with Et₂O to give the title compound (39 mg, 31%) as a brown solid. ¹H NMR (500 MHZ, DMSO-d₆) o 10.96 (s, 1H), 10.49 (t, J=5.5 Hz, 1H), 9.69 (s, 1H), 8.61 (dt, J=3.0, 0.7 Hz, 1H), 8.47-8.43 (m, 1H), 8.25-8.18 (m, 2H), 7.85-7.77 (m, 1H), 7.73-7.66 (m, 1H), 7.59 (dd, J=2.8, 1.7 Hz, 1H), 7.53 (dd, J=7.3, 1.3 Hz, 1H), 7.36-7.29 (m, 1H), 5.31 (s, 2H), 3.28-3.19 (m, 3H), 1.57-1.45 (m, 2H), 0.91 (t, J=7.4 Hz, 3H). m/z 448.2 [M+H]⁺.

### Example 30: 4-Amino-8-[6-(hydroxymethyl)-3-pyridyl]-2-oxo-N-propyl-1H-quinoline-3-carboxamide

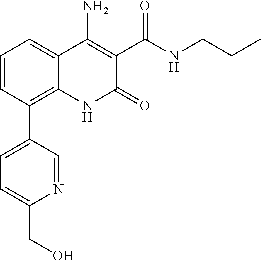 [SMILES: CCCNC(=O)c1c(N)c2cccc(-c3ccc(CO)nc3)c2[nH]c1=O]

General procedure 2 was followed using intermediate 4 (100 mg, 0.22 mmol) and (5-bromopyrid-2-yl) methanol (41 mg, 0.22 mmol). The material was purified by flash column chromatography on an ISCO system (10 g silica, elution with a 50-100% EtOAc/pet. ether gradient) to give the title compound (22 mg, 29%) as a colourless solid. ¹H NMR (500 MHZ, Chloroform-d) o 11.34 (s, 1H), 10.21 (s, 1H), 8.62 (d, J=2.1 Hz, 1H), 8.04 (s, 1H), 7.76 (dd, J=8.1, 2.1 Hz, 1H), 7.68 (d, J=8.2 Hz, 1H), 7.54-7.40 (m, 2H), 7.33 (t, J=7.9 Hz, 1H), 5.91 (s, 1H), 4.88 (s, 2H), 3.55 (s, 1H), 3.36 (app. q, J=6.6 Hz, 2H), 1.64 (app. sextet, J=7.3 Hz, 2H), 0.98 (t, J=7.4 Hz, 3H). m/z 353.2 [M+H]⁺.

### Example 31: 4-Amino-2-oxo-N-propyl-8-(pyridin-4-yl)-1,2-dihydroquinoline-3-carboxamide

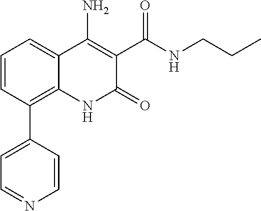 [SMILES: CCCNC(=O)c1c(N)c2cccc(-c3ccncc3)c2[nH]c1=O]

A mixture of intermediate 2 (500 mg, 1.54 mmol), pyridine-4-boronic acid hydrate (189 mg, 1.54 mmol), Pd₂(dba)₃ (35 mg, 0.04 mmol), PtBu₃·HBF₄ (45 mg, 0.15 mmol) and KF (365 mg, 6.17 mmol) in MeCN (6 mL) and water (3 mL) was degassed by sparging with N₂. The reaction mixture was heated at 95° C. for 30 min under microwave irradiation. The material was purified by flash column chromatography on an ISCO system (10 g silica, elution with a 0-100% EtOAc/pet. ether gradient) to give the title compound (270 mg, 52%) as a yellow solid. ¹H NMR (500 MHZ, DMSO-d₆) δ 10.97 (s, 1H), 10.46 (t, J=5.6 Hz, 1H), 9.59 (s, 1H), 8.72-8.67 (m, 2H), 8.28-8.15 (m, 2H), 7.53 (dd, J=7.4, 1.3 Hz, 1H), 7.51-7.46 (m, 2H), 7.34 (dd, J=8.3, 7.4 Hz, 1H), 3.29-3.20 (m, 2H), 1.57-1.46 (m, 2H), 0.91 (t, J=7.4 Hz, 3H). m/z 323.1 [M+H]⁺.

### Example 32: 4-Amino-8-[2-(hydroxymethyl)-4-pyridyl]-2-oxo-N-propyl-1H-quinoline-3-carboxamide

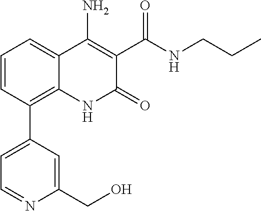 [SMILES: CCCNC(=O)c1c(N)c2cccc(-c3ccnc(CO)c3)c2[nH]c1=O]

General procedure 1 was followed using intermediate 4 (400 mg, 1.08 mmol) and 4-bromo-pyridine-2-methanol (223 mg, 1.19 mmol). The material was purified by reverse phase chromatography on an ISCO system (12 g RediSep® Rf Reversed-phase C18, elution with a 10-100% MeOH/water gradient) to give the title compound (140 mg, 35%) as a pale yellow solid. ¹H NMR (400 MHZ, Chloroform-d) δ 10.97 (s, 1H), 10.44 (d, J=5.8 Hz, 1H), 9.47 (s, 1H), 8.60 (d, J=4.9 Hz, 1H), 8.23 (d, J=6.2 Hz, 2H), 7.60-7.48 (m, 2H), 740-7.30 (m, 2H), 5.48 (t, J=5.7 Hz, 1H), 4.66 (d, J=5.8 Hz, 2H), 3.28-3.19 (m, 2H), 1.58-1.45 (m, 2H), 0.91 (t, J=7.4 Hz, 3H). m/z 353.2 [M+H]⁺.

### Example 33:4-Amino-8-(5-fluoro-2-methyl-4-pyridyl)-2-oxo-N-propyl-1H-quinoline-3-carboxamide

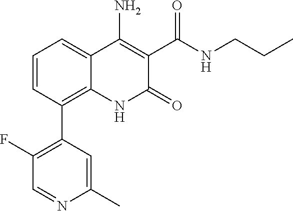 [SMILES: CCCNC(=O)c1c(N)c2cccc(-c3cc(C)ncc3F)c2[nH]c1=O]

General procedure 2 was followed using intermediate 4 (143 mg, 0.30 mmol) and 4-bromo-5-fluoro-2-methylpyridine (99 mg, 0.34 mmol). The material was purified by flash column chromatography on an ISCO system (10 g silica, elution with a 0-50% EtOAc/pet. ether gradient) to yield a solid, which was then triturated with EtOAc to give the title compound (53 mg, 53%) as a colourless solid. ¹H NMR (500 MHZ, Chloroform-d) δ 11.39 (s, 1H), 10.21 (t, J=5.1 Hz, 1H), 8.56 (s, 1H), 7.84 (s, 1H), 7.75 (d, J=8.2 Hz, 1H), 7.51 (d, J=7.3 Hz, 1H), 7.35 (t, J=7.8 Hz, 1H), 7.19 (d, J=5.6 Hz, 1H), 5.95 (s, 1H), 3.39 (dt, J=7.0, 5.8 Hz, 2H), 2.64 (s, 3H), 1.66 (sext, J=7.3 Hz, 2H), 1.01 (t, J=7.4 Hz, 3H). m/z 355.2 [M+H]⁺.

### Example 34: 4-Amino-8-(5-fluoro-2-methoxy-4-pyridyl)-2-oxo-N-propyl-1H-quinoline-3-carboxamide

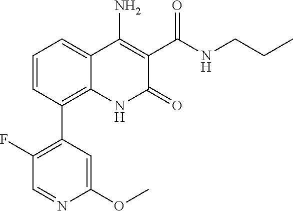 [SMILES: CCCNC(=O)c1c(N)c2cccc(-c3cc(OC)ncc3F)c2[nH]c1=O]

General procedure 3 was followed using intermediate 2 (70 mg, 0.21 mmol) and 5-fluoro-2-methoxypyridine-4-boronic acid (44 mg, 0.26 mmol at 80° C. After this time, the layers were separated and the organic layer was concentrated under reduced pressure. The material was purified by flash column chromatography on an ISCO system (10 g silica, elution with a 15-60% EtOAc/pet. ether gradient) to give the title compound (55 mg, 80%) as a colourless solid. ¹H NMR (500 MHZ, Chloroform-d) δ 11.30 (s, 1H), 10.13 (t, J=5.7 Hz, 1H), 8.32 (d, J=7.4 Hz, 1H), 8.11 (s, 1H), 7.75 (d, J=8.3 Hz, 1H), 7.47 (d, J=7.4 Hz, 1H), 7.29 (t, J=7.8 Hz, 1H), 6.74 (d, J=4.7 Hz, 1H), 6.09 (s, 1H), 3.91 (s, 3H), 3.33 (q, J=6.6 Hz, 2H), 1.60 (h, J=7.3 Hz, 2H), 0.96 (t, J=7.4 Hz, 3H). m/z 371.2 [M+H]⁺.

### Example 35: 4-Amino-8-(2-ethoxy-5-fluoropyridin-4-yl)-2-oxo-N-propyl-1,2-dihydroquinoline-3-carboxamide

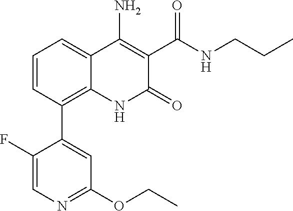 [SMILES: CCCNC(=O)c1c(N)c2cccc(-c3cc(OCC)ncc3F)c2[nH]c1=O]

General procedure 1 was followed using intermediate 4 (150 mg, 0.30 mmol) and a mixture of 2-chloro-6-ethoxy-3-fluoropyridine: 4-chloro-2-ethoxy-5-fluoropyridine 70:30 (see Example 9, 74 mg, 0.30 mmol). The material was purified by reverse phase chromatography on an ISCO system (12 g RediSep® Rf Reversed-phase C18, elution with a 10-100% MeOH/water gradient) and further purification by flash column chromatography on an ISCO system (10 g silica, elution with a 0-100% EtOAc/pet. ether gradient), followed by reverse phase chromatography on an ISCO ACCQPrep system (20 mm×150 mm C18 column, elution with a 10-100% MeOH/water gradient) to give the title compound (6 mg, 5%) as a colourless solid. ¹H NMR (500 MHZ, Chloroform-d) o 11.36 (s, 1H), 10.22 (s, 1H), 8.18 (s, 1H), 7.97 (s, 1H), 7.74 (d, J=8.3 Hz, 1H), 7.52 (d, J=7.4 Hz, 1H), 7.38-7.30 (m, 1H), 6.76-6.71 (m, 1H), 5.94 (s, 1H), 4.45-4.36 (m, 2H), 3.39 (q, J=6.6, 5.9 Hz, 2H), 1.70-1.63 (m, 2H), 1.46-1.39 (m, 3H), 1.01 (t, J=6.6 Hz, 3H). m/z 385.2 [M+H]⁺.

### Example 36: 4-Amino-2-oxo-N-propyl-8-pyrimidin-4-yl-1H-quinoline-3-carboxamide

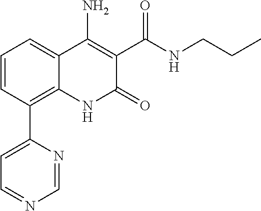 [SMILES: CCCNC(=O)c1c(N)c2cccc(-c3ccncn3)c2[nH]c1=O]

General procedure 2 was followed using intermediate 4 (143 mg, 0.27 mmol) and 4-chloropyrimidine (39 mg, 0.34 mmol). The material was purified by flash column chromatography on an ISCO system (10 g silica, elution with a 20-100% EtOAc/pet. ether gradient) to yield a solid, which was then triturated with EtOAc to give the title compound (47 mg, 51%) as a cream solid. ¹H NMR (500 MHZ, Chloroform-d) δ 13.02 (s, 1H), 11.31 (s, 1H), 10.48 (t, J=5.0 Hz, 1H), 9.39 (d, J=1.2 Hz, 1H), 8.91 (d, J=5.6 Hz, 1H), 8.15 (dd, J=7.8, 0.8 Hz, 1H), 7.88 (dd, J=5.6 Hz, 1.3 Hz, 1H) 7.82 (d, J=8.1 Hz, 1H), 7.36 (t, J=8.0 Hz, 1H), 5.84 (s, 1H), 3.42 (dt, J=6.9, 5.7 Hz, 2H), 1.69 (sext, J=7.3 Hz, 2H), 1.04 (t, J=7.4 Hz, 3H). m/z 324.1 [M+H]⁺.

### Example 37: 4-Amino-8-(5-methoxypyrimidin-4-yl)-2-oxo-N-propyl-1H-quinoline-3-carboxamide

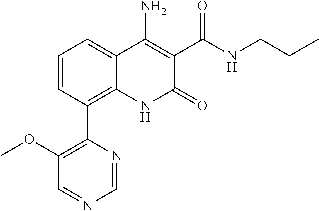 [SMILES: CCCNC(=O)c1c(N)c2cccc(-c3ncncc3OC)c2[nH]c1=O]

General procedure 2 was followed using intermediate 4 (100 mg, 0.22 mmol) and 4-chloro-5-methoxypyrimidine (31 mg, 0.22 mmol). The material was purified by flash column chromatography on an ISCO system (10 g silica, elution with a 50-100% EtOAc/pet. ether gradient) to give the title compound (23 mg, 30%) as a colourless solid. ¹H NMR (500 MHZ, Chloroform-d) o 11.25 (s, 1H), 11.20 (s, 1H), 10.40 (s, 1H), 9.01 (s, 1H), 8.61 (s, 1H), 8.24 (d, J=7.7 Hz, 1H), 7.73 (d, J=8.2 Hz, 1H), 7.29 (t, J=8.0 Hz, 1H), 5.82 (s, 1H), 4.01 (s, 3H), 3.38 (app. q, J=6.6 Hz, 2H), 1.65 (app. sextet, J=7.5 Hz, 2H), 1.00 (t, J=7.4 Hz, 3H). m/z 354.2 [M+H]⁺.

### Example 38: 4-Amino-8-(2-methylpyrimidin-4-yl)-2-oxo-N-propyl-1,2-dihydroquinoline-3-carboxamide

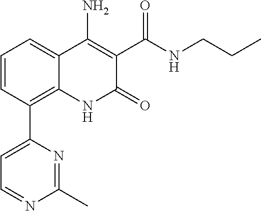 [SMILES: CCCNC(=O)c1c(N)c2cccc(-c3ccnc(C)n3)c2[nH]c1=O]

General procedure 1 was followed using intermediate 4 (400 mg, 0.81 mmol) and 4-chloro-2-methylpyrimidine (103 mg, 0.81 mmol). The material was purified by flash column chromatography on an ISCO system (10 g silica, elution with a 0-100% EtOAc/pet. ether gradient) to give the title compound (79 mg, 29%) as a brown solid. ¹H NMR (500 MHZ, Chloroform-d) δ 13.16 (s, 1H), 11.14 (s, 1H), 10.44 (s, 1H), 8.74 (dd, J=5.5, 1.8 Hz, 1H), 8.06 (d, J=7.7 Hz, 1H), 7.83 (d, J=7.7 Hz, 1H), 7.63 (d, J=5.5 Hz, 1H), 7.30-7.22 (m, 1H), 6.22 (s, 1H), 3.41-3.33 (m, 2H), 2.86 (s, 3H), 1.71-1.61 (m, 2H), 1.00 (t, J=7.4 Hz, 3H). m/z 338.2 [M+H]⁺.

### Example 39: 4-Amino-8-(5-cyano-2-methyl-pyrimidin-4-yl)-2-oxo-N-propyl-1H-quinoline-3-carboxamide

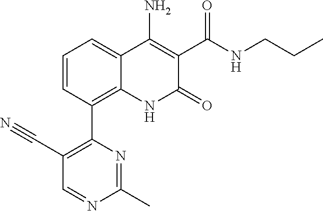 [SMILES: CCCNC(=O)c1c(N)c2cccc(-c3nc(C)ncc3C#N)c2[nH]c1=O]

General procedure 1 was followed using intermediate 4 (120 mg, 0.42 mmol) and 4-chloro-2-methylpyrimidine-5-carbonitrile (64 mg, 0.42 mmol). The material was purified by flash column chromatography on an ISCO system (10 g silica, elution with a 0-10% MeOH/CH₂Cl₂ gradient) to yield a solid, which was then triturated with MeOH to give the title compound (41 mg, 26%) as a pale yellow solid. ¹H NMR (400 MHZ, DMSO-d₆) O 11.25 (s, 1H), 11.05 (s, 1H), 9.89-9.69 (m, 1H), 9.44 (d, J=2.7 Hz, 1H), 8.88-8.58 (m, 2H), 8.46 (d, J=8.3 Hz, 1H), 7.68-7.50 (m, 1H), 3.31-3.24 (m, 2H), 2.78 (s, 3H), 1.64-1.51 (m, 2H), 0.94 (t, J=7.4 Hz, 3H). m/z 363.2 [M+H]⁺.

### Example 40: 4-Amino-8-(5-fluoro-2-methyl-pyrimidin-4-yl)-2-oxo-N-propyl-1H-quinoline-3-carboxamide

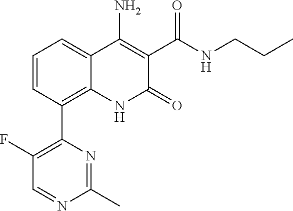 [SMILES: CCCNC(=O)c1c(N)c2cccc(-c3nc(C)ncc3F)c2[nH]c1=O]

General procedure 1 was followed using intermediate 3 (900 mg, 3.11 mmol) and 4-chloro-5-fluoro-2-methylpyrimidine (456 mg, 3.11 mmol). The material was purified by flash column chromatography on an ISCO system (10 g silica, elution with a 50-100% EtOAc/pet. ether gradient) to yield a solid, which was then triturated with MeOH to give the title compound (486 mg, 42%) as an off-white solid. ¹H NMR (500 MHZ, DMSO-d₆) δ 11.09-10.87 (m, 2H), 10.43 (t, J=5.5 Hz, 1H), 8.90 (d, J=2.9 Hz, 1H), 8.34 (d, J=8.4 Hz, 1H), 8.25 (s, 1H), 7.91 (ddd, J=7.5, 1.8, 1.2 Hz, 1H), 7.38 (dd, J=8.2, 7.6 Hz, 1H), 3.24 (td, J=7.0, 5.6 Hz, 2H), 2.70 (d, J=1.0 Hz, 3H), 1.52 (app. sextet, J=7.3 Hz, 2H), 0.91 (t, J=7.4 Hz, 3H). m/z 356.2 [M+H]⁺.

### Example 41: 4-Amino-8-(2-amino-5-fluoropyrimidin-4-yl)-2-oxo-N-propyl-1,2-dihydroquinoline-3-carboxamide

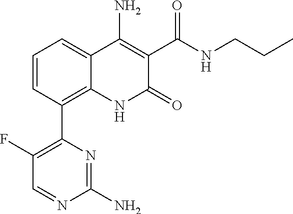 [SMILES: CCCNC(=O)c1c(N)c2cccc(-c3nc(N)ncc3F)c2[nH]c1=O]

General procedure 1 was followed using intermediate 3 (100 mg, 0.35 mmol) and 4-chloro-5-fluoropyrimidin-2-amine (51 mg, 0.35 mmol). The material was purified by flash column chromatography on an ISCO system (10 g silica, elution with a 0-100% EtOAc/pet. ether gradient) and further purification by reverse phase chromatography on an ISCO ACCQPrep system (20 mm×150 mm C18 column, elution with a 10-100% MeOH/water gradient), followed by flash column chromatography on an ISCO system (10 g silica, elution with a 0-20% MeOH/EtOAc gradient) to give the title compound (20 mg, 15%) as a pale yellow solid. ¹H NMR (500 MHZ, DMSO-d₆) δ 10.96 (s, 1H), 10.91 (s, 1H), 10.47 (t, J=5.5 Hz, 1H), 8.47 (d, J=3.0 Hz, 1H), 8.30 (d, J=8.2 Hz, 1H), 8.24 (s, 1H), 7.89-7.83 (m, 1H), 7.36 (dd, J=8.3, 7.6 Hz, 1H), 6.92 (s, 2H), 3.27-3.21 (m, 2H), 1.54-1.51 (m, 2H), 0.91 (d, J=7.5 Hz, 3H). m/z 357.2 [M+H]⁺.

### Example 42: 4-Amino-8-(5-fluoro-2-methoxy-pyrimidin-4-yl)-2-oxo-N-propyl-1H-quinoline-3-carboxamide

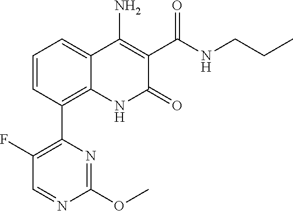 [SMILES: CCCNC(=O)c1c(N)c2cccc(-c3nc(OC)ncc3F)c2[nH]c1=O]

General procedure 1 was followed using intermediate 4 (250 mg, 0.67 mmol) and 4-chloro-5-fluoro-2-methoxypyrimidine (109 mg, 0.67 mmol). The material was purified by flash column chromatography on an ISCO system (10 g silica, elution with a 0-10% MeOH/CH₂Cl₂ gradient) to yield a solid, which was triturated with MeOH to give the title compound (85 mg, 32%) as a pale yellow solid. ¹H NMR (500 MHZ, DMSO-d₆) δ 10.94 (s, 1H), 10.42 (d, J=5.6 Hz, 1H), 8.84 (dd, J=2.5, 1.0 Hz, 1H), 8.38-8.31 (m, 1H), 7.95-7.86 (m, 1H), 7.41-7.36 (m, 1H), 3.99 (s, 3H), 3.28-3.21 (m, 2H), 1.64-1.39 (m, 2H), 0.92 (t, J=7.4 Hz, 3H). m/z 372.2 [M+H]⁺.

### Example 43: 4-Amino-8-(5-fluoro-2-(2-methoxyethoxy) pyrimidin-4-yl)-2-oxo-N-propyl-1,2-dihydroquinoline-3-carboxamide

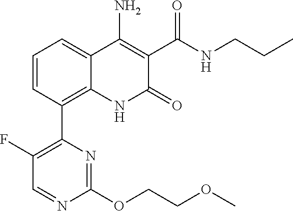 [SMILES: CCCNC(=O)c1c(N)c2cccc(-c3nc(OCCOC)ncc3F)c2[nH]c1=O]

### Step 1

A mixture of intermediate 3 (2.50 g, 8.65 mmol), 2,4-dichloro-5-fluoropyrimidine (1.52 g, 9.08 mmol), Na₂CO₃ (1.40 g, 13.0 mmol) in MeCN (75 mL) and water (30 mL) was degassed by sparging with N₂. Pd-118 (283 mg, 0.43 mmol) was added and the reaction mixture was stirred at rt for 1 h. After this time, the reaction mixture was treated with 2 M HCl (20 mL) and stirred for a further 1 h. After this time, the obtained solids were collected by vacuum filtration and the filter cake washed with water and MeOH. The solids were triturated in refluxing EtOAc for 30 min, cooled to rt and filtered under vacuum to give 4-amino-8-(2-chloro-5-fluoropyrimidin-4-yl)-2-oxo-N-propyl-1,2-dihydroquinoline-3-carboxamide (1.06 g, 31%) as a brown solid. ¹H NMR (500 MHZ, DMSO-d₆) δ 10.97 (s, 1H), 10.62 (s, 1H), 10.42 (t, J=5.6 Hz, 1H), 9.02 (d, J=1.8 Hz, 1H), 8.36 (app. dd, J=8.3, 0.7 Hz, 1H), 8.26 (s, 1H), 7.80 (dt, J=7.5, 1.3 Hz, 1H), 7.39 (dd, J=8.2, 7.5 Hz, 1H), 3.24 (td, J=6.8, 5.6 Hz, 2H), 1.52 (app. sextet, J=7.3 Hz, 2H), 0.91 (t, J=7.4 Hz, 3H). m/z 376.1, 378.1 [M+H]⁺.

### Step 2

To NaH (1.4 eq.) in THF (0.08 M), was added 2-methoxyethanol (50 μL, 0.63 mmol) and the reaction mixture was stirred for 15 min. After this time, 4-amino-8-(2-chloro-5-fluoropyrimidin-4-yl)-2-oxo-N-propyl-1,2-dihydroquinoline-3-carboxamide (50 mg, 0.13 mmol) was added and the reaction mixture was stirred at rt for 16 h. After this time, the reaction mixture was then diluted with EtOAc, quenched with water and the layers separated. The aqueous layer was extracted with EtOAc (2 x) and the combined organic extracts were dried over MgSO₄, filtered and concentrated under reduced pressure. The material was purified by flash column chromatography on an ISCO system (10 g silica, elution with a 0-10% MeOH/CH₂Cl₂ gradient), followed by flash column chromatography on an ISCO system (10 g silica, elution with a 40-100% EtOAc/CH₂Cl₂ gradient) to give the title compound (18 mg, 31%) as a yellow solid. ¹H NMR (500 MHZ, DMSO-d₆) δ 10.97 (s, 1H), 10.82 (s, 1H), 10.42 (t, J=5.5 Hz, 1H), 8.82 (d, J=2.3 Hz, 1H), 8.39-8.12 (m, 2H), 7.87 (dt, J=7.5, 1.7 Hz, 1H), 7.38 (dd, J=8.2, 7.5 Hz, 1H), 4.47-4.41 (m, 2H), 3.73-3.68 (m, 2H), 3.32 (s, 3H), 3.24 (td, J=7.0, 5.6 Hz, 2H), 1.52 (app. sextet, J=7.3 Hz, 2H), 0.91 (t, J=7.4 Hz, 3H). m/z 416.2 [M+H]⁺.

### Example 44: 4-Amino-8-(4-methoxypyrimidin-5-yl)-2-oxo-N-propyl-1H-quinoline-3-carboxamide

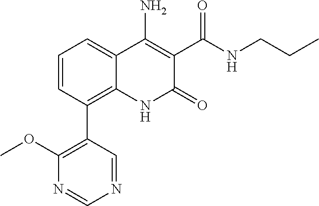 [SMILES: CCCNC(=O)c1c(N)c2cccc(-c3cncnc3OC)c2[nH]c1=O]

General procedure 2 was followed using intermediate 4 (100 mg, 0.22 mmol) and 5-bromo-4-methoxypyrimidine (41 mg, 0.22 mmol). The material was purified by flash column chromatography on an ISCO system (10 g silica, elution with a 50-100% EtOAc/pet. ether gradient) to give the title compound (35 mg, 46%) as a colourless solid. ¹H NMR (500 MHZ, Chloroform-d) δ 11.34 (s, 1H), 10.22 (s, 1H), 8.92 (s, 1H), 8.44 (s, 1H), 7.76-7.67 (m, 2H), 7.43 (d, J=7.4 Hz, 1H), 7.32 (t, J=7.8 Hz, 1H), 5.90 (s, 1H), 4.00 (s, 3H), 3.36 (app. q, J=6.8 Hz, 2H), 1.64 (app. sextet, J=7.4 Hz, 2H), 0.98 (t, J=7.4 Hz, 3H). m/z 354.2 [M+H]⁺.

### Example 45: 4-Amino-2-oxo-N-propyl-8-(pyrazin-2-yl)-1,2-dihydroquinoline-3-carboxamide

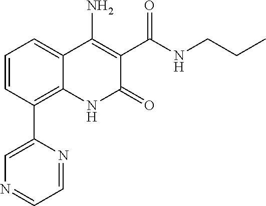 [SMILES: CCCNC(=O)c1c(N)c2cccc(-c3cnccn3)c2[nH]c1=O]

General procedure 1 was followed using intermediate 4 (200 mg, 0.40 mmol) and 2-bromopyrazine (77 mg, 0.48 mmol). The material was purified by reverse phase chromatography on an ISCO system (12 g RediSep® Rf Reversed-phase C18, elution with a 10-100% MeOH/water gradient) to give the title compound (57 mg, 41%) as a yellow solid. ¹H NMR (500 MHz, DMSO-d₆) δ 12.10 (s, 1H), 10.98 (s, 1H), 10.44 (t, J=5.6 Hz, 1H), 9.41 (s, 1H), 8.84-8.80 (m, 1H), 8.76-8.72 (m, 1H), 8.39 (d, J=7.6 Hz, 1H), 8.33 (d, J=7.6 Hz, 1H), 8.27 (s, 1H), 7.44-7.38 (m, 1H), 3.29-3.21 (m, 2H), 1.58-1.47 (m, 2H), 0.93 (t, J=7.4 Hz, 3H). m/z 324.0 [M+H]⁺.

### Example 46:4-Amino-8-(6-methylpyrazin-2-yl)-2-oxo-N-propyl-1,2-dihydroquinoline-3-carboxamide

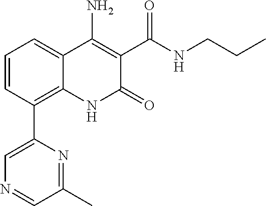 [SMILES: CCCNC(=O)c1c(N)c2cccc(-c3cncc(C)n3)c2[nH]c1=O]

General procedure 1 was followed using intermediate 4 (150 mg, 0.40 mmol) and 2-bromo-6-methylpyrazine (70 mg, 0.40 mmol). The material was purified by flash column chromatography on an ISCO system (10 g silica, elution with a 0-100% EtOAc/pet. ether gradient) to yield a solid, which was then triturated with Et₂O to give the title compound (84 mg, 59%) as a cream solid. ¹H NMR (500 MHZ, DMSO-d₆) δ 12.40 (s, 1H), 10.96 (s, 1H), 10.45 (t, J=5.5 Hz, 1H), 9.21 (s, 1H), 8.63 (s, 1H), 8.37 (dd, J=7.8, 1.1 Hz, 1H), 8.33-8.28 (m, 1H), 8.23 (s, 1H), 7.41-7.35 (m, 1H), 3.28-3.20 (m, 2H), 2.64 (s, 3H), 1.59-1.47 (m, 2H), 0.93 (t, J=7.4 Hz, 3H). m/z 338.2 [M+H]⁺.

### Example 47: 4-Amino-8-(6-methoxypyrazin-2-yl)-2-oxo-N-propyl-1,2-dihydroquinoline-3-carboxamide

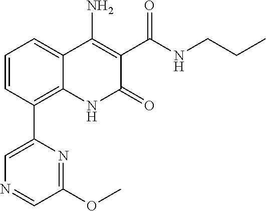 [SMILES: CCCNC(=O)c1c(N)c2cccc(-c3cncc(OC)n3)c2[nH]c1=O]

General procedure 1 was followed using intermediate 4 (150 mg, 0.4 mmol) and 2-bromo-6-methoxypyrazine (79 mg, 0.44 mmol). The material was purified by flash column chromatography on an ISCO system (10 g silica, elution with a 0-100% EtOAc/pet. ether gradient) to give the title compound (48 mg, 32%) as a pale yellow solid. ¹H NMR (500 MHz, DMSO-d₆) δ 11.98 (s, 1H), 10.98 (s, 1H), 10.42 (t, J=5.5 Hz, 1H), 8.91 (s, 1H), 8.40 (d, J=0.6 Hz, 1H), 8.34-8.28 (m, 2H), 8.25 (s, 1H), 7.38 (dd, J=8.2, 7.7 Hz, 1H), 4.08 (s, 3H), 3.28-3.20 (m, 2H), 1.58-1.48 (m, 2H), 0.92 (t, J=7.4 Hz, 3H). m/z 354.2 [M+H]⁺.

### Example 48:4-Amino-8-(3-methoxyphenyl)-2-oxo-N-propyl-1H-quinoline-3-carboxamide

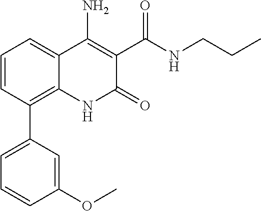 [SMILES: CCCNC(=O)c1c(N)c2cccc(-c3cccc(OC)c3)c2[nH]c1=O]

General procedure 1 was followed using intermediate 3 (100 mg, 0.35 mmol) and 3-bromoanisole (46 μL, 0.36 mmol). The material was purified by flash column chromatography on an ISCO system (10 g silica, elution with a 30-100% EtOAc/pet. ether gradient), followed by reverse phase chromatography on an ISCO system (12 g RediSep® Rf Reversed-phase C18, elution with a 10-100% MeOH/water gradient) to give the title compound (45 mg, 35%) as a tan solid. ¹H NMR (500 MHZ, DMSO-d₆) δ 10.97 (s, 1H), 10.38 (t, J=5.6 Hz, 1H), 8.69 (s, 1H), 8.24 (s, 1H), 8.18 (dd, J=8.3, 1.2 Hz, 1H), 7.53 (dd, J=7.3, 1.2 Hz, 1H), 7.47 (ddd, J=8.3, 7.4, 0.5 Hz, 1H), 7.32 (dd, J=8.3, 7.3 Hz, 1H), 7.06 (ddd, J=8.4, 2.6, 0.9 Hz, 1H), 7.04-6.99 (m, 2H), 3.81 (s, 3H), 3.22 (td, J=7.0, 5.6 Hz, 2H), 1.50 (app. sextet, J=7.3 Hz, 2H), 0.90 (t, J=7.4 Hz, 3H). m/z 352.2 [M+H]⁺.

### Example 49:4-Amino-8-(2-fluoro-5-methoxy-phenyl)-2-oxo-N-propyl-1H-quinoline-3-carboxamide

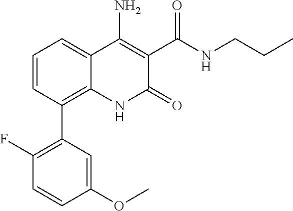 [SMILES: CCCNC(=O)c1c(N)c2cccc(-c3cc(OC)ccc3F)c2[nH]c1=O]

General procedure 3 was followed using intermediate 2 (70 mg, 0.22 mmol) and (2-fluoro-5-methoxyphenyl) boronic acid (51 mg, 0.30 mmol). After this time, the reaction mixture was concentrated under reduced pressure and the residue diluted with CH₂Cl₂ (10 mL) and water (5 mL). The layers were separated via a phase separation cartridge and the organic layer concentrated under reduced pressure. The material was purified by flash column chromatography on an ISCO system (10 g silica, elution with a 15-60% EtOAc/pet. ether gradient) to give the title compound (44 mg, 52%) as a colourless solid. ¹H NMR (500 MHZ, Chloroform-d) δ 11.27 (s, 1H), 10.27 (t, J=5.5 Hz, 1H), 8.01 (s, 1H), 7.68 (d, J=8.3 Hz, 1H), 7.48 (d, J=7.3 Hz, 1H), 7.33-7.23 (m, 1H), 7.15 (t, J=8.9 Hz, 1H), 7.00-6.91 (m, 1H), 6.80 (dd, J=5.8, 3.2 Hz, 1H), 6.00 (s, 1H), 3.80 (s, 3H), 3.41-3.27 (m, 2H), 1.62 (q, J=7.3 Hz, 2H), 0.97 (t, J=7.4 Hz, 3H). m/z 370.1 [M+H]⁺.

### Example 50: 4-Amino-8-(2,6-difluorophenyl)-2-oxo-N-propyl-1H-quinoline-3-carboxamide

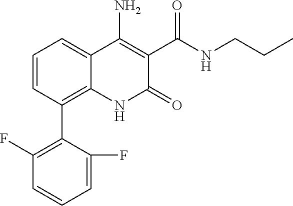 [SMILES: CCCNC(=O)c1c(N)c2cccc(-c3c(F)cccc3F)c2[nH]c1=O]

General procedure 1 was followed using intermediate 4 (100 mg, 0.28 mmol) and 1-bromo-2,6-difluorobenzene (60 mg, 0.31 mmol). The material was purified by reverse phase chromatography on an ISCO system (12 g RediSep® Rf Reversed-phase C18, elution with a 10-100% MeOH/water gradient) to give the title compound (22 mg, 21%) as a colourless solid. ¹H NMR (500 MHZ, DMSO-d₆) δ 10.95 (s, 1H), 10.49 (d, J=6.3 Hz, 1H), 10.13 (s, 1H), 8.33-8.11 (m, 2H), 7.63-7.48 (m, 2H), 7.32 (t, J=8.0 Hz, 1H), 7.23 (t, J=8.2 Hz, 2H), 3.24 (d, J=6.8 Hz, 2H), 1.51 (q, J=7.4 Hz, 2H), 0.99-0.86 (m, 3H). m/z 358.1 [M+H]⁺.

### Example 51: 4-Amino-8-(2,2-difluoro-1,3-benzodioxol-4-yl)-2-oxo-N-propyl-1H-quinoline-3-carboxamide

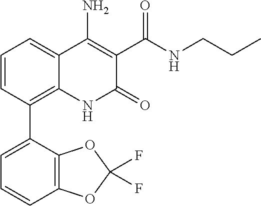 [SMILES: CCCNC(=O)c1c(N)c2cccc(-c3cccc4c3OC(F)(F)O4)c2[nH]c1=O]

General procedure 2 was followed using intermediate 4 (100 mg, 0.22 mmol) and 4-bromo-2,2-difluoro-1,3-benzodioxole (51 mg, 0.22 mmol). The material was purified by flash column chromatography on an ISCO system (10 g silica, elution with a 50-100% EtOAc/pet. ether gradient) to give the title compound (28 mg, 32%) as a colourless solid. ¹H NMR (500 MHZ, Chloroform-d) o 11.34 (s, 1H), 10.23 (s, 1H), 8.00 (s, 1H), 7.69 (d, J=8.2 Hz, 1H), 7.56 (d, J=7.2 Hz, 1H), 7.33 (t, J=7.9 Hz, 1H), 7.24-7.15 (m, 2H), 7.11 (d, J=7.8 Hz, 1H), 5.90 (s, 1H), 3.36 (q, J=6.5 Hz, 2H), 1.63 (app. sextet, J=6.7 Hz, 2H), 0.98 (t, J=6.7 Hz, 3H). m/z 402.2 [M+H]⁺.

### Example 52:4-Amino-8-(2,5-dimethyl-1H-imidazol-4-yl)-2-oxo-N-propyl-1H-quinoline-3-carboxamide

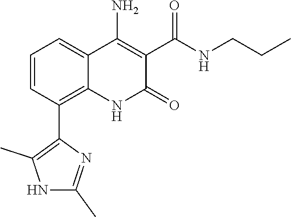 [SMILES: CCCNC(=O)c1c(N)c2cccc(-c3nc(C)[nH]c3C)c2[nH]c1=O]

A mixture of intermediate 4 (100 mg, 0.22 mmol), Pd₂(dba)₃ (17 mg, 0.02 mmol), PtBu₃·HBF₄ (13.4 mg, 0.05 mmol) and KF (143 mg, 2.47 mmol) in DMSO (10 mL) was degassed by sparging with N₂. 4-Bromo-2,5-dimethyl-1H-imidazole (38 mg, 0.22 mmol) was added and the reaction mixture heated at 135° C. for 30 min under microwave irradiation. After this time, the solids were separated by vacuum filtration, washed with MeOH and the filtrate was concentrated under reduced pressure. The material was purified by reverse phase chromatography on an ISCO system (12 g RediSep® Rf Reversed-phase C18, elution with a 10-100% MeOH/water gradient) to give the title compound (12 mg, 16%) as a yellow solid. ¹H NMR (500 MHZ, Chloroform-d) o 12.00 (s, 1H), 11.14 (s, 1H), 10.61 (s, 1H), 8.73 (s, 1H), 7.55 (d, J=7.6 Hz, 1H), 7.49 (d, J=8.4 Hz, 1H), 7.20 (t, J=8.1 Hz, 1H), 5.75 (s, 1H), 3.37 (app. q, J=7.0 Hz, 2H), 2.46 (s, 3H), 2.43 (s, 3H), 1.65 (app. sextet, J=8.1 Hz, 2H), 1.00 (t, J=7.5 Hz, 3H). m/z 340.2 [M+H]⁺.

### Example 53: 4-Amino-8-(2-methylpyrazol-3-yl)-2-oxo-N-propyl-1H-quinoline-3-carboxamide

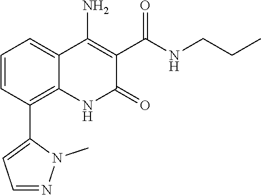 [SMILES: CCCNC(=O)c1c(N)c2cccc(-c3ccnn3C)c2[nH]c1=O]

General procedure 1 was followed using intermediate 3 (400 mg, 1.38 mmol) and 5-bromo-1-methyl-1H-pyrazole (234 mg, 1.45 mmol). The material was purified by flash column chromatography on an ISCO system (10 g silica, elution with a 0-10% MeOH/CH₂Cl₂ gradient) to yield a solid, which was then triturated with MeOH to give the title compound (157 mg, 33%) as a grey solid. ¹H NMR (500 MHZ, DMSO-d₆) δ 10.97 (s, 1H), 10.39 (t, J=5.6 Hz, 1H), 9.12 (s, 1H), 8.39-8.11 (m, 2H), 7.63-7.56 (m, 2H), 7.34 (dd, J=8.3, 7.4 Hz, 1H), 6.48 (d, J=1.9 Hz, 1H), 3.65 (s, 3H), 3.23 (td, J=6.8, 5.6 Hz, 2H), 1.50 (app. sextet, J=7.3 Hz, 2H), 0.90 (t, J=7.4 Hz, 3H). m/z 326.2 [M+H]⁺.

### Example 54: 4-Amino-8-(3,5-dimethylisoxazol-4-yl)-2-oxo-N-propyl-1H-quinoline-3-carboxamide

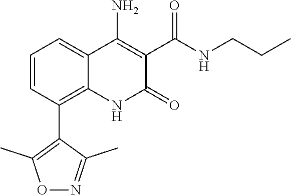 [SMILES: CCCNC(=O)c1c(N)c2cccc(-c3c(C)noc3C)c2[nH]c1=O]

General procedure 3 was followed using intermediate 2 (70 mg, 0.21 mmol) and 3,5-dimethylisoxazole-4-boronic acid (40 mg, 0.29 mmol). After this time, the reaction mixture was concentrated under reduced pressure and the residue dissolved in CH₂Cl₂ (10 mL) and water (5 mL). The layers were separated via a phase separation cartridge and the organic layer concentrated under reduced pressure. The material was purified by flash column chromatography on an ISCO system (10 g silica, elution with a 50-100% EtOAc/pet. ether gradient) to give the title compound (9 mg, 12%) as a colourless solid. 1H NMR (500 MHZ, Chloroform-d) δ 11.34 (s, 1H), 10.22 (t, J=5.8 Hz, 1H), 7.85 (s, 1H), 7.69 (d, J=8.2 Hz, 1H), 7.37 (d, J=7.1 Hz, 1H), 7.30 (t, J=7.8 Hz, 1H), 5.95 (s, 1H), 3.36 (q, J=6.6 Hz, 2H), 2.30 (s, 3H), 2.12 (s, 3H), 1.63 (q, J=7.7 Hz, 2H), 0.98 (t, J=7.4 Hz, 3H). m/z 341.2 [M+H]⁺.

### Example 55: 4-Amino-8-(4-methoxy-3-pyridyl)-N-methyl-2-oxo-1H-quinoline-3-carboxamide

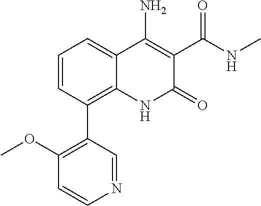

### Step 1: Synthesis of Intermediate 5: Ethyl 4-amino-8-(4-methoxy-3-pyridyl)-2-oxo-1H-quinoline-3-carboxylate

General procedure 3 was followed using intermediate 1 (600 mg, 1.93 mmol) and (4-methoxy-3-pyridinyl) boronic acid (989 mg, 5.79 mmol). The material was purified by flash column chromatography on an ISCO system (24 g silica, elution with a 0-20% EtOAc/MeOH gradient) to give intermediate 5 (420 mg, 61%) as a colourless solid. 1H NMR (500 MHZ, DMSO-d₆) δ 8.94 (s, 1H), 8.51 (d, J=5.8 Hz, 1H), 8.31 (s, 2H), 8.22 (s, 1H), 8.14 (d, J=8.2 Hz, 1H), 7.36 (dd, J=7.4, 1.2 Hz, 1H), 7.20 (t, J=7.9 Hz, 1H), 7.17 (d, J=5.9 Hz, 1H), 4.20 (qd, J=7.2, 2.2 Hz, 2H), 3.77 (s, 3H), 1.23 (t, J=7.1 Hz, 3H). m/z 340.1 [M+H]⁺.

### Step 2

General procedure 5 was followed using intermediate 5 (50 mg, 0.15 mmol) and methylamine (146 μL, 0.29 mmol). The material was purified by flash column chromatography on an ISCO system (10 g silica, elution with a 0-20% MeOH/EtOAc gradient) to give the title compound (20 mg, 41%) as a colourless solid. ¹H NMR (500 MHz, Chloroform-d) δ 11.28 (s, 1H), 10.12 (s, 1H), 8.64 (d, J=5.8 Hz, 1H), 8.38 (s, 1H), 7.79 (s, 1H), 7.68 (d, J=8.3 Hz, 1H), 7.44 (dd, J=7.3, 1.2 Hz, 1H), 7.33-7.29 (m, 1H), 6.99 (d, J=5.8 Hz, 1H), 5.93 (s, 1H), 3.85 (s, 3H), 2.94 (s, 3H). m/z 325.1 [M+H]⁺.

### Example 56: 4-Amino-N-ethyl-8-(4-methoxy-3-pyridyl)-2-oxo-1H-quinoline-3-carboxamide

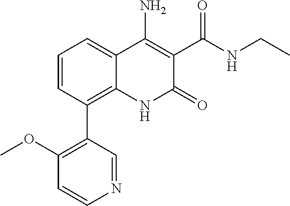 [SMILES: CCNC(=O)c1c(N)c2cccc(-c3cnccc3OC)c2[nH]c1=O]

General procedure 5 was followed using intermediate 5 (200 mg, 0.59 mmol) and ethylamine (1.47 mL, 2.95 mmol). The material was purified by flash column chromatography on an ISCO system (10 g silica, elution with a 0-10% MeOH/EtOAc gradient) to yield a solid, which was then triturated with Et₂O to give the title compound (89 mg, 45%) as a colourless solid. ¹H NMR (500 MHZ, Chloroform-d) δ 10.91 (s, 1H), 10.40 (t, J=5.6 Hz, 1H), 9.45 (s, 1H), 8.51 (d, J=5.8 Hz, 1H), 8.22 (s, 1H), 8.16 (d, J=8.3 Hz, 2H), 7.39 (d, J=7.2 Hz, 1H), 7.25 (t, J=7.8 Hz, 1H), 7.17 (d, J=5.8 Hz, 1H), 3.76 (s, 3H), 3.25 (q, J=6.5 Hz, 2H), 1.08 (t, J=7.2 Hz, 3H). m/z 339.1 [M+H]⁺.

### Example 57: 4-Amino-N-cyclopropyl-8-(4-methoxy-3-pyridyl)-2-oxo-1H-quinoline-3-carboxamide

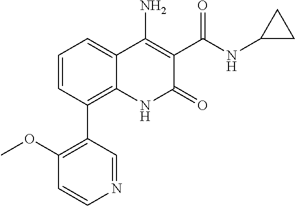 [SMILES: COc1ccncc1-c1cccc2c(N)c(C(=O)NC3CC3)c(=O)[nH]c12]

General procedure 5 was followed using intermediate 5 (40 mg, 0.12 mmol) and cyclopropylamine (41 μL, 0.59 mmol). The material was purified by flash column chromatography on an ISCO system (10 g silica, elution with a 0-5% MeOH/EtOAc gradient) to give the title compound (5 mg, 12%) as a colourless solid. ¹H NMR (500 MHZ, Chloroform-d) δ 11.28 (s, 1H), 10.23 (s, 1H), 8.63 (d, J=5.8 Hz, 1H), 8.37 (s, 1H), 7.75 (s, 1H), 7.68 (d, J=8.3 Hz, 1H), 7.43 (d, J=7.3 Hz, 1H), 7.30 (t, J=7.9 Hz, 1H), 6.98 (d, J=5.8 Hz, 1H), 5.96 (s, 1H), 3.84 (s, 3H), 2.90-2.77 (m, 1H), 0.84-0.77 (m, 2H), 0.66-0.53 (m, 2H). m/z 351.1 [M+H]⁺.

### Example 58: 4-Amino-8-(4-methoxy-3-pyridyl)-N-(oxetan-3-yl)-2-oxo-1H-quinoline-3-carboxamide

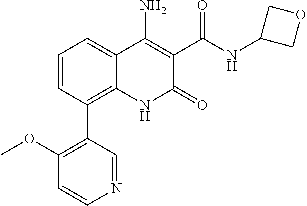 [SMILES: COc1ccncc1-c1cccc2c(N)c(C(=O)NC3COC3)c(=O)[nH]c12]

General procedure 6 was followed using intermediate 5 (50 mg, 0.15 mmol) and 3-oxetanamine (21 μL, 0.31 mmol). The material was purified by flash column chromatography on an ISCO system (10 g silica, elution with a 0-20% MeOH/CH₂Cl₂ gradient) to yield the title compound (21 mg, 35%) as a colourless solid. ¹H NMR (500 MHz, Chloroform-d) δ 11.05 (s, 1H), 10.83 (d, J=6.4 Hz, 1H), 8.65 (dd, J=5.8, 1.5 Hz, 1H), 8.38 (d, J=1.4 Hz, 1H), 7.81 (s, 1H), 7.69 (d, J=8.2 Hz, 1H), 7.46 (dd, J=7.2, 1.5 Hz, 1H), 7.40-7.28 (m, 1H), 7.00 (d, J=5.6 Hz, 1H), 6.01 (s, 1H), 5.15 (q, J=6.9 Hz, 1H), 4.95 (t, J=7.0 Hz, 2H), 4.69 (t, J=6.5 Hz, 2H), 3.86 (s, 3H). m/z 367.1 [M+H]⁺.

### Example 59: 4-Amino-N-(cyclopropylmethyl)-8-(4-methoxy-3-pyridyl)-2-oxo-1H-quinoline-3-carboxamide

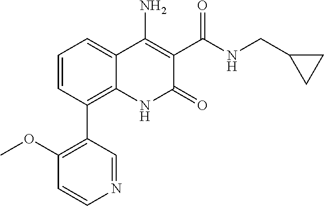 [SMILES: COc1ccncc1-c1cccc2c(N)c(C(=O)NCC3CC3)c(=O)[nH]c12]

General procedure 6 was followed using intermediate 5 (40 mg, 0.12 mmol) and cyclopropylmethylamine (51 μL, 0.59 mmol). The material was purified by flash column chromatography on an ISCO system (10 g silica, elution with a 0-20% MeOH/CH₂Cl₂ gradient) to yield a solid, which was then triturated with Et₂O to give the title compound (5 mg, 11%) as a colourless solid. ¹H NMR (500 MHZ, Chloroform-d) δ 11.28 (s, 1H), 10.32 (s, 1H), 8.64 (d, J=5.8 Hz, 1H), 8.38 (s, 1H), 7.77 (s, 1H), 7.68 (d, J=8.3 Hz, 1H), 7.44 (d, J=7.3 Hz, 1H), 7.31 (t, J=7.8 Hz, 1H), 6.99 (d, J=5.8 Hz, 1H), 5.91 (s, 1H), 3.85 (s, 3H), 3.28 (q, J=6.6 Hz, 2H), 1.12-1.03 (m, 1H), 0.56-0.48 (m, 2H), 0.30-0.23 (m, 2H). m/z 365.2 [M+H]⁺.

### Example 60: 4-Amino-N-isobutyl-8-(4-methoxy-3-pyridyl)-2-oxo-1H-quinoline-3-carboxamide

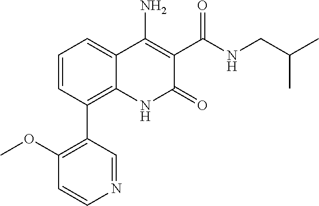 [SMILES: COc1ccncc1-c1cccc2c(N)c(C(=O)NCC(C)C)c(=O)[nH]c12]

General procedure 5 was followed using intermediate 5 (40 mg, 0.12 mmol) and isobutylamine (59 μL, 0.59 mmol). The material was purified by flash column chromatography on an ISCO system (10 g silica, elution with a 0-20% MeOH/CH₂Cl₂ gradient) to give the title compound (8 mg, 18%) as a colourless solid. ¹H NMR (500 MHZ, Chloroform-d) δ 11.29 (s, 1H), 10.34 (s, 1H), 8.63 (d, J=5.8 Hz, 1H), 8.37 (s, 1H), 7.76 (s, 1H), 7.68 (d, J=8.3 Hz, 1H), 7.44 (d, J=7.3 Hz, 1H), 7.31 (t, J=7.8 Hz, 1H), 6.98 (d, J=5.8 Hz, 1H), 5.91 (s, 1H), 3.85 (s, 3H), 3.23 (t, J=6.3 Hz, 2H), 1.89 (p, J=6.7 Hz, 1H), 0.98 (d, J=6.7 Hz, 6H). m/z 367.2 [M+H]⁺.

### Example 61: 4-Amino-N-(2,2-difluoroethyl)-8-(4-methoxy-3-pyridyl)-2-oxo-1H-quinoline-3-carboxamide

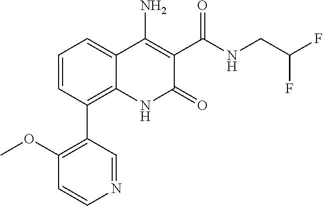 [SMILES: COc1ccncc1-c1cccc2c(N)c(C(=O)NCC(F)F)c(=O)[nH]c12]

General procedure 5 was followed using intermediate 5 (40 mg, 0.12 mmol) and 2,2-difluoroethanamine (42 μL, 0.59 mmol). The material was purified by flash column chromatography on an ISCO system (10 g silica, elution with a 0-5% MeOH/EtOAc gradient) to give the title compound (9 mg, 20%) as a colourless solid. ¹H NMR (500 MHZ, Chloroform-d) δ 11.04 (s, 1H), 10.57 (s, 1H), 8.64 (d, J=5.8 Hz, 1H), 8.38 (s, 1H), 7.80 (s, 1H), 7.69 (d, J=8.3 Hz, 1H), 7.46 (d, J=7.1 Hz, 1H), 7.33 (t, J=7.8 Hz, 1H), 6.99 (d, J=5.8 Hz, 1H), 6.10-5.80 (m, 2H), 3.85 (s, 3H), 3.84-3.69 (m, 2H). m/z 375.1 [M+H]⁺.

### Example 62: 4-Amino-8-(4-methoxypyridin-3-yl)-2-oxo-N-(propyl-d₇)-1,2-dihydroquinoline-3-carboxamide

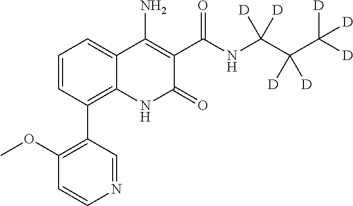 [SMILES: [2H]C([2H])([2H])C([2H])([2H])C([2H])([2H])NC(=O)c1c(N)c2cccc(-c3cnccc3OC)c2[nH]c1=O]

General procedure 6 was followed using intermediate 5 (150 mg, 0.44 mmol) and n-propyl-d₇-amine hydrochloride (91 mg, 0.88 mmol) at 50° C. The material was purified by flash column chromatography on an ISCO system (10 g silica, elution with a 0-10% MeOH/EtOAc gradient) to give the title compound (67 mg, 40%) as a yellow solid. 1H NMR (500 MHZ, DMSO-d₆) δ 10.93 (s, 1H), 10.47 (s, 1H), 9.50 (s, 1H), 8.56 (d, J=5.9 Hz, 1H), 8.28 (s, 1H), 8.22-8.09 (m, 2H), 7.42 (dd, J=7.3, 1.3 Hz, 1H), 7.28 (dd, J=8.3, 7.3 Hz, 1H), 7.22 (d, J=5.9 Hz, 1H), 3.80 (s, 3H). m/z 360.3 [M+H]⁺.

### Example 63: 4-Amino-N-(2-methoxyethyl)-8-(4-methoxy-3-pyridyl)-2-oxo-1H-quinoline-3-carboxamide

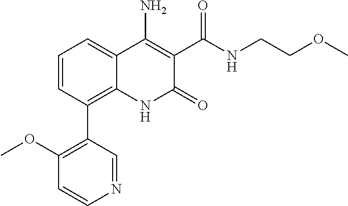

General procedure 5 was followed using intermediate 5 (40 mg, 0.12 mmol) and 2-methoxyethanamine (51 μL, 0.59 mmol). The material was purified by flash column chromatography on an ISCO system (10 g silica, elution with a 0-20% MeOH/CH₂Cl₂ gradient) to give the title compound (8 mg, 18%) as a colourless solid. ¹H NMR (500 MHZ, Chloroform-d) δ 11.22 (s, 1H), 10.44 (s, 1H), 8.63 (d, J=5.8 Hz, 1H), 8.37 (s, 1H), 7.77 (s, 1H), 7.67 (d, J=8.3 Hz, 1H), 7.44 (d, J=7.4 Hz, 1H), 7.31 (t, J=7.8 Hz, 1H), 6.98 (d, J=5.8 Hz, 1H), 5.91 (s, 1H), 3.84 (s, 3H), 3.65-3.54 (m, 4H), 3.40 (s, 3H). m/z 369.2 [M+H]⁺.

### Example 64: 4-Amino-N-(2-hydroxyethyl)-8-(4-methoxy-3-pyridyl)-2-oxo-1H-quinoline-3-carboxamide

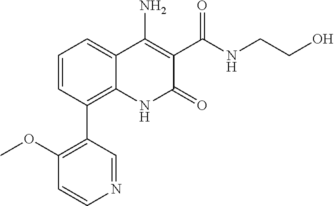 [SMILES: COc1ccncc1-c1cccc2c(N)c(C(=O)NCCO)c(=O)[nH]c12]

General procedure 6 was followed using intermediate 5 (52 mg, 0.15 mmol) and ethanolamine (18 μL, 0.31 mmol). The material was purified by flash column chromatography on an ISCO system (10 g silica, elution with a 0-20% MeOH/EtOAc gradient) to give the title compound (23 mg, 41%) as a colourless solid. ¹H NMR (500 MHz, Chloroform-d) δ 11.07 (s, 1H), 10.58 (s, 1H), 8.64 (dd, J=5.8, 1.0 Hz, 1H), 8.38 (s, 1H), 7.84 (s, 1H), 7.69 (d, J=8.3 Hz, 1H), 7.45 (dt, J=7.4, 1.1 Hz, 1H), 7.32 (dd, J=8.3, 7.3 Hz, 1H), 6.98 (d, J=5.8 Hz, 1H), 5.98 (s, 1H), 3.84 (s, 3H), 3.81 (t, J=4.9 Hz, 2H), 3.59 (dd, J=10.8, 5.7 Hz, 2H). m/z 355.1 [M+H]⁺.

### Example 65: 4-Amino-N-(2,2-difluoropropyl)-8-(4-methoxy-3-pyridyl)-2-oxo-1H-quinoline-3-carboxamide

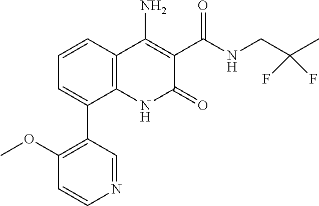 [SMILES: COc1ccncc1-c1cccc2c(N)c(C(=O)NCC(C)(F)F)c(=O)[nH]c12]

General procedure 6 was followed using intermediate 5 (60 mg, 0.18 mmol) and 2,2-difluoropropan-1-aminehydrochloride (47 mg, 0.35 mmol). The material was purified by flash column chromatography on an ISCO system (10 g silica, elution with a 0-8% MeOH/CH₂Cl₂ gradient) to give the title compound (9 mg, 13%) colourless solid. ¹H NMR (500 MHZ, DMSO-d₆) δ 10.87 (t, J=6.0 Hz, 1H), 10.72 (s, 1H), 9.61 (s, 1H), 8.51 (dd, J=5.9, 1.6 Hz, 1H), 8.30 (s, 1H), 8.25-8.15 (m, 2H), 7.41 (dd, J=7.4, 1.5 Hz, 1H), 7.27 (t, J=7.8 Hz, 1H), 7.16 (d, J=5.8 Hz, 1H), 3.86-3.68 (m, 5H), 1.60 (t, J=18.9 Hz, 3H). m/z 389.1 [M+H]⁺.

### Example 66:4-Amino-N-(3-hydroxypropyl)-8-(4-methoxy-3-pyridyl)-2-oxo-1H-quinoline-3-carboxamide

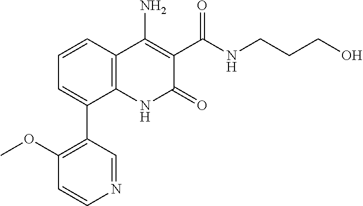 [SMILES: COc1ccncc1-c1cccc2c(N)c(C(=O)NCCCO)c(=O)[nH]c12]

General procedure 6 was followed using intermediate 5 (50 mg, 0.15 mmol) and 3-amino-1-propanol (23 μL, 0.29 mmol). The material was purified by flash column chromatography on an ISCO system (10 g silica, elution with a 0-20% MeOH/CH₂Cl₂ gradient) to give the title compound (38 mg, 66%) as a colourless solid. ¹H NMR (500 MHz, DMSO-d₆) δ10.89 (s, 1H), 10.45 (t, J=5.6 Hz, 1H), 9.46 (s, 1H), 8.51 (dd, J=5.8, 1.3 Hz, 1H), 8.22 (s, 1H), 8.15 (d, J=8.3 Hz, 2H), 7.42-7.36 (m, 1H), 7.25 (t, J=7.7 Hz, 1H), 7.16 (d, J=5.8 Hz, 1H), 4.49 (t, J=5.2 Hz, 1H), 3.76 (s, 3H), 3.47-3.39 (m, 2H), 3.31-3.24 (m, 2H), 1.61 (p, J=6.7 Hz, 2H). m/z 369.2 [M+H]⁺.

### Example 67: 4-Amino-N-(3-fluoropropyl)-8-(4-methoxy-3-pyridyl)-2-oxo-1H-quinoline-3-carboxamide

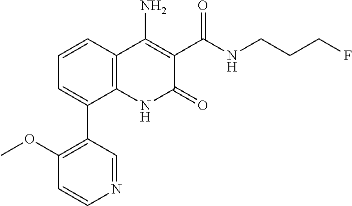 [SMILES: COc1ccncc1-c1cccc2c(N)c(C(=O)NCCCF)c(=O)[nH]c12]

General procedure 6 was followed using intermediate 5 (60 mg, 0.18 mmol) and 3-fluoro-propylaminehydrochloride (40 mg, 0.35 mmol). The material was purified by flash column chromatography on an ISCO system (10 g silica, elution with a 0-40% 20% MeOH in EtOAc/CH₂Cl₂ gradient) to give the title compound (33 mg, 48%) as a colourless solid. ¹H NMR (500 MHZ, DMSO-d₆) δ 10.86 (s, 1H), 10.52 (t, J=5.6 Hz, 1H), 9.49 (s, 1H), 8.51 (d, J=5.7 Hz, 1H), 8.32-8.07 (m, 3H), 7.51-7.34 (m, 1H), 7.26 (t, J=7.8 Hz, 1H), 7.16 (d, J=5.9 Hz, 1H), 4.53 (t, J=5.8 Hz, 1H), 4.43 (t, J=5.8 Hz, 1H), 3.76 (s, 3H), 3.51-3.24 (m, 2H), 1.86 (dq, J=26.6, 6.4 Hz, 2H). m/z 371.2 [M+H]⁺.

### Example 68: 4-Amino-N-(3,3-difluoropropyl)-8-(4-methoxy-3-pyridyl)-2-oxo-1H-quinoline-3-carboxamide

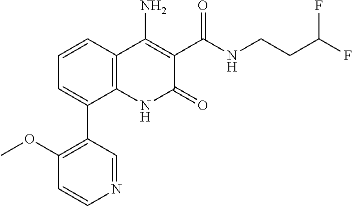 [SMILES: COc1ccncc1-c1cccc2c(N)c(C(=O)NCCC(F)F)c(=O)[nH]c12]

General procedure 6 was followed using intermediate 5 (60 mg, 0.18 mmol) and 3,3-difluoropropan-1-aminehydrochloride (47 mg, 0.35 mmol). The material was purified by flash column chromatography on an ISCO system (10 g silica, elution with a 0-40% 20% MeOH in EtOAc/CH₂Cl₂ gradient) to yield a solid, which was then triturated with Et₂O to give the title compound (4 mg, 6%) as a pale pink solid. ¹H NMR (500 MHz, Chloroform-d) δ 11.16 (s, 1H), 10.43 (t, J=5.8 Hz, 1H), 8.64 (d, J=5.8 Hz, 1H), 8.37 (s, 1H), 7.80 (s, 1H), 7.68 (d, J=8.3 Hz, 1H), 7.45 (dd, J=7.3, 1.3 Hz, 1H), 7.32 (t, J=7.8 Hz, 1H), 6.99 (d, J=5.8 Hz, 1H), 5.95 (m, 2H), 3.85 (s, 3H), 3.57 (dt, J=10.3, 6.4 Hz, 2H), 2.17 (ttd, J=17.5, 6.8, 4.5 Hz, 2H). m/z 389.0 [M+H]⁺.

### Example 69: 4-Amino-8-(4-methoxypyridin-3-yl)-2-oxo-N-(3,3,3-trifluoropropyl)-1,2-dihydroquinoline-3-carboxamide

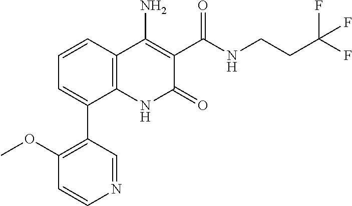 [SMILES: COc1ccncc1-c1cccc2c(N)c(C(=O)NCCC(F)(F)F)c(=O)[nH]c12]

General procedure 5 was followed using intermediate 5 (80 mg, 0.24 mmol) and 3,3,3-trifluoropropylamine hydrochloride (123 mg, 0.83 mmol). The material was purified by reverse phase chromatography on an ISCO system (12 g RediSep® Rf Reversed-phase C18, elution with a 10-100% MeOH/water gradient), followed by reverse phase chromatography on an ISCO ACCQPrep system (20 mm×150 mm C18 column, elution with a 10-100% MeOH/water gradient) to give the title compound (30 mg, 30%) as a pale yellow solid. ¹H NMR (400 MHZ, Methanol-d₄) δ 10.60 (t, J=5.9 Hz, 1H), 8.56 (d, J=5.9 Hz, 1H), 8.32 (s, 1H), 8.10 (d, J=8.3 Hz, 1H), 7.48 (d, J=7.3 Hz, 1H), 7.34 (t, J=7.8 Hz, 1H), 7.26 (d, J=5.9 Hz, 1H), 3.90 (s, 3H), 3.60 (t, J=6.6 Hz, 2H), 2.49 (m, 2H). m/z 407.1 [M+H]⁺.

### Example 70: 4-Amino-N-cyclopropyl-8-[4-(difluoromethoxy)-3-pyridyl]-2-oxo-1H-quinoline-3-carboxamide

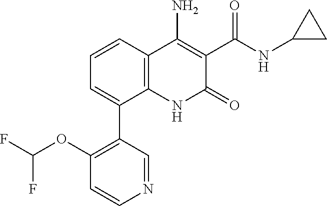 [SMILES: Nc1c(C(=O)NC2CC2)c(=O)[nH]c2c(-c3cnccc3OC(F)F)cccc12]

### Step 1

General procedure 5 was followed using intermediate 1 (0.85 g, 2.7 mmol) and cyclopropylamine (0.9 mL, 13.7 mmol). After this time, the reaction mixture was cooled to 0° C., quenched with 1 M HCl (43 mL) and stirred for 30 min. The precipitate formed was collected by vacuum filtration, washed with 1 M HCl and water and dried under vacuum to give 4-amino-8-bromo-N-cyclopropyl-2-oxo-1H-quinoline-3-carboxamide (622 mg, 67%) as an orange solid. ¹H NMR (500 MHZ, DMSO-d₆) δ 10.93 (s, 1H), 10.32 (d, J=4.0 Hz, 1H), 9.51 (s, 1H), 8.33 (s, 1H), 8.16 (d, J=8.2 Hz, 1H), 7.91 (dd, J=7.8, 1.1 Hz, 1H), 7.17 (t, J=8.0 Hz, 1H), 2.76 (tq, J=7.8, 4.0 Hz, 1H), 1.21 (s, 1H), 0.75-0.64 (m, 2H), 0.49-0.38 (m, 2H). m/z 344.0, 346.0 [M+Na]⁺.

### Step 2

A solution of B₂pin₂ (272 mg, 1.07 mmol), KOAc (234 mg, 2.38 mmol) and 4-amino-8-bromo-N-cyclopropyl-2-oxo-1H-quinoline-3-carboxamide (250 mg, 0.78 mmol) in dioxane (6.5 mL) was degassed by sparging with N₂ for 10 min. After this time, Pd(dppf)Cl₂·CH₂Cl₂ (32 mg, 0.05 mmol) was added and the reaction mixture was stirred at 90° C. for 18 h. After this time, the reaction mixture was diluted with EtOAc (50 mL) and concentrated under reduced pressure. The residue was dissolved in EtOAc (50 mL), washed with water (20 mL) and brine (20 mL), dried over MgSO₄, filtered and concentrated under reduced pressure. The crude material was triturated in pet. ether to give 4-amino-N-cyclopropyl-2-oxo-8-(4,4,5,5-tetramethyl-1,3,2-dioxaborolan-2-yl)-1H-quinoline-3-carboxamide (239.8 mg, 67%) as a brown solid. ¹H NMR (500 MHZ, Chloroform-d) δ 11.08 (s, 1H), 10.39 (s, 1H), 9.94 (s, 1H), 7.96 (d, J=6.8 Hz, 1H), 7.66 (d, J=8.1 Hz, 1H), 7.14 (t, J=7.7 Hz, 1H), 5.84 (s, 1H), 2.89-2.73 (m, 1H), 1.20 (d, J=5.3 Hz, 12H), 0.74 (td, J=7.0, 5.1 Hz, 2H), 0.59-0.47 (m, 2H).

### Step 3

General procedure 2 was followed using 3-bromo-4-(difluoromethoxy) pyridine (40 mg, 0.18 mmol) and 4-amino-N-cyclopropyl-2-oxo-8-(4,4,5,5-tetramethyl-1,3,2-dioxaborolan-2-yl)-1H-quinoline-3-carboxamide (100 mg, 0.27 mmol). The material was purified by flash column chromatography on an ISCO system (10 g silica, elution with a 0-100% EtOAc/pet. ether gradient) to give the title compound (26 mg, 35%) as a grey solid. ¹H NMR (500 MHZ, DMSO-d₆) δ 10.84 (s, 1H), 10.49 (d, J=4.0 Hz, 1H), 9.91 (s, 1H), 8.23-8.10 (m, 2H), 8.07 (d, J=8.1 Hz, 2H), 7.56 (t, J=58.7 Hz, 1H), 7.40 (dd, J=7.4, 1.2 Hz, 1H), 7.23 (t, J=7.8 Hz, 1H), 6.38 (d, J=7.6 Hz, 1H), 2.83-2.69 (m, 1H), 0.78-0.62 (m, 2H), 0.48-0.33 (m, 2H).

### Example 71: 4-Amino-8-[4-(difluoromethoxy)-3-pyridyl]-N-(2-methoxyethyl)-2-oxo-1H-quinoline-3-carboxamide

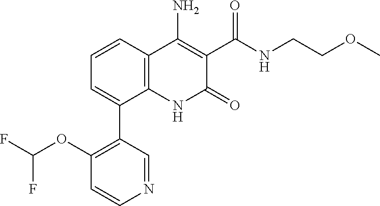 [SMILES: COCCNC(=O)c1c(N)c2cccc(-c3cnccc3OC(F)F)c2[nH]c1=O]

### Step 1

General procedure 5 was followed using intermediate 1 (1.50 g, 4.82 mmol) and 2-methoxyethanamine (1.3 mL, 24.3 mmol). After this time, the reaction mixture was cooled to 0° C. and quenched with 1 M HCl (55 mL) to afford a slurry, which was diluted with 20 mL of water. The slurry was filtered under vacuum, the filter cake washed with 0.5 M HCl and MeOH and dried under reduced pressure to give 4-amino-8-bromo-N-(2-methoxyethyl)-2-oxo-1H-quinoline-3-carboxamide (2.32 g, 135% yield) as a pale yellow solid. ¹H NMR (500 MHZ, DMSO-d₆) δ 10.92 (s, 1H), 10.43 (s, 1H), 9.46 (s, 1H), 8.29 (s, 1H), 8.16 (d, J=8.3 Hz, 1H), 7.90 (d, J=7.8 Hz, 1H), 7.16 (t, J=8.0 Hz, 1H), 3.44-3.41 (m, 4H), 3.26 (s, 3H). m/z 339.9 [M+H]⁺.

### Step 2

A solution of B₂pin₂ (896 mg, 3.53 mmol), KOAc (866 mg, 8.82 mmol) and 4-amino-8-bromo-N-(2-methoxyethyl)-2-oxo-1H-quinoline-3-carboxamide (1.00 g, 2.94 mmol) in dioxane (12 mL) was degassed by sparging with N₂ for 10 min. After this time, Pd(dppf)Cl₂·CH₂Cl₂ (120 mg, 0.15 mmol) was added and the reaction mixture stirred at 95° C. for 12 h. After this time, the reaction mixture was diluted with EtOAc and washed with water and brine. The organic layer was dried over Na₂SO₄, filtered and concentrated under reduced pressure to give 4-amino-N-(2-methoxyethyl)-2-oxo-8-(4,4,5,5-tetramethyl-1,3,2-dioxaborolan-2-yl)-1,2-dihydroquinoline-3-carboxamide (300 mg, 45%) as a brown solid, which was used without further purification.

### Step 3

General procedure 2 was followed using 4-amino-N-(2-methoxyethyl)-2-oxo-8-(4,4,5,5-tetramethyl-1,3,2-dioxaborolan-2-yl)-1,2-dihydroquinoline-3-carboxamide (105 mg, 0.27 mmol) and 3-bromo-4-(difluoromethoxy) pyridine (40 mg, 0.18 mmol). The material was purified by flash column chromatography on an ISCO system (10 g silica, elution with a 0-8% MeOH/CH₂Cl₂ gradient) to give the title compound (14 mg, 19%) as a grey solid. ¹H NMR (500 MHZ, DMSO-d₆) δ 10.83 (s, 1H), 10.57 (s, 1H), 9.88 (s, 1H), 8.14 (d, J=8.1 Hz, 2H), 8.08 (dq, J=10.0, 2.2 Hz, 2H), 7.70-7.43 (m, 1H), 7.40 (dt, J=7.4, 1.5 Hz, 1H), 7.30-7.14 (m, 1H), 6.39 (dd, J=7.6, 1.9 Hz, 1H), 3.47-3.37 (m, 4H), 3.24 (s, 3H).

### Example 72: 4-Amino-8-(1,3-dimethyl-1H-pyrazol-4-yl)-N-(oxetan-3-ylmethyl)-2-oxo-1,2-dihydroquinoline-3-carboxamide

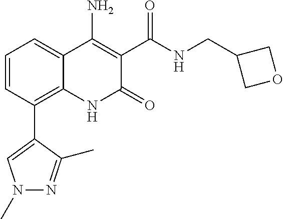 [SMILES: Cc1nn(C)cc1-c1cccc2c(N)c(C(=O)NCC3COC3)c(=O)[nH]c12]

### Step 1: Synthesis of Intermediate 6: Ethyl 4-amino-8-(1,3-dimethylpyrazol-4-yl)-2-oxo-1H-quinoline-3-carboxylate

General procedure 3 was followed using intermediate 1 (500 mg, 1.61 mmol) and 1,3-dimethyl-1H-pyrazole-4-boronic acid pinacol ester (379 mg, 1.71 mmol). The reaction mixture was filtered to remove black particulates and concentrated under reduced pressure. The material was purified by flash column chromatography on an ISCO system (10 g silica, elution with a 0-10% MeOH/CH₂Cl₂ gradient) to yield a solid, which was then triturated with Et₂O to give intermediate 6 (405 mg, 73%) as an off-white solid. ¹H NMR (500 MHZ, Chloroform-d) δ 8.06 (s, 1H), 7.57 (d, J=8.2 Hz, 1H), 7.41-7.35 (m, 2H), 7.20 (dd, J=8.3, 7.3 Hz, 1H), 4.41 (q, J=7.1 Hz, 2H), 3.93 (s, 3H), 2.14 (s, 3H), 1.42 (t, J=7.1 Hz, 3H). m/z 327.1 [M+H]⁺.

### Step 2

General procedure 5 was followed using intermediate 6 (50 mg, 0.16 mmol) and 3-aminomethyl-oxetane (67 mg, 0.77 mmol). The material was purified by flash column chromatography on an ISCO system (10 g silica, elution with a 0-10% MeOH/CH₂Cl₂ gradient), then trituration with Et₂O followed by flash column chromatography on an ISCO system (10 g silica, elution with a 0-10% MeOH/EtOAc gradient) to give the title compound (20 mg, 33%) as a colourless foam. ¹H NMR (500 MHZ, Chloroform-d) o 11.15 (s, 1H), 10.50 (s, 1H), 8.13 (s, 1H), 7.60 (d, J=8.3 Hz, 1H), 7.47-7.35 (m, 2H), 7.33-7.19 (m, 1H), 5.95 (s, 1H), 4.81 (td, J=6.8, 6.1, 1.3 Hz, 2H), 4.51 (td, J=6.1, 1.3 Hz, 2H), 3.93 (s, 3H), 3.76-3.64 (m, 2H), 3.30 (p, J=6.9 Hz, 1H), 2.14 (s, 3H). m/z 368.1 [M+H]⁺.

### Example 73: 4-Amino-8-(1,3-dimethylpyrazol-4-yl)-2-oxo-N-(3,3,3-trifluoropropyl)-1H-quinoline-3-carboxamide

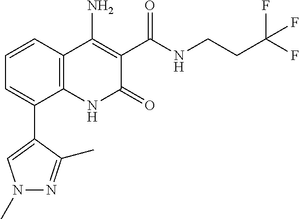 [SMILES: Cc1nn(C)cc1-c1cccc2c(N)c(C(=O)NCCC(F)(F)F)c(=O)[nH]c12]

General procedure 6 was followed using intermediate 6 (60 mg, 0.18 mmol) and 3,3,3-trifluoropropylamine (42 mg, 0.37 mmol). The material was purified by flash column chromatography on an ISCO system (10 g silica, elution with a 0-10% MeOH/CH₂Cl₂ gradient) to give the title compound (16 mg, 21%) as a colourless solid. ¹H NMR (500 MHz, Chloroform-d) δ 11.10 (s, 1H), 10.53 (t, J=5.6 Hz, 1H), 8.15 (s, 1H), 7.60 (d, J=8.3 Hz, 1H), 7.49-7.36 (m, 2H), 7.27 (dd, J=9.8, 2.1 Hz, 1H), 5.94 (s, 1H), 3.94 (s, 3H), 3.65 (q, J=7.6, 6.7 Hz, 2H), 2.45 (dddd, J=13.0, 10.7, 5.8, 3.2 Hz, 2H), 2.14 (s, 3H). m/z 394.2 [M+H]⁺.

### Example 74: 4-Amino-N-(2-cyanoethyl)-8-(1,3-dimethylpyrazol-4-yl)-2-oxo-1H-quinoline-3-carboxamide

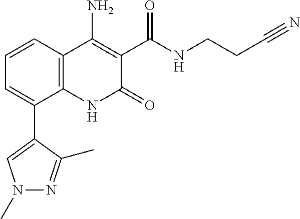 [SMILES: Cc1nn(C)cc1-c1cccc2c(N)c(C(=O)NCCC#N)c(=O)[nH]c12]

General procedure 6 was followed using intermediate 6 (60 mg, 0.18 mmol) and 3-aminopropionitrile (26 mg, 0.37 mmol). The material was purified by flash column chromatography on an ISCO system (10 g silica, elution with a 0-10% MeOH/CH₂Cl₂ gradient) to yield a solid, which was then triturated with Et₂O to give the title compound (32 mg, 47%) as a colourless solid. ¹H NMR (500 MHZ, Chloroform-d) δ 11.01 (s, 1H), 10.66 (t, J=6.2 Hz, 1H), 8.16 (s, 1H), 7.60 (d, J=8.3 Hz, 1H), 7.49-7.35 (m, 2H), 7.35-7.14 (m, 1H), 5.96 (s, 1H), 3.94 (s, 3H), 3.68 (q, J=6.6 Hz, 2H), 2.71 (td, J=6.9, 1.6 Hz, 2H), 2.14 (s, 3H). m/z 351.2 [M+H]⁺.

### Example 75: 4-Amino-N-[3-(dimethylamino) propyl]-8-(1,3-dimethylpyrazol-4-yl)-2-oxo-1H-quinoline-3-carboxamide

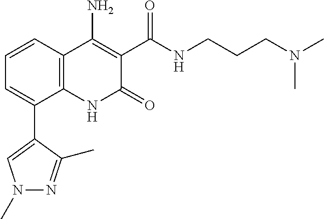

General procedure 5 was followed using intermediate 6 (50 mg, 0.15 mmol) and 3-(dimethylamino)-1-propylamine (96 μL, 0.77 mmol). The reaction mixture was concentrated under reduced pressure and the residue was suspended in DMSO (3 mL) and loaded onto an SCX-2 column. The cartridge was flushed with MeOH, and then the compound was released from the column with 2 M NH₃ in methanol. The filtrate was concentrated under reduced pressure and the material was purified by reverse phase chromatography on an ISCO ACCQPrep system (20 mm×150 mm C18 column, elution with a 10-100% MeOH/water gradient) to give the title compound (7 mg, 11%) as a yellow solid. ¹H NMR (500 MHZ, Chloroform-d) δ 11.21 (s, 1H), 10.35 (t, J=5.0 Hz, 1H), 8.13 (s, 1H), 7.60 (d, J=8.3 Hz, 1H), 7.49-7.34 (m, 2H), 7.32-7.10 (m, 1H), 5.91 (s, 1H), 3.93 (s, 3H), 3.45 (q, J=6.6 Hz, 2H), 2.49 (t, J=7.6 Hz, 2H), 2.32 (s, 6H), 2.14 (s, 3H), 1.83 (app. h, J=5.7, 4.3 Hz, 2H). m/z 383.2 [M+H]⁺.

### Example 76: 4-Amino-8-(2-methoxy-3-pyridyl)-1-methyl-2-oxo-N-propyl-quinoline-3-carboxamide

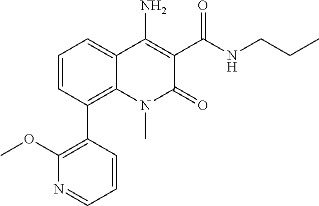 [SMILES: CCCNC(=O)c1c(N)c2cccc(-c3cccnc3OC)c2n(C)c1=O]

### Step 1

To 3-bromo-2-fluorobenzonitrile (4.00 g, 20.0 mmol) in EtOH (20 mL), was added 33% ethanolic methylamine (91.8 mL, 243 mmol) and the reaction mixture was stirred at 85° C. for 1 h and a further 16 h at rt. After this time, the reaction mixture was concentrated under reduced pressure and the residue partitioned between EtOAc (10 mL) and sat. aq. NaHCO₃ (5 mL). The layers were separated and the organic layer was washed with brine, passed through a phase separator cartridge and concentrated under reduced pressure to give 3-bromo-2-(methylamino) benzonitrile (3.55 g, 80%) as a yellow solid. ¹H NMR (500 MHz, Chloroform-d) δ 7.58 (dt, J=7.8, 1.5 Hz, 1H), 7.40 (dd, J=7.8, 1.6 Hz, 1H), 6.56 (td, J=7.8, 1.4 Hz, 1H), 4.89 (s, 1H), 3.33 (dd, J=5.5, 1.4 Hz, 3H). m/z 212.9 [M+H]⁺.

### Step 2: Synthesis of Intermediate 7: Ethyl 4-amino-8-bromo-1-methyl-2-oxo-quinoline-3-carboxylate

To 3-bromo-2-(methylamino) benzonitrile (2.00 g, 9.48 mmol) and diethyl malonate (2.01 mL, 13.3 mmol) in toluene (40 mL) at 0° C., was slowly added tin (IV) chloride (1.57 mL, 13.3 mmol) and the reaction mixture was stirred at rt for 20 min, followed by reflux for 16 h. After this time, the reaction mixture was cooled to 0° C., sat. aq. Na₂CO₃ was added (40 mL) and stirred for 20 min. CH₂Cl₂ (60 mL) was added and the layers separated. The aqueous layer was extracted with CH₂Cl₂ (60 mL) and the combined organic extracts were dried over Na₂SO₄ and concentrated under reduced pressure. The material was purified by flash column chromatography on an ISCO system (40 g silica, elution with a 0-70% EtOAc/pet. ether gradient) to give intermediate 7 (1.44 g, 44%) as a colourless solid. ¹H NMR (500 MHZ, Chloroform-d) δ 7.84 (d, J=7.7 Hz, 1H), 7.62 (dd, J=8.1, 1.3 Hz, 1H), 7.51 (s, 2H), 7.07 (t, J=7.9 Hz, 1H), 4.39 (q, J=7.1 Hz, 2H), 3.77 (s, 3H), 1.41 (t, J=7.1 Hz, 3H). m/z 326.9 [M+H]⁺.

### Step 3

General procedure 3 was followed using intermediate 7 (150 mg, 1.85 mmol) and (2-methoxypyridin-3-yl) boronic acid (106 mg, 0.69 mmol). The reaction mixture was diluted with EtOAc (20 mL), washed with water (10 mL), passed through a phase separator cartridge and concentrated under reduced pressure. The material was purified by flash column chromatography on an ISCO system (10 g silica, elution with a 0-100% EtOAc/pet. ether gradient) to give ethyl 4-amino-8-(2-methoxy-3-pyridyl)-1-methyl-2-oxo-quinoline-3-carboxylate (170 mg, 99%) as a colourless solid. ¹H NMR (500 MHZ, DMSO-d₆) δ 8.23 (dt, J=4.9, 1.9 Hz, 1H), 8.21-8.09 (m, 3H), 7.66 (dt, J=7.3, 1.9 Hz, 1H), 7.44 (dt, J=7.5, 1.6 Hz, 1H), 7.27 (td, J=7.7, 1.9 Hz, 1H), 7.10 (ddd, J=7.0, 5.0, 2.0 Hz, 1H), 4.23 (app. qt, J=7.1, 1.7 Hz, 2H), 3.81 (s, 3H), 2.75 (s, 3H), 1.26 (t, J=7.1 Hz, 3H). m/z 354.0 [M+H]⁺.

### Step 4

General procedure 5 was followed using ethyl 4-amino-8-(2-methoxy-3-pyridyl)-1-methyl-2-oxo-quinoline-3-carboxylate (30 mg, 0.085 mmol) and propylamine (35 μL, 0.42 mmol). The material was purified by flash column chromatography on an ISCO system (10 g silica, elution with a 0-10% MeOH/CH₂Cl₂ gradient) to give the title compound (15 mg, 46%) as a colourless solid. ¹H NMR (500 MHZ, DMSO-d₆) δ 10.92 (s, 1H), 10.46 (t, J=5.5 Hz, 1H), 8.44-7.87 (m, 3H), 7.71 (dd, J=7.3, 1.9 Hz, 1H), 7.48 (dd, J=7.4, 1.3 Hz, 1H), 7.33 (t, J=7.7 Hz, 1H), 7.12 (dd, J=7.3, 5.0 Hz, 1H), 3.79 (s, 3H), 3.28-3.17 (m, 2H), 2.83 (s, 3H), 1.52 (app. sextet, J=7.2 Hz, 2H), 0.91 (t, J=7.4 Hz, 3H). m/z 367.0 [M+H]⁺.

### Example 77: 4-Amino-8-(4-methoxy-3-pyridyl)-1-methyl-2-oxo-N-propyl-quinoline-3-carboxamide

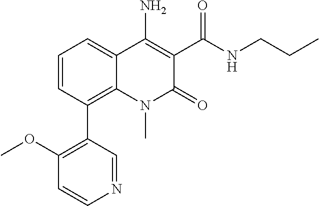 [SMILES: CCCNC(=O)c1c(N)c2cccc(-c3cnccc3OC)c2n(C)c1=O]

### Step 1: Synthesis of Intermediate 8: Ethyl 4-amino-8-(4-methoxy-3-pyridyl)-1-methyl-2-oxo-quinoline-3-carboxylate

General procedure 3 was followed using intermediate 7 (600 mg, 1.85 mmol) and (4-methoxypyridin-3-yl) boronic acid hydrate (710 mg, 4.15 mmol). The reaction mixture was diluted with EtOAc (20 mL), washed with water and brine, dried over MgSO₄, filtered and concentrated under reduced pressure. The material was purified by flash column chromatography on an ISCO system (24 g silica, elution with a 0-20% MeOH/EtOAc gradient) to give intermediate 8 (490 mg, 75%) as a yellow solid. ¹H NMR (500 MHZ, DMSO-d₆) δ 8.49 (d, J=5.8 Hz, 1H), 8.31 (s, 1H), 8.22-8.11 (m, 3H), 7.47-7.38 (m, 1H), 7.27 (t, J=7.7 Hz, 1H), 7.15 (d, J=5.8 Hz, 1H), 4.26-4.12 (m, 2H), 3.77 (s, 3H), 2.74 (s, 3H), 1.24 (t, J=7.1 Hz, 3H). m/z 354.0 [M+H]⁺.

### Step 2

General procedure 5 was followed using intermediate 8 (30 mg, 0.085 mmol) and propylamine (35 μL, 0.42 mmol). The material was purified by flash column chromatography on an ISCO system (10 g silica, elution with a 0-10% MeOH/CH₂Cl₂ gradient) to give the title compound (25 mg, 76%) as a colourless solid. ¹H NMR (500 MHz, DMSO-d₆) δ 10.91 (s, 1H), 10.46 (t, J=5.6 Hz, 1H), 8.52 (d, J=5.7 Hz, 1H), 8.35 (s, 1H), 8.29-7.92 (m, 2H), 7.47 (dd, J=7.4, 1.3 Hz, 1H), 7.34 (t, J=7.7 Hz, 1H), 7.16 (d, J=5.8 Hz, 1H), 3.78 (s, 3H), 3.27-3.16 (m, 2H), 2.84 (s, 3H), 1.52 (app. sextet, J=7.2 Hz, 2H), 0.91 (t, J=7.4 Hz, 3H). m/z 367.1 [M+H]⁺.

### Example 78: 4-Amino-N-(cyclopropylmethyl)-8-(4-methoxy-3-pyridyl)-1-methyl-2-oxo-quinoline-3-carboxamide

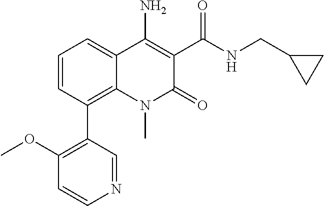 [SMILES: COc1ccncc1-c1cccc2c(N)c(C(=O)NCC3CC3)c(=O)n(C)c12]

General procedure 5 was followed using intermediate 8 (60 mg, 0.17 mmol) and cyclopropylmethylamine (70 μL, 0.85 mmol). The material was purified by flash column chromatography on an ISCO system (10 g silica, elution with a 0-5% MeOH/CH₂Cl₂ gradient) to give the title compound (41 mg, 60%) as a colourless solid. ¹H NMR (500 MHz, Chloroform-d) δ 11.17 (s, 1H), 10.54 (t, J=4.1 Hz, 1H), 8.56 (s, 1H), 8.43 (s, 1H), 7.72 (d, J=8.2 Hz, 1H), 7.45 (d, J=7.4 Hz, 1H), 7.30 (t, J=7.7 Hz, 1H), 6.89 (d, J=5.7 Hz, 1H), 5.86 (s, 1H), 3.81 (s, 3H), 3.39-3.20 (m, 2H), 3.03 (s, 3H), 1.19-0.99 (m, 1H), 0.63-0.44 (m, 2H), 0.41-0.08 (m, 2H). m/z 379.2 [M+H]⁺.

### Example 79: 4-Amino-N-isobutyl-8-(4-methoxy-3-pyridyl)-1-methyl-2-oxo-quinoline-3-carboxamide

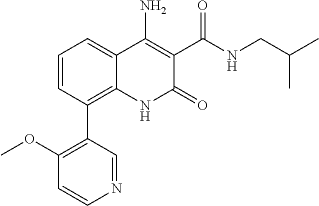 [SMILES: COc1ccncc1-c1cccc2c(N)c(C(=O)NCC(C)C)c(=O)[nH]c12]

General procedure 5 was followed using intermediate 8 (62 mg, 0.18 mmol) and isobutylamine (88 μL, 0.88 mmol). The material was purified by flash column chromatography on an ISCO system (10 g silica, elution with a 0-5% MeOH/CH₂Cl₂ gradient) to give the title compound (48 mg, 68%) as a colourless solid. ¹H NMR (500 MHz, Chloroform-d) δ 11.13 (s, 1H), 10.55 (t, J=5.5 Hz, 1H), 8.57 (s, 1H), 8.44 (s, 1H), 7.73 (d, J=8.2 Hz, 1H), 7.45 (d, J=7.4 Hz, 1H), 7.31 (dd, J=8.2, 7.3 Hz, 1H), 6.90 (d, J=5.6 Hz, 1H), 5.86 (s, 1H), 3.82 (s, 3H), 3.33-3.19 (m, 2H), 3.04 (s, 3H), 2.04-1.78 (app. septet, J=6.8 Hz, 1H), 1.01 (d, J=6.7, 6H). m/z 381.2 [M+H]⁺.

### Example 80: 4-Amino-8-(1,3-dimethylpyrazol-4-yl)-1-methyl-2-oxo-N-propyl-quinoline-3-carboxamide

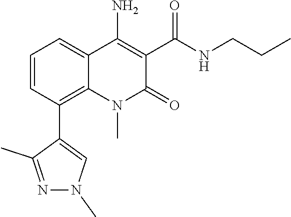 [SMILES: CCCNC(=O)c1c(N)c2cccc(-c3cn(C)nc3C)c2n(C)c1=O]

### Step 1: Synthesis of Intermediate 9: Ethyl 4-amino-8-(1,3-dimethylpyrazol-4-yl)-1-methyl-2-oxo-quinoline-3-carboxylate

General procedure 3 was followed using intermediate 7 (150 mg, 0.46 mmol) and 1,3-dimethyl-1H-pyrazole-4-boronic acid pinacol ester (154 mg, 0.69 mmol). The material was purified by flash column chromatography on an ISCO system (24 g silica, elution with a 0-10% MeOH/EtOAc gradient) to give intermediate 9 (140 mg, 85%) as a yellow solid. ¹H NMR (500 MHZ, DMSO-d₆) δ 8.16 (s, 2H), 8.10 (d, J=8.1 Hz, 1H), 7.69 (s, 1H), 7.42 (dd, J=7.4, 1.3 Hz, 1H), 7.25 (t, J=7.7 Hz, 1H), 4.22 (q, J=7.1 Hz, 2H), 3.81 (s, 2H), 2.85 (s, 3H), 1.94 (s, 3H), 1.26 (t, J=7.1 Hz, 3H). m/z 341.0 [M+H]⁺.

### Step 2

General procedure 5 was followed using intermediate 9 (29 mg, 0.085 mmol) and propylamine (35 μL, 0.42 mmol). The material was purified by flash column chromatography on an ISCO system (10 g silica, elution with a 0-10% MeOH/CH₂Cl₂ gradient) to give the title compound (25 mg, 79%) as a colourless solid. ¹H NMR (500 MHz, DMSO-d₆) δ 10.89 (s, 1H), 10.47 (t, J=5.5 Hz, 1H), 8.39-7.86 (m, 2H), 7.70 (s, 1H), 7.45 (dd, J=7.4, 1.3 Hz, 1H), 7.30 (t, J=7.7 Hz, 1H), 3.81 (s, 3H), 3.23 (q, J=6.7 Hz, 2H), 2.93 (s, 3H), 1.92 (s, 3H), 1.52 (app. sextet, J=7.2 Hz, 2H), 0.91 (t, J=7.4 Hz, 3H). m/z 354.0 [M+H]⁺.

### Example 81: 4-Amino-N-(cyclopropylmethyl)-8-(1,3-dimethylpyrazol-4-yl)-1-methyl-2-oxo-quinoline-3-carboxamide

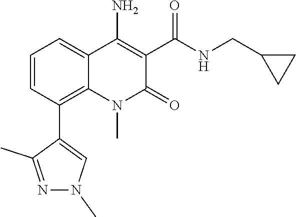 [SMILES: Cc1nn(C)cc1-c1cccc2c(N)c(C(=O)NCC3CC3)c(=O)n(C)c12]

General procedure 6 was followed using intermediate 9 (50 mg, 0.15 mmol) and cyclopropylmethylamine (60 μL, 0.73 mmol). The material was purified by flash column chromatography on an ISCO system (10 g silica, elution with a 0-5% MeOH/CH₂Cl₂ gradient) to give the title compound (41 mg, 73%) as a colourless solid. ¹H NMR (500 MHz, Chloroform-d) δ 11.15 (s, 1H), 10.54 (s, 1H), 7.63 (dt, J=8.2, 1.3 Hz, 1H), 7.43 (dt, J=7.4, 1.3 Hz, 1H), 7.29 (s, 1H), 7.30-7.23 (m, 1H), 3.90 (s, 3H), 5.81 (s, 1H), 3.28 (app. t, J=6.1 Hz, 2H), 3.11 (s, 3H), 2.12 (s, 3H), 1.18-1.01 (m, 1H), 0.68-0.46 (m, 2H), 0.29-0.26 (m, 2H). m/z 366.2 [M+H]⁺.

### Example 82: 4-Amino-8-(1,3-dimethylpyrazol-4-yl)-N-isobutyl-1-methyl-2-oxo-quinoline-3-carboxamide

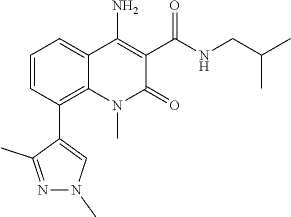 [SMILES: Cc1nn(C)cc1-c1cccc2c(N)c(C(=O)NCC(C)C)c(=O)n(C)c12]

General procedure 5 was followed using intermediate 9 (60 mg, 0.18 mmol) and isobutylamine (88 μL, 0.88 mmol). The material was purified by flash column chromatography on an ISCO system (10 g silica, elution with a 0-5% MeOH/CH₂Cl₂ gradient) to give the title compound (47 mg, 69%) as a colourless solid. ¹H NMR (500 MHz, Chloroform-d) δ 11.15 (s, 1H), 10.55 (s, 1H), 7.64 (dd, J=8.2, 1.5 Hz, 1H), 7.44 (dd, J=7.3, 1.4 Hz, 1H), 7.30-7.26 (m, 2H), 5.81 (s, 1H), 3.91 (s, 3H), 3.26 (dd, J=6.8, 5.7 Hz, 2H), 3.12 (s, 3H), 2.14 (s, 3H), 1.99-1.87 (m, 1H), 1.01 (d, J=6.7 Hz, 6H). m/z 368.2 [M+H]⁺.

### Example 83: 4-Amino-8-(1,3-dimethylpyrazol-4-yl)-1-ethyl-2-oxo-N-propyl-quinoline-3-carboxamide

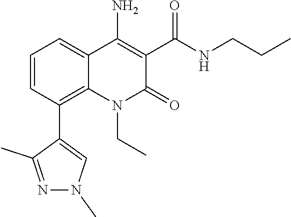 [SMILES: CCCNC(=O)c1c(N)c2cccc(-c3cn(C)nc3C)c2n(CC)c1=O]

### Step 1

To 3-bromo-2-fluorobenzonitrile (500 mg, 2.50 mmol) in EtOH (2 mL) was added 70% aq. ethylamine (1.00 mL, 17.5 mmol) and the reaction mixture was stirred at 85° C. for 2 h, followed by rt for 16 h. After this time, the reaction mixture was concentrated under reduced pressure and the residue partitioned between EtOAc and sat. aq. NaHCO₃. The layers were separated and the organic layer was washed with brine, passed through a phase separator cartridge and concentrated under reduced pressure to give 3-bromo-2-(ethylamino) benzonitrile (560 mg, 90%) as a yellow oil. ¹H NMR (500 MHZ, Chloroform-d) δ 7.59 (d, J=7.7 Hz, 1H), 7.40 (d, J=7.9 Hz, 1H), 7.26 (s, 1H), 6.57 (d, J=7.9 Hz, 1H), 4.67 (s, 1H), 3.72 (m, 2H), 1.33 (t, J=7.2 Hz, 3H). m/z 224.9, 226.9 [M+H]⁺.

### Step 2

To 3-bromo-2-(ethylamino) benzonitrile (560 mg, 2.49 mmol) and diethyl malonate (0.53 mL, 3.48 mmol) in toluene (10 mL) at 0° C., was slowly added tin (IV) chloride (0.41 mL, 3.48 mmol) and the reaction mixture was stirred at rt for 2 h and then at reflux for 3 h. After this time, the reaction mixture was cooled to 0° C., sat aq. K₂CO₃ (16 mL) was added and the resulting suspension stirred for 1 h. After this time, the reaction mixture was diluted with CH₂Cl₂ (200 mL) and water (50 mL), the layers separated and the aqueous phase extracted with CH₂Cl₂ (2×50 mL). The combined organic extracts were washed with brine (50 mL), passed through a phase separator cartridge and concentrated under reduced pressure. The material was purified by flash column chromatography on an ISCO system (24 g silica, elution with a 0-10% CH₂Cl₂/MeOH gradient) to give ethyl 4-amino-8-bromo-1-ethyl-2-oxo-quinoline-3-carboxylate (430 mg, 48%) as a colourless solid. ¹H NMR (500 MHz, DMSO-d₆) δ8.20-8.07 (m, 3H), 7.92 (dd, J=7.8, 1.3 Hz, 1H), 7.15 (t, J=7.9 Hz, 1H), 4.27 (q, J=6.9 Hz, 2H), 4.20 (q, J=7.1 Hz, 2H), 1.25-1.20 (m, 6H). m/z 338.9, 340.9 [M+H]⁺.

### Step 3

General procedure 3 was followed using 4-amino-8-bromo-1-diethyl-2-oxo-quinoline-3-carboxamide (90 mg, 0.27 mmol) and 1,3-dimethyl-1H-pyrazole-4-boronic acid pinacol ester (71 mg, 0.32 mmol). The material was purified by reverse phase chromatography on an ISCO system (12 g RediSep® Rf Reversed-phase C18, elution with a 10-100% MeOH/water gradient) to give ethyl 4-amino-8-(1,3-dimethylpyrazol-4-yl)-1-ethyl-2-oxo-quinoline-3-carboxylate (71 mg, 72%) as a colourless oil. ¹H NMR (500 MHZ, Chloroform-d) δ 7.64 (dd, J=8.1, 1.5 Hz, 1H), 7.34 (dd, J=7.3, 1.7 Hz, 1H), 7.31 (s, 1H), 7.22-7.16 (m, 1H), 4.38 (q, J=7.1 Hz, 2H), 3.99 (q, J=6.9 Hz, 2H), 3.87 (s, 3H), 2.11 (s, 3H), 1.40 (t, J=7.1 Hz, 3H), 0.83-0.69 (m, 3H). m/z 355.2 [M+H]⁺.

### Step 4

General procedure 5 was followed using ethyl 4-amino-8-(1,3-dimethylpyrazol-4-yl)-1-ethyl-2-oxo-quinoline-3-carboxylate (50 mg, 0.14 mmol) and propylamine (58 μL, 0.71 mmol). The material was purified by reverse phase chromatography on an ISCO system (12 g RediSep® Rf Reversed-phase C18, elution with a 10-100% MeOH/water gradient) to give the title compound (33 mg, 60%) as a colourless solid. ¹H NMR (500 MHZ, Chloroform-d) δ 11.16 (s, 1H), 10.52 (t, J=5.6 Hz, 1H), 7.64 (dd, J=8.1, 1.5 Hz, 1H), 7.36 (dd, J=7.4, 1.4 Hz, 1H), 7.33 (s, 1H), 7.23 (t, J=7.7 Hz, 1H), 5.81 (s, 1H), 4.03 (q, J=7.0 Hz, 2H), 3.90 (s, 3H), 3.36 (td, J=7.1, 5.6 Hz, 2H), 2.09 (s, 3H), 1.64 (app. sextet, J=7.3 Hz, 2H), 0.99 (t, J=7.4 Hz, 3H), 0.79 (t, J=7.0 Hz, 3H). m/z 368.2 [M+H]⁺.

### Example 84: 4-Amino-8-(4-methoxypyridin-3-yl)-2-oxo-N-propyl-1,2-dihydro-1,7-naphthyridine-3-carboxamide

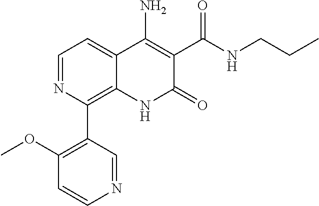 [SMILES: CCCNC(=O)c1c(N)c2ccnc(-c3cnccc3OC)c2[nH]c1=O]

### Step 1

To 3-amino-2-chloroisonicotinic acid (900 mg, 5.22 mmol) in THF (35 mL), was added CDI (1.27 g, 7.82 mmol) and the reaction mixture was stirred at 60° C. for 16 h. After this time, ammonium acetate (1.61 g, 20.9 mmol) was added and the reaction mixture was stirred at 60° C. for a further 1 h. After this time, the reaction mixture was concentrated under reduced pressure and the residue suspended in water (25 mL). The solids were collected via vacuum filtration and washed with water to give 3-amino-2-chloro-pyridine-4-carboxamide (549 mg, 58%) as a yellow solid. The organic filtrate was treated with 2 M NaOH (5 mL) and extracted with EtOAc (25 mL). The organic extract was dried over MgSO₄, filtered and concentrated under reduced pressure to give 3-amino-2-chloro-pyridine-4-carboxamide (240 mg, 20%) as a yellow solid. Both batches were combined and used in the next step. ¹H NMR (500 MHZ, DMSO-d₆) δ 8.18 (s, 1H), 7.68 (s, 1H), 7.61 (d, J=5.0 Hz, 1H), 7.51 (d, J=5.1 Hz, 1H), 6.76 (s, 2H). m/z 172.1 [M+H]⁺.

To 3-amino-2-chloro-pyridine-4-carboxamide (679 mg, 3.96 mmol) in THF (8 mL), at 0° C. was added Et₃N (2.21 mL, 15.8 mmol) and POCl₃ (0.41 mL, 4.35 mmol), and the reaction mixture was stirred at rt for 2 h. After this time, sat. aq. NaHCO₃ (20 mL) was added and the reaction mixture stirred for 15 min. The reaction mixture was extracted with CH₂Cl₂ (3×20 mL) and the combined organic extracts dried over MgSO₄, filtered and concentrated under reduced pressure. The material was purified by flash column chromatography on an ISCO system (24 g silica, elution with a 0-50% EtOAc/pet. ether gradient) to give 3-amino-2-chloro-pyridine-4-carbonitrile (528 mg, 82%) as a yellow solid. ¹H NMR (500 MHZ, Chloroform-d) δ 7.82 (d, J=5.0 Hz, 1H), 7.23 (d, J=5.0 Hz, 1H), 4.91 (s, 2H). m/z 154.0 [M+H]⁺.

### Step 3

To 3-amino-2-chloro-pyridine-4-carbonitrile (500 mg, 3.26 mmol) in toluene (15 mL), was added ethyl malonyl chloride (0.63 mL, 4.88 mmol) and the reaction mixture stirred under reflux for 2 h. After this time, the reaction mixture was diluted with EtOAc (25 mL) and washed with sat. aq. NaHCO₃ (25 mL). The aqueous layer was further extracted with EtOAc (25 mL) and the combined organic extracts were dried over MgSO₄, filtered and concentrated under reduced pressure. The crude material was triturated with Et₂O to give ethyl 3-[(2-chloro-4-cyano-3-pyridyl) amino]-3-oxo-propanoate (614 mg, 67%) as an off-white solid. ¹H NMR (500 MHZ, Chloroform-d) δ 9.92 (s, 1H), 8.41 (dd, J=4.9, 0.3 Hz, 1H), 7.54 (dd, J=4.9, 0.4 Hz, 1H), 4.32 (q, J=7.2 Hz, 2H), 3.63 (s, 2H), 1.36 (t, J=7.2 Hz, 3H). m/z 268.1 [M+H]⁺.

### Step 4

General procedure 1 was followed with ethyl 3-[(2-chloro-4-cyano-3-pyridyl) amino]-3-oxo-propanoate (200 mg, 0.75 mmol) and (4-methoxypyridin-3-yl) boronic acid hydrate (255 mg, 1.49 mmol) to give ethyl 4-amino-8-(4-methoxy-3-pyridyl)-2-oxo-1H-1,7-naphthyridine-3-carboxylate (99 mg, 37%) as a yellow solid. ¹H NMR (500 MHZ, Chloroform-d) δ 8.67 (d, J=5.8 Hz, 1H), 8.57-8.49 (m, 2H), 7.81 (s, 1H), 7.45 (d, J=5.5 Hz, 1H), 7.00 (d, J=5.9 Hz, 1H), 4.43 (q, J=7.1 Hz, 2H), 3.88 (s, 3H), 1.42 (t, J=7.1 Hz, 3H). m/z 341.2 [M+H]⁺.

### Step 5

General procedure 6 was followed using ethyl 4-amino-8-(4-methoxy-3-pyridyl)-2-oxo-1H-1,7-naphthyridine-3-carboxylate (99 mg, 0.29 mmol) and propylamine (0.12 mL, 1.45 mmol). The material was purified by flash column chromatography on an ISCO system (10 g silica, elution with a 0-10% MeOH/CH₂Cl₂ gradient) to give the title compound (66 mg, 61%) as a yellow solid. ¹H NMR (500 MHZ, Chloroform-d) δ 11.31 (s, 1H), 10.15 (t, J=5.0 Hz, 1H), 8.68 (d, J=5.8 Hz, 1H), 8.59-8.50 (m, 2H), 7.90 (s, 1H), 7.49 (d, J=5.5 Hz, 1H), 7.01 (d, J=5.9 Hz, 1H), 5.96 (s, 1H), 3.89 (s, 3H), 3.37 (td, J=7.0, 5.7 Hz, 2H), 1.69-1.61 (m, 2H), 0.99 (t, J=7.4 Hz, 3H). m/z 354.2 [M+H]⁺.

### Example 85: 4-Amino-8-[2-fluoro-5-(2-pyridylmethoxy)phenyl]-2-oxo-N-propyl-1H-quinoline-3-carboxamide

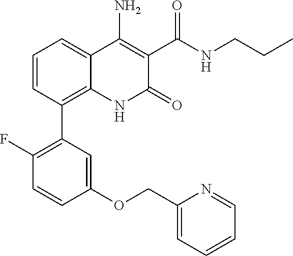 [SMILES: CCCNC(=O)c1c(N)c2cccc(-c3cc(OCc4ccccn4)ccc3F)c2[nH]c1=O]

### Step 1

General procedure 7 was followed using 3-bromo-4-fluorophenol (300 mg, 1.57 mmol) and 2-(chloromethyl)pyridine hydrochloride (309 mg, 1.88 mmol) in MeCN to give 2-[(3-bromo-4-fluoro-phenoxy)methyl]pyridine (412 mg, 84%) as an orange solid. ¹H NMR (400 MHZ, Chloroform-d) δ 8.62 (d, J=4.9 Hz, 1H), 7.77-7.69 (m, 1H), 7.49 (d, J=7.8 Hz, 1H), 7.34-7.14 (m, 2H), 7.12-6.96 (m, 1H), 6.94-6.86 (m, 1H), 5.17 (d, J=5.3 Hz, 2H). m/z 284.0 [M+H]⁺.

### Step 2

General procedure 1 was followed using intermediate 4 (400 mg, 1.08 mmol) and 2-[(3-bromo-4-fluoro-phenoxy)methyl]pyridine (338 mg, 1.08 mmol). The material was purified by flash column chromatography on an ISCO system (10 g silica, elution with a 0-10% MeOH/CH₂Cl₂ gradient) to yield a solid, which was then triturated with EtOAc to give the title compound (269 mg, 53%) as a colourless solid. ¹H NMR (400 MHZ, DMSO-d₆) δ 10.95 (s, 1H), 10.47 (s, 1H), 9.45 (s, 1H), 8.58 (d, J=4.8 Hz, 1H), 8.28-8.17 (m, 2H), 7.90-7.82 (m, 1H), 7.60-7.48 (m, 2H), 7.43-7.23 (m, 3H), 7.21-7.11 (m, 1H), 7.09 (d, J=5.8 Hz, 1H), 5.20 (d, J=3.9 Hz, 2H), 3.30-3.19 (m, 2H), 1.58-1.44 (m, 2H), 0.90 (t, J=7.4 Hz, 3H). m/z 447.2 [M+H]⁺.

### Example 86: 4-Amino-8-[2-methyl-5-(2-pyridylmethoxy)phenyl]-2-oxo-N-propyl-1H-quinoline-3-carboxamide

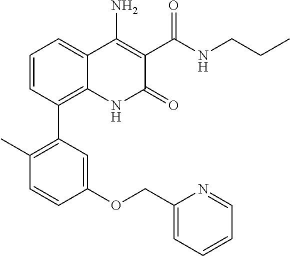 [SMILES: CCCNC(=O)c1c(N)c2cccc(-c3cc(OCc4ccccn4)ccc3C)c2[nH]c1=O]

### Step 1

General procedure 7 was followed using 3-bromo-4methylphenol (100 mg, 0.53 mmol) and 2-(chloromethyl)pyridine hydrochloride (105 mg, 0.64 mmol) in acetone to give 2-[(3-bromo-4-methyl-phenoxy)methyl]pyridine (140 mg, 63%) as an orange oil. m/z 280.0 [M+H]⁺.

### Step 2

General procedure 1 was followed using intermediate 4 (80 mg, 0.22 mmol) and 2-[(3-bromo-4-methyl-phenoxy)methyl]pyridine (60 mg, 0.22 mmol). The material was purified by flash column chromatography on an ISCO system (10 g silica, elution with a 0-10% MeOH/CH₂Cl₂ gradient) to yield a solid, which was then triturated with EtOAc to give the title compound (29 mg, 29%) as a yellow solid. ¹H NMR (400 MHZ, Chloroform-d) δ 10.97 (s, 1H), 10.38 (t, J=5.5 Hz, 1H), 8.56 (d, J=4.8 Hz, 1H), 8.40 (s, 1H), 8.27 (s, 1H), 8.19 (d, J=8.2 Hz, 1H), 7.89-7.79 (m, 1H), 7.54 (d, J=7.7 Hz, 1H), 7.42 (d, J=7.2 Hz, 1H), 7.38-7.26 (m, 3H), 7.12-7.02 (m, 1H), 6.90 (d, J=2.9 Hz, 1H), 5.17 (s, 2H), 3.26-3.15 (m, 2H), 1.92 (s, 3H), 1.62-1.39 (m, 2H), 0.89 (t, J=7.1 Hz, 3H). m/z 443.3 [M+H]⁺.

### Example 87: 4-Amino-2-oxo-N-propyl-8-[3-(2-pyridylmethoxy)phenyl]-1H-quinoline-3-carboxamide

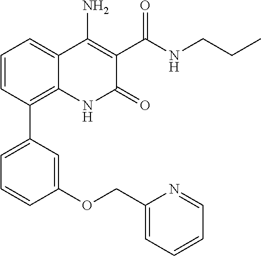 [SMILES: CCCNC(=O)c1c(N)c2cccc(-c3cccc(OCc4ccccn4)c3)c2[nH]c1=O]

### Step 1

General procedure 7 was followed using 3-bromophenol (150 mg, 0.87 mmol) and 2-(chloromethyl)pyridine hydrochloride (171 mg, 1.04 mmol) in MeCN. The material was purified by reverse phase chromatography on an ISCO system (12 g RediSep® Rf Reversed-phase C18, elution with a 10-100% MeOH/water gradient) to give 2-[(3-bromophenoxy)methyl]pyridine (193 mg, 74%) as a yellow oil. ¹H NMR (400 MHZ, Chloroform-d) δ 8.63 (d, J=4.9 Hz, 1H), 7.75 (td, J=7.7, 1.8 Hz, 1H), 7.51 (d, J=7.8 Hz, 1H), 7.27-7.23 (m, 1H), 7.22-7.07 (m, 3H), 7.02-6.86 (m, 1H), 5.21 (s, 2H). m/z 266.0 [M+H]⁺

### Step 2

General procedure 1 was followed using intermediate 4 (100 mg, 0.27 mmol) and 2-[(3-bromophenoxy)methyl]pyridine (71 mg, 0.27 mmol). The material was purified by flash column chromatography on an ISCO system (10 g silica, elution with a 0-10% MeOH/CH₂Cl₂ gradient), followed by reverse phase chromatography on an ISCO system (12 g RediSep® Rf Reversed-phase C18, elution with a 10-100% MeOH/water gradient) to give the title compound (55 mg, 45%) as a colourless solid. ¹H NMR (400 MHZ, DMSO-d₆) δ 10.96 (s, 1H), 10.39 (t, J=5.5 Hz, 1H), 8.75 (s, 1H), 8.63-8.55 (m, 1H), 8.24 (s, 1H), 8.18 (dd, J=8.4, 1.3 Hz, 1H), 7.86 (td, J=7.7, 1.7 Hz, 1H), 7.60-7.55 (m, 1H), 7.55-7.46 (m, 2H), 7.39-7.30 (m, 2H), 7.18-7.12 (m, 2H), 7.08-7.04 (m, 1H), 5.25 (s, 2H), 3.27-3.17 (m, 2H), 1.59-1.42 (m, 2H), 0.90 (t, J=7.4 Hz, 3H). m/z 429.3 [M+H]⁺.

### Example 88: 4-Amino-8-[2-fluoro-5-[(6-methyl-2-pyridyl)methoxy]phenyl]-2-oxo-N-propyl-1H-quinoline-3-carboxamide

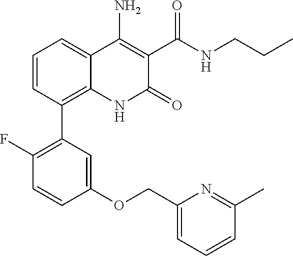 [SMILES: CCCNC(=O)c1c(N)c2cccc(-c3cc(OCc4cccc(C)n4)ccc3F)c2[nH]c1=O]

### Step 1

General procedure 7 was followed using 3-bromo-4-fluorophenol (100 mg, 0.52 mmol) and 2-(bromomethyl)-6-methylpyridine (107 mg, 0.58 mmol) in MeCN. The material was purified by flash column chromatography on an ISCO system (10 g silica, elution with a 0-80% EtOAc/pet. ether gradient) to give 2-[(3-bromo-4-fluoro-phenoxy)methyl]-6-methyl-pyridine (75 mg, 46%) as a pale yellow oil. ¹H NMR (500 MHZ, Chloroform-d) δ 7.61 (t, 1H, J=7.7 Hz), 7.27 (d, 1H, J=7.9 Hz), 7.18 (dd, 1H, J=5.5, 3.0 Hz), 7.10 (d, 1H, J=7.7 Hz), 7.06-6.98 (m, 1H), 6.87 (dt, 1H, J=9.1, 3.4 Hz), 5.11 (s, 2H), 2.57 (s, 3H).

### Step 2

General procedure 2 was followed using intermediate 4 (107 mg, 0.20 mmol) and 2-[(3-bromo-4-fluoro-phenoxy)methyl]-6-methyl-pyridine (75 mg, 0.25 mmol). The material was purified by flash column chromatography on an ISCO system (10 g silica, elution with a 0-100% EtOAc/pet. ether gradient) to yield a solid, which was then triturated with EtOAc to give the title compound (28 mg, 28%) as a cream solid. ¹H NMR (500 MHZ, Chloroform-d) δ 11.29 (s, 1H), 10.27 (t, 1H, J=5.6 Hz), 7.93 (s, 1H), 7.70-7.59 (m, 2H), 7.47 (dd, 1H, J=7.4, 1.2 Hz), 7.34-7.25 (m, 2H), 7.16 (t, 1H, J=8.9 Hz), 7.10 (d, 1H, J=7.7 Hz), 7.05 (dt, 1H, J=9.1, 3.6 Hz), 6.94 (dd, 1H, J=5.8, 3.1 Hz), 5.90 (s, 1H), 5.16 (s, 2H), 3.43-3.30 (m, 2H), 2.56 (s, 3H), 1.64 (sext, 2H, J=7.3 Hz), 0.99 (t, 3H, J=7.4 Hz). m/z 461.2 [M+H]⁺.

### Example 89: 4-Amino-8-[2-fluoro-5-[(6-methoxy-2-pyridyl)methoxy]phenyl]-2-oxo-N-propyl-1H-quinoline-3-carboxamide

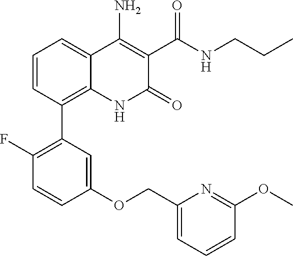 [SMILES: CCCNC(=O)c1c(N)c2cccc(-c3cc(OCc4cccc(OC)n4)ccc3F)c2[nH]c1=O]

### Step 1

General procedure 7 was followed using (6-methoxypyridin-2-yl) methanol (200 mg, 1.44 mmol) and 3-bromo-4-fluorophenol (245 mg, 1.28 mmol) in MeCN. The material was purified by flash column chromatography on an ISCO system (10 g silica, elution with a 0-5% EtOAc/pet. ether gradient) to give 2-[(3-bromo-4-fluoro-phenoxy)methyl]-6-methoxy-pyridine (381 mg, 81%) as a pink solid. ¹H NMR (500 MHZ, Chloroform-d) δ 7.61-7.55 (m, 1H), 7.25-7.20 (m, 1H), 7.07-6.99 (m, 2H), 6.92-6.85 (m, 1H), 6.67 (d, J=8.3 Hz, 1H), 5.04 (s, 2H), 3.94 (s, 3H).

### Step 2

General procedure 2 was followed using intermediate 4 (200 mg, 0.38 mmol) and 2-[(3-bromo-4-fluoro-phenoxy)methyl]-6-methoxy-pyridine (147 mg, 0.47 mmol). The material was purified by flash column chromatography on an ISCO system (10 g silica, elution with a 0-50% EtOAc/pet. ether gradient) to yield a solid, which was then triturated with EtOAc to give the title compound (125 mg, 66%) as a colourless solid. ¹H NMR (500 MHz, Chloroform-d) δ 11.32 (s, 1H), 10.30 (t, J=5.4 Hz, 1H), 7.97 (s, 1H), 7.69 (dt, J=8.2, 1.0 Hz, 1H), 7.63 (dd, J=8.3, 7.3 Hz, 1H), 7.51 (dd, J=7.3, 1.2 Hz, 1H), 7.32 (dd, J=8.3, 7.4 Hz, 1H), 7.19 (t, J=8.9 Hz, 1H), 7.13-7.07 (m, 2H), 6.98 (dd, J=5.8, 3.1 Hz, 1H), 6.70 (dd, J=8.2, 0.8 Hz, 1H), 5.91 (s, 1H), 5.12 (s, 2H), 3.93 (s, 3H), 3.39 (q, J=7.6, 6.4 Hz, 2H), 1.66 (sext, J=7.3 Hz, 2H), 1.01 (t, J=7.4 Hz, 3H). m/z 477.2 [M+H]⁺.

### Example 90: 4-Amino-8-[2-fluoro-5-[[6-(trifluoromethyl)-2-pyridyl]methoxy]phenyl]-2-oxo-N-propyl-1H-quinoline-3-carboxamide

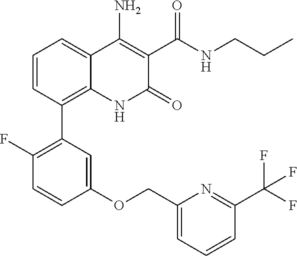 [SMILES: CCCNC(=O)c1c(N)c2cccc(-c3cc(OCc4cccc(C(F)(F)F)n4)ccc3F)c2[nH]c1=O]

### Step 1

General procedure 7 was followed using 3-bromo-4-fluorophenol (100 mg, 0.52 mmol) and (6-(trifluoromethyl)pyridin-2-yl) methanol (120 mg, 0.68 mmol) in MeCN. The material was purified by reverse phase chromatography on an ISCO system (12 g RediSep® Rf Reversed-phase C18, elution with a 10-100% MeOH/water gradient) to give 2-[(3-bromo-4-fluoro-phenoxy)methyl]-6-(trifluoromethyl)pyridine (81 mg, 40%) as an orange solid. ¹H NMR (400 MHZ, Chloroform-d) δ 7.98-7.92 (m, 1H), 7.75 (d, J=7.9 Hz, 1H), 7.66 (d, J=7.7 Hz, 1H), 7.22 (dd, J=5.5, 3.1 Hz, 1H), 7.11-7.04 (m, 1H), 6.91 (dt, J=9.2, 3.5 Hz, 1H), 5.24 (s, 2H). m/z 352.0 [M+H]⁺.

### Step 2

General procedure 1 was followed using intermediate 3 (70 mg, 0.24 mmol) and 2-[(3-bromo-4-fluoro-phenoxy)methyl]-6-(trifluoromethyl)pyridine (85 mg, 0.24 mmol). The material was purified by flash column chromatography on an ISCO system (10 g silica, elution with a 0-10% MeOH/CH₂Cl₂ gradient) to yield a solid, which was then triturated with MeOH to give the title compound (16 mg, 12%) as a colourless solid. ¹H NMR (400 MHz, DMSO-d₆) δ 10.96 (s, 1H), 10.47 (s, 1H), 9.45 (s, 1H), 8.30-8.11 (m, 3H), 7.90 (t, J=6.8 Hz, 2H), 7.51 (d, J=7.3 Hz, 1H), 7.38-7.25 (m, 2H), 7.25-7.07 (m, 2H), 5.32 (s, 2H), 3.29-3.17 (m, 2H), 1.59-1.43 (m, 2H), 0.91 (t, J=7.4 Hz, 3H). m/z 515.2 [M+H]⁺.

### Example 91: 4-Amino-8-(5-((6-cyanopyridin-2-yl)methoxy)-2-fluorophenyl)-2-oxo-N-propyl-1,2-dihydroquinoline-3-carboxamide

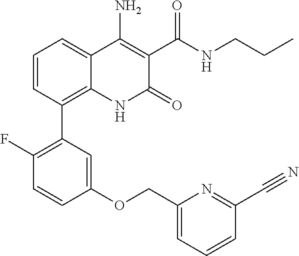 [SMILES: CCCNC(=O)c1c(N)c2cccc(-c3cc(OCc4cccc(C#N)n4)ccc3F)c2[nH]c1=O]

### Step 1

General Procedure 7 was followed using 3-bromo-4-fluorophenol (150 mg, 0.79 mmol) and 6-(chloromethyl)-2-pyridinecarbonitrile (180 mg, 1.18 mmol) in MeCN. The material was purified by flash column chromatography on an ISCO system (10 g silica, elution with a 0-100% EtOAc/pet. ether gradient) to give 6-[(3-bromo-4-fluoro-phenoxy)methyl]pyridine-2-carbonitrile (190 mg, 71%) as a colourless solid. ¹H NMR (400 MHz, Chloroform-d) δ 7.91 (app t, J=7.9 Hz, 1H), 7.77 (d, J=8.0 Hz, 1H), 7.68 (d, J=7.7 Hz, 1H), 7.18 (dd, J=5.5, 3.1 Hz, 1H), 7.07 (t, J=8.5 Hz, 1H), 6.89 (dt, J=9.1, 3.4 Hz, 1H), 5.19 (s, 2H); m/z 308.9 [M+H]⁺.

### Step 2

General procedure 1 was followed using intermediate 4 (175 mg, 0.47 mmol) and 6-[(3-bromo-4-fluoro-phenoxy)methyl]pyridine-2-carbonitrile (174 mg, 0.57 mmol). The material was purified by flash column chromatography on an ISCO system (10 g silica, elution with a 0-100% EtOAc/pet. ether gradient) to give the title compound (84 mg, 36%) as a colourless solid. ¹H NMR (500 MHZ, DMSO-d₆) δ 10.96 (s, 1H), 10.45 (d, J=5.7 Hz, 1H), 9.45 (s, 1H), 8.22 d, J=8.3 Hz, 2H), 8.13 (app t, J=7.8 Hz, 1H), 8.02 (d, J=7.6 Hz, 1H), 7.90 (d, J=7.9 Hz, 1H), 7.51 (d, J=7.3 Hz, 1H), 7.31 (ddd, J=11.1, 7.1, 2.2 Hz, 2H), 7.18 (dt, J=6.9, 3.0 Hz, 1H), 7.12 (dt, J=5.4, 2.4 Hz, 1H), 5.28 (d, J=1.8 Hz, 2H), 3.28-3.20 (m, 2H), 1.51 (h, J=7.3 Hz, 2H), 0.91 (t, J=7.4 Hz, 3H); m/z 472.2 [M+H]⁺.

### Example 92: 4-Amino-8-[2-fluoro-5-[(5-fluoro-2-pyridyl)methoxy]phenyl]-2-oxo-N-propyl-1H-quinoline-3-carboxamide

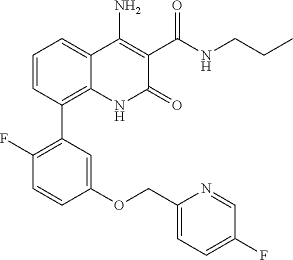 [SMILES: CCCNC(=O)c1c(N)c2cccc(-c3cc(OCc4ccc(F)cn4)ccc3F)c2[nH]c1=O]

### Step 1

General procedure 7 was followed using 3-bromo-4-fluorophenol (986 mg, 5.16 mmol) and 5-fluoro-2-hydroxymethylpyridine (820 mg, 6.45 mmol) in MeCN to give 2-[(3-bromo-4-fluoro-phenoxy)methyl]-5-fluoro-pyridine (1.63 g, 83%) as a brown solid. ¹H NMR (500 MHZ, Chloroform-d) δ 8.46 (d, J=2.8 Hz, 1H), 7.49 (ddq, J=8.7, 4.5, 0.7 Hz, 1H), 7.44 (td, J=8.3, 2.8 Hz, 1H), 7.17 (dd, J=5.5, 3.0 Hz, 1H), 7.03 (dd, J=9.1, 8.0 Hz, 1H), 6.87 (ddd, J=9.1, 3.8, 3.0 Hz, 1H), 5.12 (s, 2H). m/z 302.0 [M+H]⁺.

### Step 2

General procedure 1 was followed using intermediate 3 (400 mg, 1.08 mmol) and 2-[(3-bromo-4-fluoro-phenoxy)methyl]-5-fluoro-pyridine (340 mg, 1.13 mmol). The reaction mixture was concentrated under reduced pressure and then EtOAc was added until a solid precipitated out from the mixture. The solid formed was collected via vacuum filtration and further purified by flash column chromatography on an ISCO system (10 g silica, elution with a 0-10% MeOH/EtOAc gradient) to give the title compound (145 mg, 28%) as an off-white solid. ¹H NMR (500 MHZ, DMSO-d₆) δ 10.95 (s, 1H), 10.45 (t, J=5.6 Hz, 1H), 9.39 (s, 1H), 8.58 (dd, J=2.9, 0.7 Hz, 1H), 8.34-8.08 (m, 2H), 7.78 (td, J=8.8, 2.9 Hz, 1H), 7.65 (dd, J=8.7, 4.6 Hz, 1H), 7.50 (dd, J=7.3, 1.3 Hz, 1H), 7.34-7.26 (m, 2H), 7.16 (ddd, J=9.1, 4.0, 3.2 Hz, 1H), 7.09 (dd, J=6.0, 3.2 Hz, 1H), 5.19 (s, 2H), 3.23 (td, J=7.0, 5.6 Hz, 2H), 1.50 (app. sextet, J=7.3 Hz, 2H), 0.90 (t, J=7.4 Hz, 3H). m/z 465.2 [M+H]⁺.

### Example 93: 4-Amino-8-[3-[(5-fluoro-2-pyridyl)methoxy]phenyl]-2-oxo-N-propyl-1H-quinoline-3-carboxamide

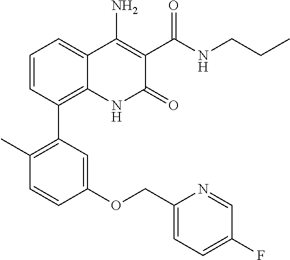 [SMILES: CCCNC(=O)c1c(N)c2cccc(-c3cc(OCc4ccc(F)cn4)ccc3C)c2[nH]c1=O]

### Step 1

General procedure 7 was followed using 3-bromo-4-methylphenol (180 mg, 0.96 mmol) and 5-fluoro-2-hydroxymethylpyridine (150 mg, 1.18 mmol) in MeCN. The material was purified by reverse phase chromatography on an ISCO system (12 g RediSep® Rf Reversed-phase C18, elution with a 10-100% MeOH/water gradient) to give 2-[(3-bromo-4-methyl-phenoxy)methyl]-5-fluoro-pyridine (108 mg, 34%) as a pink solid. ¹H NMR (400 MHz, Chloroform-d) δ 8.48 (d, J=2.9 Hz, 1H), 7.53 (dd, J=8.7, 4.5 Hz, 1H), 7.45 (td, J=8.3, 2.9 Hz, 1H), 7.21 (d, J=2.7 Hz, 1H), 7.15 (d, J=8.4 Hz, 1H), 6.85 (dd, J=8.4, 2.7 Hz, 1H), 5.16 (s, 2H), 2.35 (s, 3H). m/z 298.0 [M+H]⁺.

### Step 2

General procedure 1 was followed intermediate 4 (100 mg, 0.27 mmol) and 2-[(3-bromo-4-methyl-phenoxy)methyl]-5-fluoro-pyridine (80 mg, 0.27 mmol). The material was purified by flash column chromatography on an ISCO system (10 g silica, elution with a 0-10% MeOH/CH₂Cl₂ gradient), followed by reverse phase chromatography on an ISCO system (12 g RediSep® Rf Reversed-phase C18, elution with a 10-100% MeOH/water gradient) to give the title compound (71 mg, 53%) as a colourless solid. 1H NMR (400 MHZ, DMSO-d₆) δ 10.97 (s, 1H), 10.45-10.29 (m, 1H), 8.63-8.51 (m, 1H), 8.38 (s, 1H), 8.33-8.13 (m, 2H), 7.84-7.71 (m, 1H), 7.67-7.57 (m, 1H), 7.47-7.38 (m, 1H), 7.36-7.26 (m, 2H), 7.13-7.02 (m, 1H), 6.90 (d, J=3.0 Hz, 1H), 5.17 (s, 2H), 3.22 (q, J=6.5 Hz, 2H), 1.92 (s, 3H), 1.56-1.44 (m, 2H), 0.96-0.82 (m, 3H). m/z 461.2 [M+H]⁺.

### Example 94: 4-Amino-8-[3-[(5-fluoro-2-pyridyl)methoxy]phenyl]-2-oxo-N-propyl-1H-quinoline-3-carboxamide

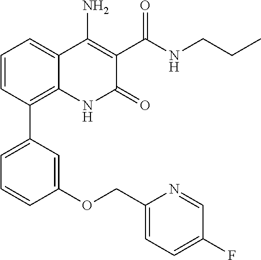 [SMILES: CCCNC(=O)c1c(N)c2cccc(-c3cccc(OCc4ccc(F)cn4)c3)c2[nH]c1=O]

### Step 1

General procedure 7 was followed using 3-bromophenol (160 mg, 0.92 mmol) and 5-fluoro-2-hydroxymethylpyridine (150 mg, 1.18 mmol) in MeCN. The material was purified by reverse phase chromatography on an ISCO system (12 g RediSep® Rf Reversed-phase C18, elution with a 10-100% MeOH/water gradient) to give 2-[(3-bromophenoxy)methyl]-5-fluoro-pyridine (106 mg, 39%) as a yellow oil. ¹H NMR (400 MHZ, Chloroform-d) δ 8.48 (d, J=2.9 Hz, 1H), 7.62-7.42 (m, 2H), 7.24-7.09 (m, 3H), 7.01-6.83 (m, 1H), 5.18 (d, J=3.6 Hz, 2H). m/z 284.0 [M+H]⁺.

### Step 2

General procedure 1 was followed using intermediate 4 (100 mg, 0.27 mmol) and 2-[(3-bromophenoxy)methyl]-5-fluoro-pyridine (76 mg, 0.27 mmol). The material was purified by flash column chromatography on an ISCO system (10 g silica, elution with a 0-10% MeOH/CH₂Cl₂ gradient), followed by and reverse phase chromatography on an ISCO system (12 g RediSep® Rf Reversed-phase C18, elution with a 10-100% MeOH/water gradient) to give the title compound (59 mg, 43%) as a colourless solid. ¹H NMR (400 MHz, DMSO-d₆) δ 10.96 (s, 1H), 10.39 (s, 1H), 8.71 (s, 1H), 8.59 (dt, J=2.9, 0.7 Hz, 1H), 8.24 (s, 1H), 8.18 (dd, J=8.4, 1.3 Hz, 1H), 7.79 (td, J=8.8, 2.9 Hz, 1H), 7.66 (ddd, J=8.7, 4.6, 0.6 Hz, 1H), 7.57-7.45 (m, 2H), 7.33 (dd, J=8.3, 7.3 Hz, 1H), 7.19-7.11 (m, 2H), 7.08-7.04 (m, 1H), 5.25 (s, 2H), 3.26-3.20 (m, 2H), 1.57-1.44 (m, 2H), 0.91 (t, J=7.4 Hz, 3H). m/z 447.2 [M+H]⁺.

### Example 95: 4-Amino-8-[2-fluoro-5-[(5-methyl-2-pyridyl)methoxy]phenyl]-2-oxo-N-propyl-1H-quinoline-3-carboxamide

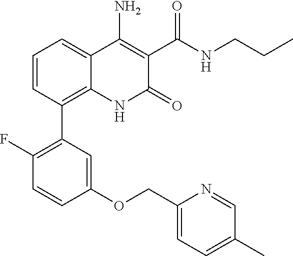 [SMILES: CCCNC(=O)c1c(N)c2cccc(-c3cc(OCc4ccc(C)cn4)ccc3F)c2[nH]c1=O]

### Step 1

General procedure 7 was followed using 3-bromo-4-fluorophenol (300 mg, 1.57 mmol) and 2-(chloromethyl)-5-methylpyridine hydrochloride (308 mg, 1.73 mmol) in acetone to give 2-[(3-bromo-4-fluoro-phenoxy)methyl]-5-methyl-pyridine (381 mg, 78%) as a brown solid. ¹H NMR (500 MHZ, DMSO-d₆) δ 8.45 (s, 1H), 7.55 (dd, J=7.9, 2.1 Hz, 1H), 7.37 (d, J=7.9 Hz, 1H), 7.21-7.16 (m, 1H), 7.03 (d, J=8.8 Hz, 1H), 6.93-6.86 (m, 1H), 5.13 (s, 2H), 2.37 (s, 3H). m/z 298.0 [M+H]⁺.

### Step 2

General procedure 1 was followed using intermediate 3 (150 mg, 0.52 mmol) and 2-[(3-bromo-4-fluoro-phenoxy)methyl]-5-methyl-pyridine (184 mg, 0.62 mmol). The material was purified by flash column chromatography on an ISCO system (10 g silica, elution with a 0-5% MeOH/CH₂Cl₂ gradient) to yield a solid, which was then triturated with EtOAc to give the title compound (92 mg, 37%) as a colourless solid. ¹H NMR (500 MHz, Chloroform-d) δ 11.28 (s, 1H), 10.29 (t, J=5.7 Hz, 1H), 8.43 (s, 1H), 8.01 (s, 1H), 7.70 (d, J=8.3 Hz, 1H), 7.55 (d, J=8.0 Hz, 1H), 7.48 (d, J=7.3 Hz, 1H), 7.40 (d, J=7.9 Hz, 1H), 7.32-7.25 (m, 1H), 7.16 (td, J=9.0, 1.6 Hz, 1H), 7.09-7.03 (m, 1H), 6.94 (t, J=4.7 Hz, 1H), 6.06 (s, 1H), 5.17 (s, 2H), 3.38 (q, J=6.6 Hz, 2H), 2.36 (s, 3H), 1.65 (qd, J=7.3, 1.5 Hz, 2H), 1.00 (td, J=7.4, 1.6 Hz, 3H). m/z 461.2 [M+H]⁺.

### Example 96: 4-Amino-8-[2-fluoro-5-[(5-methoxy-2-pyridyl)methoxy]phenyl]-2-oxo-N-propyl-1H-quinoline-3-carboxamide

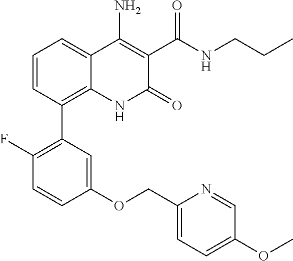 [SMILES: CCCNC(=O)c1c(N)c2cccc(-c3cc(OCc4ccc(OC)cn4)ccc3F)c2[nH]c1=O]

### Step 1

General procedure 7 was followed using 3-bromo-4-fluorophenol (170 mg, 0.89 mmol) and (5-methoxypyridine-2-yl) methanol (159 mg, 1.08 mmol) in MeCN. The material was purified by reverse phase chromatography on an ISCO system (12 g RediSep® Rf Reversed-phase C18, elution with a 10-100% MeOH/water gradient) to give 2-[(3-bromo-4-fluoro-phenoxy)methyl]-5-methoxy-pyridine (67 mg, 22%) as a brown oil. ¹H NMR (400 MHz, Chloroform-d) δ 8.30 (d, J=3.0 Hz, 1H), 7.40 (d, J=8.5 Hz, 1H), 7.27-7.12 (m, 2H), 7.09-6.96 (m, 1H), 6.92-6.80 (m, 1H), 5.09 (s, 2H), 3.87 (s, 3H). m/z 314.0 [M+H]⁺.

### Step 2

General procedure 1 was followed using intermediate 4 (80 mg, 0.22 mmol) and 2-[(3-bromo-4-fluoro-phenoxy)methyl]-5-methoxy-pyridine (67 mg, 0.22 mmol). The material was purified by flash column chromatography on an ISCO system (10 g silica, elution with a 0-10% MeOH/CH₂Cl₂ gradient), followed by reverse phase chromatography on an ISCO system (12 g RediSep® Rf Reversed-phase C18, elution with a 10-100% MeOH/water gradient) to give the title compound (13 mg, 11%) as a yellow solid. ¹H NMR (500 MHZ, DMSO-d₆) δ 10.95 (s, 1H), 10.46 (t, J=5.5 Hz, 1H), 9.37 (s, 1H), 8.29 (dd, J=3.0, 0.7 Hz, 1H), 8.22 (d, J=8.2 Hz, 1H), 7.54-7.48 (m, 2H), 7.44 (dd, J=8.6, 3.0 Hz, 1H), 7.35-7.25 (m, 2H), 7.19-7.11 (m, 1H), 7.07 (dd, J=6.0, 3.1 Hz, 1H), 5.13 (s, 2H), 3.84 (s, 3H), 3.27-3.19 (m, 2H), 1.57-1.44 (m, 2H), 0.91 (t, J=7.4 Hz, 3H). m/z 477.2

[M+H]⁺.

### Example 97: 4-Amino-8-[5-[(5-chloro-2-pyridyl)methoxy]-2-fluoro-phenyl]-2-oxo-N-propyl-1H-quinoline-3-carboxamide

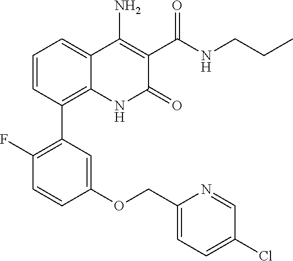 [SMILES: CCCNC(=O)c1c(N)c2cccc(-c3cc(OCc4ccc(Cl)cn4)ccc3F)c2[nH]c1=O]

### Step 1

General procedure 7 was followed using 3-bromo-4-fluorophenol (120 mg, 0.63 mmol) and 5-chloro-2-(chloromethyl)pyridine (122 mg, 0.75 mmol) in MeCN to give 2-[(3-bromo-4-fluoro-phenoxy)methyl]-5-chloro-pyridine (190 mg, 81%) as a brown solid. 1H NMR (400 MHZ, Chloroform-d) δ 8.57 (d, J=2.5 Hz, 1H), 7.78-7.65 (m, 1H), 7.45 (dd, J=8.4, 4.2 Hz, 1H), 7.17 (dd, J=5.6, 3.0 Hz, 1H), 7.12-6.95 (m, 1H), 6.93-6.78 (m, 1H), 5.13 (s, 2H). m/z 317.9 [M+H]⁺.

### Step 2

General procedure 1 was followed using intermediate 4 (80 mg, 0.22 mmol) and 2-[(3-bromo-4-fluoro-phenoxy)methyl]-5-chloro-pyridine (85 mg, 0.22 mmol). The material was purified by flash column chromatography on an ISCO system (10 g silica, elution with a 0-10% MeOH/CH₂Cl₂ gradient) to yield a solid, which was then triturated with MeOH to give the title compound (45 mg, 33%) as a yellow solid. ¹H NMR (500 MHZ, DMSO-d₆) O 10.95 (s, 1H), 10.46 (t, J=5.5 Hz, 1H), 9.40 (s, 1H), 8.67-8.58 (m, 1H), 8.24-8.18 (m, 2H), 7.99 (dd, J=8.4, 2.5 Hz, 1H), 7.61 (dd, J=8.4, 0.8 Hz, 1H), 7.51 (dd, J=7.4, 1.3 Hz, 1H), 7.37-7.25 (m, 2H), 7.20-7.13 (m, 1H), 7.09 (dd, J=6.0, 3.2 Hz, 1H), 5.21 (s, 2H), 3.28-3.16 (m, 2H), 1.56-1.47 (m, 2H), 0.91 (t, J=7.4 Hz, 3H). m/z 481.2 [M+H]⁺.

### Example 98: 4-Amino-8-[2-fluoro-5-[[5-(trifluoromethyl)-2-pyridyl]methoxy]phenyl]-2-oxo-N-propyl-1H-quinoline-3-carboxamide

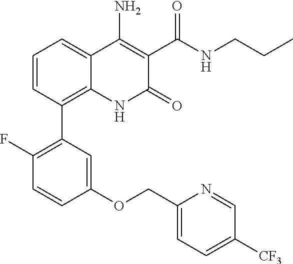 [SMILES: CCCNC(=O)c1c(N)c2cccc(-c3cc(OCc4ccc(C(F)(F)F)cn4)ccc3F)c2[nH]c1=O]

### Step 1

General procedure 7 was followed using 3-bromo-4-fluorophenol (180 mg, 0.94 mmol) and (5-(trifluoromethyl)pyridin-2-yl) methanol (180 mg, 1.01 mmol) in acetone. The material was purified by flash column chromatography on an ISCO system (10 g silica, elution with a 0-50% EtOAc/pet. ether gradient) to give 2-[(3-bromo-4-fluoro-phenoxy)methyl]-5-(trifluoromethyl)pyridine (65 mg, 15%) as an orange solid. ¹H NMR (400 MHZ, Chloroform-d) δ 8.89 (d, J=2.2 Hz, 1H), 8.00 (dd, J=8.2, 2.4 Hz, 1H), 7.67 (d, J=8.2 Hz, 1H), 7.21 (dd, J=5.5, 3.1 Hz, 1H), 7.11-7.04 (m, 1H), 6.94-6.87 (m, 1H), 5.24 (s, 2H). m/z 352.0 [M+H]⁺.

### Step 2

General procedure 1 was followed using intermediate 4 (70 mg, 0.19 mmol) and 2-[(3-bromo-4-fluoro-phenoxy)methyl]-5-(trifluoromethyl)pyridine (66 mg, 0.19 mmol). The material was purified by flash column chromatography on an ISCO system (10 g silica, elution with a 0-10% MeOH/CH₂Cl₂ gradient), followed by reverse phase chromatography on an ISCO system (12 g RediSep® Rf Reversed-phase C18, elution with a 10-100% MeOH/water gradient) to give the title compound (22 mg, 22%) as a tan solid. ¹H NMR (400 MHZ, DMSO-d₆) δ 10.94 (s, 1H), 10.47 (s, 1H), 9.42 (s, 1H), 8.99 (s, 1H), 8.33-8.12 (m, 3H), 7.80 (d, J=8.2 Hz, 1H), 7.51 (d, J=7.3 Hz, 1H), 7.37-7.26 (m, 2H), 7.22-7.06 (m, 2H), 5.33 (d, J=5.5 Hz, 2H), 3.28-3.19 (m, 2H), 1.58-1.44 (m, 2H), 0.91 (t, J=7.4 Hz, 3H). m/z 515.2 [M+H]⁺.

### Example 99: 4-Amino-8-[5-[(5-cyano-2-pyridyl)methoxy]-2-fluoro-phenyl]-2-oxo-N-propyl-1H-quinoline-3-carboxamide

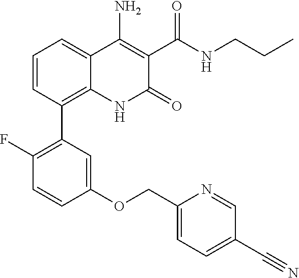 [SMILES: CCCNC(=O)c1c(N)c2cccc(-c3cc(OCc4ccc(C#N)cn4)ccc3F)c2[nH]c1=O]

### Step 1

General procedure 7 was followed using 3-bromo-4-fluorophenol (100 mg, 0.52 mmol) and 6-bromomethyl-nicotinonitrile (113 mg, 0.58 mmol) in MeCN. The material was purified by flash column chromatography on an ISCO system (10 g silica, elution with a 0-20% EtOAc/pet. ether gradient) to give 6-[(3-bromo-4-fluoro-phenoxy)methyl]pyridine-3-carbonitrile (117 mg, 69%) as a colourless solid. ¹H NMR (500 MHZ, Chloroform-d) δ 8.91-8.80 (m, 1H), 8.01 (dd, J=8.2, 2.1 Hz, 1H), 7.71-7.61 (m, 1H), 7.17 (dd, J=5.5, 3.0 Hz, 1H), 7.06 (dd, J=9.1, 7.9 Hz, 1H), 6.88 (dt, J=9.1, 3.2 Hz, 1H), 5.20 (s, 2H). m/z 307.0 [M+H]⁺.

### Step 2

General procedure 2 was followed using intermediate 4 (143 mg, 0.27 mmol) and 6-[(3-bromo-4-fluoro-phenoxy)methyl]pyridine-3-carbonitrile (104 mg, 0.34 mmol). The material was purified by flash column chromatography on an ISCO system (10 g silica, elution with a 0-75% EtOAc/pet. ether gradient) to yield a solid, which was then triturated with EtOAc to give the title compound (26 mg, 19%) as a cream solid. ¹H NMR (500 MHZ, Chloroform-d) δ 11.31 (s, 1H), 10.23 (t, J=5.6 Hz, 1H), 8.86 (d, J=2.1 Hz, 1H), 8.02 (dd, J=8.3, 2.1 Hz, 1H), 7.95 (s, 1H), 7.68 (m, 2H), 7.48 (d, J=7.4 Hz, 1H), 7.30 (t, J=7.8 Hz, 1H), 7.19 (t, J=8.8 Hz, 1H), 7.05 (dt, J=9.1, 3.5 Hz, 1H), 6.93 (dd, J=5.7, 3.1 Hz, 1H), 5.93 (s, 1H), 5.25 (s, 2H), 3.36 (q, J=6.7 Hz, 2H), 1.66-1.60 (m, 2H), 0.98 (t, J=7.4 Hz, 3H). m/z 472.2 [M+H]⁺.

### Example 100: 4-Amino-8-[2-fluoro-5-[(4-methoxy-2-pyridyl)methoxy]phenyl]-2-oxo-N-propyl-1H-quinoline-3-carboxamide

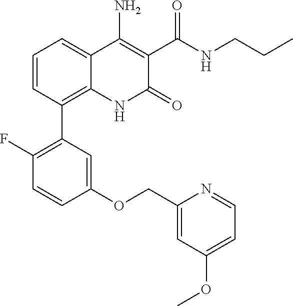 [SMILES: CCCNC(=O)c1c(N)c2cccc(-c3cc(OCc4cc(OC)ccn4)ccc3F)c2[nH]c1=O]

### Step 1

General procedure 7 was followed using 3-bromo-4-fluorophenol (123 mg, 0.64 mmol) and 4-methoxy-2-hydroxymethylpyridine (100 mg, 0.72 mmol) in MeCN. The material was purified by flash column chromatography on an ISCO system (10 g silica, elution with a 0-50% EtOAc/pet. ether gradient) to give 2-[(3-bromo-4-fluoro-phenoxy)methyl]-4-methoxy-pyridine (312 mg, 27%) as a cream solid. ¹H NMR (500 MHZ, Chloroform-d) δ 8.41 (d, J=5.7 Hz, 1H), 7.18 (dd, J=5.6, 3.0 Hz, 1H), 7.07-6.98 (m, 2H), 6.88 (dt, J=9.1, 3.4 Hz, 1H), 6.76 (dd, J=5.8, 2.5 Hz, 1H), 5.10 (s, 2H), 3.86 (s, 3H). m/z 312.0 [M+H]⁺.

### Step 2

General procedure 2 was followed using intermediate 4 (85 mg, 0.16 mmol) and 2-[(3-bromo-4-fluoro-phenoxy)methyl]-4-methoxy-pyridine (63 mg, 0.20 mmol). The material was purified by flash column chromatography on an ISCO system (10 g silica, elution with a 0-100% EtOAc/pet. ether gradient) to yield a solid, which was then triturated with EtOAc and pet. ether to give the title compound (25 mg, 31%) as a cream solid. ¹H NMR (500 MHZ, Chloroform-d) δ 11.29 (s, 1H), 10.27 (t, J=5.6 Hz, 1H), 8.40 (d, J=5.8 Hz, 1H), 7.95 (s, 1H), 7.66 (d, J=8.3 Hz, 1H), 7.48 (d, J=7.4 Hz, 1H), 7.29 (t, J=7.8 Hz, 1H), 7.16 (t, J=8.9 Hz, 1H), 7.10-7.03 (m, 2H), 6.94 (dd, J=5.9, 3.1 Hz, 1H), 6.76 (dd, J=5.8, 2.5 Hz, 1H), 5.90 (s, 1H), 5.15 (s, 2H), 3.87 (s, 3H), 3.37 (q, J=6.6 Hz, 2H), 1.68-1.61 (m, 2H), 0.99 (t, J=7.4 Hz, 3H). m/z 477.2 [M+H]⁺.

### Example 101: 4-Amino-8-[5-[(4-cyano-2-pyridyl)methoxy]-2-fluoro-phenyl]-2-oxo-N-propyl-1H-quinoline-3-carboxamide

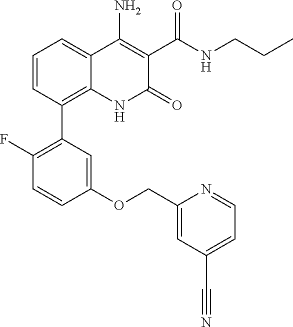 [SMILES: CCCNC(=O)c1c(N)c2cccc(-c3cc(OCc4cc(C#N)ccn4)ccc3F)c2[nH]c1=O]

### Step 1

General procedure 7 was followed using 3-bromo-4-fluorophenol (100 mg, 0.52 mmol) and 2-(chloromethyl)pyridine-4-carbonitrile hydrochloride (109 mg, 0.58 mmol) in MeCN to give 2-[(3-bromo-4-fluoro-phenoxy)methyl]pyridine-4-carbonitrile (107 mg, 63%) as a colourless solid. ¹H NMR (500 MHZ, Chloroform-d) δ 8.78 (dd, J=5.0, 0.9 Hz, 1H), 7.76 (dd, J=1.7, 0.9 Hz, 1H), 7.49 (dd, J=4.9, 1.4, 0.7 Hz, 1H), 7.19 (dd, J=5.5, 3.1 Hz, 1H), 7.07 (dd, J=9.0, 7.9 Hz, 1H), 6.90 (ddd, J=9.0, 3.7, 3.0 Hz, 1H), 5.19 (s, 2H). m/z 307.0 [M+H]⁺.

### Step 2

General procedure 2 was followed using intermediate 4 (143 mg, 0.27 mmol) and 2-[(3-bromo-4-fluoro-phenoxy)methyl]pyridine-4-carbonitrile (104 mg, 0.34 mmol). The material was purified by flash column chromatography on an ISCO system (10 g silica, elution with a 0-80% EtOAc/pet. ether gradient) to yield a solid, which was then triturated with EtOAc to give the title compound (70 mg, 52%) as a colourless solid. ¹H NMR (500 MHz, Chloroform-d) δ 11.31 (s, 1H), 10.25 (t, J=5.6 Hz, 1H), 8.78 (d, J=4.9 Hz, 1H), 7.98 (s, 1H), 7.80 (s, 1H), 7.68 (d, J=8.2 Hz, 1H), 7.52-7.47 (m, 2H), 7.31 (t, J=7.8 Hz, 1H), 7.20 (t, J=8.9 Hz, 1H), 7.07 (dt, J=9.1, 3.5 Hz, 1H), 6.97 (dd, J=5.6, 3.2 Hz, 1H), 5.92 (s, 1H), 5.23 (s, 2H), 3.36 (dt, J=7.6, 6.2 Hz, 2H), 1.64 (sext, J=7.3 Hz, 2H), 0.98 (t, J=7.4 Hz, 3H). m/z 472.2 [M+H]⁺.

### Example 102: 4-Amino-8-(2-fluoro-5-((4-(hydroxymethyl)pyridin-2-yl)methoxy)phenyl)-2-oxo-N-propyl-1,2-dihydroquinoline-3-carboxamide

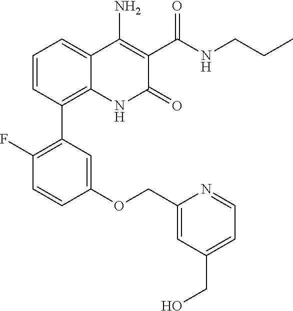 [SMILES: CCCNC(=O)c1c(N)c2cccc(-c3cc(OCc4cc(CO)ccn4)ccc3F)c2[nH]c1=O]

### Step 1

To methyl 4-(hydroxymethyl) picolinate (400 mg, 2.39 mmol) in DMF (5 mL), was added imidazole (326 mg, 4.79 mmol) and TBDMSCl (397 mg, 2.63 mmol) and the reaction mixture was stirred at rt for 5 h. After this time, water (20 mL) was added and the reaction mixture extracted with Et₂O (20 mL). The organic extract was washed with water, dried over MgSO₄, filtered and concentrated under reduced pressure. The material was purified by flash column chromatography on an ISCO system (10 g silica, elution with a 0-100% EtOAc/pet. ether gradient) to give methyl 4-[[tert-butyl(dimethyl)silyl]oxymethyl]pyridine-2-carboxylate (583 mg, 82%) as a colourless oil. ¹H NMR (500 MHz, Chloroform-d) δ 8.69 (dd, J=4.9, 0.8 Hz, 1H), 8.07 (dq, J=1.7, 0.8 Hz, 1H), 7.48 (ddt, J=4.9, 1.8, 0.9 Hz, 1H), 4.80 (t, J=1.0 Hz, 2H), 4.00 (s, 3H), 0.96 (s, 9H), 0.12 (s, 6H). m/z 282.1 [M+H]⁺.

### Step 2

To methyl 4-[[tert-butyl(dimethyl)silyl]oxymethyl]pyridine-2-carboxylate (580 mg, 2.06 mmol) in EtOH (10 mL), was added 2 M lithium borohydride in THF (1.03 mL, 2.06 mmol) and the reaction mixture was stirred at rt for 1 h. After this time, a further portion of 2 M lithium borohydride in THF (1.03 mL, 2.06 mmol) was added and the reaction mixture stirred for a further 22 h. After this time, the reaction mixture was quenched with sat. aq. NaHCO₃ (20 mL) and extracted with CH₂Cl₂ (3×20 mL). The combined organic extracts were washed with water, dried over MgSO₄, filtered and concentrated under reduced pressure to give (4-(((tert-butyldimethylsilyl)oxy)methyl)pyridin-2-yl) methanol (525 mg, 90%) as a colourless oil. ¹H NMR (400 MHZ, Chloroform-d) δ 8.48 (d, J=5.1 Hz, 1H), 7.20 (s, 1H), 7.16 (d, J=5.2 Hz, 1H), 4.75 (s, 4H), 0.95 (s, 9H), 0.11 (s, 6H). m/z 254.2 [M+H]⁺.

### Step 3

General procedure 7 was followed using 3-bromo-4-fluorophenol (290 mg, 1.52 mmol) and (4-(((tert-butyldimethylsilyl)oxy)methyl)pyridin-2-yl) methanol (525 mg, 1.86 mmol) in MeCN. The crude alkylated product was dissolved in THF (5 mL) and the solution cooled to 0° C. A solution of 1 M TBAF in THF (1.52 mL, 1.52 mmol) was added and the reaction mixture stirred at rt for 3 h. After this time, sat. aq. NH₄Cl (20 mL) was added and extracted with EtOAc (3×20 mL). The combined organic extracts were washed with water (20 mL) and brine (20 mL), dried over MgSO₄, filtered and concentrated under reduced pressure. The material was purified by flash column chromatography on an ISCO system (10 g silica, elution with a 50-100% EtOAc/pet. ether gradient) to give [2-[(3-bromo-4-fluoro-phenoxy)methyl]-4-pyridyl] methanol (460 mg, 90%) as an orange solid. ¹H NMR (400 MHz, Chloroform-d) δ 8.56 (dd, J=5.1, 0.8 Hz, 1H), 7.50-7.47 (m, 1H), 7.26-7.24 (m, 1H), 7.18 (dd, J=5.5, 3.0 Hz, 1H), 7.03 (dd, J=9.0, 8.0 Hz, 1H), 6.89 (ddd, J=9.0, 3.8, 3.0 Hz, 1H), 5.15 (s, 2H), 4.77 (dt, J=5.9, 0.9 Hz, 2H), 1.97 (t, J=5.9 Hz, 1H). m/z 314.0 [M+H]⁺.

### Step 4

General procedure 1 was followed using intermediate 4 (100 mg, 0.27 mmol) and [2-[(3-bromo-4-fluoro-phenoxy)methyl]-4-pyridyl] methanol (88 mg, 0.28 mmol). The material was purified by flash column chromatography on an ISCO system (10 g silica, elution with a 0-10% MeOH/EtOAc gradient) to give the title compound (69 mg, 51%) as a colourless solid. ¹H NMR (500 MHZ, DMSO-d₆) δ 10.95 (s, 1H), 10.46 (t, J=5.6 Hz, 1H), 9.41 (s, 1H), 8.49 (dd, J=5.0, 0.8 Hz, 1H), 8.33-8.10 (m, 2H), 7.55-7.48 (m, 2H), 7.35-7.25 (m, 3H), 7.16 (ddd, J=9.0, 4.0, 3.1 Hz, 1H), 7.08 (dd, J=6.0, 3.2 Hz, 1H), 5.45 (t, J=5.7 Hz, 1H), 5.18 (s, 2H), 4.56 (d, J=5.7 Hz, 2H), 3.23 (td, J=6.9, 5.6 Hz, 2H), 1.51 (app. sextet, J=7.3 Hz, 2H), 0.90 (t, J=7.4 Hz, 3H). m/z 477.3 [M+H]⁺.

### Example 103: 4-Amino-8-[2-fluoro-5-[(3-methyl-2-pyridyl)methoxy]phenyl]-2-oxo-N-propyl-1H-quinoline-3-carboxamide

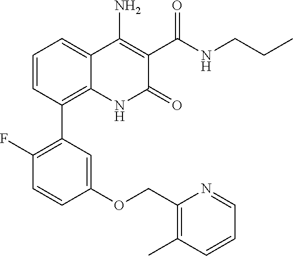 [SMILES: CCCNC(=O)c1c(N)c2cccc(-c3cc(OCc4ncccc4C)ccc3F)c2[nH]c1=O]

### Step 1

General procedure 7 was followed using 3-bromo-4-fluorophenol (420 mg, 2.20 mmol) and 2-hydroxymethyl-3-methylpyridine (308 mg, 1.73 mmol) in acetone to give 2-[(3-bromo-4-fluoro-phenoxy)methyl]-3-methyl-pyridine (600 mg, 88%) as a yellow solid. 1H NMR (500 MHZ, DMSO-d₆) δ 8.47 (d, J=4.7 Hz, 1H), 7.55 (d, J=7.6 Hz, 1H), 7.26-7.20 (m, 2H), 7.09-7.01 (m, 1H), 6.99-6.91 (m, 1H), 5.18 (s, 2H), 2.44 (s, 3H). m/z 298.0 [M+H]⁺.

### Step 2

General procedure 1 was followed using intermediate 3 (150 mg, 0.52 mmol) and 2-[(3-bromo-4-fluoro-phenoxy)methyl]-3-methyl-pyridine (184 mg, 0.62 mmol). The material was purified by reverse phase chromatography on an ISCO system (12 g RediSep® Rf Reversed-phase C18, elution with a 10-100% MeOH/water gradient) to yield a solid, which was then triturated with EtOAc to give the title compound (38 mg, 15%) as a colourless solid. ¹H NMR (500 MHZ, Chloroform-d) δ 10.96 (s, 1H), 10.47 (t, J=5.6 Hz, 1H), 9.41 (s, 1H), 8.39 (dd, J=4.8, 1.7 Hz, 1H), 8.25-8.18 (m, 2H), 7.67 (dd, J=7.6, 1.7 Hz, 1H), 7.51 (dd, J=7.4, 1.2 Hz, 1H), 7.35-7.23 (m, 3H), 7.17 (ddd, J=9.0, 4.1, 3.1 Hz, 1H), 7.10 (dd, J=6.0, 3.1 Hz, 1H), 5.21 (s, 2H), 3.24 (q, J=6.6 Hz, 2H), 2.40 (s, 3H), 1.51 (q, J=7.2 Hz, 2H), 0.91 (t, J=7.4 Hz, 3H). m/z 461.2 [M+H]⁺.

### Example 104: 4-Amino-8-(5-fluoro-2-((pyridin-2-yloxy)methyl)pyridin-4-yl)-2-oxo-N-propyl-1,2-dihydroquinoline-3-carboxamide

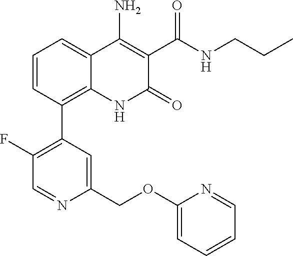 [SMILES: CCCNC(=O)c1c(N)c2cccc(-c3cc(COc4ccccn4)ncc3F)c2[nH]c1=O]

### Step 1

General procedure 4 was followed using 4-chloro-5-fluoro-2-pyridinemethanol (149 mg, 0.92 mmol) and 2-pyridone (70 mg, 0.74 mmol). The material was purified by flash column chromatography on an ISCO system (10 g silica, elution with a 0-100% EtOAc/pet. ether gradient) to give 4-chloro-5-fluoro-2-(2-pyridyloxymethyl)pyridine (35 mg, 20%) as a colourless solid. ¹H NMR (500 MHZ, Chloroform-d) δ 8.48 (s, 1H), 8.17 (dd, J=5.1, 2.3 Hz, 1H), 7.67-7.60 (m, 1H), 7.57 (dd, J=5.8, 1.9 Hz, 1H), 6.96-6.87 (m, 2H), 5.48 (s, 2H). m/z 239.1 [M+H]⁺.

### Step 2

General procedure 1 was followed using intermediate 4 (70 mg, 0.14 mmol) and 4-chloro-5-fluoro-2-(2-pyridyloxymethyl)pyridine (35 mg, 0.15 mmol). The material was purified by reverse phase chromatography on an ISCO system (12 g RediSep® Rf Reversed-phase C18, elution with a 10-100% MeOH/water gradient), followed by flash column chromatography on an ISCO system (10 g silica, elution with a 0-100% EtOAc/pet. ether gradient) to give the title compound (17 mg, 26%) as a colourless solid. ¹H NMR (500 MHZ, Chloroform-d) o 11.39 (s, 1H), 10.22 (s, 1H), 8.67 (s, 1H), 8.22-8.16 (m, 1H), 7.83 (s, 1H), 7.74 (d, J=8.3 Hz, 1H), 7.67-7.57 (m, 1H), 7.56-7.48 (m, 2H), 7.39-7.31 (m, 1H), 6.97-6.90 (m, 1H), 6.87 (d, J=8.3 Hz, 1H), 5.94 (s, 1H), 5.58 (s, 2H), 3.43-3.34 (m, 2H), 1.71-1.62 (m, 2H), 1.01 (t, J=7.4 Hz, 3H). m/z 448.2 [M+H]⁺.

### Example 105: 4-Amino-8-[2-fluoro-5-(pyridazin-3-ylmethoxy)phenyl]-2-oxo-N-propyl-1H-quinoline-3-carboxamide

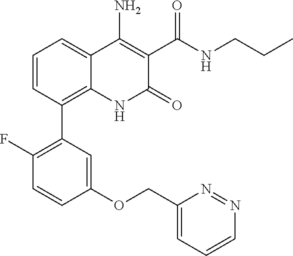 [SMILES: CCCNC(=O)c1c(N)c2cccc(-c3cc(OCc4cccnn4)ccc3F)c2[nH]c1=O]

### Step 1

General procedure 7 was followed using 3-bromo-4-fluorophenol (180 mg, 0.94 mmol) and 3-hydroxymethylpyridazine (150 mg, 1.36 mmol) in MeCN. The material was purified by reverse phase chromatography on an ISCO system (12 g RediSep® Rf Reversed-phase C18, elution with a 10-100% MeOH/water gradient) to give 3-[(3-bromo-4-fluoro-phenoxy)methyl]pyridazine (75 mg, 27%) as an orange solid. ¹H NMR (400 MHZ, Chloroform-d) δ 9.20 (d, J=4.9 Hz, 1H), 7.73 (d, J=8.4 Hz, 1H), 7.56 (dd, J=8.5, 4.9 Hz, 1H), 7.23 (dd, J=5.5, 3.0 Hz, 1H), 7.15-7.01 (m, 1H), 6.99-6.86 (m, 1H), 5.42 (s, 2H). m/z 285.0 [M+H]⁺.

### Step 2

General procedure 1 was followed using intermediate 4 (100 mg, 0.27 mmol) and 3-[(3-bromo-4-fluoro-phenoxy)methyl]pyridazine (76 mg, 0.27 mmol). The material was purified by flash column chromatography on an ISCO system (10 g silica, elution with a 0-10% MeOH/CH₂Cl₂ gradient) to yield a solid, which was then triturated with MeOH to give the title compound (18 mg, 14%) as a colourless solid. ¹H NMR (400 MHZ, DMSO-d₆) δ 10.96 (s, 1H), 10.48 (d, J=5.7 Hz, 1H), 9.51 (s, 1H), 9.23 (d, J=5.0 Hz, 1H), 8.29-8.17 (m, 2H), 7.88 (d, J=8.3 Hz, 1H), 7.78 (dd, J=8.4, 4.9 Hz, 1H), 7.51 (d, J=7.3 Hz, 1H), 7.37-7.27 (m, 2H), 7.25-7.17 (m, 1H), 7.16-7.10 (m, 1H), 5.44 (s, 2H), 3.28-3.17 (m, 2H), 1.51 (q, J=7.3 Hz, 2H), 0.91 (t, J=7.4 Hz, 3H). m/z 448.2 [M+H]⁺.

### Example 106: 4-Amino-8-[2-fluoro-5-(pyridazin-4-ylmethoxy)phenyl]-2-oxo-N-propyl-1H-quinoline-3-carboxamide

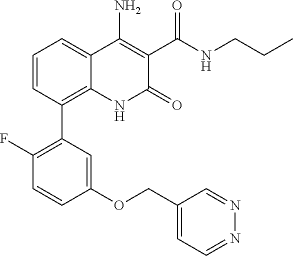 [SMILES: CCCNC(=O)c1c(N)c2cccc(-c3cc(OCc4ccnnc4)ccc3F)c2[nH]c1=O]

### Step 1

General procedure 7 was followed using 3-bromo-4-fluorophenol (160 mg, 0.84 mmol) and pyridazin-4-ylmethanol (120 mg, 1.09 mmol) in MeCN to give 3-[(3-bromo-4-fluoro-phenoxy)methyl]pyridazine (63 mg, 24%) as a brown solid. m/z 285.0 [M+H]⁺.

### Step 2

General procedure 1 was followed using intermediate 4 (80 mg, 0.22 mmol) and 4-[(3-bromo-4-fluoro-phenoxy)methyl]pyridazine (61 mg, 0.22 mmol). The material was purified by flash column chromatography on an ISCO system (10 g silica, elution with a 0-10% MeOH/CH₂Cl₂ gradient) to yield a solid, which was then triturated with MeOH to give the title compound (15 mg, 14%) as a cream solid. ¹H NMR (500 MHZ, DMSO-d₆) δ 10.87 (s, 1H), 10.38 (t, J=5.6 Hz, 1H), 9.41 (s, 1H), 9.23 (t, J=1.3 Hz, 1H), 9.16 (dd, J=5.3, 1.3 Hz, 1H), 8.17-8.08 (m, 2H), 7.68-7.64 (m, 1H), 7.41 (dd, J=7.4, 1.2 Hz, 1H), 7.26-7.20 (m, 2H), 7.12-7.06 (m, 1H), 7.03 (dd, J=5.9, 3.2 Hz, 1H), 5.20 (s, 2H), 3.19-3.11 (m, 2H), 1.49-1.35 (m, 2H), 0.82 (t, J=7.4 Hz, 3H). m/z 448.2 [M+H]⁺.

### Example 107: 4-Amino-8-[2-fluoro-5-(pyrimidin-5-ylmethoxy)phenyl]-2-oxo-N-propyl-1H-quinoline-3-carboxamide

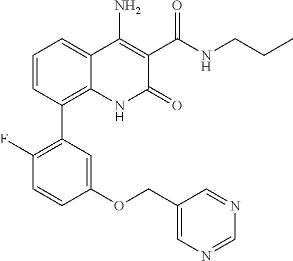 [SMILES: CCCNC(=O)c1c(N)c2cccc(-c3cc(OCc4cncnc4)ccc3F)c2[nH]c1=O]

### Step 1

General procedure 7 was followed using 3-bromo-4-fluorophenol (100 mg, 0.52 mmol) and 5-(chloromethyl) pyrimidine hydrochloride (95 mg, 0.58 mmol). The material was purified by flash column chromatography on an ISCO system (10 g silica, elution with a 0-67% EtOAc/pet. ether gradient) to give 5-[(3-bromo-4-fluoro-phenoxy)methyl] pyrimidine (101 mg, 64%) as a colourless solid. ¹H NMR (500 MHZ, Chloroform-d) δ 9.23 (s, 1H), 8.82 (s, 2H), 7.18 (dd, J=5.5, 3.0 Hz, 1H), 7.08 (dd, J=9.0, 7.9 Hz, 1H), 6.89 (ddd, J=9.1, 3.7, 3.0 Hz, 1H), 5.04 (s, 2H). m/z 283.0 [M+H]⁺.

### Step 2

General procedure 2 was followed using intermediate 4 (105 mg, 0.20 mmol) and 5-[(3-bromo-4-fluoro-phenoxy)methyl] pyrimidine (70 mg, 0.25 mmol). The material was purified by flash column chromatography on an ISCO system (10 g silica, elution with a 0-100% EtOAc/pet. ether gradient) to yield a solid, which was then triturated with Et₂O to give the title compound (71 mg, 77%) as a cream solid. ¹H NMR (500 MHZ, Chloroform-d) δ 11.32 (s, 1H), 10.23 (t, J=5.7 Hz, 1H), 9.23 (s, 1H), 8.84 (s, 2H), 7.99 (s, 1H), 7.68 (d, J=8.0 Hz, 1H), 7.49 (dd, J=7.3, 1.2 Hz, 1H), 7.31 (dd, J=8.3, 7.4 Hz, 1H), 7.22 (t, J=8.9 Hz, 1H), 7.11-7.05 (m, 1H), 6.94 (dd, J=5.7, 3.1 Hz, 1H), 5.92 (s, 1H), 5.08 (s, 2H), 3.44-3.27 (m, 2H), 1.64 (sext, J=7.3 Hz, 2H), 0.98 (t, J=7.4 Hz, 3H). m/z 448.2 [M+H]⁺.

### Example 108: 4-Amino-8-[2-fluoro-5-(3-pyridylmethoxy)phenyl]-2-oxo-N-propyl-1H-quinoline-3-carboxamide

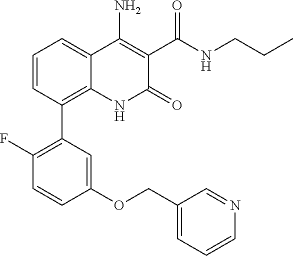 [SMILES: CCCNC(=O)c1c(N)c2cccc(-c3cc(OCc4cccnc4)ccc3F)c2[nH]c1=O]

### Step 1

General procedure 7 was followed using 3-bromo-4-fluorophenol (100 mg, 0.52 mmol) and 3-(chloromethyl)pyridine hydrochloride (94 mg, 0.58 mmol) in MeCN. The material was purified by flash column chromatography on an ISCO system (10 g silica, elution with a 0-67% EtOAc/pet. ether gradient) to give 3-[(3-bromo-4-fluoro-phenoxy)methyl]pyridine (72 mg, 44%) as a colourless oil. ¹H NMR (500 MHZ, Chloroform-d) δ 8.67 (s, 1H), 8.61 (d, J=4.8 Hz, 1H,), 7.80-7.70 (m, 1H), 7.34 (dd, J=7.6, 4.9 Hz, 1H), 7.19-7.14 (m, 1H), 7.09-7.02 (m, 1H), 6.91-6.83 (m, 1H), 5.03 (s, 2H).

### Step 2

General procedure 2 was followed using intermediate 4 (135 mg, 0.25 mmol) and 3-[(3-bromo-4-fluoro-phenoxy)methyl]pyridine (90 mg, 0.32 mmol). The material was purified by flash column chromatography on an ISCO system (10 g silica, elution with a 0-67% EtOAc/pet. ether gradient) to yield a solid, which was then triturated with EtOAc to give the title compound (35 mg, 29%) as a cream solid. ¹H NMR (500 MHZ, Chloroform-d) δ 11.31 (s, 1H), 10.25 (t, J=6.0 Hz, 1H), 8.69 (s, 1H), 8.85-8.57 (m, 1H), 7.96 (s, 1H), 7.79 (d, J=7.8 Hz, 1H), 7.68 (d, J=8.3 Hz, 1H), 7.49 (d, J=7.3 Hz, 1H), 7.35 (t, J=6.5 Hz, 1H), 7.30 (t, J=7.8 Hz, 1H), 7.19 (t, J=8.8 Hz, 1H), 7.11-7.03 (m, 1H), 6.96-6.88 (m, 1H), 5.91 (s, 1H), 5.08 (s, 2H), 3.37 (, q, J=6.8 Hz, 2H), 1.69-1.62 (m, 2H), 1.05-0.96 (m, 3H). m/z 447.2 [M+H]⁺.

### Example 109: 4-Amino-8-[5-[(2-cyano-3-pyridyl)methoxy]-2-fluoro-phenyl]-2-oxo-N-propyl-1H-quinoline-3-carboxamide

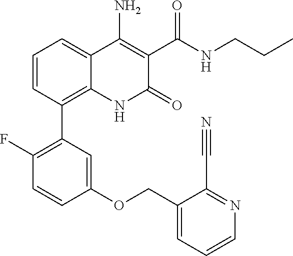 [SMILES: CCCNC(=O)c1c(N)c2cccc(-c3cc(OCc4cccnc4C#N)ccc3F)c2[nH]c1=O]

### Step 1

General procedure 7 was followed using 3-bromo-4-fluorophenol (100 mg, 0.53 mmol) and 3-(bromomethyl)pyridine-2-carbonitrile (113 mg, 0.58 mmol) in MeCN to give 3-[(3-bromo-4-fluoro-phenoxy)methyl]pyridine-2-carbonitrile (72 mg, 44%) as a cream solid. ¹H NMR (500 MHZ, Chloroform-d) δ 8.70 (dd, J=4.7, 1.6 Hz, 1H), 8.03 (ddt, J=8.1, 1.7, 0.8 Hz, 1H), 7.59 (dd, J=8.0, 4.7 Hz, 1H), 7.21 (dd, J=5.5, 3.0 Hz, 1H), 7.08 (dd, J=9.0, 7.9 Hz, 1H), 6.92 (ddd, J=9.0, 3.7, 3.0 Hz, 1H), 5.24 (s, 2H). m/z 307.0 [M+H]⁺.

### Step 2

General procedure 2 was followed using intermediate 4 (143 mg, 0.27 mmol) and 3-[(3-bromo-4-fluoro-phenoxy)methyl]pyridine-2-carbonitrile (104 mg, 0.34 mmol). The material was purified by flash column chromatography on an ISCO system (10 g silica, elution with a 0-80% EtOAc/pet. ether gradient) to yield a solid, which was then triturated with EtOAc to give the title compound (54 mg, 40%) as a colourless solid. ¹H NMR (500 MHz, Chloroform-d): δ 11.31 (s, 1H), 10.24 (t, J=5.9 Hz, 1H), 8.71 (d, J=4.7 Hz, 1H), 8.07 (d, J=8.1 Hz, 1H,), 7.94 (s, 1H), 7.69 (d, J=8.3 Hz, 1H), 7.60 (dd, J=8.1, 4.8 Hz, 1H), 7.51 (d, J=7.4 Hz, 1H), 7.32 (t, J=7.9 Hz, 1H), 7.23 (t, J=8.9 Hz, 1H), 7.14-7.07 (m, 1H), 7.01-6.93 (m, 1H), 5.91 (s, 1H), 5.29 (s, 2H), 3.37 (q, J=6.5 Hz, 2H), 1.63 (sext, J=7.8 Hz, 2H), 0.99 (t, J=7.4 Hz, 3H). m/z 472.2 [M+H]⁺.

### Example 110: 4-Amino-8-[2-fluoro-5-[(6-methyl-3-pyridyl)methoxy]phenyl]-2-oxo-N-propyl-1H-quinoline-3-carboxamide

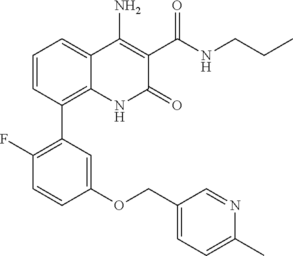 [SMILES: CCCNC(=O)c1c(N)c2cccc(-c3cc(OCc4ccc(C)nc4)ccc3F)c2[nH]c1=O]

### Step 1

General procedure 7 was followed using 3-bromo-4-fluorophenol (422 mg, 2.21 mmol) and 5-(bromomethyl)-2-methylpyridine hydrobromide (649 mg, 2.43 mmol) in MeCN to give 5-[(3-bromo-4-fluoro-phenoxy)methyl]-2-methyl-pyridine (368 mg, 49%) as a colourless solid. ¹H NMR (400 MHZ, Chloroform-d) δ 8.53 (s, 1H), 7.63 (d, J=7.0 Hz, 1H), 7.19 (d, J=8.0 Hz, 1H), 7.15 (dd, J=4.8, 3.1 Hz, 1H), 7.04 (t, J=8.5 Hz, 1H), 6.86 (dt, J=8.7, 3.4 Hz, 1H), 4.98 (s, 2H), 2.58 (s, 3H). m/z 298.0 [M+H]⁺.

### Step 2

General procedure 1 was followed using intermediate 3 (439 mg, 1.18 mmol) and 5-[(3-bromo-4-fluoro-phenoxy)methyl]-2-methyl-pyridine (368 mg, 1.24 mmol). The reaction mixture was concentrated under reduced pressure and EtOAc was added until a grey solid precipitated out from the mixture. The solids were collected via vacuum filtration and the material was purified by flash column chromatography on an ISCO system (10 g silica, elution with a 0-10% MeOH/EtOAc gradient) to give the title compound (286 mg, 50%) as a colourless solid. ¹H NMR (500 MHZ, DMSO-d₆) δ 10.95 (s, 1H), 10.45 (t, J=5.6 Hz, 1H), 9.39 (s, 1H), 8.53 (dd, J=2.3, 0.8 Hz, 1H), 8.32-8.10 (m, 2H), 7.75 (dd, J=7.9, 2.3 Hz, 1H), 7.50 (dd, J=7.4, 1.2 Hz, 1H), 7.34-7.24 (m, 3H), 7.14 (ddd, J=9.0, 4.0, 3.2 Hz, 1H), 7.07 (dd, J=6.0, 3.1 Hz, 1H), 5.12 (s, 2H), 3.23 (td, J=6.9, 5.6 Hz, 2H), 2.47 (s, 3H), 1.50 (app. sextet, J=7.3 Hz, 2H), 0.90 (t, J=7.4 Hz, 3H). m/z 461.2 [M+H]⁺.

### Example 111: 4-Amino-2-oxo-N-propyl-8-(6-(pyridin-3-ylmethoxy) pyridin-2-yl)-1,2-dihydroquinoline-3-carboxamide

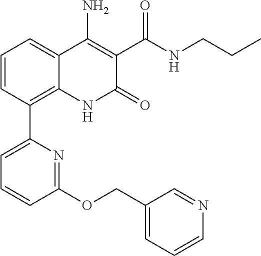 [SMILES: CCCNC(=O)c1c(N)c2cccc(-c3cccc(OCc4cccnc4)n3)c2[nH]c1=O]

### Step 1

General procedure 4 was followed using 6-bromopyridin-2-ol (174 mg, 1.00 mmol) and 3-pyridinemethanol (0.13 mL, 1.30 mmol). The concentrated residue was partitioned between 1 M HCl (10 mL) and EtOAc (10 mL) and the layers separated. The aqueous layer was washed with EtOAc, basified with 2 M NaOH to pH 8-9 and extracted with EtOAc (3×10 mL). The combined organic extracts were dried over MgSO₄, filtered and concentrated under reduced pressure. The material was purified by flash column chromatography on an ISCO system (10 g silica, elution with a 0-100% EtOAc/pet. ether gradient) to give 2-bromo-6-(3-pyridylmethoxy) pyridine (170 mg, 61%) as a colourless oil. ¹H NMR (500 MHZ, Chloroform-d) δ 8.72 (s, 1H), 8.58 (d, J=4.5 Hz, 1H), 7.80 (d, J=7.7 Hz, 1H), 7.44 (t, J=7.8 Hz, 1H), 7.31 (dd, J=7.8, 4.9 Hz, 1H), 7.09 (d, J=7.4 Hz, 1H), 6.74 (d, J=8.1 Hz, 1H), 5.39 (s, 2H). m/z 266.9 [M+H]⁺.

### Step 2

General procedure 1 was followed using intermediate 4 (100 mg, 0.27 mmol) and 2-bromo-6-(3-pyridylmethoxy) pyridine (75 mg, 0.28 mmol). The precipitate from the reaction mixture was collected by vacuum filtration and washed with water and EtOAc to give the title compound (65 mg, 53%) as a grey solid. ¹H NMR (500 MHZ, DMSO-d₆) δ 12.81 (s, 1H), 10.97 (s, 1H), 10.43 (s, 1H), 8.76 (s, 1H), 8.57 (s, 1H), 8.26 (d, J=7.4 Hz, 3H), 7.97 (t, J=7.9 Hz, 2H), 7.79 (d, J=7.3 Hz, 1H), 7.45 (s, 1H), 7.35 (t, J=7.5 Hz, 1H), 7.02 (d, J=8.0 Hz, 1H), 5.53 (s, 2H), 3.23 (q, J=7.0 Hz, 2H), 1.51 (app. sextet, J=6.8, 6.1 Hz, 2H), 0.89 (t, J=7.5 Hz, 3H). m/z 430.1 [M+H]⁺.

### Example 112: 4-Amino-8-[2-fluoro-5-[(6-methyl-3-pyridyl)oxymethyl]phenyl]-2-oxo-N-propyl-1H-quinoline-3-carboxamide

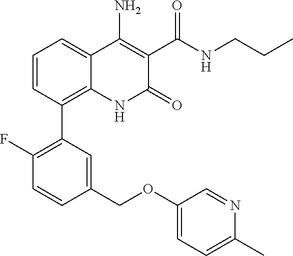 [SMILES: CCCNC(=O)c1c(N)c2cccc(-c3cc(COc4ccc(C)nc4)ccc3F)c2[nH]c1=O]

### Step 1

General procedure 4 was followed using 5-hydroxy-2-methylpyridine (150 mg, 1.37 mmol) and 3-bromo-4-fluorobenzyl alcohol (352 mg, 1.71 mmol) The material was purified by reverse phase chromatography on an ISCO system (12 g RediSep® Rf Reversed-phase C18, elution with a 10-100% MeOH/water gradient) to give 5-[(3-bromo-4-fluoro-phenyl)methoxy]-2-methyl-pyridine (284 mg, 63%) as a yellow oil. ¹H NMR (500 MHz, Chloroform-d) δ 8.17 (s, 1H), 7.64-7.57 (m, 1H), 7.34-7.26 (m, 1H), 7.20-7.04 (m, 3H), 4.97 (s, 2H), 2.44 (s, 3H). m/z 298.0 [M+H]⁺.

### Step 2

General procedure 1 was followed using intermediate 4 (250 mg, 0.67 mmol) and 5-[(3-bromo-4-fluoro-phenyl)methoxy]-2-methyl-pyridine (244 mg, 0.74 mmol). The material was purified by flash column chromatography on an ISCO system (10 g silica, elution with a 0-100% EtOAc/pet. ether gradient) to yield a solid, which was then triturated with EtOAc to give the title compound (133 mg, 41%) as an off-white solid. 1H NMR (500 MHZ, DMSO-d₆) δ 10.96 (s, 1H), 10.50-10.44 (m, 1H), 9.56 (s, 1H), 8.26-8.20 (m, 2H), 7.64-7.56 (m, 1H), 7.54-7.48 (m, 2H), 7.42-7.34 (m, 2H), 7.37-7.29 (m, 1H), 7.19 (d, J=8.5 Hz, 1H), 5.18 (s, 2H), 3.27-3.20 (m, 2H), 2.40 (s, 3H), 1.51 (m, 2H), 0.91 (t, J=7.4 Hz, 3H). m/z 461.2 [M+H]⁺.

### Example 113: 4-Amino-8-[2-fluoro-5-(4-pyridylmethoxy)phenyl]-2-oxo-N-propyl-1H-quinoline-3-carboxamide

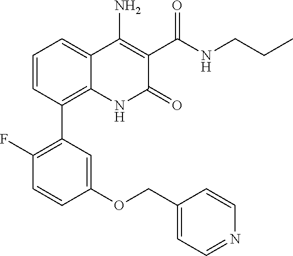 [SMILES: CCCNC(=O)c1c(N)c2cccc(-c3cc(OCc4ccncc4)ccc3F)c2[nH]c1=O]

### Step 1

General procedure 7 was followed using 3-bromo-4-fluorophenol (150 mg, 0.79 mmol) and 4-(chloromethyl)pyridine hydrochloride (86 mg, 0.52 mmol) in MeCN to give 4-[(3-bromo-4-fluoro-phenoxy)methyl]pyridine (105 mg, 68%) as a colourless solid. ¹H NMR (500 MHZ, Chloroform-d) δ 8.68-8.58 (m, 2H), 7.36-7.29 (m, 2H), 7.15 (dd, J=5.5, 3.0 Hz, 1H), 7.05 (dd, J=9.0, 8.0 Hz, 1H), 6.86 (ddd, J=9.0, 3.7, 3.0 Hz, 1H), 5.04 (s, 2H). m/z 282.0 [M+H]⁺.

### Step 2

General procedure 2 was followed using intermediate 4 (143 mg, 0.27 mmol) and 4-[(3-bromo-4-fluoro-phenoxy)methyl]pyridine (95 mg, 0.34 mmol). The material was purified by flash column chromatography on an ISCO system (10 g silica, elution with a 0-100% EtOAc/pet. ether gradient) to yield a solid, which was then triturated with 3:1 Et₂O:MeOH to give the title compound (20 mg, 16%) as a cream solid. ¹H NMR (500 MHZ, Chloroform-d) δ 11.30 (s, 1H), 10.24 (t, J=5.6 Hz, 1H), 8.63 (d, J=5.0 Hz, 2H), 8.03 (s, 1H), 7.68 (d, J=8.3 Hz, 1H), 7.48 (d, J=7.3 Hz, 1H), 7.35 (d, J=5.0 Hz, 2H), 7.30 (t, J=7.9 Hz, 1H), 7.18 (t, J=8.8 Hz, 1H), 7.03 (dt, J=8.7, 3.6 Hz, 1H), 6.91 (dd, J=5.8, 3.1 Hz, 1H), 5.94 (s, 1H), 5.09 (s, 2H), 3.36 (q, J=6.7 Hz, 2H), 1.63 (sext, J=7.4 Hz, 2H), 0.98 (t, J=7.4 Hz, 3H). m/z 447.2 [M+H]⁺.

### Example 114: 4-Amino-8-[5-[(2-cyano-4-fluoro-phenyl)methoxy]-2-fluoro-phenyl]-2-oxo-N-propyl-1H-quinoline-3-carboxamide

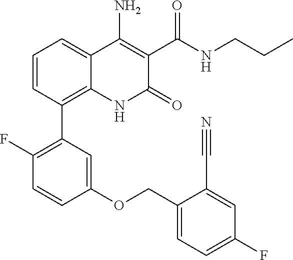 [SMILES: CCCNC(=O)c1c(N)c2cccc(-c3cc(OCc4ccc(F)cc4C#N)ccc3F)c2[nH]c1=O]

### Step 1

General procedure 7 was followed using 3-bromo-4-fluorophenol (200 mg, 1.05 mmol) and 2-(bromomethyl)-5-fluorobenzonitrile (246 mg, 1.15 mmol) in acetone to give 2-[(3-bromo-4-fluoro-phenoxy)methyl]-5-fluoro-benzonitrile (365 mg, 100%) as a yellow solid. ¹H NMR (500 MHZ, DMSO-d₆) δ 7.95 (dd, J=8.6, 2.8 Hz, 1H), 7.85-7.76 (m, 1H), 7.66 (t, J=8.8 Hz, 1H), 7.48-7.42 (m, 1H), 7.35 (td, J=8.8, 2.2 Hz, 1H), 7.17-7.05 (m, 1H), 5.24 (s, 2H). m/z 323.9 [M+H]⁺.

### Step 2

General procedure 1 was followed using intermediate 4 (100 mg, 0.27 mmol) and 2-[(3-bromo-4-fluoro-phenoxy)methyl]-5-fluoro-benzonitrile (113 mg, 0.32 mmol). The material was purified by reverse phase chromatography on an ISCO system (12 g RediSep® Rf Reversed-phase C18, elution with a 10-100% MeOH/water gradient) to give the title compound (26 mg, 19%) as a colourless solid. ¹H NMR (500 MHZ, Chloroform-d) δ 11.32 (s, 1H), 10.27 (d, J=6.0 Hz, 1H), 8.02 (s, 1H), 7.70 (d, J=8.5 Hz, 2H), 7.52 (d, J=7.4 Hz, 1H), 7.44 (d, J=7.9 Hz, 1H), 7.40 (t, J=8.5 Hz, 1H), 7.36-7.30 (m, 1H), 7.26-7.19 (m, 1H), 7.10 (dd, J=9.2, 3.4 Hz, 1H), 6.98 (dd, J=5.9, 2.9 Hz, 1H), 5.97 (s, 1H), 5.22 (s, 2H), 3.38 (q, J=6.8 Hz, 2H), 1.74-1.47 (m, 2H), 1.01 (td, J=7.5, 2.1 Hz, 3H). m/z 489.1 [M+H]⁺.

### Example 115: 4-Amino-8-[5-[(4-cyano-2-methoxy-phenyl)methoxy]-2-fluoro-phenyl]-2-oxo-N-propyl-1H-quinoline-3-carboxamide

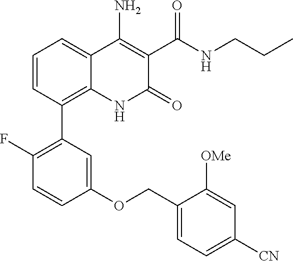 [SMILES: CCCNC(=O)c1c(N)c2cccc(-c3cc(OCc4ccc(C#N)cc4OC)ccc3F)c2[nH]c1=O]

### Step 1

General procedure 7 was followed using 3-bromo-4-fluorophenol (100 mg, 0.52 mmol) and 4-(bromomethyl)-3-methoxybenzonitrile (142 mg, 0.63 mmol) in MeCN to give 4-[(3-bromo-4-fluoro-phenoxy)methyl]-3-methoxy-benzonitrile (195 mg, 100%) as an orange solid. ¹H NMR (400 MHZ, Chloroform-d) δ 7.55 (d, J=7.8 Hz, 1H), 7.31 (d, J=7.8 Hz, 1H), 7.16 (dd, J=5.5, 3.0 Hz, 1H), 7.13 (s, 1H), 7.05 (dd, J=9.1, 8.0 Hz, 1H), 6.87 (dt, J=9.1, 3.5 Hz, 1H), 5.07 (s, 2H), 3.91 (s, 3H). m/z 335.9 [M−H]⁻.

### Step 2

General procedure 1 was followed using intermediate 4 (100 mg, 0.27 mmol) and 4-[(3-bromo-4-fluoro-phenoxy)methyl]-3-methoxy-benzonitrile (106 mg, 0.28 mmol). The material was purified by reverse phase chromatography on an ISCO system (12 g RediSep® Rf Reversed-phase C18, elution with a 10-100% MeOH/water gradient), followed by flash column chromatography on an ISCO system (10 g silica, elution with a 50-100% EtOAc/pet. ether gradient) to give the title compound (5.9 mg, 4%) as a colourless solid. ¹H NMR (500 MHZ, DMSO-d₆) δ 10.95 (s, 1H), 10.46 (t, J=5.6 Hz, 1H), 9.41 (s, 1H), 8.32-8.10 (m, 2H), 7.63 (d, J=7.8 Hz, 1H), 7.53 (d, J=1.5 Hz, 1H), 7.50 (dd, J=7.4, 1.2 Hz, 1H), 7.47 (dd, J=7.7, 1.5 Hz, 1H), 7.33-7.26 (m, 2H), 7.13 (ddd, J=9.1, 4.0, 3.3 Hz, 1H), 7.06 (dd, J=6.0, 3.1 Hz, 1H), 5.14 (s, 2H), 3.88 (s, 2H), 3.23 (td, J=6.8, 5.6 Hz, 3H), 1.51 (app. sextet, J=7.3 Hz, 2H), 0.90 (t, J=7.4 Hz, 3H). m/z 501.2 [M+H]⁺.

### Example 116: 4-Amino-8-(2-fluoro-5-(furan-3-ylmethoxy)phenyl)-2-oxo-N-propyl-1,2-dihydroquinoline-3-carboxamide

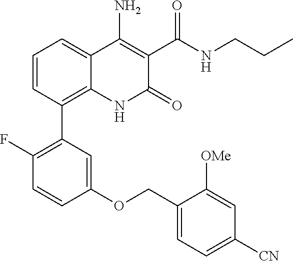 [SMILES: CCCNC(=O)c1c(N)c2cccc(-c3cc(OCc4ccc(C#N)cc4OC)ccc3F)c2[nH]c1=O]

### Step 1

General procedure 4 was followed using 3-bromo-4-fluorophenol (200 mg, 1.05 mmol) and 3-furanmethanol (134 mg, 1.36 mmol). The material was purified by flash column chromatography on an ISCO system (10 g silica, elution with a 0-100% EtOAc/pet. ether gradient) to give 3-[(3-bromo-4-fluoro-phenoxy)methyl] furan (198 mg, 66%) as a colourless oil. ¹H NMR (400 MHZ, Chloroform-d) δ 7.52 (s, 1H), 7.47 (s, 1H), 7.16 (dd, J=5.6, 3.0 Hz, 1H), 7.06 (app t, J=8.5 Hz, 1H), 6.92-6.83 (m, 1H), 6.49 (s, 1H), 4.91 (s, 2H).

### Step 2

General procedure 1 was followed using intermediate 4 (112 mg, 0.30 mmol) and 3-[(3-bromo-4-fluoro-phenoxy)methyl]furan (98 mg, 0.36 mmol). The material was purified by flash column chromatography on an ISCO system (10 g silica, elution with a 0-100% EtOAc/pet. ether gradient) to give the title compound (62 mg, 45%) as a colourless solid. ¹H NMR (400 MHZ, Methanol-d₄) δ 8.13 (d, J=8.3 Hz, 1H), 7.61 (s, 1H), 7.57 (d, J=7.3 Hz, 1H), 7.51 (s, 1H), 7.38 (app t, J=7.8 Hz, 1H), 7.25 (t, J=9.1 Hz, 1H), 7.16 (dt, J=8.8, 3.7 Hz, 1H), 7.07-7.00 (m, 1H), 6.54 (s, 1H), 5.01 (s, 2H), 3.38-3.34 (m, 2H), 1.63 (m, 2H), 1.01 (t, J=7.4 Hz, 3H). m/z 436.3 [M+H]⁺.

### Example 117: 4-Amino-8-[2-fluoro-5-[(4-methylthiazol-5-yl)methoxy]phenyl]-2-oxo-N-propyl-1H-quinoline-3-carboxamide

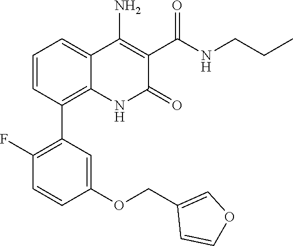 [SMILES: CCCNC(=O)c1c(N)c2cccc(-c3cc(OCc4ccoc4)ccc3F)c2[nH]c1=O]

### Step 1

General procedure 7 was followed using 3-bromo-4-fluorophenol (170 mg, 0.89 mmol) and (4-methylthiazol-5-yl) methanol (150 mg, 1.16 mmol) in MeCN. The material was purified by reverse phase chromatography on an ISCO system (12 g RediSep® Rf Reversed-phase C18, elution with a 10-100% MeOH/water gradient) to give 5-[(3-bromo-4-fluoro-phenoxy)methyl]-4-methyl-thiazole (140 mg, 44%) as an orange solid. ¹H NMR (400 MHZ, Chloroform-d) δ 8.75 (s, 1H), 7.17 (dd, J=5.5, 3.0 Hz, 1H), 7.08 (dd, J=9.0, 8.0 Hz, 1H), 6.95-6.79 (m, 1H), 5.16 (s, 2H), 2.51 (s, 3H). m/z 303.9 [M+H] *.

### Step 2

General procedure 1 was followed using intermediate 4 (100 mg, 0.27 mmol) and 5-[(3-bromo-4-fluoro-phenoxy)methyl]-4-methyl-thiazole (81 mg, 0.27 mmol). The material was purified by flash column chromatography on an ISCO system (10 g silica, elution with a 0-10% MeOH/CH₂Cl₂ gradient), followed by reverse phase chromatography on an ISCO system (12 g RediSep® Rf Reversed-phase C18, elution with a 10-100% MeOH/water gradient) to give the title compound (39 mg, 30%) as an orange solid. 1H NMR (500 MHZ, DMSO-d₆) δ 10.96 (s, 1H), 10.49-10.42 (m, 1H), 9.41 (s, 1H), 9.01 (d, J=2.2 Hz, 1H), 8.27-8.18 (m, 2H), 7.52 (d, J=7.3, 1.3 Hz, 1H), 7.37-7.24 (m, 2H), 7.19-7.07 (m, 2H), 5.31 (s, 2H), 3.24 (q, J=6.4 Hz, 2H), 2.41 (s, 3H), 1.59-1.43 (m, 2H), 0.91 (t, J=7.4 Hz, 3H). m/z 467.2 [M+H]⁺.

### Example 118: 4-Amino-8-[2-fluoro-5-[(4-methylthiazol-2-yl)methoxy]phenyl]-2-oxo-N-propyl-1H-quinoline-3-carboxamide

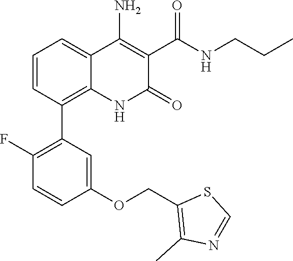 [SMILES: CCCNC(=O)c1c(N)c2cccc(-c3cc(OCc4scnc4C)ccc3F)c2[nH]c1=O]

### Step 1

General procedure 7 was followed using 3-bromo-4-fluorophenol (300 mg, 1.57 mmol) and (4-methyl-1,3-thiazol-2-yl) methanol (240 mg, 1.86 mmol) in acetone to give 2-[(3-bromo-4-fluoro-phenoxy)methyl]-4-methyl-thiazole (413 mg, 70%) as a brown oil. ¹H NMR (400 MHZ, Chloroform-d) δ 7.12 (dd, J=5.9, 3.1 Hz, 1H), 6.99-6.93 (m, 1H), 6.88 (d, J=10.9 Hz, 2H), 5.20 (s, 2H), 2.40 (s, 3H). m/z 304.0 [M+H]⁺.

### Step 2

General procedure 1 was followed using intermediate 4 (100 mg, 0.27 mmol) and 2-[(3-bromo-4-fluoro-phenoxy)methyl]-4-methyl-thiazole (112 mg, 0.30 mmol). The material was purified by reverse phase chromatography on an ISCO system (12 g RediSep® Rf Reversed-phase C18, elution with a 10-100% MeOH/water gradient) to give the title compound (39 mg, 29%) as a yellow solid. ¹H NMR (400 MHZ, Chloroform-d) δ 10.28 (t, J=5.7 Hz, 1H), 7.95 (s, 1H), 7.69 (d, J=8.2 Hz, 1H), 7.50 (d, J=7.4 Hz, 1H), 7.36-7.29 (m, 1H), 7.20 (dd, J=9.8, 7.9 Hz, 1H), 7.15-7.08 (m, 1H), 7.02-6.91 (m, 2H), 5.34 (dd, J=16.5, 2.0 Hz, 2H), 3.42-3.36 (m, 2H), 2.48 (d, J=1.8 Hz, 3H), 1.73-1.61 (m, 2H), 1.01 (t, J=7.4, Hz, 3H). m/z 467.2 [M+H]⁺.

### Example 119: 4-Amino-8-(2-fluoro-5-(thiazol-4-ylmethoxy)phenyl)-2-oxo-N-propyl-1,2-dihydroquinoline-3-carboxamide

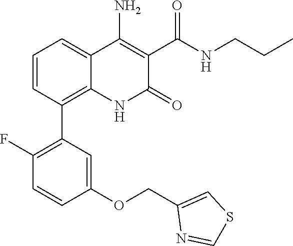 [SMILES: CCCNC(=O)c1c(N)c2cccc(-c3cc(OCc4cscn4)ccc3F)c2[nH]c1=O]

### Step 1

General procedure 4 was followed using 3-bromo-4-fluorophenol (100 mg, 0.52 mmol) and 4-hydroxymethylthiazole (90 mg, 0.78 mmol). The material was purified by flash column chromatography on an ISCO system (10 g silica, elution with a 0-40% EtOAc/pet. ether gradient) to give 4-[(3-bromo-4-fluoro-phenoxy)methyl] thiazole (120 mg, 75% yield) as a colourless solid. ¹H NMR (600 MHZ, Chloroform-d) δ 8.84 (s, 1H), 7.39 (s, 1H), 7.23-7.13 (m, 1H), 7.04 (t, J=8.6, 2.6 Hz, 1H), 6.99-6.84 (m, 1H), 5.22 (s, 2H). m/z 287.8, 289.7 [M+H]⁺.

### Step 2

General procedure 8 was followed using intermediate 4 (40 mg, 0.11 mmol), 4-[(3-bromo-4-fluoro-phenoxy)methyl] thiazole (24 mg, 0.08 mmol). The material was purified by flash column chromatography on an ISCO system (10 g silica, elution with a 0-10% MeOH/CH₂Cl₂ gradient) and further purification by flash column chromatography on an ISCO system (10 g silica, elution with a 0-10% MeOH/EtOAc gradient), followed by trituration with Et₂O to give the title compound (14 mg, 24%) as a colourless solid. ¹H NMR (600 MHz, Chloroform-d) δ 11.30 (s, 1H), 10.28 (s, 1H), 8.86 (s, 1H), 7.96 (s, 1H), 7.67 (d, J=8.3 Hz, 1H), 7.49 (d, J=7.3 Hz, 1H), 7.42 (s, 1H), 7.30 (td, J=8.0, 2.6 Hz, 1H), 7.18 (td, J=9.1, 2.6 Hz, 1H), 7.12-7.06 (m, 1H), 6.99-6.94 (m, 1H), 5.89 (s, 1H), 5.27 (s, 2H), 3.37 (q, J=7.1 Hz, 2H), 1.70-1.58 (m, 2H), 0.99 (t, J=7.5, 2.6 Hz, 3H). m/z 453.0 [M+H]⁺.

### Example 120: 4-Amino-8-(2-fluoro-5-((2-methylthiazol-4-yl)methoxy)phenyl)-2-oxo-N-propyl-1,2-dihydroquinoline-3-carboxamide

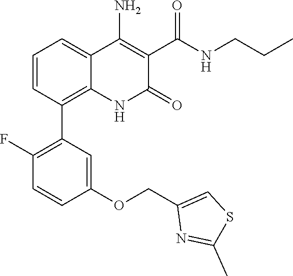 [SMILES: CCCNC(=O)c1c(N)c2cccc(-c3cc(OCc4csc(C)n4)ccc3F)c2[nH]c1=O]

### Step 1

General procedure 7 was followed using (2-methyl-1,3-thiazol-4-yl) methanol (254 mg, 1.96 mmol) and 3-bromo-4-fluorophenol (300 mg, 1.57 mmol) in MeCN. The material was purified by reverse phase chromatography on an ISCO system (12 g RediSep® Rf Reversed-phase C18, elution with a 10-100% MeOH/water gradient) to give 4-[(3-bromo-4-fluoro-phenoxy)methyl]-2-methyl-thiazole (271 mg, 49%) as a yellow oil. ¹H NMR (400 MHz, Chloroform-d) δ 7.18-7.11 (m, 2H), 7.05-6.96 (m, 1H), 6.91-6.82 (m, 1H), 5.06 (s, 2H), 2.71 (s, 3H). m/z 303.9 [M+H]⁺.

### Step 2

General procedure 1 was followed using intermediate 4 (270 mg, 0.73 mmol) and 4-[(3-bromo-4-fluoro-phenoxy)methyl]-2-methyl-thiazole (258 mg, 0.73 mmol). The material was purified by flash column chromatography on an ISCO system (10 g silica, elution with a 0-100% EtOAc/pet. ether gradient) to give the title compound (161 mg, 46%) as a yellow solid. ¹H NMR (500 MHZ, DMSO-d₆) δ 10.96 (s, 1H), 10.47 (t, J=5.5 Hz, 1H), 9.39 (s, 1H), 8.25-8.19 (m, 2H), 7.57 (d, J=0.7 Hz, 1H), 7.52 (s, 1H), 7.36-7.24 (m, 2H), 7.19-7.12 (m, 1H), 7.08 (dd, J=6.0, 3.1 Hz, 1H), 5.13 (s, 2H), 3.28-3.20 (m, 2H), 2.66 (s, 3H), 1.57-1.50 (m, 2H), 0.91 (t, J=7.4 Hz, 3H). m/z 467.2 [M+H]⁺.

### Example 121: 4-Amino-8-[2-fluoro-5-(oxazol-4-ylmethoxy)phenyl]-2-oxo-N-propyl-1H-quinoline-3-carboxamide

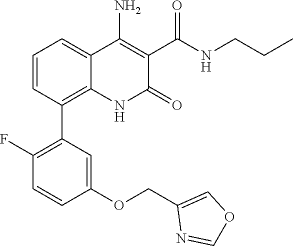 [SMILES: CCCNC(=O)c1c(N)c2cccc(-c3cc(OCc4cocn4)ccc3F)c2[nH]c1=O]

### Step 1

General procedure 7 was followed using 3-bromo-4-fluorophenol (256 mg, 1.34 mmol) and 1,3-oxazol-4-ylmethanol (173 mg, 1.75 mmol) in MeCN to give 4-[(3-bromo-4-fluoro-phenoxy)methyl] oxazole (423 mg, 99%) as a yellow solid. ¹H NMR (500 MHZ, Chloroform-d) δ 7.91 (s, 1H), 7.72 (s, 1H), 7.19-7.15 (m, 1H), 7.04 (ddt, J=9.6, 7.8, 1.1 Hz, 1H), 6.92-6.86 (m, 1H), 4.99 (s, 2H). m/z 274.0 [M+H]⁺.

### Step 2

General procedure 1 was followed using intermediate 3 (364 mg, 1.26 mmol) and 4-[(3-bromo-4-fluoro-phenoxy)methyl] oxazole (423 mg, 1.32 mmol). The material was purified by flash column chromatography on an ISCO system (10 g silica, elution with a 0-100% EtOAc/pet. ether gradient) to yield a solid, which was then triturated with MeOH to give the title compound (156 mg, 27%) as a grey solid. ¹H NMR (500 MHZ, DMSO-d₆) δ 10.95 (s, 1H), 10.46 (t, J=5.6 Hz, 1H), 9.40 (s, 1H), 8.40 (d, J=1.0 Hz, 1H), 8.25-8.15 (m, 3H), 7.51 (dd, J=7.4, 1.2 Hz, 1H), 7.34-7.24 (m, 2H), 7.14 (ddd, J=9.0, 4.0, 3.1 Hz, 1H), 7.09 (dd, J=6.0, 3.1 Hz, 1H), 5.04 (s, 2H), 3.23 (td, J=6.8, 5.6 Hz, 2H), 1.50 (app. sextet, J=7.3 Hz, 2H), 0.90 (t, J=7.4 Hz, 3H). m/z 437.2 [M+H]⁺.

### Example 122:4-Amino-8-(2-fluoro-5-((2-methyloxazol-4-yl)methoxy)phenyl)-2-oxo-N-propyl-1,2-dihydroquinoline-3-carboxamide

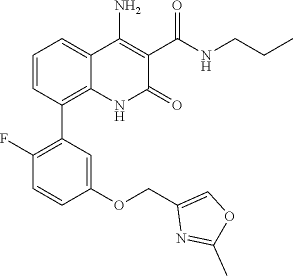 [SMILES: CCCNC(=O)c1c(N)c2cccc(-c3cc(OCc4coc(C)n4)ccc3F)c2[nH]c1=O]

### Step 1

General procedure 7 was followed using (2-methyloxazol-4-yl) methanol (222 mg, 1.96 mmol) and 3-bromo-4-fluorophenol (300 mg, 1.57 mmol) in MeCN. The material was purified by flash column chromatography on an ISCO system (10 g silica, elution with a 0-100% EtOAc/pet. ether gradient) to give 4-[(3-bromo-4-fluoro-phenoxy)methyl]-2-methyl-oxazole (428 mg, 76%) as a yellow glass. ¹H NMR (500 MHZ, Chloroform-d) δ 7.61-7.57 (m, 1H), 7.22-7.16 (m, 1H), 7.09-7.02 (m, 1H), 6.95-6.88 (m, 1H), 4.93 (s, 2H), 2.50 (s, 3H). m/z 288.0 [M+H]⁺.

### Step 2

General procedure 1 was followed using intermediate 3 (375 mg, 1.01 mmol) and 4-[(3-bromo-4-fluoro-phenoxy)methyl]-2-methyl-oxazole (397 mg, 1.11 mmol). The material was purified by flash column chromatography on an ISCO system (10 g silica, elution with a 0-100% EtOAc/pet. ether gradient) to give the title compound (260 mg, 56%) as a cream solid. ¹H NMR (500 MHZ, DMSO-d₆) δ 10.96 (s, 1H), 10.47 (t, J=5.6 Hz, 1H), 9.39 (s, 1H), 8.25-8.19 (m, 2H), 8.06 (s, 1H), 7.52 (dd, J=7.4, 1.2 Hz, 1H), 7.36-7.25 (m, 2H), 7.17-7.10 (m, 1H), 7.09-7.04 (m, 1H), 4.96 (s, 2H), 3.28-3.20 (m, 2H), 2.41 (s, 3H), 1.57-1.46 (m, 2H), 0.90 (t, J=7.4 Hz, 3H). m/z 451.2 [M+H]⁺

### Example 123: 4-Amino-8-[2-fluoro-5-[(2-isopropyloxazol-4-yl)methoxy]phenyl]-2-oxo-N-propyl-1H-quinoline-3-carboxamide

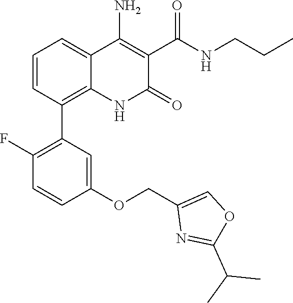 [SMILES: CCCNC(=O)c1c(N)c2cccc(-c3cc(OCc4coc(C(C)C)n4)ccc3F)c2[nH]c1=O]

### Step 1

General procedure 7 was followed using 3-bromo-4-fluorophenol (150 mg, 0.78 mmol) and 4-(chloromethyl)-2-(propan-2-yl)-1,3-oxazole (150 mg, 0.94 mmol) in MeCN to give 4-[(3-bromo-4-fluoro-phenoxy)methyl]-2-isopropyl-oxazole (240 mg, 89%) as a yellow oil. ¹H NMR (400 MHZ, Chloroform-d) δ 7.60 (s, 1H), 7.19 (dd, J=5.5, 3.0 Hz, 1H), 7.05 (t, J=8.5 Hz, 1H), 6.90 (dt, J=9.1, 3.5 Hz, 1H), 4.94 (s, 2H), 3.11 (m, 1H), 1.38 (d, J=6.9 Hz, 6H). m/z 316.0 [M+H]⁺.

### Step 2

General procedure 1 was followed using intermediate 3 (100 mg, 0.35 mmol) and 4-[(3-bromo-4-fluoro-phenoxy)methyl]-2-isopropyl-oxazole (109 mg, 0.35 mmol). The material was purified by flash column chromatography on an ISCO system (10 g silica, elution with a 0-10% MeOH/CH₂Cl₂ gradient), followed by reverse phase chromatography on an ISCO system (12 g RediSep® Rf Reversed-phase C18, elution with a 10-100% MeOH/water gradient) to give the title compound (46 mg, 26%) as a yellow solid. ¹H NMR (400 MHZ, DMSO-d₆) δ 10.96 (s, 1H), 10.47 (t, J=5.6 Hz, 1H), 9.40 (s, 1H), 8.22 (d, J=8.2 Hz, 1H), 8.08 (d, J=1.6 Hz, 1H), 7.52 (dd, J=7.4, 1.2 Hz, 1H), 7.35-7.25 (m, 2H), 7.18-7.12 (m, 1H), 7.08 (dd, J=6.0, 3.1 Hz, 1H), 4.97 (s, 2H), 3.28-3.19 (m, 2H), 3.12-3.02 (m, 1H), 1.57-1.45 (m, 2H), 1.27 (d, J=7.0 Hz, 6H), 0.91 (t, J=7.4 Hz, 3H). m/z 479.2 [M+H]⁺.

### Example 124: 8-(5-((1H-Imidazol-4-yl)methoxy)-2-fluorophenyl)-4-amino-2-oxo-N-propyl-1,2-dihydroquinoline-3-carboxamide

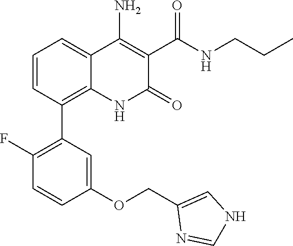 [SMILES: CCCNC(=O)c1c(N)c2cccc(-c3cc(OCc4c[nH]cn4)ccc3F)c2[nH]c1=O]

### Step 1

To 4-imidazolemethanol (500 mg, 5.1 mmol) and trityl chloride (1563 mg, 5.6 mmol) in DMF (3 mL), was added Et₃N (1.78 mL, 12.74 mmol) and the reaction mixture stirred at rt for 16 h. After this time, the reaction mixture was poured into ice-cold water (10 mL) and the solids collected by filtration under vacuum. The filter cake was washed with water and dried under reduced pressure to give (1-tritylimidazol-4-yl) methanol (1.66 g, 86%) as a cream solid. ¹H NMR (500 MHZ, Chloroform-d) δ 7.45 (s, 1H), 7.37-7.35 (m, 9H), 7.17-7.14 (m, 6H), 6.80 (s, 1H), 4.60 (s, 2H). m/z 244.1 (trityl cation), 363.2 [M+Na]⁺.

### Step 2: Synthesis of Intermediate 10: 4-Amino-8-(2-fluoro-5-hydroxy-phenyl)-2-oxo-N-propyl-1H-quinoline-3-carboxamide

General procedure 1 was followed using intermediate 4 (1.00 g, 2.84 mmol) and 3-bromo-4-fluorophenol (598 mg, 3.13 mmol). The precipitate formed was collected by vacuum filtration and washed with EtOAc (3×5 mL) to give intermediate 10 (562 mg, 53%) as a grey solid. ¹H NMR (500 MHZ, DMSO-d₆) δ 10.96 (s, 1H), 10.47-10.39 (m, 1H), 9.17-9.13 (m, 1H), 8.20 (d, J=8.6 Hz, 1H), 7.49 (d, J=7.3 Hz, 1H), 7.31 (t, J=8.1 Hz, 1H), 7.15 (t, J=9.3 Hz, 1H), 6.89-6.83 (m, 1H), 6.72 (d, J=5.9 Hz, 1H), 3.30-3.18 (m, 2H), 1.57-1.45 (m, 2H), 0.91 (t, J=7.7 Hz, 3H). m/z 356.1 [M+H] *.

### Step 3

General procedure 4 was followed using intermediate 10 (200 mg, 0.56 mmol) and (1-tritylimidazol-4-yl) methanol (266 mg, 0.70 mmol). The material was purified by flash column chromatography on an ISCO system (10 g silica, elution with a 0-20% MeOH/CH₂Cl₂ gradient), followed by reverse phase chromatography on an ISCO system (12 g RediSep® Rf Reversed-phase C18, elution with a 10-100% MeOH/water gradient) to give 4-amino-8-[2-fluoro-5-[(1-tritylimidazol-4-yl)methoxy]phenyl]-2-oxo-N-propyl-1H-quinoline-3-carboxamide (245 mg, 58%) as a brown solid. ¹H NMR (500 MHZ, Chloroform-d) δ 11.19 (s, 1H), 10.21 (s, 1H), 7.92 (s, 1H), 7.62-7.56 (m, 1H), 7.40-7.32 (m, 2H), 7.27-7.23 (m, 10H), 7.09-7.04 (m, 7H), 6.99 (s, 1H), 6.87 (dd, J=5.8, 3.0 Hz, 1H), 6.85-6.82 (m, 1H), 5.87 (s, 1H), 4.92 (s, 2H), 3.31-3.27 (m, 2H), 1.55 (h, J=7.3 Hz, 2H), 0.91 (d, J=7.3 Hz, 3H). m/z 436.1 [M (free base)+H]⁺.

### Step 4

To 4-amino-8-[2-fluoro-5-[(1-tritylimidazol-4-yl)methoxy]phenyl]-2-oxo-N-propyl-1H-quinoline-3-carboxamide (245 mg, 0.32 mmol) in MeOH (5 mL) was added 4N HCl in dioxane (20 mL) and the reaction mixture was stirred at 70° C. for 16 h. After this time, the reaction mixture was concentrated under reduced pressure and the residue partitioned between EtOAc (10 mL) and water (10 mL). The aqueous layer was basified to pH 12 using 2 M NaOH, and extracted with EtOAc (3×10 mL). The combined organic extracts were dried over Na₂SO₄, filtered and concentrated under reduced pressure. The material was purified by flash column chromatography on an ISCO system (10 g silica, elution with a 0-20% MeOH/CH₂Cl₂ gradient) to give the title compound (51 mg, 34%) as a pale yellow solid. ¹H NMR (500 MHz, DMSO-d₆) δ 10.96 (s, 1H), 10.46 (s, 1H), 9.34 (s, 1H), 8.46 (s, 1H), 8.25-8.17 (m, 2H), 7.44-7.38 (m, 1H), 7.36-7.30 (m, 1H), 7.24 (s, 1H), 5.38 (t, J=5.0 Hz, 1H), 4.59 (d, J=5.0 Hz, 2H), 3.28-3.21 (m, 2H), 2.01 (s, 3H), 1.50 (h, J=7.3 Hz, 2H), 0.90 (t, J=7.4 Hz, 3H). m/z 436.3 [M+H]⁺.

### Example 125: 4-Amino-8-(2-fluoro-5-((1-methyl-1H-imidazol-5-yl)methoxy)phenyl)-2-oxo-N-propyl-1,2-dihydroquinoline-3-carboxamide

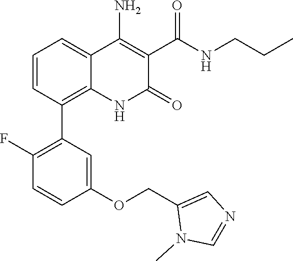 [SMILES: CCCNC(=O)c1c(N)c2cccc(-c3cc(OCc4cncn4C)ccc3F)c2[nH]c1=O]

General procedure 4 was followed using intermediate 10 (100 mg, 0.28 mmol) and (1-methyl-1H-imidazol-5-yl) methanol (41 mg, 0.37 mmol). The material was purified by flash column chromatography on an ISCO system (10 g silica, elution with a 0-100% EtOAc/pet. ether gradient, then a 0-10% MeOH/EtOAc gradient) to give the title compound (38 mg, 28%) as a cream solid. ¹H NMR (500 MHZ, DMSO-d₆) δ 10.95 (s, 1H), 10.46 (t, J=5.6 Hz, 1H), 9.39 (s, 1H), 8.30-8.07 (m, 2H), 7.65 (s, 1H), 7.51 (d, J=7.3 Hz, 1H), 7.35-7.25 (m, 2H), 7.16 (dt, J=9.0, 3.6 Hz, 1H), 7.09 (dd, J=6.0, 3.1 Hz, 1H), 7.02 (s, 1H), 5.11 (s, 2H), 3.65 (s, 3H), 3.23 (q, J=6.5 Hz, 2H), 1.50 (app. sextet, J=7.2 Hz, 2H), 0.90 (t, J=7.4 Hz, 3H). m/z 450.1 [M+H]⁺.

### Example 126: 4-Amino-8-(2-fluoro-5-((1-methyl-1H-imidazol-4-yl)methoxy)phenyl)-N-propyl-1,2-dihydroquinoline-3-carboxamide hydrochloride

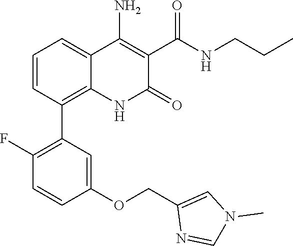 [SMILES: CCCNC(=O)c1c(N)c2cccc(-c3cc(OCc4cn(C)cn4)ccc3F)c2[nH]c1=O]

General procedure 4 was followed using intermediate 10 (60 mg, 0.16 mmol) and (1-methyl-1H-imidazol-4-yl) methanol (22.4 mg, 0.20 mmol). The material was purified by flash column chromatography on an ISCO system (10 g silica, elution with a 0-100% EtOAc/pet. ether gradient) and further purification by reverse phase chromatography on an ISCO system (12 g RediSep® Rf Reversed-phase C18, elution with a 10-100% MeOH/water gradient), followed by reverse phase chromatography on an ISCO ACCQPrep system (20 mm×150 mm C18 column, elution with a 10-100% MeOH/water gradient) to give the title compound (8 mg, 11%) as a yellow solid. ¹H NMR (500 MHZ, Methanol-d₄) δ 8.83 (s, 1H), 8.06-7.98 (m, 1H), 7.57 (s, 1H), 7.49-7.43 (m, 1H), 7.31-7.23 (m, 1H), 7.23-7.16 (m, 1H), 7.13-7.06 (m, 1H), 7.00 (dd, J=5.8, 3.1 Hz, 1H), 5.15-5.11 (m, 2H), 3.85 (s, 3H), 3.21-3.17 (m, 2H), 1.50 (m, 2H), 0.88 (t, J=7.4 Hz, 3H).

### Example 127: 4-Amino-8-[2-fluoro-5-(1H-pyrazol-3-ylmethoxy)phenyl]-2-oxo-N-propyl-1H-quinoline-3-carboxamide

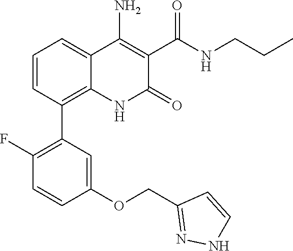 [SMILES: CCCNC(=O)c1c(N)c2cccc(-c3cc(OCc4cc[nH]n4)ccc3F)c2[nH]c1=O]

### Step 1

General procedure 7 was followed using 3-bromo-4-fluorophenol (100 mg, 0.52 mmol) and 3-(chloromethyl)-1H-pyrazole hydrochloride (96 mg, 0.63 mmol) in MeCN to give 3-[(3-bromo-4-fluoro-phenoxy)methyl]-1H-pyrazole (140 mg, 86%) as a yellow oil. m/z 272.9 [M+H]⁺.

### Step 2

General procedure 1 was followed using intermediate 4 (100 mg, 0.27 mmol) and 3-[(3-bromo-4-fluoro-phenoxy)methyl]-1H-pyrazole (73 mg, 0.27 mmol). The material was purified by flash column chromatography on an ISCO system (10 g silica, elution with a 0-10% MeOH/CH₂Cl₂ gradient), followed by reverse phase chromatography on an ISCO system (12 g RediSep® Rf Reversed-phase C18, elution with a 10-100% MeOH/water gradient) to give the title compound (22 mg, 18%) as a colourless solid. 1H NMR (400 MHZ, Chloroform-d) δ 12.11 (s, 1H), 11.37 (s, 1H), 10.37 (t, J=5.7 Hz, 1H), 9.15 (s, 1H), 7.71 (d, J=8.3 Hz, 1H), 7.64-7.53 (m, 2H), 7.46 (s, 1H), 7.38-7.30 (m, 1H), 7.17-7.05 (m, 1H), 7.01-6.88 (m, 1H), 6.41 (d, J=2.3 Hz, 1H), 6.00 (s, 1H), 5.33 (d, J=13.0 Hz, 1H), 5.13 (d, J=12.9 Hz, 1H), 3.41 (q, J=6.7 Hz, 2H), 1.75-1.65 (m, 2H), 1.02 (t, J=7.4 Hz, 3H). m/z 436.2 [M+H]⁺.

### Example 128: 4-Amino-8-[2-fluoro-5-[(1-methylpyrazol-3-yl)methoxy]phenyl]-2-oxo-N-propyl-1H-quinoline-3-carboxamide

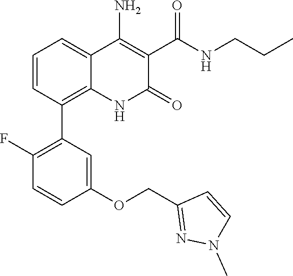 [SMILES: CCCNC(=O)c1c(N)c2cccc(-c3cc(OCc4ccn(C)n4)ccc3F)c2[nH]c1=O]

### Step 1

General procedure 7 was followed using 3-bromo-4-fluorophenol (150 mg, 0.79 mmol) and 3-(chloromethyl)-1-methyl-1H-pyrazole (123 mg, 0.94 mmol) in MeCN to give 3-[(3-bromo-4-fluoro-phenoxy)methyl]-1-methyl-pyrazole (220 mg, 79%) as a yellow oil. ¹H

NMR (400 MHz, Chloroform-d) δ 7.34 (d, J=2.3 Hz, 1H), 7.18 (dd, J=5.6, 3.0 Hz, 1H), 7.06-6.95 (m, 1H), 6.94-6.84 (m, 1H), 6.30 (d, J=2.3 Hz, 1H), 5.01 (s, 2H), 3.89 (s, 3H). m/z 287.0 [M+H]⁺.

### Step 2

General procedure 1 was followed using intermediate 4 (100 mg, 0.27 mmol) and 3-[(3-bromo-4-fluoro-phenoxy)methyl]-1-methyl-pyrazole (77 mg, 0.27 mmol). The material was purified by flash column chromatography on an ISCO system (10 g silica, elution with a 0-10% MeOH/CH₂Cl₂ gradient), followed by reverse phase chromatography on an ISCO system (12 g RediSep® Rf Reversed-phase C18, elution with a 10-100% MeOH/water gradient) to give the title compound (22 mg, 17%) as a yellow solid. ¹H NMR (400 MHZ, DMSO-d₆) δ 10.96 (s, 1H), 10.46 (s, 1H), 9.35 (s, 1H), 8.27-8.16 (m, 2H), 7.67 (d, J=2.2 Hz, 1H), 7.51 (dd, J=7.3, 1.2 Hz, 1H), 7.35-7.24 (m, 2H), 7.17-7.11 (m, 1H), 7.05 (dd, J=6.0, 3.1 Hz, 1H), 6.32 (d, J=2.1 Hz, 1H), 5.01 (s, 2H), 3.83 (s, 3H), 3.28-3.19 (m, 2H), 1.55-1.47 (m, 2H), 0.91 (t, J=7.4 Hz, 3H). m/z 450.2 [M+H]⁺.

### Example 129: 4-Amino-8-[2-fluoro-5-[(1-methyltriazol-4-yl)methoxy]phenyl]-2-oxo-N-propyl-1H-quinoline-3-carboxamide

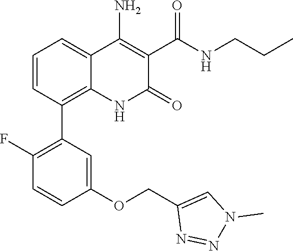 [SMILES: CCCNC(=O)c1c(N)c2cccc(-c3cc(OCc4cn(C)nn4)ccc3F)c2[nH]c1=O]

### Step 1

General procedure 7 was followed using 3-bromo-4-fluorophenol (120 mg, 0.63 mmol) and 4-(chloromethyl)-1-methyl-1H-1,2,3-triazole hydrochloride (127 mg, 0.75 mmol) in MeCN to give 4-[(3-bromo-4-fluoro-phenoxy)methyl]-1-methyl-triazole (140 mg, 68%) as a yellow oil. ¹H NMR (400 MHZ, Chloroform-d) δ 7.62 (s, 1H), 7.17 (dd, J=5.5, 3.0 Hz, 1H), 7.04 (dd, J=9.1, 8.0 Hz, 1H), 6.93-6.84 (m, 1H), 5.16 (s, 2H), 4.12 (s, 3H). m/z 288.0 [M+H]⁺.

### Step 2

General procedure 1 was followed using intermediate 4 (80 mg, 0.27 mmol) and 4-[(3-bromo-4-fluoro-phenoxy)methyl]-1-methyl-triazole (77 mg, 0.27 mmol). The material was purified by flash column chromatography on an ISCO system (10 g silica, elution with a 0-10% MeOH/CH₂Cl₂ gradient) to yield a solid, which was then triturated with MeOH to give the title compound (19 mg, 15%) as a cream solid. ¹H NMR (500 MHZ, DMSO-d₆) δ 10.96 (s, 1H), 10.47 (t, J=5.6 Hz, 1H), 9.40 (s, 1H), 8.26-8.19 (m, 1H), 7.52 (dd, J=7.4, 1.2 Hz, 1H), 7.37-7.24 (m, 2H), 7.19-7.12 (m, 1H), 7.09 (dd, J=6.0, 3.1 Hz, 1H), 5.17 (s, 2H), 4.06 (s, 3H), 3.28-3.20 (m, 2H), 1.59-1.43 (m, 2H), 0.91 (t, J=7.4 Hz, 3H). m/z 451.2 [M+H]⁺.

### Example 130: 4-Amino-8-[2-fluoro-5-[(5-methyl-1,2,4-oxadiazol-3-yl)methoxy]phenyl]-2-oxo-N-propyl-1H-quinoline-3-carboxamide

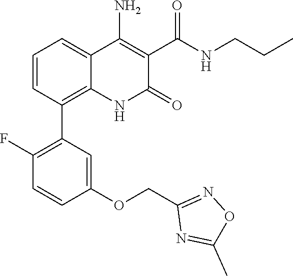 [SMILES: CCCNC(=O)c1c(N)c2cccc(-c3cc(OCc4noc(C)n4)ccc3F)c2[nH]c1=O]

### Step 1

General procedure 7 was followed using 3-bromo-4-fluorophenol (100 mg, 0.52 mmol) and 3-(chloromethyl)-5-methyl-1,2,4-oxadiazole (76 mg, 0.58 mmol) in acetone. The material was purified by flash column chromatography on an ISCO system (10 g silica, elution with a 0-10% MeOH/CH₂Cl₂ gradient) to give 3-[(3-bromo-4-fluoro-phenoxy)methyl]-5-methyl-1,2,4-oxadiazole (104 mg, 66%) as a colourless solid. ¹H NMR (400 MHZ, Chloroform-d) δ 7.26-7.21 (m, 1H), 7.10-7.04 (m, 1H), 7.00-6.89 (m, 1H), 5.14 (s, 2H), 2.65 (s, 3H). m/z 288.9 [M−H]⁺.

### Step 2

General procedure 1 was followed using intermediate 4 (100 mg, 0.27 mmol) and 3-[(3-bromo-4-fluoro-phenoxy)methyl]-5-methyl-1,2,4-oxadiazole (85 mg, 0.30 mmol). The material was purified by reverse phase chromatography on an ISCO system (12 g RediSep® Rf Reversed-phase C18, elution with a 10-100% MeOH/water gradient) to give the title compound (26 mg, 20%) as a yellow solid. ¹H NMR (400 MHZ, Chloroform-d) δ 10.30 (s, 1H), 7.95 (s, 1H), 7.70 (d, J=8.2 Hz, 1H), 7.50 (d, J=7.4 Hz, 1H), 7.33 (d, J=7.8 Hz, 1H), 7.25-7.11 (m, 2H), 7.00 (dd, J=5.7, 3.1 Hz, 1H), 5.20 (s, 2H), 3.39 (q, J=6.6 Hz, 2H), 2.66 (s, 3H), 1.73-1.62 (m, 2H), 1.01 (t, J=7.4 Hz, 3H). m/z 452.2 [M+H]⁺.

### Example 131: 4-Amino-8-(2-fluoro-5-((5-methyl-1,3,4-oxadiazol-2-yl)methoxy)phenyl)-2-oxo-N-propyl-1,2-dihydroquinoline-3-carboxamide

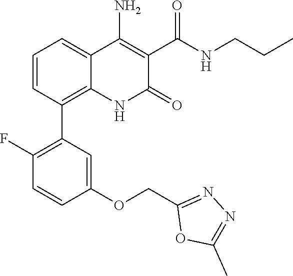 [SMILES: CCCNC(=O)c1c(N)c2cccc(-c3cc(OCc4nnc(C)o4)ccc3F)c2[nH]c1=O]

### Step 1

General procedure 7 was followed using 3-bromo-4-fluorophenol (242 mg, 1.26 mmol) and 2-(chloromethyl)-5-methyl-1,3,4-oxadiazole (112 mg, 0.84 mmol). The material was purified by flash column chromatography on an ISCO system (10 g silica, elution with a 0-20% MeOH/CH₂Cl₂ gradient) to give 2-[(3-bromo-4-fluoro-phenoxy)methyl]-5-methyl-1,3,4-oxadiazole (137 mg, 54%) as a colourless solid. ¹H NMR (600 MHZ, Chloroform-d) δ 7.23-7.17 (m, 1H), 7.10-7.01 (m, 1H), 6.97-6.88 (m, 1H), 5.18 (s, 2H), 2.57 (s, 3H). m/z 288.9 [M+H]⁺.

### Step 2

General procedure 8 was followed using intermediate 4 (59 mg, 0.16 mmol), 2-[(3-bromo-4-fluoro-phenoxy)methyl]-5-methyl-1,3,4-oxadiazole (35 mg, 0.12 mmol). The material was purified by flash column chromatography on an ISCO system (10 g silica, elution with a 0-10% MeOH/CH₂Cl₂ gradient) and further purification by flash column chromatography on an ISCO system (10 g silica, elution with a 0-10% MeOH/EtOAc gradient), followed by trituration with Et₂O to give the title compound (14 mg, 24%) as a colourless solid. ¹H NMR (600 MHZ, Chloroform-d) δ 11.31 (s, 1H), 10.25 (s, 1H), 7.90 (s, 1H), 7.68 (d, J=8.3 Hz, 1H), 7.48 (d, J=7.3 Hz, 1H), 7.30 (t, J=8.2 Hz, 1H), 7.20 (t, J=9.0 Hz, 1H), 7.15-7.06 (m, 1H), 7.02-6.88 (m, 1H), 5.90 (s, 1H), 5.24 (s, 2H), 3.37 (t, J=7.2 Hz, 2H), 2.59 (s, 3H), 1.64 (q, J=8.2 Hz, 2H), 0.99 (t, J=9.1 Hz, 3H). m/z 452.0 [M+H]⁺.

### Example 132: 4-Amino-8-[2-fluoro-5-[[5-(trifluoromethyl)-1,3,4-oxadiazol-2-yl]methoxy]phenyl]-2-oxo-N-propyl-1H-quinoline-3-carboxamide

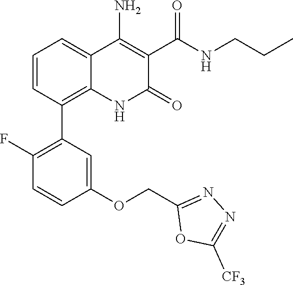 [SMILES: CCCNC(=O)c1c(N)c2cccc(-c3cc(OCc4nnc(C(F)(F)F)o4)ccc3F)c2[nH]c1=O]

### Step 1

General procedure 7 was followed using 3-bromo-4-fluorophenol (150 mg, 0.79 mmol) and 2-(chloromethyl)-5-(trifluoromethyl)-1,3,4-oxadiazole (176 mg, 0.94 mmol) in MeCN to give 2-[(3-bromo-4-fluoro-phenoxy)methyl]-5-(trifluoromethyl)-1,3,4-oxadiazole (240 mg, 72%) as an orange solid. ¹H NMR (400 MHZ, Chloroform-d) δ 7.24 (dd, J=5.5, 3.1 Hz, 1H), 7.10 (dd, J=9.1, 7.8 Hz, 1H), 7.01-6.88 (m, 1H), 5.33 (s, 2H). m/z 342.9 [M+H]⁺.

### Step 2

General procedure 1 was followed using intermediate 4 (100 mg, 0.27 mmol) and 2-[(3-bromo-4-fluoro-phenoxy)methyl]-5-(trifluoromethyl)-1,3,4-oxadiazole (92 mg, 0.27 mmol). The material was purified by flash column chromatography on an ISCO system (10 g silica, elution with a 0-10% MeOH/CH₂Cl₂ gradient) to yield a solid, which was then triturated with MeOH to give the title compound (49 mg, 32%) as a colourless solid. ¹H NMR (500 MHZ, DMSO-d₆) δ 10.95 (s, 1H), 10.47 (s, 1H), 9.51 (s, 1H), 8.31-8.12 (m, 2H), 7.51 (dd, J=7.4, 1.2 Hz, 1H), 7.39-7.28 (m, 2H), 7.27-7.21 (m, 1H), 7.19 (dd, J=5.9, 3.2 Hz, 1H), 5.59 (s, 2H), 3.27-3.20 (m, 2H), 1.56-1.47 (m, 2H), 0.91 (t, J=7.4 Hz, 3H). m/z 506.2 [M+H]⁺.

### Example 133: 8-(5-((1H-Benzo[d]imidazol-5-yl)methoxy)-2-fluorophenyl)-4-amino-2-oxo-N-propyl-1,2-dihydroquinoline-3-carboxamide

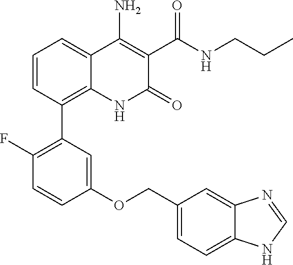 [SMILES: CCCNC(=O)c1c(N)c2cccc(-c3cc(OCc4ccc5[nH]cnc5c4)ccc3F)c2[nH]c1=O]

### Step 1

General procedure 4 was followed using 1H-benzimidazol-5-ylmethanol (116 mg, 0.78 mmol) and 3-bromo-4-fluorophenol (150 mg, 0.78 mmol). The material was purified by flash column chromatography on an ISCO system (10 g silica, elution with a 50-100% EtOAc/pet. ether gradient) was followed to give 5-[(3-bromo-4-fluoro-phenoxy)methyl]-1H-benzimidazole (115 mg, 41%) as a colourless oil. ¹H NMR (600 MHZ, Chloroform-d) 8.08 (s, 1H), 7.80-7.60 (m, 2H), 7.35 (d, J=8.5 Hz, 1H), 7.18 (t, J=3.3 Hz, 1H), 7.03 (t, J=9.1 Hz, 1H), 6.89 (dd, J=8.9, 3.7 Hz, 1H), 5.15 (s, 2H). m/z 321.1, 323.0 [M+H]⁺.

### Step 2

General procedure 2 was followed using intermediate 4 (150 mg, 0.40 mmol) and 5-[(3-bromo-4-fluoro-phenoxy)methyl]-1H-benzimidazole (130 mg, 0.40 mmol The material was purified by flash column chromatography on an ISCO system (10 g silica, elution with a 0-10% MeOH/EtOAc gradient) to give the title compound (50 mg, 25%) as a colourless solid. ¹H NMR (600 MHZ, Chloroform-d) δ 11.33 (s, 1H), 10.24 (s, 1H), 8.06 (s, 1H), 7.98 (s, 1H), 7.87-7.54 (m, 3H), 7.50 (d, J=7.4 Hz, 1H), 7.32 (dt, J=15.8, 8.1 Hz, 2H), 7.13 (t, J=8.9 Hz, 1H), 7.03 (d, J=9.0 Hz, 1H), 6.96 (d, J=4.7 Hz, 1H), 5.95 (s, 1H), 5.23 (s, 2H), 3.39 (q, J=6.6 Hz, 2H), 1.65 (q, J=7.4 Hz, 2H), 1.00 (t, J=7.4 Hz, 3H). m/z 486.2 [M+H]⁺.

### Example 134: 4-Amino-8-[2-fluoro-5-[2-(1,2,4-triazol-4-yl) ethoxy]phenyl]-2-oxo-N-propyl-1H-quinoline-3-carboxamide

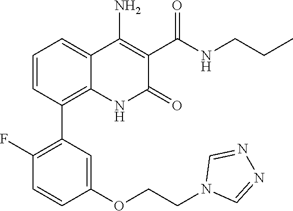 [SMILES: CCCNC(=O)c1c(N)c2cccc(-c3cc(OCCn4cnnc4)ccc3F)c2[nH]c1=O]

### Step 1

General procedure 4 was followed using 3-bromo-4-fluorophenol (96 mg, 0.50 mmol) and 2-(4H-1,2,4-triazol-4-yl) ethanol (74 mg, 0.65 mmol). The material was purified by reverse phase chromatography on an ISCO system (12 g RediSep® Rf Reversed-phase C18, elution with a 10-100% MeOH/water gradient) to give 4-[2-(3-bromo-4-fluoro-phenoxy) ethyl]-1,2,4-triazole (64 mg, 40%) as a yellow solid. ¹H NMR (500 MHZ, Chloroform-d) δ 8.29 (s, 2H), 7.08-7.02 (m, 2H), 6.78 (ddd, J=9.0, 3.6, 3.0 Hz, 1H), 4.42 (dd, J=5.4, 4.4 Hz, 2H), 4.19 (dd, J=5.4, 4.4 Hz, 2H). m/z 288.0 [M+H]⁺.

### Step 2

General procedure 1 was followed using intermediate 4 (75 mg, 0.20 mmol) and 4-[2-(3-bromo-4-fluoro-phenoxy) ethyl]-1,2,4-triazole (64 mg, 0.20 mmol). The material was purified by reverse phase chromatography on an ISCO system (12 g RediSep® Rf Reversed-phase C18, elution with a 10-100% MeOH/water gradient), followed by flash column chromatography on an ISCO system (10 g silica, elution with a 10% MeOH/CH₂Cl₂ gradient) were followed to give the title compound (13 mg, 13%) as an off-white solid. ¹H NMR (500 MHZ, DMSO-d₆) δ 10.95 (s, 1H), 10.46 (t, J=5.6 Hz, 1H), 9.46 (s, 1H), 8.56 (s, 2H), 8.29-8.10 (m, 2H), 7.49 (dd, J=7.3, 1.3 Hz, 1H), 7.36-7.22 (m, 2H), 7.07 (ddd, J=9.0, 4.0, 3.1 Hz, 1H), 6.99 (dd, J=6.0, 3.1 Hz, 1H), 4.44 (t, J=5.0 Hz, 2H), 4.29 (app. q, J=4.8, 4.4 Hz, 2H), 3.23 (td, J=6.9, 5.6 Hz, 2H), 1.50 (app. sextet, J=7.3 Hz, 2H), 0.90 (t, J=7.4 Hz, 3H). m/z 451.2 [M+H]⁺.

### Example 135: 4-Amino-8-[2-fluoro-5-(tetrahydropyran-4-ylmethoxy)phenyl]-2-oxo-N-propyl-1H-quinoline-3-carboxamide

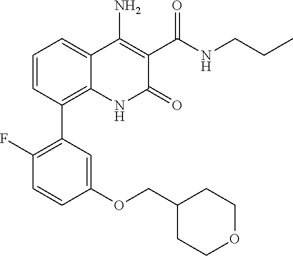 [SMILES: CCCNC(=O)c1c(N)c2cccc(-c3cc(OCC4CCOCC4)ccc3F)c2[nH]c1=O]

### Step 1

General procedure 7 was followed using 3-bromo-4-fluorophenol (100 mg, 0.52 mmol) and 4-(bromomethyl)tetrahydro-2H-pyran (112 mg, 0.63 mmol) in MeCN. The material was purified by reverse phase chromatography on an ISCO system (12 g RediSep® Rf Reversed-phase C18, elution with a 10-100% MeOH/water gradient) to give 4-[(3-bromo-4-fluoro-phenoxy)methyl] tetrahydropyran (110 mg, 69%) as a yellow oil. ¹H NMR (400 MHZ, Chloroform-d) δ 7.16-6.93 (m, 2H), 6.91-6.67 (m, 1H), 4.04 (dd, J=11.5, 4.7 Hz, 2H), 3.77 (d, J=6.4 Hz, 2H), 3.56-3.28 (m, 2H), 2.17-1.92 (m, 1H), 1.86-1.62 (m, 2H), 1.58-1.28 (m, 2H).

### Step 2

General procedure 1 was followed using intermediate 4 (100 mg, 0.27 mmol) and 4-[(3-bromo-4-fluoro-phenoxy)methyl] tetrahydropyran (78 mg, 0.27 mmol). The material was purified by flash column chromatography on an ISCO system (10 g silica, elution with a 0-10% MeOH/CH₂Cl₂ gradient), followed by reverse phase chromatography on an ISCO system (12 g RediSep® Rf Reversed-phase C18, elution with a 10-100% MeOH/water gradient) to give the title compound (70 mg, 52%) as a yellow solid. ¹H NMR (500 MHZ, DMSO-d₆) δ 10.95 (s, 1H), 10.49-10.41 (m, 1H), 9.37 (s, 1H), 8.24-8.18 (m, 2H), 7.52 (dd, J=7.3, 1.2 Hz, 1H), 7.32 (dd, J=8.3, 7.4 Hz, 1H), 7.29-7.20 (m, 1H), 7.10-7.02 (m, 1H), 6.95 (dd, J=6.0, 3.1 Hz, 1H), 3.91-3.77 (m, 4H), 3.31-3.27 (m, 2H), 3.24 (q, J=6.6 Hz, 2H), 2.06-1.93 (m, 1H), 1.71-1.64 (m, 2H), 1.57-1.46 (m, 2H), 1.39-1.25 (m, 2H), 0.91 (t, J=7.4 Hz, 3H). m/z 454.3 [M+H]⁺.

### Example 136: 4-Amino-8-[2-fluoro-5-(oxetan-3-ylmethoxy)phenyl]-2-oxo-N-propyl-1H-quinoline-3-carboxamide

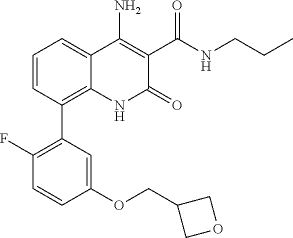 [SMILES: CCCNC(=O)c1c(N)c2cccc(-c3cc(OCC4COC4)ccc3F)c2[nH]c1=O]

### Step 1

General procedure 7 was followed using 3-bromo-4-fluorophenol (150 mg, 0.79 mmol) and oxetane-3-methanol (100 mg, 1.13 mmol) in MeCN. The material was purified by reverse phase chromatography on an ISCO system (12 g RediSep® Rf Reversed-phase C18, elution with a 10-100% MeOH/water gradient) to give 3-[(3-bromo-4-fluoro-phenoxy)methyl]oxetane (78 mg, 36%) as a colourless oil. ¹H NMR (500 MHZ, Chloroform-d) δ 7.14-7.08 (m, 1H), 7.09-7.01 (m, 1H), 6.86-6.80 (m, 1H), 4.92-4.85 (m, 2H), 4.59-4.52 (m, 2H), 4.16 (d, J=6.7 Hz, 2H), 3.54-3.24 (m, 1H).

### Step 2

General procedure 1 was followed using intermediate 3 (85 mg, 0.29 mmol) and 3-[(3-bromo-4-fluoro-phenoxy)methyl] oxetane (77 mg, 0.29 mmol). The material was purified by flash column chromatography on an ISCO system (10 g silica, elution with a 0-10% MeOH/CH₂Cl₂ gradient), followed by reverse phase chromatography on an ISCO system (12 g RediSep® Rf Reversed-phase C18, elution with a 10-100% MeOH/water gradient) to give the title compound (21 mg, 16%) as a pale yellow solid. ¹H NMR (400 MHz, Chloroform-d) δ 11.28 (s, 1H), 10.28 (t, J=7.3 Hz, 1H), 7.97 (s, 1H), 7.70 (d, J=8.2 Hz, 1H), 7.52 (d, J=7.3 Hz, 1H), 7.37-7.30 (m, 1H), 7.24-7.16 (m, 1H), 7.08-6.98 (m, 1H), 6.87 (dd, J=5.8, 3.2 Hz, 1H), 5.94 (s, 1H), 4.91 (t, J=7.0 Hz, 2H), 4.59 (t, J=6.0 Hz, 2H), 4.22 (d, J=6.6 Hz, 2H), 3.56-3.32 (m, 3H), 1.72-1.62 (m, 2H), 1.01 (t, J=7.4 Hz, 3H). m/z 426.2 [M+H]⁺.

### Example 137:4-Amino-8-[2-fluoro-5-[(4-methylpiperazin-1-yl)methyl]phenyl]-2-oxo-N-propyl-1H-quinoline-3-carboxamide

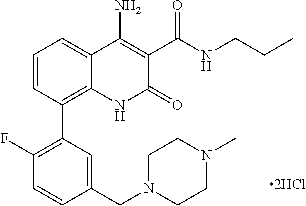 [SMILES: CCCNC(=O)c1c(N)c2cccc(-c3cc(CN4CCN(C)CC4)ccc3F)c2[nH]c1=O]

### Step 1

General procedure 9 was followed using 2-bromo-4-(bromomethyl)-1-fluorobenzene (1.00 g, 3.73 mmol) and 1-methylpiperazine (0.41 mL, 3.73 mmol). The reaction mixture was washed with water and brine, dried over MgSO₄, filtered and concentrated under reduced pressure to give 1-[(3-bromo-4-fluoro-phenyl)methyl]-4-methyl-piperazine (290 mg, 26%) as a colourless oil. ¹H NMR (600 MHZ, Chloroform-d) δ 7.53 (dd, J=6.8, 2.0 Hz, 1H), 7.25-7.16 (m, 1H), 7.04 (t, J=8.4 Hz, 1H), 3.44 (s, 2H), 2.45 (s, 8H), 2.29 (s, 3H). m/z 287.1, 289.1 [M+H]⁺.

### Step 2

General procedure 8 was followed using intermediate 4 (300 mg, 0.69 mmol) and 1-[(3-bromo-4-fluoro-phenyl)methyl]-4-methyl-piperazine (217 mg, 0.76 mmol). The material was purified by flash column chromatography on an ISCO system (10 g silica, elution with a 0-20% MeOH/EtOAc gradient) to give the title compound (260 mg, 80%) as a colourless solid. The compound was treated with 2 M HCl in Et₂O to obtain the corresponding 2×HCl salt. ¹H NMR (600 MHZ, Methanol-d₄) 0 8.14 (d, J=8.3 Hz, 1H), 7.79 (t, J=6.4 Hz, 1H), 7.71 (d, J=6.6 Hz, 1H), 7.61 (d, J=7.3 Hz, 1H), 7.46 (t, J=8.9 Hz, 1H), 7.38 (t, J=7.8 Hz, 1H), 4.49 (s, 2H), 4.04-3.43 (m, 8H), 3.33-3.31 (m, 2H), 3.02 (s, 3H), 1.61 (app. sextet, J=7.3 Hz, 2H), 0.98 (t, J=7.4 Hz, 3H). m/z 452.2 [M+H]⁺ (free base).

### Example 138: 4-Amino-8-(3-fluoro-6-((4-methylpiperazin-1-yl)methyl) pyridin-2-yl)-2-oxo-N-propyl-1,2-dihydroquinoline-3-carboxamide

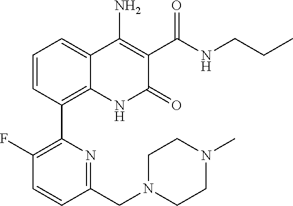 [SMILES: CCCNC(=O)c1c(N)c2cccc(-c3nc(CN4CCN(C)CC4)ccc3F)c2[nH]c1=O]

### Step 1

To 2-bromo-6-(bromomethyl)-3-fluoropyridine (2.3 g, 5.28 mmol) in CH₂Cl₂ (10 mL) was added 1-methylpiperazine (2.65 g, 26.4 mmol) and the reaction mixture was stirred at rt for 16 h. After this time, the reaction mixture was concentrated under reduced pressure and the material was purified by flash column chromatography on an ISCO system (10 g silica, elution with a 0-20% MeOH/CH₂Cl₂ gradient) to give 1-[(6-bromo-5-fluoro 2-pyridyl)methyl]-4-methyl-piperazine (1070 mg, 67%) as a brown oil. ¹H NMR (500 MHz, Chloroform-d) δ 7.62-7.29 (m, 2H), 3.73-3.41 (m, 2H), 2.86-2.35 (m, 8H), 2.26 (s, 3H). m/z 288.0, 290.8 [M+H]⁺.

### Step 2

General procedure 8 was followed using intermediate 4 (323 mg, 0.87 mmol) and 5-[(3-bromo-4-fluoro-phenoxy)methyl]-1-ethyl-1,2,4-triazole (200 mg, 0.69 mmol). The material was purified by flash column chromatography on an ISCO system (10 g silica, elution with a 0-20% MeOH/CH₂Cl₂+1% NH₃ gradient) to give the title compound (251 mg, 76%) as a brown foam. ¹H NMR (400 MHZ, Chloroform-d) δ 11.56 (s, 1H), 11.22 (s, 1H), 10.44 (s, 1H), 8.02 (d, J=7.5 Hz, 1H), 7.71 (s, 1H), 7.58 (dd, J=22.8, 12.6 Hz, 1H), 7.35-7.22 (m, 2H), 6.10-5.63 (m, 1H), 3.96-3.72 (m, 2H), 3.46-3.35 (m, 2H), 2.81-2.39 (m, 6H), 2.30 (s, 3H), 1.94-1.58 (m, 4H), 1.43-1.16 (m, 3H). m/z 453.1 [M+H]⁺.

### Example 139: 4-Amino-8-(2-fluoro-5-((6-methyl-2,6-diazaspiro[3.4]octan-2-yl)methyl)phenyl)-2-oxo-N-propyl-1,2-dihydroquinoline-3-carboxamide

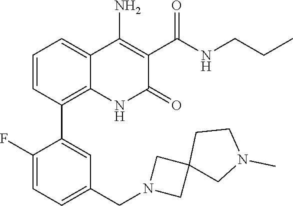 [SMILES: CCCNC(=O)c1c(N)c2cccc(-c3cc(CN4CC5(CCN(C)C5)C4)ccc3F)c2[nH]c1=O]

### Step 1

General procedure 9 was followed using 2-bromo-4-(bromomethyl)-1-fluorobenzene (150 mg, 0.56 mmol) and tert-butyl-2,6-diazaspiro[3.4]octan-6-carboxylate (119 mg, 0.57 mmol). The reaction mixture was concentrated under reduced pressure and the residue diluted with CH₂Cl₂ (50 mL) and washed with brine (30 mL). The organic layer was dried over Na₂SO₄, filtered and concentrated under reduced pressure to give tert-butyl 2-[(3-bromo-4-fluoro-phenyl)methyl]-2,7-diazaspiro[3.4]octane-7-carboxylate (215 mg, 91%) as a colourless oil. ¹H NMR (600 MHZ, Chloroform-d) δ 7.47 (d, J=6.7 Hz, 1H), 7.17 (dd, J=8.4, 4.9 Hz, 1H), 7.04 (t, J=8.4 Hz, 1H), 3.54 (s, 2H), 3.42 (s, 2H), 3.32 (dt, J=26.8, 7.2 Hz, 2H), 3.15 (dq, J=14.5, 8.3 Hz, 4H), 2.02 (dt, J=31.4, 7.0 Hz, 2H), 1.45 (s, 9H). m/z 398.8, 400.9 [M+H]⁺.

### Step 2

A mixture of tert-butyl 2-[(3-bromo-4-fluoro-phenyl)methyl]-2,7-diazaspiro[3.4]octane-7-carboxylate (110 mg, 0.27 mmol) and a formaldehyde solution (0.08 mL, 1.10 mmol) in formic acid (1.04 mL) was heated at 95° C. for 2 h. After this time, the reaction mixture was cooled to rt, 1 M NaOH solution (3 mL) was added and the aqueous layer was extracted with CH₂Cl₂ (3×5 mL). The combined organic extracts were dried over Na₂SO₄, filtered and concentrated under reduced pressure to give 2-[(3-bromo-4-fluoro-phenyl)methyl]-7-methyl-2,7-diazaspiro[3.4]octane (70 mg, 77%) as a pale yellow oil. ¹H NMR (400 MHZ, Methanol-d₄) δ 7.54 (dd, J=6.7, 2.2 Hz, 1H), 7.29-7.23 (m, 1H), 7.13 (t, J=8.6 Hz, 1H), 3.55 (s, 2H), 3.28-3.16 (m, 4H), 2.71 (s, 2H), 2.52 (t, J=7.1 Hz, 2H), 2.31 (s, 3H), 2.04 (t, J=7.1 Hz, 2H). m/z 313.0, 315.0 [M+H]⁺.

### Step 3

General procedure 2 was followed using intermediate 4 (150 mg, 0.28 mmol) and 2-[(3-bromo-4-fluoro-phenyl)methyl]-7-methyl-2,7-diazaspiro[3.4]octane (88 mg, 0.28 mmol). The material was purified by flash column chromatography on an ISCO system (10 g silica, elution with a 0-10% MeOH/CH₂Cl₂+1% NH₃ gradient) to give the title compound (25 mg, 18%) as a pale-yellow solid. ¹H NMR (400 MHZ, Methanol-d₄) δ 8.09 (dd, J=8.4, 1.3 Hz, 1H), 7.52 (dd, J=7.3, 1.3 Hz, 1H), 7.50-7.43 (m, 1H), 7.38-7.31 (m, 2H), 7.27 (dd, J=9.6, 8.5 Hz, 1H), 3.69 (s, 2H), 3.39-3.33 (m, 2H), 3.30 (d, J=2.6 Hz, 4H), 2.90 (s, 2H), 2.71 (t, J=7.2 Hz, 2H), 2.44 (s, 3H), 2.12 (t, J=7.2 Hz, 2H), 1.59 (h, J=7.3 Hz, 2H), 0.96 (t, J=7.4 Hz, 3H). m/z 478.1 [M+H]⁺.

### Example 140: 4-Amino-8-(2-fluoro-5-((7-methyl-2,7-diazaspiro[3.5]nonan-2-yl)methyl)phenyl)-2-oxo-N-propyl-1,2-dihydroquinoline-3-carboxamide

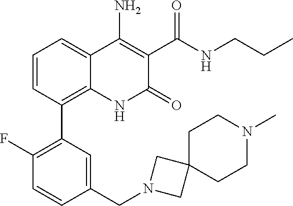 [SMILES: CCCNC(=O)c1c(N)c2cccc(-c3cc(CN4CC5(CCN(C)CC5)C4)ccc3F)c2[nH]c1=O]

### Step 1

General procedure 9 was followed using 2-bromo-4-(bromomethyl)-1-fluorobenzene (200 mg, 0.75 mmol) and 2-methyl-2-propanyl 2,7-diazaspiro[3.5]nonane-7-carboxylate (169 mg, 0.74 mmol). The reaction mixture was concentrated under reduced pressure and the residue diluted with CH₂Cl₂ (50 mL) and washed with brine (30 mL). The organic layer was dried over Na₂SO₄, filtered and concentrated under reduced pressure to give tert-butyl 2-[(3-bromo-4-fluoro-phenyl)methyl]-2,7-diazaspiro[3.5]nonane-7-carboxylate (285 mg, 88%) as a colourless oil. ¹H NMR (600 MHZ, Chloroform-d) δ 7.47 (d, J=5.5 Hz, 1H), 7.24-7.14 (m, 1H), 7.04 (t, J=8.4 Hz, 1H), 3.57 (s, 2H), 3.32 (t, J=5.6 Hz, 4H), 3.02 (s, 4H), 1.70 (t, J=5.7 Hz, 4H), 1.44 (s, 9H). m/z 412.7, 414.7 [M+H]⁺.

### Step 2

A mixture of tert-butyl 2-[(3-bromo-4-fluoro-phenyl)methyl]-2,7-diazaspiro[3.5]nonane-7-carboxylate (285 mg, 0.69 mmol) and formaldehyde solution (0.08 mL, 2.76 mmol) in formic acid (2.60 mL) was heated at 95° C. for 2 h. After this time, the reaction mixture was cooled to rt, 1 M NaOH solution (3 mL) was added and the aqueous layer was extracted with CH₂Cl₂ (3×5 mL). The combined organic extracts were dried over Na₂SO₄, filtered and concentrated under reduced pressure to give 2-[(3-bromo-4-fluoro-phenyl)methyl]-7-methyl-2,7-diazaspiro[3.5]nonane (132 mg, 56%) as a yellow oil. ¹H NMR (600 MHZ, Chloroform-d) δ 7.47 (dd, J=6.8, 2.0 Hz, 1H), 7.20-7.14 (m, 1H), 7.04 (t, J=8.4 Hz, 1H), 3.56 (s, 2H), 2.99 (s, 4H), 2.44-2.24 (m, 4H), 2.23 (s, 3H), 1.78 (t, J=5.5 Hz, 4H). m/z 327.1, 329.0 [M+H]⁺.

### Step 3

General procedure 2 was followed using intermediate 4 (150 mg, 0.28 mmol) and 2-[(3-bromo-4-fluoro-phenyl)methyl]-7-methyl-2,7-diazaspiro[3.5]nonane (92 mg, 0.28 mmol). The material was purified by flash column chromatography on an ISCO system (10 g silica, elution with a 20-100% EtOAc/pet. ether gradient) to give the title compound (10 mg, 7%) as a brown solid. ¹H NMR (600 MHZ, Methanol-d₄) δ 8.15-8.09 (m, 1H), 7.57-7.53 (m, 1H), 7.51 (d, J=6.8 Hz, 1H), 7.41-7.34 (m, 2H), 7.31 (t, J=9.0 Hz, 1H), 3.82 (s, 2H), 3.32-3.28 (m, 2H), 3.26 (s, 4H), 2.86-2.55 (m, 4H), 2.49 (s, 3H), 1.91 (s, 4H), 1.66-1.56 (m, 2H), 0.98 (t, J=7.6 Hz, 3H). m/z 492.2 [M+H]⁺.

### Example 141: 8-(5-((7-Oxa-2-azaspiro[3.5]nonan-2-yl)methyl)-2-fluorophenyl)-4-amino-N-propyl-1,2-dihydroquinoline-3-carboxamide hydrochloride

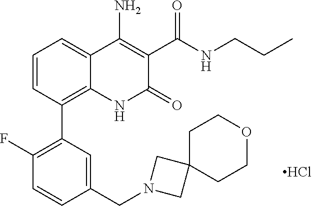 [SMILES: CCCNC(=O)c1c(N)c2cccc(-c3cc(CN4CC5(CCOCC5)C4)ccc3F)c2[nH]c1=O ; Cl]

### Step 1

General procedure 9 was followed using 2-bromo-4-(bromomethyl)-1-fluorobenzene (200 mg, 0.75 mmol) and 7-oxa-2-azaspiro[3.5]nonane (95 mg, 0.75 mmol). The material was purified by flash column chromatography on an ISCO system (10 g silica, elution with a 0-20% MeOH/CH₂Cl₂ gradient) to give 2-[(3-bromo-4-fluoro-phenyl)methyl]-7-oxa-2-azaspiro[3.5]nonane (122 mg, 49%) as a colourless gum. ¹H NMR (600 MHZ, Chloroform-d) δ 7.47 (d, J=6.7 Hz, 1H), 7.22-7.13 (m, 1H), 7.04 (td, J=8.4, 2.8 Hz, 1H), 3.64-3.49 (m, 6H), 3.04 (s, 4H), 1.83-1.71 (m, 4H). m/z 315.8 [M+H]⁺.

### Step 2

General procedure 8 was followed using intermediate 4 (40 mg, 0.11 mmol) and 2-[(3-bromo-4-fluoro-phenyl)methyl]-7-oxa-2-azaspiro[3.5]nonane (26 mg, 0.08 mmol). The material was purified by flash column chromatography on an ISCO system (10 g silica, elution with a 0-50% MeOH/CH₂Cl₂ gradient), then acidification using 2 N HCl in Et₂O to give the title compound (23 mg, 51%) as a brown solid. ¹H NMR (600 MHZ, Methanol-d₄) o 8.15 (d, J=8.3 Hz, 1H), 7.77-7.65 (m, 1H), 7.65-7.55 (m, 2H), 7.46 (app. t, J=8.6 Hz, 1H), 7.42-7.32 (m, 1H), 4.47 (s, 2H), 4.14-3.92 (m, 3H), 3.74-3.63 (m, 2H), 3.63-3.55 (m, 2H), 3.37-3.22 (m, 3H), 1.95-1.81 (m, 4H), 1.68-1.53 (m, 2H), 1.06-0.94 (m, 3H). m/z 477.2 [M+H]⁺ (free base).

### Example 142: 4-Amino-8-(2-fluoro-5-((7-methyl-2,7-diazaspiro[4.4]nonan-2-yl)methyl)phenyl)-2-oxo-N-propyl-1,2-dihydroquinoline-3-carboxamide

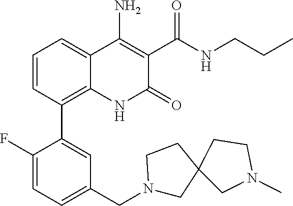 [SMILES: CCCNC(=O)c1c(N)c2cccc(-c3cc(CN4CCC5(CCN(C)C5)C4)ccc3F)c2[nH]c1=O]

### Step 1

To 2-bromo-4-(bromomethyl)-1-fluorobenzene (200 mg, 0.75 mmol) in CH₂Cl₂ (3 mL) was added tert-butyl 2,7-diazaspiro[4.4]nonane-2-carboxylate (507 mg, 2.24 mmol) and the reaction mixture was stirred at rt for 16 h. After this time, the reaction mixture was concentrated under reduced pressure and the material was purified by flash column chromatography on an ISCO system (24 g silica, elution with a 0-20% MeOH/CH₂Cl₂ gradient) to give tert-butyl 7-[(3-bromo-4-fluoro-phenyl)methyl]-2,7-diazaspiro[4.4]nonane-2-carboxylate (317 mg, 98%) as a colourless oil. ¹H NMR (500 MHZ, Chloroform-d) δ 7.52 (d, J=6.8 Hz, 1H), 7.23-7.15 (m, 1H), 7.11-6.95 (m, 1H), 3.51 (m, 2H), 3.44-3.03 (m, 4H), 2.78-2.18 (m, 4H), 1.93-1.56 (m, 4H), 1.53-1.38 (m, 9H). m/z 412.9, 414.9 [M+H]⁺.

### Step 2

A mixture of tert-butyl 7-[(3-bromo-4-fluoro-phenyl)methyl]-2,7-diazaspiro[4.4]nonane-2-carboxylate (150 mg, 0.36 mmol) and formaldehyde solution (0.11 mL, 1.45 mmol) in formic acid (1.37 mL) was heated at 95° C. for 2 h. After this time, the reaction mixture was cooled to rt, 1 M NaOH solution (3 mL) was added and the aqueous layer was extracted with CH₂Cl₂ (3×5 mL). The combined organic extracts were dried over Na₂SO₄, filtered and concentrated under reduced pressure to give 2-[(3-bromo-4-fluoro-phenyl)methyl]-7-methyl-2,7-diazaspiro[4.4]nonane (98 mg, 78%) as a pale yellow oil. ¹H NMR (400 MHZ, Methanol-d₄) δ 7.58 (dd, J=6.8, 2.1 Hz, 1H), 7.38-7.24 (m, 1H), 7.13 (t, J=8.6 Hz, 1H), 3.56 (s, 2H), 2.66-2.51 (m, 6H), 2.45 (dd, J=9.5, 6.2 Hz, 2H), 2.30 (s, 3H), 1.95-1.75 (m, 4H). m/z 327.0, 329.0 [M+H]⁺.

### Step 3

General procedure 2 was followed using intermediate 4 (143 mg, 0.27 mmol) and 2-[(3-bromo-4-fluoro-phenyl)methyl]-7-methyl-2,7-diazaspiro[3.5]nonane (88 mg, 0.27 mmol). The material was purified by flash column chromatography on an ISCO system (10 g silica, elution with a 0-10% MeOH/CH₂Cl₂+1% NH₃ gradient) to give the title compound (12 mg, 9%) as a beige solid. ¹H NMR (600 MHZ, Methanol-d₄) 0 8.11 (d, J=8.3 Hz, 1H), 7.56 (d, J=7.3 Hz, 1H), 7.52 (s, 1H), 7.40 (d, J=7.1 Hz, 1H), 7.37 (t, J=7.8 Hz, 1H), 7.29 (t, J=9.0 Hz, 1H), 3.71 (q, J=13.2 Hz, 2H), 2.97 (s, 3H), 2.81 (dd, J=46.3, 9.3 Hz, 2H), 2.70 (t, J=10.2 Hz, 2H), 2.59 (s, 4H), 2.09-1.86 (m, 4H), 1.66-1.56 (m, 4H), 0.98 (t, J=7.4 Hz, 3H). m/z 492.2 [M+H]⁺.

### Example 143: 4-Amino-8-(2-fluoro-5-((8-methyl-2,8-diazaspiro[4.5]decan-2-yl)methyl)phenyl)-2-oxo-N-propyl-1,2-dihydroquinoline-3-carboxamide

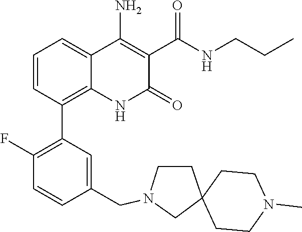 [SMILES: CCCNC(=O)c1c(N)c2cccc(-c3cc(CN4CCC5(CCN(C)CC5)C4)ccc3F)c2[nH]c1=O]

### Step 1

General procedure 9 was followed using 2-bromo-4-(bromomethyl)-1-fluorobenzene (873 mg, 3.26 mmol) and 8-methyl-2,8-diazaspiro[4.5]decane (503 mg, 3.26 mmol). The material was purified by flash column chromatography on an ISCO system (24 g silica, elution with a 0-20% MeOH/CH₂Cl₂ gradient) to give 2-[(3-bromo-4-fluoro-phenyl)methyl]-8-methyl-2,8-diazaspiro[4.5]decane (325 mg, 28%) as a colourless solid. ¹H NMR (400 MHZ, Chloroform-d) δ 7.48 (d, J=6.9 Hz, 1H), 7.19 (d, J=7.2 Hz, 1H), 7.04 (td, J=8.4, 1.9 Hz, 1H), 3.52 (s, 2H), 3.34-2.75 (m, 4H), 2.68 (s, 3H), 2.60 (t, J=7.1 Hz, 2H), 2.41 (s, 2H), 2.24-1.84 (m, 4H), 1.69 (t, J=7.2 Hz, 2H). m/z 341.1, 343.1 [M+H]⁺.

### Step 2

General procedure 8 was followed using intermediate 4 (40 mg, 0.10 mmol) and 2-[(3-bromo-4-fluoro-phenyl)methyl]-8-methyl-2,8-diazaspiro[4.5]decane (28 mg, 0.08 mmol). The material was purified by flash column chromatography on an ISCO system (10 g silica, elution with a 0-20% MeOH/CH₂Cl₂ gradient) to give the title compound (7 mg, 16%) as a brown solid. ¹H NMR (600 MHZ, Chloroform-d) δ 11.29 (s, 1H), 10.27 (s, 1H), 7.94 (s, 1H), 7.66 (d, J=8.3 Hz, 1H), 7.50 (d, J=7.5 Hz, 1H), 7.43 (s, 1H), 7.34-7.27 (m, 2H), 7.19 (t, J=9.5 Hz, 1H), 5.89 (s, 1H), 3.64 (s, 1H), 3.60 (s, 2H), 3.40-3.30 (m, 2H), 2.64-2.52 (m, 2H), 2.49-2.12 (m, 8H), 1.76-1.63 (m, 8H), 1.06-0.92 (m, 3H). m/z 504.2 [M−H]⁻.

### Example 144: 4-Amino-8-(2-fluoro-5-((2-methyl-2,7-diazaspiro[3.5]nonan-7-yl)methyl)phenyl)-2-oxo-N-propyl-1,2-dihydroquinoline-3-carboxamide

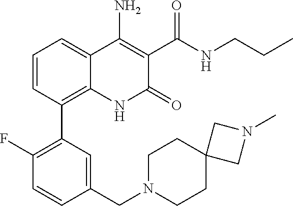 [SMILES: CCCNC(=O)c1c(N)c2cccc(-c3cc(CN4CCC5(CC4)CN(C)C5)ccc3F)c2[nH]c1=O]

### Step 1

General procedure 9 was followed using 2-bromo-4-(bromomethyl)-1-fluorobenzene (200 mg, 0.74 mmol) and tert-butyl-2,7-diazaspiro[3.5]nonan-2-carboxylate (169 mg, 0.74 mmol). The reaction mixture was concentrated under reduced pressure and the residue diluted with CH₂Cl₂ (50 mL) and washed with brine (30 mL). The organic layer was dried over Na₂SO₄, filtered and concentrated under reduced pressure to give tert-butyl 7-[(3-bromo-4-fluoro-phenyl)methyl]-2,7-diazaspiro[3.5]nonane-2-carboxylate (290 mg, 89%) as a pale yellow oil. ¹H NMR (600 MHZ, Methanol-d₄) 7.60 (dd, J=6.7, 2.0 Hz, 1H), 7.31 (t, J=6.6 Hz, 1H), 7.15 (t, J=8.5 Hz, 1H), 3.60 (s, 4H), 3.45 (s, 2H), 2.37 (s, 4H), 1.76 (t, J=5.5 Hz, 4H), 1.42 (s, 9H).

### Step 2

A mixture of tert-butyl 7-[(3-bromo-4-fluoro-phenyl)methyl]-2,7-diazaspiro[3.5]nonane-2-carboxylate (150 mg, 0.36 mmol) and formaldehyde solution (0.1 mL, 1.45 mmol) in formic acid (1.37 mL) was heated at 95° C. for 2 h. After this time, the reaction mixture was cooled to rt, 1 M NaOH solution (3 mL) was added and the aqueous layer extracted with CH₂Cl₂ (3×5 mL). The combined organic extracts were dried over Na₂SO₄, filtered and concentrated under reduced pressure to give 7-[(3-bromo-4-fluoro-phenyl)methyl]-2-methyl-2,7-diazaspiro[3.5]nonane (100 mg, 80%) as a pale yellow oil. ¹H NMR (400 MHZ, Methanol-d₄) δ 7.57 (dd, J=6.8, 2.1 Hz, 1H), 7.32-7.23 (m, 1H), 7.12 (t, J=8.6 Hz, 1H), 3.41 (s, 2H), 3.05 (s, 3H), 2.32 (s, 8H), 1.73 (t, J=5.6 Hz, 4H). m/z 327.1, 329.0 [M+H]⁺.

### Step 3

General procedure 2 was followed using intermediate 4 (105 mg, 0.28 mmol) and 7-[(3-bromo-4-fluoro-phenyl)methyl]-2-methyl-2,7-diazaspiro[3.5]nonane (92 mg, 0.28 mmol). The material was purified by flash column chromatography on an ISCO system (10 g silica, elution with a 20-100% EtOAc/pet. ether gradient) to give the title compound (30 mg, 20%) as a colourless solid. ¹H NMR (600 MHZ, Methanol-d₄) δ 8.11 (dd, J=8.5, 2.5 Hz, 1H), 7.54 (d, J=7.6 Hz, 1H), 7.52-7.48 (m, 1H), 7.39-7.34 (m, 2H), 7.28 (t, J=9.2 Hz, 1H), 3.57 (s, 2H), 3.51 (s, 4H), 3.32-3.28 (m, 2H), 2.63 (s, 3H), 2.53-2.35 (m, 4H), 1.87-1.79 (m, 4H), 1.60 (q, J=9.0 Hz, 2H), 0.98 (t, J=7.5 Hz, 3H). m/z 492.2 [M+H]⁺.

### Example 145: 4-Amino-8-(5-((1-ethyl-1H-1,2,4-triazol-3-yl)methyl)-2-fluorophenyl)-2-oxo-N-propyl-1,2-dihydroquinoline-3-carboxamide

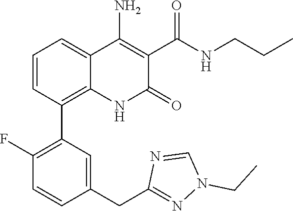 [SMILES: CCCNC(=O)c1c(N)c2cccc(-c3cc(Cc4ncn(CC)n4)ccc3F)c2[nH]c1=O]

### Step 1

A solution of (3-bromo-4-fluorophenyl) acetonitrile (1150 mg, 5.37 mmol) in sulfuric acid (5 mL) was stirred at 60° C. for 1 h. After this time, the solution was cooled to rt and sat. aq. NaHCO₃ (5 mL) was added dropwise. The aqueous layer was extracted with EtOAc (3×5 mL) and the combined organic extracts were washed with brine (5 mL), dried over Na₂SO₄, filtered and concentrated under reduced pressure to give 2-(3-bromo-4-fluoro-phenyl) acetamide (1090 mg, 83%) as a colourless solid. ¹H NMR (600 MHZ, Chloroform-d) δ 7.56-7.40 (m, 1H), 7.23-7.13 (m, 1H), 7.10 (app. t, J=8.2 Hz, 1H), 5.79 (s, 1H), 5.46 (s, 1H), 3.52 (s, 2H). m/z 231.8, 233.8 [M+H]⁺.

### Step 2

To 2-(3-bromo-4-fluoro-phenyl) acetamide (759 mg, 3.27 mmol) in dioxane (4 mL) was added N, N-dimethylformamide-dimethylacetal (467 mg, 3.92 mmol) and the reaction mixture was stirred at 50° C. for 90 min. After this time, the reaction mixture was concentrated under reduced pressure and the residue triturated with Et₂O to give (NE)-2-(3-bromo-4-fluoro-phenyl)-N-(dimethylaminomethylene) acetamide (559 mg, 56%) as a colourless solid. ¹H NMR (600 MHZ, Chloroform-d) δ 8.40 (s, 1H), 7.57-7.48 (m, 1H), 7.24-7.17 (m, 1H), 7.10-6.98 (m, 1H), 3.67 (s, 2H), 3.14-3.09 (m, 3H), 3.09-3.05 (m, 3H). m/z 286.8, 288.8 [M+H] *.

### Step 3

To (NE)-2-(3-bromo-4-fluoro-phenyl)-N-(dimethylaminomethylene) acetamide (439 mg, 1.53 mmol) in acetic acid (13 mL) was added ethylhydrazine dihydrochloride (203 mg, 1.53 mmol) and the reaction mixture stirred at 90° C. for 16 h. After this time, the reaction mixture was added dropwise into sat. aq. NaHCO₃ (20 mL) under vigorous stirring and the aqueous layer was extracted with EtOAc (2×30 mL). The combined organic extracts were dried over Na₂SO₄, filtered and concentrated under reduced pressure to give regioisomers 3-[(3-bromo-4-fluoro-phenyl)methyl]-1-ethyl-1,2,4-triazole and 5-[(3-bromo-4-fluoro-phenyl)methyl]-1-ethyl-1,2,4-triazole (359 mg, 78%) as a pale brown solid in 1:3 ratio. 1H NMR of major isomer (600 MHZ, Chloroform-d) δ 7.86 (d, J=2.9 Hz, 1H), 7.41 (d, J=6.3 Hz, 1H), 7.10-7.00 (m, 2H), 4.11 (d, J=2.8 Hz, 2H), 4.06 (qd, J=7.3, 2.4 Hz, 2H), 1.34 (td, J=7.4, 2.6 Hz, 3H). m/z 284.0, 286.0 [M+H]⁺.

### Step 4

General procedure 8 was followed using intermediate 4 (609 mg, 1.64 mmol), 3-[(3-bromo-4-fluoro-phenyl)methyl]-1-ethyl-1,2,4-triazole (359 mg, 1.26 mmol). The material was purified by flash column chromatography on an ISCO system (10 g silica, elution with a 0-50% MeOH/CH₂Cl₂ gradient) to yield a solid, which was then triturated with Et₂O. A further purification by supercritical fluid chromatography (35:65 MeOH:CO₂ (0.2% v/v NH₃)) was followed to give the title compound (30 mg, 5%) as a cream solid. ¹H NMR (600 MHZ, Chloroform-d) δ 11.27 (s, 1H), 10.30 (d, J=5.9 Hz, 1H), 8.01 (s, 1H), 7.98 (d, J=2.9 Hz, 1H), 7.65 (d, J=8.3 Hz, 1H), 7.51-7.47 (m, 1H), 7.47-7.40 (m, 1H), 7.33 (d, J=6.8 Hz, 1H), 7.28 (d, J=6.0 Hz, 1H), 7.17 (td, J=9.0, 2.7 Hz, 1H), 5.91 (s, 1H), 4.21-4.14 (m, 2H), 4.14-4.05 (m, 2H), 3.36 (q, J=7.0 Hz, 2H), 1.66 (s, 2H), 1.51 (t, J=7.5 Hz, 3H), 0.98 (t, J=7.6 Hz, 3H). m/z 449.1 [M+H]⁺.

### Biological Data

The compounds shown in Table 1 exhibited the following activity in the α2-GABA_{A}R Ki and α2-GABA_{A}R Relative Efficacy assays described herein:

TABLE 1

| | | | α2- | α3- |
|---|---|---|---|---|
| | | | GABA_{A}R | GABA_{A}R |
| | | | Relative | Relative |
| | α2- | α3- | Efficacy | Efficacy |
| | GABA_{A}R | GABA_{A}R | (vs. | (vs. |
| Example | Ki (nM) | Ki (nM) | Diazepam) | Diazepam) |
| Example 1 | 0.79 | | 0.13 | |
| Example 2 | 3.33 | 1.59 | 0.34 | |
| Example 3 | 1.99 | | 0.29 | |
| Example 4 | 1.26 | 1.65 | 0.40 | |
| Example 5 | 5.66 | 16.14 | 0.42 | |
| Example 6 | 0.87 | | 0.10 | |
| Example 7 | 0.59 | 1.17 | 0.16 | 0.43 |
| Example 8 | 0.38 | 0.19 | 0.24 | |
| Example 9 | 0.25 | | 0.23 | |
| Example 10 | 1.50 | | 0.22 | |
| Example 11 | 2.13 | 4.38 | 0.32 | 0.24 |
| Example 12 | 8.17 | | 0.38 | |
| Example 13 | 6.05 | 7.60 | 0.34 | |
| Example 14 | 2.13 | 6.02 | 0.45 | |
| Example 15 | 4.09 | | 0.17 | |
| Example 16 | 24.09 | | 0.34 | |
| Example 17 | 1.64 | 8.74 | 0.33 | 0.42 |
| Example 18 | 9.77 | 27.9 | 0.29 | 0.21 |
| Example 19 | 21.47 | | 0.23 | |
| Example 20 | 245.46 | | 0.16 | |
| Example 21 | 29.77 | | 0.12 | |
| Example 22 | 13.57 | | 0.19 | |
| Example 23 | 2.31 | 7.21 | 0.33 | |
| Example 24 | 4.11 | | 0.21 | |
| Example 25 | 3.40 | | 0.15 | 0.10 |
| Example 26 | 5.58 | | 0.15 | |
| Example 27 | 22.75 | 10.59 | 0.28 | |
| Example 28 | 9.07 | | 0.2 | |
| Example 29 | 6.66 | | 0.22 | |
| Example 30 | 4.76 | | 0.43 | |
| Example 31 | 3.28 | | 0.24 | |
| Example 32 | 3.09 | 6.86 | 0.24 | 0.29 |
| Example 33 | 0.67 | | 0.39 | |
| Example 34 | 0.68 | 1.58 | 0.49 | 0.47 |
| Example 35 | 2.45 | | 0.52 | |
| Example 36 | 3.69 | | 0.05 | |
| Example 37 | 7.55 | | 0.78 | |
| Example 38 | 1.09 | | 0.18 | |
| Example 39 | 9.31 | | −0.06 | |
| Example 40 | 1.19 | 5.06 | 0.29 | 0.31 |
| Example 41 | 0.40 | | 0.13 | |
| Example 42 | 0.56 | 0.64 | 0.25 | 0.21 |
| Example 43 | 1.47 | | 0.14 | |
| Example 44 | 18.58 | | 0.30 | |
| Example 45 | 5.04 | | 0.11 | |
| Example 46 | 2.37 | | 0.02 | |
| Example 47 | 0.23 | | 0.08 | |
| Example 48 | 1.84 | | 0.39 | |
| Example 49 | 0.43 | | 0.26 | |
| Example 50 | 0.21 | | 0.34 | |
| Example 51 | 1.71 | 1.63 | 0.31 | |
| Example 52 | 6.61 | | 0.53 | 0.41 |
| Example 53 | 8.05 | 18.45 | 0.17 | |
| Example 54 | 20.03 | | 0.07 | |
| Example 55 | 159.15 | | | |
| Example 56 | 6.62 | | 0.34 | |
| Example 57 | 73.30 | | | |
| Example 58 | 153.78 | | | |
| Example 59 | 2.01 | | 0.27 | |
| Example 60 | 15.39 | | 0.65 | |
| Example 61 | 106.59 | | | |
| Example 62 | 6.85 | 5.41 | 0.28 | 0.44 |
| Example 63 | 24.46 | | 0.65 | |
| Example 64 | 84.56 | | 0.34 | |
| Example 65 | 55.19 | | | |
| Example 66 | 18.59 | | | |
| Example 67 | 3.74 | | 0.54 | |
| Example 68 | 4.88 | | 0.54 | |
| Example 69 | 26.52 | 60.52 | 0.6 | 0.59 |
| Example 70 | >500 | | | |
| Example 71 | >500 | | | |
| Example 72 | >500 | | | |
| Example 73 | 3.08 | | 0.48 | |
| Example 74 | >500 | | | |
| Example 75 | 171.25 | | | |
| Example 76 | 6.23 | | 0.42 | |
| Example 77 | 2.77 | | | |
| Example 78 | 2.11 | | 0.70 | |
| Example 79 | 2.51 | | 0.81 | |
| Example 80 | 4.44 | | 0.6 | |
| Example 81 | 2.19 | | 0.81 | |
| Example 82 | 6.66 | | 0.31 | |
| Example 83 | 15.74 | | 0.40 | |
| Example 84 | 75.55 | | | |
| Example 85 | 0.30 | 0.70 | 0.32 | 0.42 |
| Example 86 | 1.71 | | 0.28 | |
| Example 87 | 1.78 | | 0.24 | |
| Example 88 | 1.97 | 1.37 | 0.26 | |
| Example 89 | 1.55 | | 0.3 | |
| Example 90 | 3.00 | | 0.18 | |
| Example 91 | 0.46 | 1.30 | 0.18 | 0.29 |
| Example 92 | 0.48 | 0.81 | 0.21 | 0.26 |
| Example 93 | 1.63 | 1.60 | 0.34 | |
| Example 94 | 2.42 | | 0.28 | |
| Example 95 | 0.44 | | 0.22 | |
| Example 96 | 0.92 | | 0.24 | |
| Example 97 | 0.72 | | 0.25 | |
| Example 98 | 0.61 | | 0.29 | |
| Example 99 | 0.24 | | 0.28 | |
| Example 100 | 0.07 | | 0.18 | |
| Example 101 | 0.19 | | 0.31 | |
| Example 102 | 0.70 | | 0.33 | |
| Example 103 | 1.68 | | 0.37 | |
| Example 104 | 2.08 | | 0.27 | |
| Example 105 | 0.21 | | 0.19 | |
| Example 106 | 0.41 | | 0.22 | |
| Example 107 | 0.20 | | 0.17 | |
| Example 108 | 0.39 | | 0.32 | |
| Example 109 | 0.17 | | 0.21 | |
| Example 110 | 0.59 | 0.97 | 0.25 | 0.30 |
| Example 111 | 0.23 | 0.41 | 0.27 | 0.47 |
| Example 112 | 0.57 | | 0.11 | |
| Example 113 | 0.12 | | 0.24 | |
| Example 114 | 0.73 | | 0.38 | |
| Example 115 | 2.01 | | 0.34 | |
| Example 116 | 1.17 | | 0.42 | |
| Example 117 | 1.60 | | 0.23 | |
| Example 118 | 0.50 | | 0.29 | |
| Example 119 | 0.31 | | 0.38 | |
| Example 120 | 1.19 | 1.18 | 0.21 | |
| Example 121 | 0.30 | 0.61 | 0.35 | |
| Example 122 | 0.89 | 3.56 | 0.33 | |
| Example 123 | 1.71 | | 0.2549 | |
| Example 124 | 0.98 | 1.55 | 0.28 | 0.34 |
| Example 125 | 0.37 | | 0.22 | |
| Example 126 | 1.19 | | 0.27 | |
| Example 127 | 0.78 | | 0.24 | |
| Example 128 | 1.08 | | 0.24 | |
| Example 129 | 0.65 | | 0.25 | |
| Example 130 | 0.65 | | 0.18 | |
| Example 131 | 0.30 | | 0.40 | |
| Example 132 | 0.66 | 1.19 | 0.24 | |
| Example 133 | 1.29 | | 0.23 | |
| Example 134 | 0.17 | | 0.04 | |
| Example 135 | 0.70 | | 0.20 | |
| Example 136 | 0.90 | | 0.24 | |
| Example 137 | 3.00 | 8.07 | 0.42 | 0.53 |
| Example 138 | 1.87 | 4.99 | 0.17 | 0.42 |
| Example 139 | 1.08 | | 0.18 | |
| Example 140 | 1.88 | | 0.34 | |
| Example 141 | 2.61 | 16.42 | 0.36 | |
| Example 142 | 3.60 | 6.35 | 0.24 | 0.27 |
| Example 143 | 1.80 | 10.10 | 0.32 | 0.11 |
| Example 144 | 1.99 | 5.43 | 0.41 | |
| Example 145 | 1.29 | | 0.28 | |

### Rat Brain Receptor Occupancy

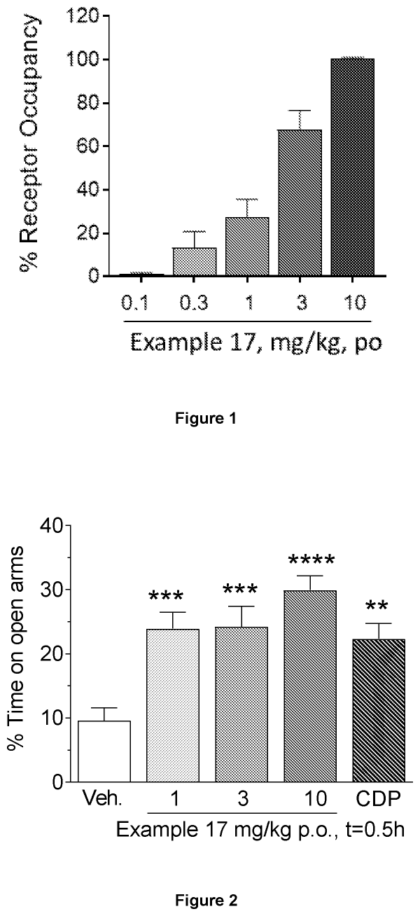

The compound of Example 17 was tested in the rat brain occupancy assay described herein. For doses of the compound in the range 0.1 to 10 mg/kg (p.o.), the occupancy of rat brain benzodiazepine binding sites was dose-dependent, with the dose of 10 mg/kg estimated to fully occupy the brain GABA_{A} receptors (101+0.5%, mean±SEM, n=6 rats, see FIG. 1).

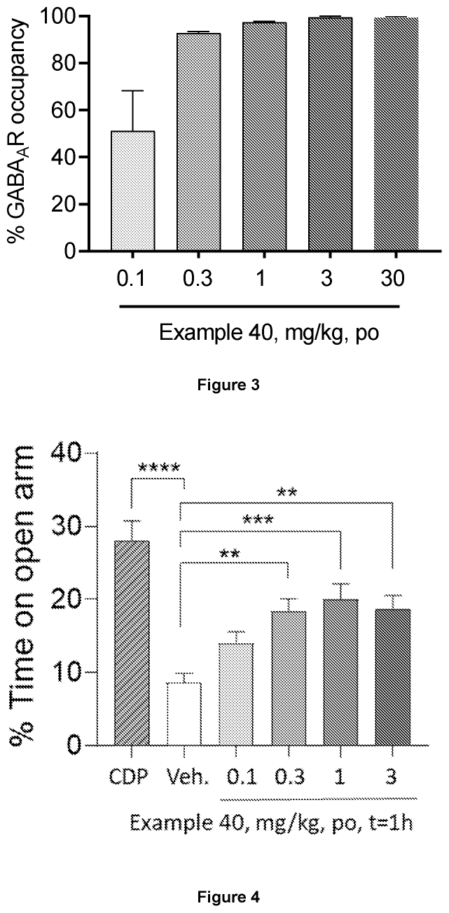

The compound of Example 40 was tested in this assay. For doses of the compound in the range 0.1 to 10 mg/kg (p.o.), the occupancy of rat brain benzodiazepine binding sites (i.e., the inhibition of in vivo [³H] flumazenil binding) was dose-dependent, with the dose of 1 mg/kg estimated to occupy the brain GABA_{A} receptors at 97.3±2.0% (mean±SEM, n=7 rats, see FIG. 3).

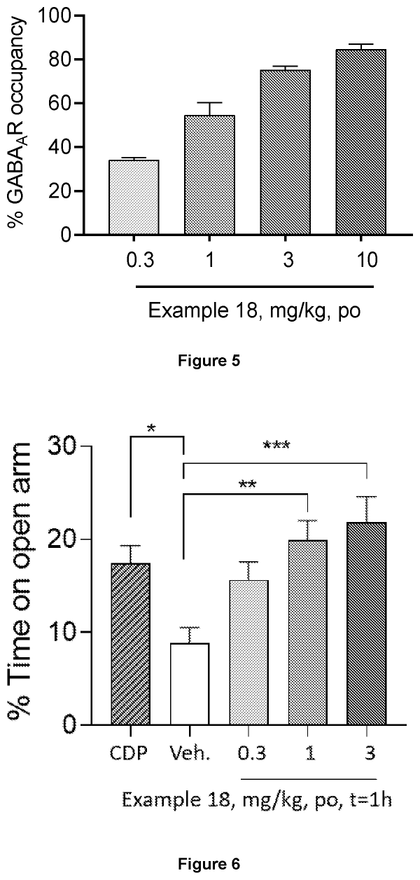

The compound of Example 18 was tested in this assay. For doses of the compound in the range 0.3 to 10 mg/kg (p.o.), the occupancy of rat brain benzodiazepine binding sites (i.e., the inhibition of in vivo [³H] flumazenil binding) was dose-dependent (see FIG. 5).

### Elevated Plus Maze Assay

Example 17 was tested in rats in the elevated plus maze assay described herein. The percent time spent on open arms by Example 17 treated rats (1, 3, and 10 mg/kg p.o.) was significantly higher than for vehicle (0.5% methylcellulose/0.1% Tween 80) treated rats (n=13-15/group, Mean±SEM). Chlordiazepoxide (CDP; 5 mg/kg i.p.) was used as reference. The compound of Example 17 exhibited anxiolytic-like effects in this assay (see FIG. 2).

The compound of Example 40 was tested in this assay and showed anxiolytic-like effects (see FIG. 4). The percent time spent on open arms by Example 40 treated rats (0.3, 1 and 3 mg/kg p.o.) is significantly higher than for vehicle (0.5% methylcellulose/0.1% Tween 80) treated rats (n=13-15/group, Mean±SEM). Chlordiazepoxide (CDP; 5 mg/kg i.p.) was used as reference.

The compound of Example 18 was tested in this assay and showed anxiolytic-like effects (see FIG. 6). The percent time spent on open arms by Example 18 treated rats (1 and 3 mg/kg p.o.) is significantly higher than for vehicle (0.5% methylcellulose/0.1% Tween 80) treated rats (n=13-15/group, Mean±SEM). Chlordiazepoxide (CDP; 5 mg/kg i.p.) was used as reference.

1. A compound of the formula (I), or a pharmaceutically acceptable salt thereof: 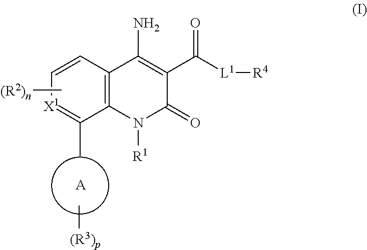
wherein:
X¹ is CH or N;
Ring A is a phenyl or a 5- or 6-membered heteroaryl;
R¹ is selected from: H, C₁₋₄ alkyl, C₁₋₄ haloalkyl, C₃₋₆ cycloalkyl and C₃₋₆ cycloalkyl-C₁₋₄ alkyl-;
each R² is independently selected from: halo, C₁₋₄ alkyl and C₁₋₄ haloalkyl;
each R³ is independently selected from: halo, —CN, —NO₂, ═O, C₁₋₆ alkyl, C₁₋₆ haloalkyl, C₂₋₆ alkenyl, C₂₋₆ alkynyl, —OR⁵, —S(O)ₓR⁵, —NR^{a1}R⁵, —C(O)R⁵, —OC(O)R⁵, —C(O)OR⁵, —NR^{a1}C(O)R⁵, —C(O)NR^{a1}R⁵, —NR^{a1}C(O)OR⁵, —OC(O)NR⁵R^{a1}, —NR^{a1}SO₂R⁵, —SO₂NR^{a1}R⁵ and -L²-Q²,
wherein said C₁₋₆ alkyl, C₂₋₆ alkenyl and C₂₋₆ alkynyl is optionally substituted by one or more substituents selected from: halo, —CN, —OR^{a2}, —S(O),R^{a2} and —NR^{a2}R^{b2}
or two R³ groups attached to the same or adjacent ring atoms in Ring A together form a C₃₋₆ cycloalkyl or 4- to 6-membered heterocyclic spiro or fused ring, wherein said C₃₋₆ cycloalkyl or 4- to 6-membered heterocyclic is optionally substituted with one or more substituents selected from: halo, ═O, C₁₋₄ alkyl and C₁₋₄ haloalkyl;
R⁵ is selected from: H, C₁₋₆ alkyl and C₁₋₆ haloalkyl, wherein said C₁₋₆ alkyl is optionally substituted by one or more substituents selected from: halo, —CN, —OR^{a3}, —S(O)ₓR^{a3} and —NR^{a3}R^{b3;}
L² is a bond or is selected from: *—[CR⁶R⁷]ₐ—NR⁸—, *—NR⁸—[CR⁶R⁷]ₐ—, *—[CR⁶R⁷]ₐ—O—, *—O—[CR⁶R⁷]ₐ—, *—C(O)—[CR⁶R⁷]ₐ—, *—[CR⁶R⁷]ₐ—C(O)—, *—S(O)ₓ—[CR⁶R⁷]ₐ—, *—[CR⁶R⁷]ₐ—S(O)ₓ— and —[CR⁶R⁷]ₐ—, wherein * indicates the bond to Ring A,
each R⁶ and R⁷ is independently selected from: H, halo, C₁₋₃ alkyl and C₁₋₃ haloalkyl,
or two R⁶ and R⁷ attached to the same carbon atom form a C₃₋₆ cycloalkyl,
a is 0 to 4,
R⁸ is selected from: H, C₁₋₄ alkyl and C₁₋₄ haloalkyl;
Q² is selected from: C₃₋₆ cycloalkyl, 4- to 12-membered heterocyclyl, C₆₋₁₀ aryl and 5- to 12-membered heteroaryl,
wherein said C₃₋₆ cycloalkyl and 4- to 12-membered heterocyclyl is optionally substituted by one or more R⁹,
wherein said C₆₋₁₀ aryl and 5- to 12-membered heteroaryl is optionally substituted by one or more R¹⁰;
L¹ is a bond or is selected from: O and NH;
R⁴ is selected from: H, C₁₆ alkyl, C₁₋₆ haloalkyl, C₂₋₆ alkenyl, C₂₋₆ alkynyl and -L³-Q³,
wherein said C₁₋₆ alkyl, C₂₋₆ alkenyl and C₂₋₆ alkynyl is optionally substituted by one or more substituents selected from: halo, —CN, ═O, —OR^{a8}, —S(O)ₓR^{a8} and —NR^{a8}R^{b8;}
L³ is a bond or —[CR¹¹R¹²]_{b}—,
each R¹¹ and R¹² is independently selected from: H, halo, C₁₋₃ alkyl and C₁₋₃ haloalkyl,
or two R¹¹ and R¹² attached to the same carbon atom form a C₃₋₆ cycloalkyl,
b is 1 to 4,
Q³ is selected from: C₃₋₆ cycloalkyl, 4- to 7-membered heterocyclyl, phenyl and 5- or 6-membered heteroaryl,
wherein said C₃₋₆ cycloalkyl and 4- to 7-membered heterocyclyl is optionally substituted by one or more R¹³,
wherein said phenyl and 5- or 6-membered heteroaryl is optionally substituted by one or more R¹⁴;
each R⁹ and R¹³ is independently selected from: halo, ═O, —CN, —NO₂, C₁₋₄ alkyl, C₁₋₄ haloalkyl, —OR^{a4}, —S(O)₂R^{a4}, —NR^{a4}R^{b4}, —C(O)R^{a4}, —OC(O)R^{a4}, —C(O)OR^{a4}, —NR^{a4}C(O)R^{b4}, —C(O)NR^{a4}R^{b4}, —NR^{a4}C(O)OR^{b4}, —OC(O)NR^{a4}R^{b4}, —NR^{a4}SO₂R^{b4} and —SO₂NR^{a4}R^{b4};
wherein said C₁₋₄ alkyl is optionally substituted by 1 or 2 substituents selected from: halo, —CN, —OR^{a5}, —NR^{a5}R^{b5} and —SO₂R^{a5};
each R¹⁰ and R¹⁴ is independently selected from: halo, ═O, —CN, —NO₂, C₁₋₄ alkyl, C₁₋₄ haloalkyl, —OR^{a6}, —S(O)₂R^{a6}, —NR^{a6}R^{b6}, —C(O)R^{a6}, —OC(O)R^{a6}, —C(O)OR^{a6}, —NR^{a6}C(O)R^{b6}, —C(O)NR^{a6}R^{b6}, —NR^{a6}C(O)OR^{b6}, —OC(O)NR^{a6}R^{b6}, —NR^{a6}SO₂R^{b6} and —SO₂NR^{a6}R^{b6};
wherein said C₁₋₄ alkyl is optionally substituted by 1 or 2 substituents selected from: halo, —CN, —OR^{a7}, —NR^{a7}R^{b7} and —SO₂R^{a7;}
R^{a1}, R^{a2}, R^{b2}, R^{a3}, R^{b3}, R^{a4}, R^{b4}, R^{a5}, R^{b5}, R^{a6}, R^{b6}, R^{a7}, R^{b7}, R^{a8} and R^{b8} are at each occurrence independently selected from: H, C₁₋₄ alkyl and C₁₋₄ haloalkyl,
or any —NR^{a1}R⁵, —NR^{a2}R^{b2}, —NR^{a3}R^{b3}, —NR^{a4}R^{b4}, —NR^{a5}R^{b5}, —NR^{a6}R^{b6}, —NR^{a7}R^{b7} or —NR^{a8}R^{b8} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl;
n is 0, 1, 2 or 3;
p is 0, 1, 2, 3, 4 or 5; and
x at each occurrence is independently 0, 1 or 2.

2. The compound of claim 1, wherein X¹ is CH.

3. The compound of claim 1, wherein R¹ is H.

4. The compound of claim 1, wherein n is 0 or 1 and R² is halo.

5. The compound of claim 1, wherein Ring A is selected from: phenyl, pyrrolyl, imidazolyl, pyrazolyl, oxazolyl, isoxazolyl, furanyl, thiophenyl, thiazolyl, isothiazolyl, triazolyl, oxadiazolyl, thiadiazolyl, pyridyl, pyrimidyl, pyridazinyl and pyrazinyl, wherein Ring A is optionally substituted by p R³ groups.

6. The compound of claim 1, wherein Ring A is selected from: 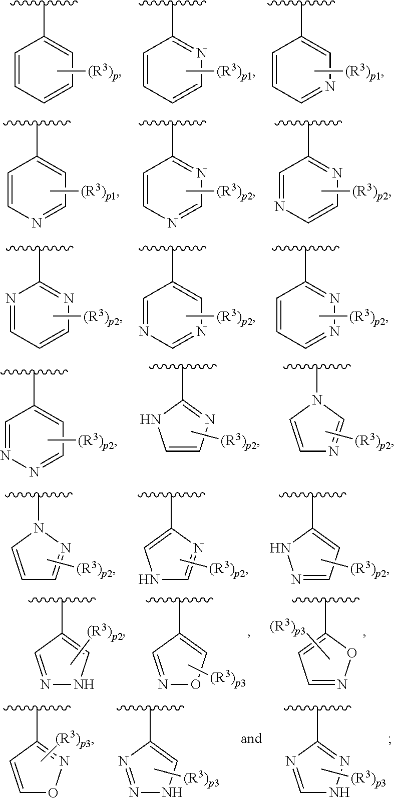
wherein p1 is selected from 0, 1, 2, 3 and 4;
p2 is selected from 0, 1, 2 and 3; and
p3 is selected from 0, 1 and 2.

7. The compound of claim 1, wherein Ring A is of the formula: 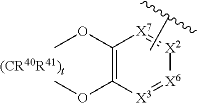
wherein
each R⁴⁰ and each R⁴¹ are independently selected from: H, halo, C₁₋₄ alkyl and C₁₋₄ haloalkyl;
t is 1, 2 or 3; and
X², X³, X⁶ and X⁷ are each independently selected from: N, CH and CR³.

8. The compound of claim 1, wherein Ring A is substituted with one R³ selected from -L²-Q² and optionally one or more R³ substituents selected from: halo, —CN, C₁₋₄ alkyl, C₁₋₄ haloalkyl, —OR⁵, —NR^{a1}R⁵,
wherein said C₁₋₄ alkyl is optionally substituted by one or more substituents selected from: —OR^{a2} and —NR^{a2}R^{b2},
R⁵ is selected from: H, C₁₋₄ alkyl and C₁₋₄ haloalkyl, wherein said C₁₋₄ alkyl is optionally substituted by one or more substituents selected from: —OR^{a3} and —NR^{a3}R^{b3}.

9. The compound of claim 1, wherein L² is selected from: *—(CH₂)₂—O—, *—CH₂—O—, *—O—(CH₂)₂—, *—O—CH₂—, *—(CH₂)₂—NH—, *—CH₂—NH—, *—NH—(CH₂)₂—, *—NH—CH₂—, —CH₂— and —(CH₂)₂—, wherein * indicates the bond to Ring A; and
Q² is selected from: C₃₋₆ cycloalkyl, 4- to 12-membered heterocyclyl, phenyl and 5- to 10-membered heteroaryl, wherein said C₃₋₆ cycloalkyl and 4- to 12-membered heterocyclyl is optionally substituted by one or more R⁹, and said phenyl and 5- to 10-membered heteroaryl is optionally substituted by one or more R¹⁰.

10. The compound of claim 1, wherein Ring A is selected from: 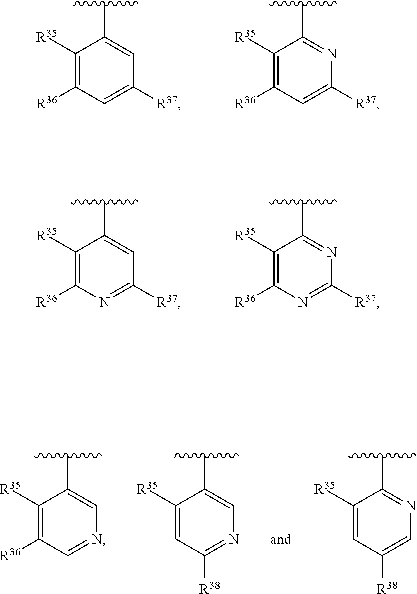
R³⁵, R³⁶ and R³⁸ are independently selected from: H, halo, C₁₋₄ alkyl, —C₁₋₄ alkyl-OR^{a2}, C₁₋₄ haloalkyl, —O—C₁₋₄ alkyl and —O—C₁₋₄ haloalkyl;
R³⁷ is C₁₋₄ alkyl, —C₁₋₄ alkyl-OR^{a2}, C₁₋₄ haloalkyl, —O—C₁₋₄ alkyl, —O—C₁₋₄ alkyl-OR^{a3}, —O—C₁₋₄ haloalkyl and -L²-Q²;
L² is selected from: —(CH₂)₂—, —CH₂—, —(CH₂)₂—O—*, —CH₂—O—*, —O—(CH₂)₂—*, —O—CH₂—*, —(CH₂)₂—NR⁸—*, —CH₂—NR⁸—*, —NR⁸—(CH₂)₂—* and —NR⁸—CH₂—*, wherein R⁸ is selected from H and methyl, and * indicates the bond to Q².

11. The compound of claim 10, wherein R³⁷ is selected from: —C₁₋₄ alkyl-OR^{a2}, —O—C₁₋₄ alkyl, —O—C₁₋₄ alkyl-OH, —O—C₁₋₄ alkyl-O—C₁₋₃ alkyl and -L²-Q².

12. The compound of claim 10, wherein R³⁷ is -L²-Q².

13. The compound of claim 10, wherein R³⁶ is C₁₋₄ alkyl.

14. The compound of claim 10, wherein R³⁵ is selected from: halo, C₁₋₄ alkyl and —O—C₁₋₄ alkyl (e.g. R³⁵ is F, or R³⁵ is —OMe).

15. The compound of claim 1, wherein -L²-Q² is selected from: 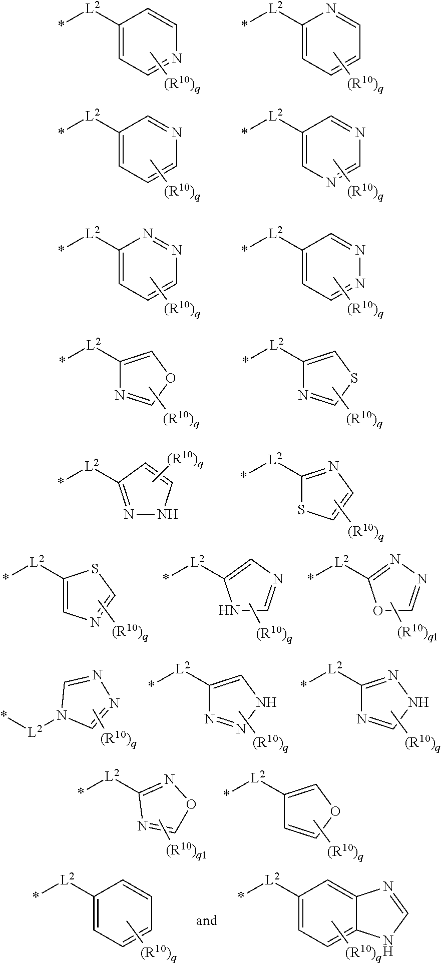
wherein q is 0, 1 or 2; q1 is 0 or 1; and
*indicates the point of attachment to Ring A.

16. The compound of claim 1, wherein L² is selected from: —CH₂—O—*, and —O—CH₂—*.

17. The compound of claim 1, wherein -L²- is selected from: —(CH₂)₂—, —CH₂—, —(CH₂)₂—O—*, —CH₂—O—*, —O—(CH₂)₂—*, —O—CH₂—*, —(CH₂)₂—NR⁸—*, —CH₂—NR⁸—*, —NR⁸—(CH₂)₂—* and —NR⁸—CH₂—*, wherein R⁸ is selected from H and methyl, and * indicates the bond to Q²;
Q² is selected from: azetidinyl, pyrrolidinyl, imidazolidinyl, piperidinyl, piperazinyl, morpholinyl, homopiperidinyl, homopiperazinyl, 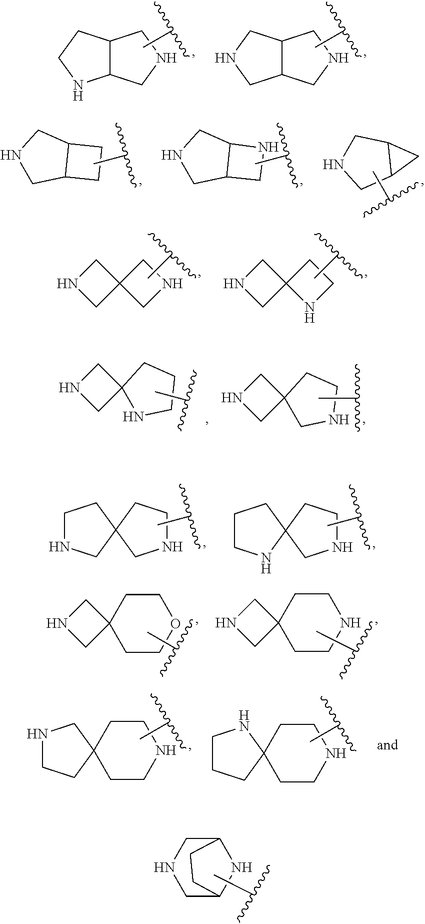
wherein each Q² is optionally substituted by one or more R⁹ and wherein Q² is bonded to L² by any available ring carbon or ring nitrogen atom.

18. The compound of claim 1, wherein the compound of formula (I) is of the formula (XXVI), or a pharmaceutically acceptable salt thereof: 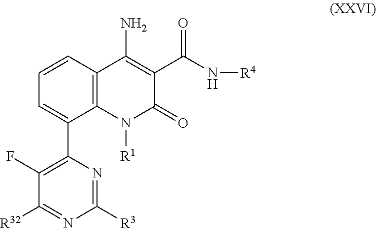
wherein R³² is selected from: H and R³;
optionally wherein:
(i) R³² is H; and R³ is selected from: C₁₋₄ alkyl and —OC₁₋₄ alkyl; or
(ii) R³² is H; and R³ is methyl.

19. The compound of claim 1, wherein the compound of formula (I) is of the formula (XXVII), or a pharmaceutically acceptable salt thereof: 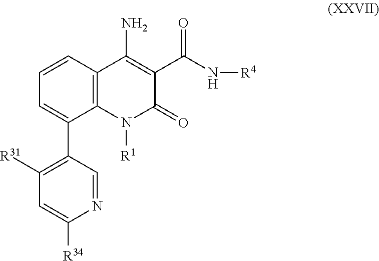
wherein R³¹ is H or R³ and
R³⁴ is R³;
optionally wherein:
(i) R³¹ is selected from halo, C₁₋₄ alkyl and —OC₁₋₄ alkyl, and R³⁴ is C₁₋₄ alkyl; or
(ii) R³¹ is selected from F and methoxy, and R³⁴ is methyl; or
(iii) R³¹ is methoxy and R³⁴ is methyl.

20. The compound of claim 1, wherein the compound of formula (I) is of the formula (XXIX), or a pharmaceutically acceptable salt thereof: 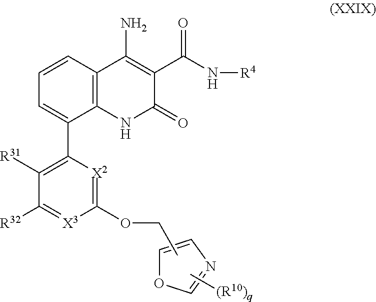
wherein
X² and X³ are each independently N or CH;
R³¹ and R³² are each independently selected from: H and R³;
q is 0, 1 or 2;
optionally wherein R³¹ and R³² are independently selected from: H, halo, C₁₋₄ alkyl, C₁₋₄ haloalkyl, —O—C₁₋₄ alkyl and —O—C₁₋₄ haloalkyl, for example wherein R³¹ is selected from: halo and C₁₋₄ alkyl, and R³² is selected from: H, halo, C₁₋₄ alkyl, C₁₋₄ haloalkyl, —O—C₁₋₄ alkyl and —O—C₁₋₄ haloalkyl.

21. The compound of claim 1, wherein R⁴ is selected from: C₁₋₆ alkyl, C₁₋₆ haloalkyl, —C₁₋₆ alkyl-CN, —C₁₋₆ alkyl-OR^{a8}, —C₁₋₄ alkyl-C₁₋₆ cycloalkyl, C₁₋₆ cycloalkyl, —C₁₋₄ alkyl-oxetanyl, oxetanyl, —C₁₋₄ alkyl-tetrahydrofuranyl, tetrahydrofuranyl, —C₁₋₄ alkyl-tetrahydropyranyl and —C₁₋₄ alkyl-tetrahydropyranyl, wherein any C₁₆ cycloalkyl, oxetanyl, tetrahydrofuranyl and tetrahydropyranyl in R⁴ is optionally substituted by one or two substituents selected from halo, ═O, and C₁₋₄ alkyl.

22. The compound of claim 1, wherein R⁴ is selected from:

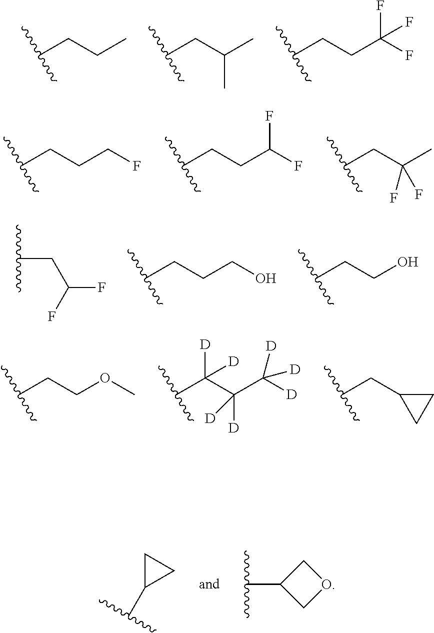

23. The compound of claim 1, wherein R⁴ is —CH₂CH₂CH₃.

24. The compound according to claim 1, wherein the compound is selected from Compound List 1 in the description, or a pharmaceutically acceptable salt thereof.

25. A pharmaceutical composition comprising a compound of claim 1, or a pharmaceutically acceptable salt thereof, and a pharmaceutically acceptable excipient.

26. (canceled)

27. (canceled)

28. (canceled)

29. (canceled)

30. A method of preventing or treating a disease or medical disorder mediated by α2-GABA_{A} and/or α3-GABA_{A} receptors in a subject, the method comprising administering to the subject an effective amount of a compound of claim 1, or a pharmaceutically acceptable salt thereof.

31. A method of preventing or treating a medical disorder selected from: an anxiety disorder, a mood disorder, pain, a neurodegenerative disorder, a neurodevelopmental disorder, a cognitive disorder a psychiatric disorder and anxiety associated with a medical disorder, the method comprising administering to the subject an effective amount of a compound of claim 1, or a pharmaceutically acceptable salt thereof.
